(19) United States
(12) Patent Application Publication
(10) Pub. No.: US 2025/0236609 A1
(43) Pub. Date: Jul. 24, 2025

(54) VPS34 INHIBITORS AND METHODS OF USE THEREOF

(71) Applicant: Deciphera Pharmaceuticals, LLC, Waltham, MA (US)

(72) Inventors: Kristen L. Stueven, Waltham, MA (US); Jeffery Zwicker, Waltham, MA (US); Yu Mi Ahn, Waltham, MA (US); Daniel L. Flynn, Waltham, MA (US)

(21) Appl. No.: 18/980,378

(22) Filed: Dec. 13, 2024

Related U.S. Application Data
(60) Provisional application No. 63/622,680, filed on Jan. 19, 2024.
(60) Provisional application No. 63/609,562, filed on Dec. 13, 2023.

(51) Int. Cl. C07D 401/14; A61K 31/444; A61K 31/4545; A61K 31/496; A61K 31/497; A61K 31/501; A61K 31/506; A61K 31/5377; C07D 213/75; C07D 405/14; C07D 413/14

(52) U.S. Cl. CPC C07D 401/14; A61K 31/444; A61K 31/4545; A61K 31/496; A61K 31/497; A61K 31/501; A61K 31/506; A61K 31/5377; C07D 213/75; C07D 405/14; C07D 413/14

(57) ABSTRACT

The present disclosure, in part, provide compounds of Formula (I) that are VPS34 inhibitors, compositions, and methods of use thereof, such as in methods of treating cancer, diabetes, inflammatory diseases, neurodegenerative disorders, cardiovascular disorders, autoimmune diseases and viral infections:

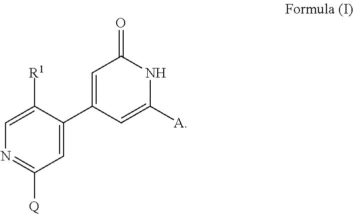

### CROSS-REFERENCE

This application claims priority to U.S. Provisional Application No. 63/609,562 filed Dec. 13, 2023 and U.S. Provisional Application No. 63/622,680 filed Jan. 19, 2024, the contents of each of which are incorporated herein by reference in their entireties.

### REFERENCE TO A SEQUENCE LISTING XML

This application contains a Sequence Listing which has been submitted electronically in XML format. The Sequence Listing XML is incorporated herein by reference. Said XML file, created on Jan. 7, 2025, is named DCP-127US_SL.xml and is 3,055 bytes in size.

### BACKGROUND

Autophagy is a cellular process enabling the recycling of cytoplasmic components via the formation of double-membraned vesicles termed autophagosomes. In addition to autophagy-mediated vesicular trafficking, the endosomal vesicular trafficking process also enables the sequestration and destruction/recycling of cellular components. Due to its cytoprotective role, defective autophagy has been implicated in several human pathologies including cancer. Cancer cells have been shown to upregulate autophagy in response to stress factors present in the tumor microenvironment (TME) such as hypoxia and, nutrient deprivation, as well as a resistance mechanism to anti-cancer therapy. Recently it has become known that the autophagy pathway and/or the endosomal pathway can be upregulated in various cancers to degrade proteins that would otherwise trigger a productive anti-tumor immune response. Thus, there is a need to block the immunosuppressive effects of autophagy and endosomal trafficking in the tumor environment as a potential therapeutic strategy to improve cancer immunotherapy.

Recent work has demonstrated that activation of the STING pathway in the innate tumor environment is required for induction of a productive CD8+ T cell response against tumor-derived antigens in vivo. STING (stimulator of interferon genes) is a transmembrane protein localized to the endoplasmic reticulum that is activated in response to binding of cyclic dinucleotides (CDNs), resulting in a downstream signaling cascade involving TBK1 kinase activation, IRF-3 phosphorylation, and production of IFN-3 and other cytokines. IFN-3 is the major cytokine induced in response to activating STING, either by exogenous CDNs produced by bacterial infection or through binding of a structurally distinct endogenous CDNs produced by a host cyclic GMPAMP synthetase (cGAS). These observations suggest that direct activation of the STING pathway in the tumor environment by specific agonists could be an effective therapeutic strategy to promote broad tumor-initiated T cell priming against an individual's tumor antigen repertoire. Exogenously administered STING agonists are being developed as vaccine adjuvants, as well as direct anti-tumor therapeutics effects. In addition to the administration of exogenous STING agonists, an alternative approach that modulates and upregulates endogenous STING signaling in the tumor environment may be a more preferred method of treatment.

The lipid kinase vacuolar protein sorting 34 (VPS34) is a target for blocking autophagy- or endosomal pathway-mediated immunosuppression. VPS34, also known as class III phosphoinositide 3-kinase (PIK3C3), regulates autophagy initiation and other vesicular trafficking processes, including playing a key role in endosomal trafficking. VPS34 phosphorylates phosphatidyl inositol (PI) to phosphatidyl inositol-3 phosphate (PI3P), which is required for proteins to form complexes via the FYVE domain of client proteins recruited to vesicles. PI3P is found on early and late endosomes and is required for autophagosome formation. Inhibition of VPS34, and therefore disruption of P3IP production, results in the inhibition of the autophagy pathway. VPS34 has also been linked to the delayed degradation of STING in murine cells through endosomal trafficking, leading to elongated activated STING activity.

VPS34 inhibitors have shown antiproliferative effects in vitro in both single agent and in combination with other anticancer therapies, including anti-PD1 immunotherapy and STING agonists. However, the development of VPS34 inhibitors as anticancer therapeutics has been limited by the high conservation of residues in the active site compared to other PI3K family members, and it has been demonstrated clinically that isoform-selective PI3K inhibitors have reduced toxicity compared to pan-PI3K or dual PI3K/mTOR inhibitors. Therefore, it is beneficial to provide novel, potent VPS34 inhibitors with a high degree of selectivity compared to PI3K family members.

### SUMMARY

The present disclosure, in part, provide compounds that are VPS34 inhibitors, compositions, and methods of use thereof, such as in methods of treating cancer, diabetes, inflammatory diseases, neurodegenerative disorders, cardiovascular disorders, autoimmune diseases, and viral infections.

Provided herein, in some embodiments, are compounds of Formula (I):

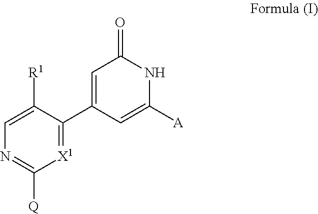

or a pharmaceutically acceptable salt, enantiomer, stereoisomer, or tautomer thereof, wherein:
X¹ is selected from the group consisting of CH and N;
A is selected from the group consisting of cycloalkyl, heterocyclyl, aryl, heteroaryl, and —NHSO₂R³;
Q is selected from the group consisting of H, —NHR², and —NHC(O)LR⁴;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R³ is selected from the group consisting of alkyl, amino, cycloalkyl, heterocyclyl, aryl and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl; and
L is selected from the group consisting of a bond and alkyl, wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

Also described herein, in some embodiments, are pharmaceutical compositions comprising a compound described herein (e.g., a compound of the disclosure as described herein), or a pharmaceutically acceptable salt, enantiomer, stereoisomer, or tautomer thereof, and a pharmaceutically acceptable carrier or excipient.

In some embodiments, provided herein are methods of treating cancer in a patient in need thereof, comprising administering to the patient a therapeutically effective amount of the compound described herein (e.g., a compound of the disclosure as described herein), or a pharmaceutically acceptable salt, enantiomer, stereoisomer, or tautomer thereof, or of the pharmaceutical composition described herein (e.g., a pharmaceutical composition comprising a compound of the disclosure as described herein, or a pharmaceutically acceptable salt, enantiomer, stereoisomer, or tautomer thereof).

In some embodiments, provided herein are methods of treating type II diabetes in a patient in need thereof, comprising administering to the patient a therapeutically effective amount of the compound described herein (e.g., a compound of the disclosure as described herein), or a pharmaceutically acceptable salt, enantiomer, stereoisomer, or tautomer thereof, or of the pharmaceutical composition described herein (e.g., a pharmaceutical composition comprising a compound of the disclosure as described herein, or a pharmaceutically acceptable salt, enantiomer, stereoisomer, or tautomer thereof).

In some embodiments, provided herein are methods of treating a disease selected from inflammatory diseases, neurodegenerative disorders, autoimmune diseases and viral infections in a patient in need thereof, comprising administering to the patient a therapeutically effective amount of the compound described herein (e.g., a compound of the disclosure as described herein), or a pharmaceutically acceptable salt, enantiomer, stereoisomer, or tautomer thereof, or of the pharmaceutical composition described herein (e.g., a pharmaceutical composition comprising a compound of the disclosure as described herein, or a pharmaceutically acceptable salt, enantiomer, stereoisomer, or tautomer thereof).

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The definitions set forth in this application are intended to clarify terms used throughout this application.

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent.

Combinations of substituents, positions of substituents and/or variables are permissible only if such combinations result in stable compounds.

As used herein, the singular forms “a”, “an”, and “the” encompass plural references unless the context clearly indicates otherwise.

As used herein, the term “herein” means the entire application.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to instances wherein the alkyl may be substituted as well as wherein the alkyl is not substituted.

It is understood that substituents and substitution patterns on the disclosed compounds can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen atoms in a given structure with the radical of a specified substituent including, but not limited to: hydroxyl, hydroxyalkyl, alkoxy, halogen, alkyl, aryl, cycloalkyl, heterocyclyl, amino, aminoalkyl, cyano, haloalkyl, haloalkoxy, —OC(═O)—CH₂-Oalkyl. Preferably, “optionally substituted” refers to the replacement of one to four hydrogen atoms in a given structure with the substituents mentioned above. More preferably, one to three hydrogen atoms are replaced by the substituents as mentioned above. It is understood that the substituent can be further substituted.

As used herein, the term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched, and unbranched, carbocyclic, and heterocyclic, aromatic, and non-aromatic substituents of organic compounds.

The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms.

Substituents can include any substituents described herein, for example, Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a heteroaralkyl, or an aromatic or heteroaromatic moiety. It will be understood by those skilled in the art that substituents can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN, and the like. Unless specifically stated as “unsubstituted,” references to chemical moieties herein are understood to include substituted variants. For example, reference to an “aryl” group or moiety implicitly includes both substituted and unsubstituted variants.

As used herein, the term “alkyl” refers to a straight chained or branched non-aromatic hydrocarbon which is completely saturated. Typically, a straight chained or branched alkyl group has from 1 to about 20 carbon atoms, preferably from 1 to about 10, e.g., may be C₁-C₁₀alkyl or e.g., C₁-C₆alkyl unless otherwise defined. Examples of straight chained and branched alkyl groups include, but are not limited to, methyl, ethyl, 1-propyl (n-propyl), 2-propyl, n-butyl, sec-butyl, tertbutyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. Moreover, the term “alkyl” used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. The “alkyl” group may be optionally substituted.

The term “Cₓ-C_{y}” when used in conjunction with a chemical moiety, such as, acyl, acyloxy, alkyl, alkenyl, alkynyl, or alkoxy is meant to include groups that contain from x to y carbons in the chain. For example, the term “Cₓ-C_{y}” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal.

As used herein, the term “hydrocarbyl” refers to a group that is bonded through a carbon atom that does not have a ═O or ═S substituent, and typically has at least one carbon-hydrogen bond and a primarily carbon backbone but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not.

Hydrocarbyl groups include, but are not limited to aryl, heteroaryl, carbocycle, heterocyclyl, alkyl, alkenyl, alkynyl, and combinations thereof. The “hydrocarbyl” group may be optionally substituted.

As used herein, the term “alkoxy” refers to a straight or branched, saturated aliphatic (alkyl) hydrocarbon radical bonded to an oxygen atom that is attached to a core structure. Preferably, alkoxy groups have one to six carbon atoms, i.e., may be C₁-C₆ alkoxy. Examples of alkoxy groups include but are not limited to methoxy, ethoxy, propoxy, isopropoxy, butoxy, isobutoxy, tert-butoxy, pentoxy, 3-methyl butoxy, and the like. The “alkoxy” group may be optionally substituted.

As used herein, the term “alkoxyalkyl” refers to an alkyl group (as defined above) substituted with an alkoxy group and may be represented by the general formula alkyl-O-alkyl. Examples of alkoxyalkyl groups include but are not limited to methyl-O-ethylene-, ethyl-O-ethylene-. The “alkoxyalkyl” group may be optionally substituted.

As used herein, the term “haloalkyl” refers to alkyl group (as defined above) is substituted with one or more halogens. A monohaloalkyl radical, for example, may have a chlorine, bromine, iodine, or fluorine atom. Dihalo and polyhaloalkyl radicals may have two or more of the same or different halogen atoms. Examples of haloalkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl and the like. The “haloalkyl” group may be optionally substituted.

As used herein, the term “haloalkoxy” refers to radicals wherein one or more of the hydrogen atoms of the alkoxy group are substituted with one or more halogens.

Representative examples of “haloalkoxy” groups include, but not limited to, difluoromethoxy (—OCHF₂), trifluoromethoxy (—OCF₃) or trifluoroethoxy (—OCH₂CF₃). The “haloalkoxy” group may be optionally substituted.

As used herein, the term “haloalkoxyalkyl” refers to an alkyl group (as defined above) substituted with a haloalkoxy group and may be represented by the general formula alkyl-O-haloalkyl. Examples of haloalkoxyalkyl groups include but are not limited to methyl-O-difluoroethylene-, ethyl-O-difluoroethylene-. The “haloalkoxyalkyl” group may be optionally substituted.

As used herein, the term “aryl” includes substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings (fused rings) wherein at least one of the rings is aromatic, e.g., the other cyclic rings can be cycloalkyls, cycloalkenyls, cycloalkynyls, aryls, heteroaryls, and/or heterocyclyls. The term “fused” means that the second ring is attached or formed by having two adjacent atoms in common with the first ring. The term “fused” is equivalent to the term “condensed”. Examples of aryl groups include but are not limited to phenyl, naphthyl, phenanthryl, phenol, aniline, or indanyl and the like. Unless otherwise specified, all aryl groups described herein may be optionally substituted.

As used herein, the terms “polycyclyl”, “polycycle”, and “polycyclic” refer to two or more rings (e.g., cycloalkyls, cycloalkenyls, cycloalkynyls, aryls, heteroaryls, and/or heterocyclyls) in which one or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

As used herein, the term “arylalkyl” refers to an alkyl group substituted with an aryl group. Aryl and/or alkyl of arylalkyls can be further substituted as defined above for aryl and alkyl, respectively.

As used herein, the term “acyl” refers to a group —C(═O)—R^{W} wherein R^{W} is optionally substituted alkyl. Examples of “acyl” include, but are not limited to, instances where R^{W} is C₁-C₁₀alkyl (C₁-C₁₀acyl) or C₁-C₆-alkyl (C₁-C₆acyl). In some embodiments, each occurrence of the optionally substituted substituent is independently selected from the group consisting of H, OH, alkoxy, cyano, F, and amino. Additional examples of “acyl” include —C(═O)—CH₃, —C(═O)—CH₂—CH₃, —C(═O)—CH₂—CH₂—CH₃, or —C(═O)—CH(CH₃)₂.

As used herein, the term “carbamoyl” refers to a group represented by

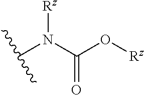

wherein R^{z} independently represents a hydrogen or optionally substituted hydrocarbyl group, or R^{z} groups taken together with the —N—C(═O)—O-moiety to which they are attached complete a heterocycle having from 5 to 8 atoms in the ring structure which may be optionally substituted.

As used herein, the terms “amine” and “amino” refer to both unsubstituted and substituted amines and salts thereof, e.g., a moiety that can be represented by

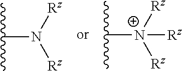

wherein R^{z} independently represent a hydrogen or optionally substituted hydrocarbyl group, or R^{z} groups are taken together with the N atom to which they are attached complete a heterocycle having from 4 to 8 atoms in the ring structure which may be optionally substituted.

As used herein, the terms “amide” and “amido” each refer to a group represented

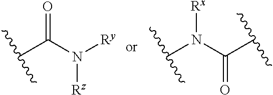

wherein R^{x}, R^{y}, and R^{z} each independently represents a hydrogen or optionally substituted hydrocarbyl group, or R^{y}, and R^{z} groups are taken together with the N atom to which they are attached complete a heterocycle having from 4 to 8 atoms in the ring structure which may be optionally substituted.

As used herein, the term “sulfonamide” is represented by:

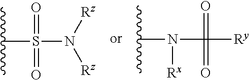

wherein R^{x}, R^{y} and R^{z}, at each occurrence, independently represents a hydrogen, optionally substituted hydrocarbyl group, or R^{z} groups taken together with the N atom to which they are attached complete a heterocycle having from 4 to 8 atoms in the ring structure which may be optionally substituted.

As used herein, the term “sulfone” refers to the group —S(O)₂—RU wherein Ru represents an optionally substituted hydrocarbyl.

As used herein, the term “aminoalkyl” refers to an alkyl group substituted with an amino group.

As used herein, the term “amidoalkyl” refers to an alkyl group substituted with an amido group.

As used herein, the term “cycloalkyl” alone or in combination with other term(s) refers to a cyclic hydrocarbon which is completely saturated. “Cycloalkyl” includes monocyclic, bicyclic, and tricyclic rings. Typically, a monocyclic cycloalkyl group has from 3 to about 10 carbon atoms, more typically 3 to 8 carbon atoms (e.g., C₃-C₁₀cycloalkyl or e.g., C₃-C₆cycloalkyl) unless otherwise defined. Examples of monocyclic cycloalkyls include cyclopropyl, cyclobutyl, cyclopentyl, cyclohexyl, cycloheptyl, and the like. The second ring of a bicyclic cycloalkyl or, the second or third rings of a tricyclic cycloalkyl, may be selected from saturated, unsaturated, and aromatic rings. Cycloalkyl includes bicyclic and tricyclic molecules in which one, two or three or more atoms are shared between the two rings. The term “fused cycloalkyl” refers to a bicyclic or tricyclic cycloalkyl in which each of the rings shares two adjacent atoms with the other ring. The second ring of a fused bicyclic cycloalkyl or, the second or third rings of a fused tricyclic cycloalkyl, may be selected from saturated, unsaturated, and aromatic rings. A “cycloalkenyl” group is a cyclic hydrocarbon containing one or more double bonds. Cycloalkyls can be further substituted with alkyls, alkenyls, alkoxys, alkylthios, aminoalkyls, carbonyl-substituted alkyls, —CF₃, —CN, and the like. A cycloalkyl may alternatively be polycyclic with more than two rings. Examples of polycyclic cycloalkyls include bridged, fused, and spirocyclic carbocyclyls.

As used herein, the terms “carbocycle,” or “carbocyclic” include bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, 4,5-naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

As used interchangeably herein, the term “cycloalkylalkyl” or “carbocyclylalkyl” refers to an alkyl group substituted with a cycloalkyl group. Carbocyclyl and/or alkyl of carbocyclylalkyls can be further substituted as defined above for cycloalkyl and alkyl, respectively.

As used herein, the term “cyano” refers to —CN group.

As used herein, the term “hydroxy” or “hydroxyl” refers to —OH group.

As used herein, the term “cyanoalkyl” refers to an alkyl group substituted with a cyano group.

As used herein, the term “hydroxyalkyl” refers to an alkyl group substituted with a hydroxy group.

As used herein, the term “halide”, “halo” or “halogen” alone or in combination with other term(s) means chloro, fluoro, bromo, and iodo.

As used herein, the term “heteroatom” refers an atom of any element other than carbon or hydrogen. Exemplary heteroatoms are nitrogen (N), oxygen (O), sulfur (S), and silicon (Si).

As used herein, the terms “heterocyclyl”, “heterocycloalkyl”, “heterocycle”, and “heterocyclic” refer to a non-aromatic, saturated or partially saturated, including monocyclic, polycyclic (e.g., bicyclic, tricyclic) bridged, or fused, ring system of 3 to 15 member having at least one heteroatom or heterogroup selected from O, N, S, S(O), S(O)₂, NH, or C(O) with the remaining ring atoms being independently selected from the group consisting of carbon, oxygen, nitrogen, and sulfur. Examples of “heterocyclyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, 2-azabicyclo[2.2.2]octanyl, azocinyl, chromanyl, xanthenyl and N-oxides thereof. Attachment of a heterocycloalkyl substituent can occur via either a carbon atom or a heteroatom. A heterocycloalkyl group can be optionally substituted with one or more suitable groups by one or more aforesaid groups. Preferably “heterocyclyl” refers to 4- to 6-membered ring selected from the group consisting of, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl and N-oxides thereof. More preferably, “heterocycloalkyl” includes azetidinyl, pyrrolidinyl, morpholinyl and piperidinyl. All heterocycloalkyl are optionally substituted by one or more aforesaid groups.

As used herein, “heterocyclylalkyl” refers to an alkyl group substituted with a heterocyclyl. If the heterocyclyl is a nitrogen-containing heterocyclyl, the heterocyclyl is optionally attached to the alkyl radical at the nitrogen atom. Heterocyclyl and/or alkyl of heterocyclylalkyls can be further substituted as defined above for heterocyclyl and alkyl, respectively.

As used herein, the term “heteroaryl” refers to substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also refers to substituted or unsubstituted aromatic or partly aromatic ring systems containing at least one heteroatom and having two or more cyclic rings (bicyclic, tricyclic, or polycyclic), containing 8 to 20 ring atoms, suitably 5 to 10 ring atoms, which may be linked covalently, or fused in which two or more atoms are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. The rings may contain an N or S atom, wherein the N or S atom is optionally oxidized, or the N atom is optionally quaternized. All heteroaryls are optionally substituted. Any suitable ring position of the heteroaryl moiety may be covalently linked to a defined chemical structure. Examples of heteroaryl include, but are not limited to: furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, thiadiazolyl, triazolyl, pyridyl, pyrimidinyl, pyrazinyl, pyridazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, alpha-carboline, indolizinyl, benzoisothiazolyl, benzoxazolyl, pyrrolopyridyl, furopyridinyl, purinyl, benzothiadiazolyl, benzoxadiazolyl, benzotriazolyl, benzotriadiazolyl, 7-azaindolyl, 7-azaindazolyl, pyrrolopyridinyl, pyrrolopyrimidinyl, oxazolonepyridinyl, oxazolonepyrimidinyl, imidazolonepyridinyl, imidazolonepyrimidinyl, pyrazolopyridinyl, pyrazolopyrimidinyl, tetrahydronaphthyridinyl, tetrahydropyridolpyriminyl, dihydronaphthyridinonyl, naphthyridinonyl, oxazinanonepyridinyl, oxazinanonepyrimidinyl, carbazolyl, dibenzothienyl, acridinyl, and the like.

As used herein, “heteroarylalkyl” refers to an alkyl group substituted with a heteroaryl. If the heteroaryl is a nitrogen-containing heteroaryl, the heteroaryl is optionally attached to the alkyl radical at the nitrogen atom. Heteroaryl and/or alkyl of heteroarylalkyls can be further substituted as defined above for heteroaryl and alkyl, respectively.

The compounds of the disclosure may contain one or more chiral centers and, therefore, exist as stereoisomers. The term “stereoisomers” when used herein consist of all enantiomers or diastereomers. These compounds may be designated by the symbols “R” or “S,” depending on the configuration of substituents around the stereogenic carbon atom, but the skilled artisan will recognize that a structure may denote a chiral center implicitly. These compounds may also be designated by “(+)” and “(−)” based on their optical rotation properties.

The presently described compounds encompasses various stereoisomers of these compounds and mixtures thereof. Mixtures of enantiomers or diastereomers may be designated by the symbol “(±)” in nomenclature, but the skilled artisan will recognize that a structure may denote a chiral center implicitly.

Individual enantiomers and diastereomers of the disclosed compounds can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations and may involve the use of chiral auxiliaries. For examples, see Carreira and Kvaerno, Classics in Stereoselective Synthesis, Wiley-VCH: Weinheim, 2009.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into the disclosed compounds include isotopes of hydrogen, carbon, nitrogen, oxygen, phosphorus, sulfur, fluorine, and chlorine, such as ²H, ³H, ¹³C ¹⁴C, ¹⁵N, ¹⁸O, ¹⁷O, ³¹P ³²P, ³⁵S, ¹⁸F, and ³⁶Cl, respectively. For example, a compound of the disclosure may have one or more H atom replaced with deuterium.

A “combination therapy” is a treatment that includes the administration of two or more therapeutic agents, e.g., a compound of the disclosure and a STING agonist, to a patient in need thereof.

“Disease,” “disorder,” and “condition” are used interchangeably herein.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisate, fumarate, gluconate, glucoronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

In the present specification, the term “therapeutically effective amount” means the amount of the subject compound that will elicit the biological or medical response of a tissue, system, or animal, (e.g., mammal or human) that is being sought by the researcher, veterinarian, medical doctor or other clinician. The compounds described herein are administered in therapeutically effective amounts to treat a disorder.

“Treating” includes any effect, e.g., lessening, reducing, modulating, or eliminating, that results in the improvement of the condition, disease, disorder, and the like.

As used herein, “prophylaxis” contemplates any effect that occurs before a subject begins to suffer from the specified condition, disease, disorder, and the like, and includes preventing said condition, disease, disorder, or one or more symptoms associated with the condition, disease, and disorder or preventing its recurrence.

### Compounds

In some embodiments, described herein is a compound of Formula (I):

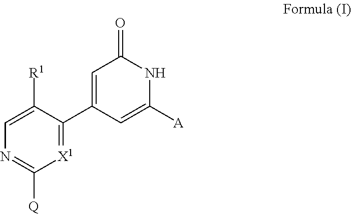

or a pharmaceutically acceptable salt, enantiomer, stereoisomer, or tautomer thereof, wherein:
X¹ is selected from the group consisting of CH and N;
A is selected from the group consisting of cycloalkyl, heterocyclyl, aryl, heteroaryl, and —NHSO₂R³;
Q is selected from the group consisting of H, —NHR², and —NHC(O)LR⁴;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R³ is selected from the group consisting of alkyl, amino, cycloalkyl, heterocyclyl, aryl and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl; and
L is selected from the group consisting of a bond and alkyl, wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, described herein is a compound of Formula (I-A):

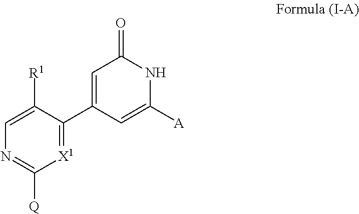

or a pharmaceutically acceptable salt, enantiomer, stereoisomer, or tautomer thereof, wherein:
X¹ is selected from the group consisting of CH and N;
A is heterocyclyl;
Q is selected from the group consisting of H, —NHR², and —NHC(O)LR⁴;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl; and
L is selected from the group consisting of a bond and alkyl, wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, Q is selected from the group consisting of —NHR² and —NHC(O)LR⁴.

In some embodiments, described herein is a compound of Formula (I-B):

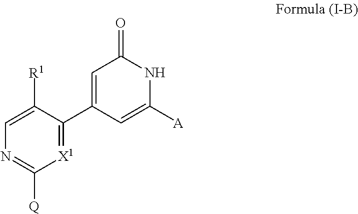

or a pharmaceutically acceptable salt, enantiomer, stereoisomer, or tautomer thereof, wherein:
X¹ is selected from the group consisting of CH and N;
A is heteroaryl;
Q is selected from the group consisting of H, —NHR², and —NHC(O)LR⁴;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl; and
L is selected from the group consisting of a bond and alkyl, wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, Q is selected from the group consisting of —NHR² and —NHC(O)LR⁴.

In some embodiments, described herein is a compound of Formula (I-C):

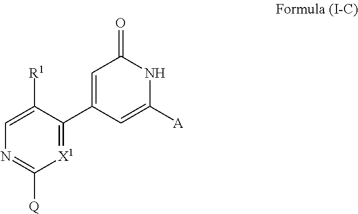

or a pharmaceutically acceptable salt, enantiomer, stereoisomer, or tautomer thereof, wherein:
X¹ is selected from the group consisting of CH and N;
A is selected from the group consisting of cycloalkyl, heterocyclyl, aryl, heteroaryl, and —NHSO₂R³;
Q is —NHR²;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl; and
R³ is selected from the group consisting of alkyl, amino, cycloalkyl, heterocyclyl, aryl and heteroaryl, wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, described herein is a compound of Formula (I-D):

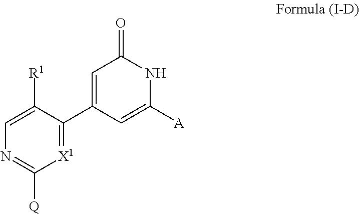

or a pharmaceutically acceptable salt, enantiomer, stereoisomer, or tautomer thereof, wherein:
X¹ is selected from the group consisting of CH and N;
A is selected from the group consisting of cycloalkyl, heterocyclyl, aryl, heteroaryl, and —NHSO₂R³;
Q is —NHC(O)LR⁴;
R¹ is selected from the group consisting of alkyl, haloalkyl, halide, cycloalkyl, and amino;
R³ is selected from the group consisting of alkyl, amino, cycloalkyl, heterocyclyl, aryl and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl; and
L is selected from the group consisting of a bond and alkyl, wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, described herein is a compound of Formula (I-E):

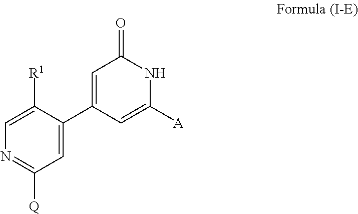

or a pharmaceutically acceptable salt, enantiomer, stereoisomer, or tautomer thereof, wherein:
A is heterocyclyl;
Q is selected from the group consisting of H, —NHR², and —NHC(O)LR⁴;
L is selected from the group consisting of a bond and alkyl;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl; and
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl, wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, described herein is a compound of Formula (I-F):

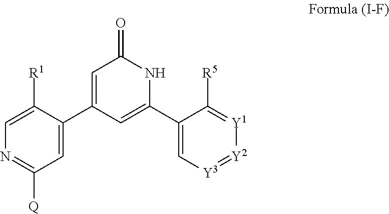

or a pharmaceutically acceptable salt, enantiomer, stereoisomer, or tautomer thereof, wherein:
Y¹ and Y³ are selected from the group consisting of CH and N;
Y² is selected from the group consisting of CR⁶ and N;
provided that not more than one of Y¹, Y², and Y³ is N;
Q is selected from the group consisting of H, —NHR², and —NHC(O)LR⁴;
L is selected from the group consisting of a bond and alkyl;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl;
R⁵ is selected from the group consisting of alkyl, cycloalkyl, haloalkyl, and halogen;
R⁶ is selected from the group consisting of NHS(O)₂R⁷ and NHC(O)R⁸;
R⁷ is selected from the group consisting of alkyl, amino, cycloalkyl, heterocyclyl, alkylamino, aryl, and heteroaryl; and
R⁸ is selected from the group consisting of alkyl, amino, cycloalkyl, heterocyclyl, alkylamino, aryl, and heteroaryl, wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, described herein is a compound of Formula (I-G):

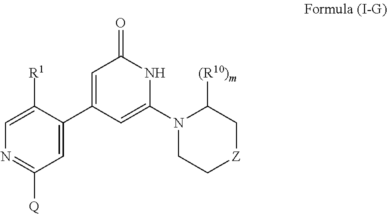

or a pharmaceutically acceptable salt, enantiomer, stereoisomer, or tautomer thereof, wherein:
Z is selected from the group consisting of CH₂ and NR⁹;
Q is selected from the group consisting of H, —NHR², and —NHC(O)LR⁴;
L is selected from the group consisting of a bond and alkyl;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl;
R⁹ is selected from the group consisting of S(O)₂R³¹ and C(O)R³²;
R¹⁰ is selected from the group consisting of H, alkyl, cycloalkyl, and haloalkyl; or two occurrences of R¹⁰ taken together with the carbon atom to which they are attached form an optionally substituted C₃-C₅cycloalkyl ring;
R³¹ is selected from the group consisting of alkyl, amino, cycloalkyl, heterocyclyl, alkylamino, aryl, and heteroaryl; and
R³² is selected from the group consisting of alkyl, amino, cycloalkyl, heterocyclyl, alkylamino, aryl, and heteroaryl; and
m is 1 or 2,
   wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, described herein is a compound of Formula (I-H):

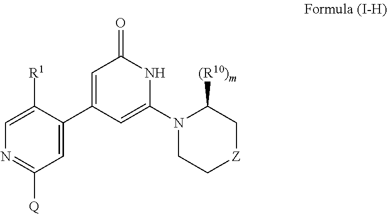

or a pharmaceutically acceptable salt, enantiomer, stereoisomer, or tautomer thereof, wherein:
Z is selected from the group consisting of CH₂ and NR⁹;
Q is selected from the group consisting of H, —NHR², and —NHC(O)LR⁴;
L is selected from the group consisting of a bond and alkyl;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl;
R⁹ is selected from the group consisting of S(O)₂R³¹ and C(O)R³²;
R¹⁰ is selected from the group consisting of H, alkyl, cycloalkyl, and haloalkyl; or two occurrences of R¹⁰ taken together with the carbon atom to which they are attached form an optionally substituted C₃-C₅cycloalkyl ring;
R³¹ is selected from the group consisting of alkyl, amino, cycloalkyl, heterocyclyl, alkylamino, aryl, and heteroaryl; and
R³² is selected from the group consisting of alkyl, amino, cycloalkyl, heterocyclyl, alkylamino, aryl, and heteroaryl; and
m is 1 or 2,
   wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, described herein is a compound of Formula (I-I):

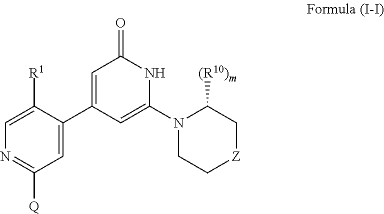

or a pharmaceutically acceptable salt, enantiomer, stereoisomer, or tautomer thereof, wherein:
Z is selected from the group consisting of CH₂ and NR⁹;
Q is selected from the group consisting of H, —NHR², and —NHC(O)LR⁴;
L is selected from the group consisting of a bond and alkyl;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl;
R⁹ is selected from the group consisting of S(O)₂R³¹ and C(O)R³²;
R¹⁰ is selected from the group consisting of H, alkyl, cycloalkyl, and haloalkyl; or wherein two occurrences of R¹⁰ taken together with the carbon atom to which they are attached form an optionally substituted C₃-C₅cycloalkyl ring;
R³¹ is selected from the group consisting of alkyl, amino, cycloalkyl, heterocyclyl, alkylamino, aryl, and heteroaryl; and
R³² is selected from the group consisting of alkyl, amino, cycloalkyl, heterocyclyl, alkylamino, aryl, and heteroaryl; and
m is 1 or 2,
   wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, described herein is a compound of Formula (I-J):

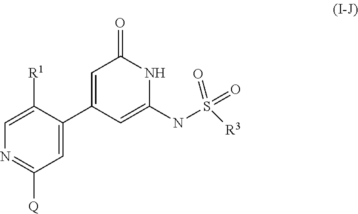

or a pharmaceutically acceptable salt, enantiomer, stereoisomer, or tautomer thereof, wherein:
Q is selected from the group consisting of H, —NHR², and —NHC(O)LR⁴;
L is selected from the group consisting of a bond and alkyl;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R³ is selected from the group consisting of alkyl, amino, cycloalkyl, heterocyclyl, aryl and heteroaryl; and
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl,
wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

In some embodiments, X¹ is CH. In other embodiments, X¹ is N.

In some embodiments, R¹ is selected from the group consisting of alkyl, halide and amino. For example, in some embodiments, R¹ is CH₃, —NH₂, F, or Cl. In some embodiments, R¹ is CH₃. In some embodiments, R¹ is —NH₂. In some embodiments, R¹ is selected from the group consisting of F and C1.

In some embodiments, Q is —NHR².

In some embodiments, R² is heteroaryl. For example, in some embodiments, R² is

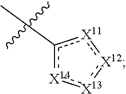

wherein [symbol] is a single bond or a double bond; X¹¹ is selected from the group consisting of N, CR″, NR²¹, S, and O; X¹² is selected from the group consisting of N, CR¹², NR²², S, and O; X¹³ is selected from the group consisting of N, CR¹³, NR²³, S, and O; X¹⁴ is selected from the group consisting of N, CR¹⁴, NR²⁴, S, and O; and wherein R¹¹, R¹², R¹³, R¹⁴, R²¹, R²², R²³ and R²⁴ are each independently selected from the group consisting of H, amino, halogen, cyano, alkyl, cycloalkyl and alkylamino. In some embodiments, R¹¹, R¹², R¹³ and R¹⁴ are each independently selected from the group consisting of H, C₁-C₆alkyl, and C₁-C₆alkylamino. In some embodiments, R¹¹, R¹², R¹³ and R¹⁴ are each independently selected from the group consisting of H, CH₃, and —(CH₂)₂N(CH₃)₂. In some embodiments, R²¹, R²², R²³ and
R²⁴ are each independently selected from the group consisting of H, C1-C6alkyl, and C₁-C₆alkylamino. In some embodiments, R²¹, R²², R²³ and R²⁴ are each independently selected from the group consisting of H, CH₃, and —(CH₂)₂N(CH₃)₂.

In some embodiments, R² is selected from the group consisting of

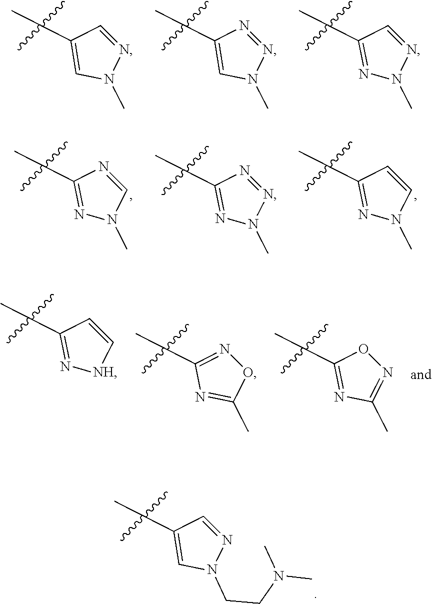

In some embodiments, R² is

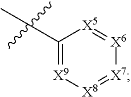

X⁵ is selected from the group consisting of N and CR¹⁵; X⁶ is selected from the group consisting of N and CR¹⁶; X⁷ is selected from the group consisting of N and CR¹⁷; X⁸ is selected from the group consisting of N and CR¹⁸; X⁹ is selected from the group consisting of N and CR¹⁹; wherein not more than three of X⁵, X⁶, X⁷, X⁸, and X⁹ is nitrogen; and wherein R¹⁵, R¹⁶, R¹⁷, R¹⁸ and R¹⁹ are each independently selected from the group consisting of H, amino, halogen, cyano, alkyl, cycloalkyl and alkylamino. In some embodiments, R¹⁵, R¹⁶, R¹⁷, R¹⁸ and R¹⁹ are each independently selected from the group consisting of H, amino, halogen, and cyano. In some embodiments, R¹⁵, R¹⁶, R¹⁷, R¹⁸ and R¹⁹ are each independently selected from the group consisting of H and C1-C6alkyl. In some embodiments, R¹⁵, R¹⁶, R¹⁷, R¹⁸ and R¹⁹ are each independently selected from the group consisting of H and CH₃.

In some embodiments, R² is selected from the group consisting of

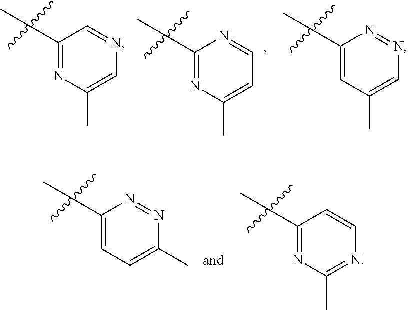

In some embodiments, R² is selected from the group consisting of

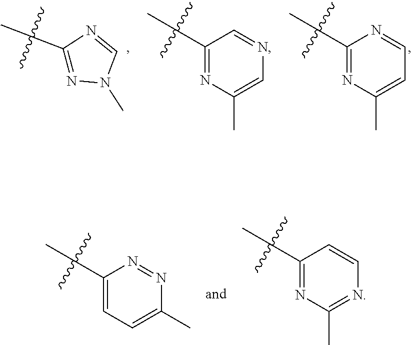

In some embodiments, L is selected from the group consisting of a bond and alkyl. In some embodiments, L is selected from the group consisting of a bond and C₁-C₆ alkyl. In some embodiments, L is selected from the group consisting of a bond and C₁-C₄ alkyl.

In some embodiments, Q is NHC(O)LR⁴.

In some embodiments, L is a bond.

In some embodiments, R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl. In some embodiments, each of cycloalkyl, heterocyclyl, aryl, heteroaryl, O-cycloalkyl, O-heterocyclyl, O-aryl, O-heteroaryl, NH-cycloalkyl, NH-heterocyclyl, NH-aryl, or NH-heteroaryl is optionally substituted with one or more occurrences of a substituent independently selected from the group consisting of alkyl and halogen. In some embodiments, R⁴ is selected from the group consisting of H, alkyl, cycloalkyl, heterocyclyl, aryl, heteroaryl, alkoxy, amino, O-cycloalkyl, O-heterocyclyl, NH-cycloalkyl, NH-heterocyclyl, and NH-heteroaryl. In some embodiments, each of cycloalkyl, heterocyclyl, aryl, heteroaryl, O-cycloalkyl, O-heterocyclyl, NH-cycloalkyl, NH-heterocyclyl, or NH-heteroaryl is optionally substituted with one or more occurrences of a substituent independently selected from the group consisting of alkyl and halogen. In some embodiments, R⁴ is selected from the group consisting of alkyl, alkoxy, amino, alkylamino cycloalkyl, heterocyclyl, aryl, heteroaryl, O-cycloalkyl, and O-heterocyclyl. In some embodiments, each of heterocyclyl, heteroaryl, O-cycloalkyl, or O-heterocyclyl is optionally substituted with one or more occurrences of a substituent independently selected from the group consisting of alkyl and halogen. In some embodiments, R⁴ is selected from the group consisting of alkyl, alkoxy, amino, cycloalkyl, heterocyclyl, heteroaryl, O-cycloalkyl, and O-heterocyclyl. In some embodiments, each of heterocyclyl, heteroaryl, O-cycloalkyl, or O-heterocyclyl is optionally substituted with one or more occurrences of a substituent independently selected from the group consisting of alkyl and halogen. In some embodiments, R⁴ is selected from the group consisting of alkyl, alkoxy, amino, alkylamino cycloalkyl, heterocyclyl, aryl, heteroaryl, and O-heterocyclyl. In some embodiments, each of heterocyclyl, heteroaryl, and O-heterocyclyl is optionally substituted with one or more occurrences of a substituent independently selected from the group consisting of alkyl and halogen. In some embodiments, R⁴ is selected from the group consisting of alkyl, alkoxy, amino, cycloalkyl, heterocyclyl, heteroaryl, and O-heterocyclyl. In some embodiments, each of heterocyclyl, heteroaryl, and O-heterocyclyl is optionally substituted with one or more occurrences of a substituent independently selected from the group consisting of alkyl and halogen.

In some embodiments, LR⁴ is selected from the group consisting of methyl, methoxy, —N(H)CH₃,

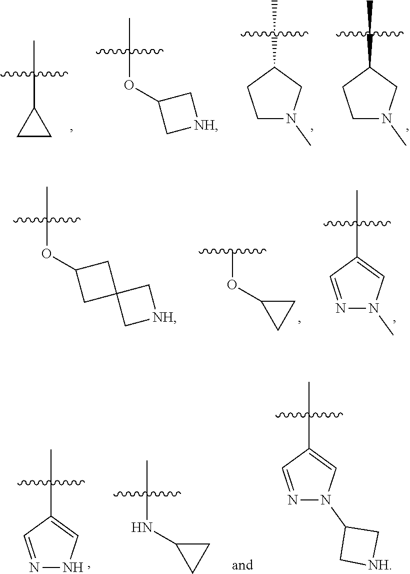

In some embodiments, LR⁴ is selected from the group consisting of methyl, methoxy, and

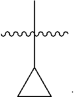 [SMILES: *C1CC1]

In some embodiments, L is alkyl.

In some embodiments, R⁴ is selected from the group consisting of alkoxy, amino, cycloalkyl, heterocyclyl, aryl, and heteroaryl, wherein heterocyclyl is optionally substituted with one or more occurrences of a substituent independently selected from the group consisting of alkyl and halogen. In some embodiments, R⁴ is selected from the group consisting of amino and heterocyclyl. In some embodiments, LR⁴ is selected from the group consisting of

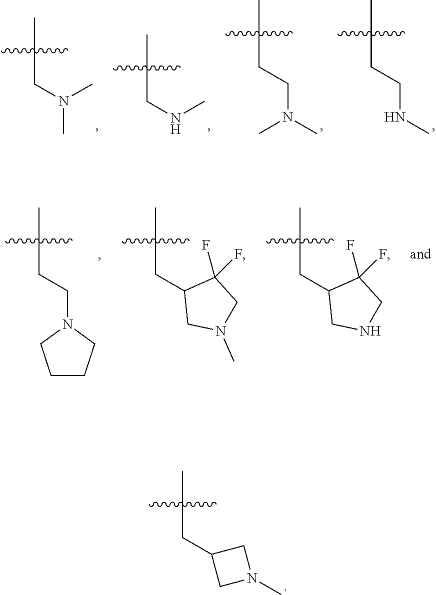

In some embodiments, A is selected from the group consisting of aryl, heteroaryl, heterocyclyl, and —NHSO₂R³.

In some embodiments, A is —NHSO₂R³, and wherein R³ is phenyl optionally substituted with one or more occurrences of R³³, wherein each occurrence of R³³ is independently selected from the group consisting of H, amino, halogen, cyano, alkyl, cycloalkyl and alkylamino. In some embodiments, A is —NHSO₂R³, and wherein R³ is phenyl optionally substituted with one or more occurrences of R³³, wherein each occurrence of R³³ is independently selected from the group consisting of H, amino, and halogen.

In some embodiments, A is selected from the group consisting of aryl and heteroaryl.

In some embodiments, A is

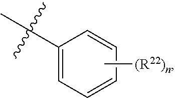

wherein n is 0, 1, 2 or 3; and each occurrence of R²² is independently selected from the group consisting of alkyl, haloalkyl, cycloalkyl and sulfonamide. In some embodiments, A is

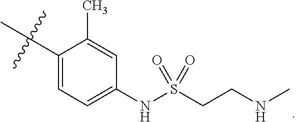 [SMILES: *c1ccc(NS(=O)(=O)CCNC)cc1C]

In some embodiments, A is selected from the group consisting of

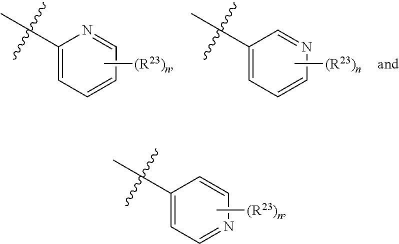

wherein n is 0, 1, 2 or 3; and each occurrence of R²³ is independently selected from the group consisting of alkyl, haloalkyl, and cycloalkyl.

In some embodiments, A is selected from the group consisting of

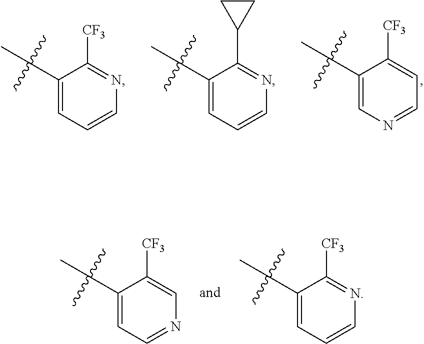 [SMILES: *c1cccnc1C(F)(F)F ; *c1cccnc1C1CC1 ; *c1cnccc1C(F)(F)F ; *c1ccncc1C(F)(F)F ; *c1cccnc1C(F)(F)F]

In some embodiments, A is selected from the group consisting of

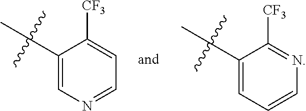 [SMILES: *c1cnccc1C(F)(F)F ; *c1cccnc1C(F)(F)F]

In some embodiments, A is heterocyclyl. In some embodiments, A is selected from the group consisting of

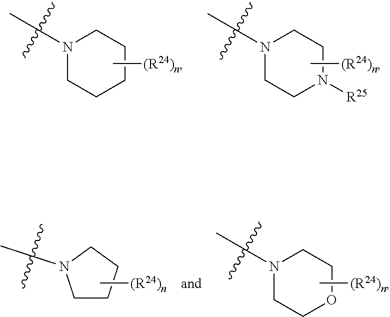

wherein n is 0, 1, 2 or 3; each occurrence of R²⁴ is independently selected from the group consisting of alkyl, haloalkyl, and cycloalkyl; R²⁵ is selected from the group consisting of alkyl, cycloalkyl, C(O)—R²⁶, SO₂—R²⁷; R²⁶ is selected from the group consisting of alkyl, cycloalkyl, aryl, and heteroaryl; and R²⁷ is selected from the group consisting of alkyl, cycloalkyl, aryl, and heteroaryl.

In some embodiments, A is selected from the group consisting of

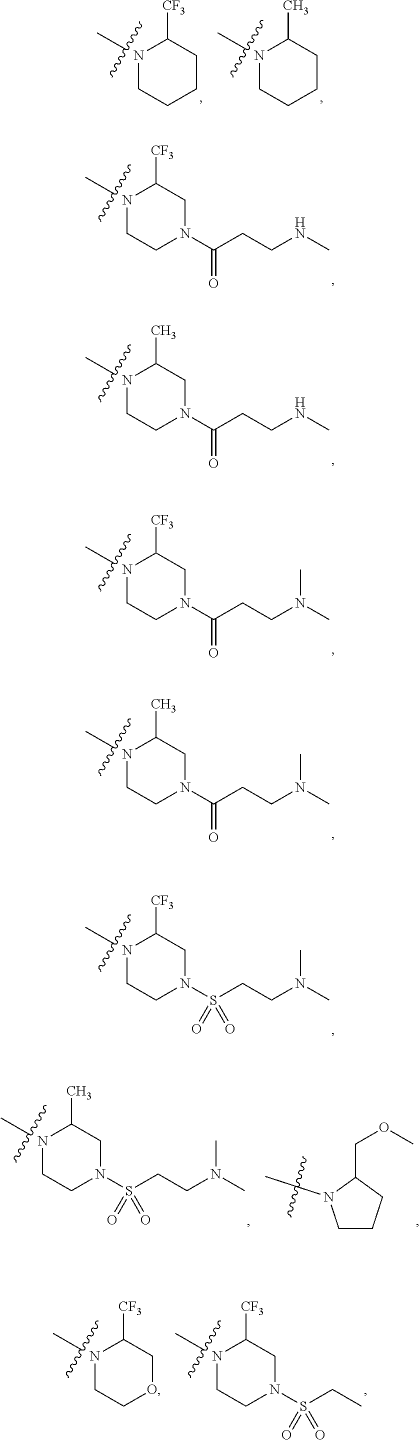

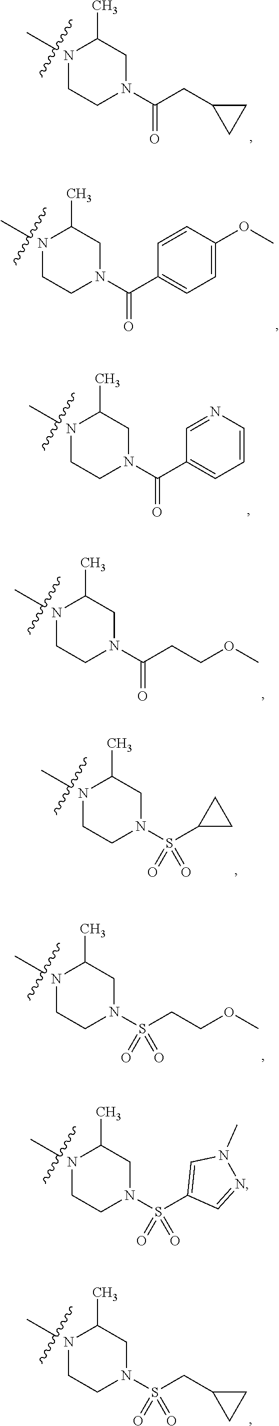

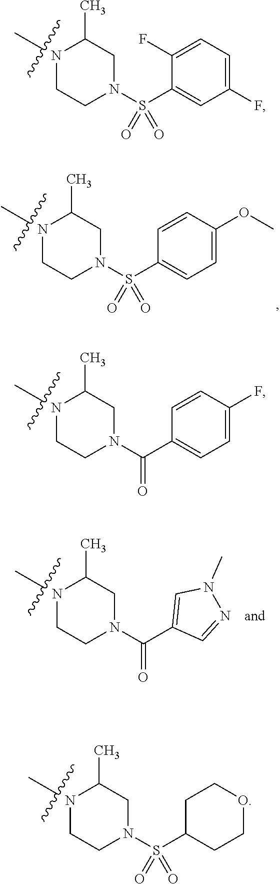

In some embodiments, A is selected from the group consisting of

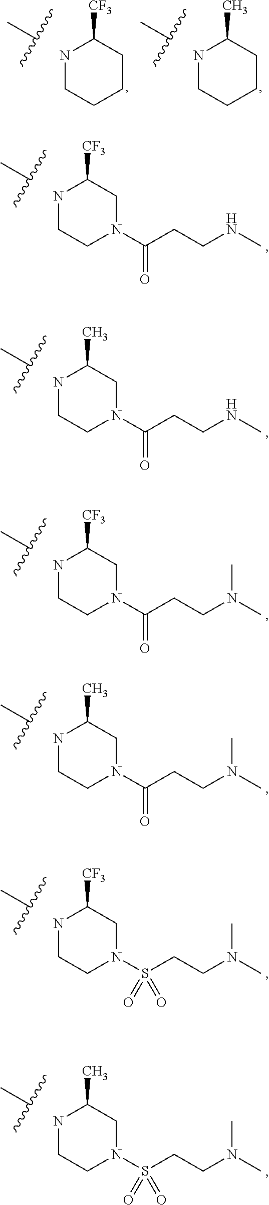

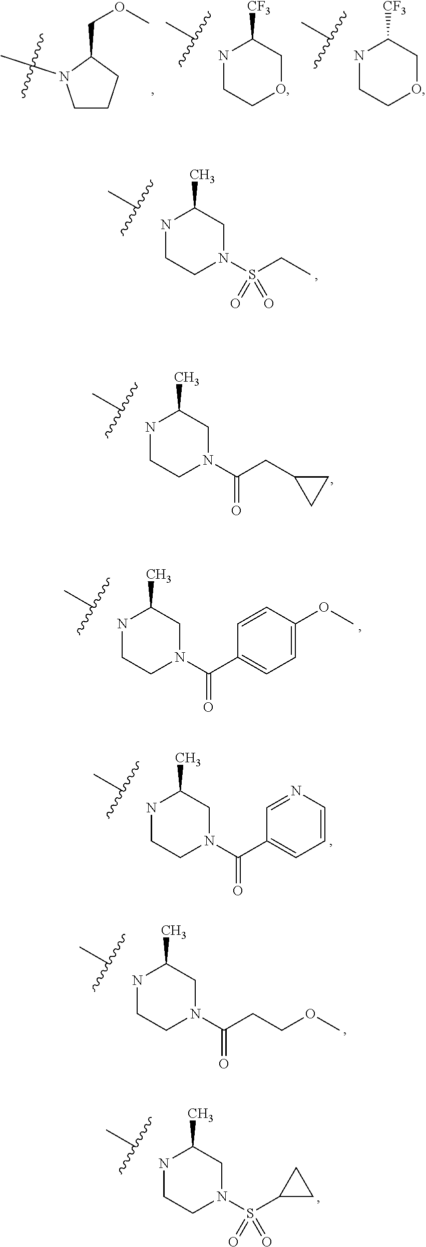

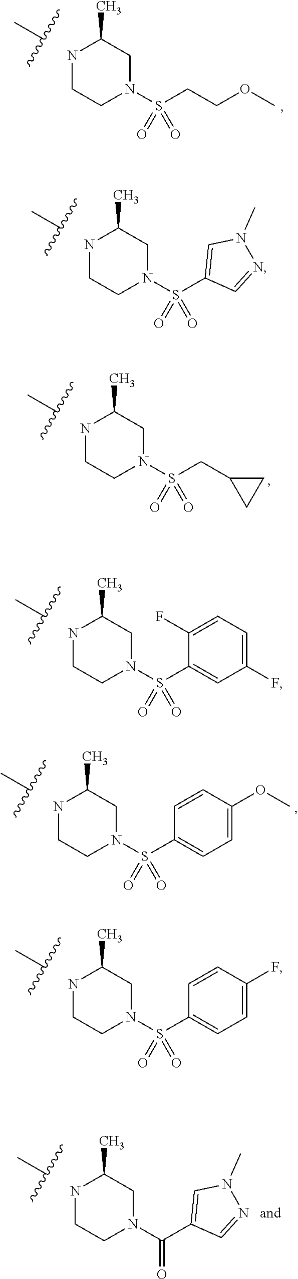

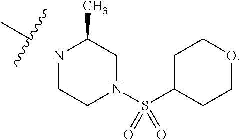

In some embodiments, A is selected from the group consisting of

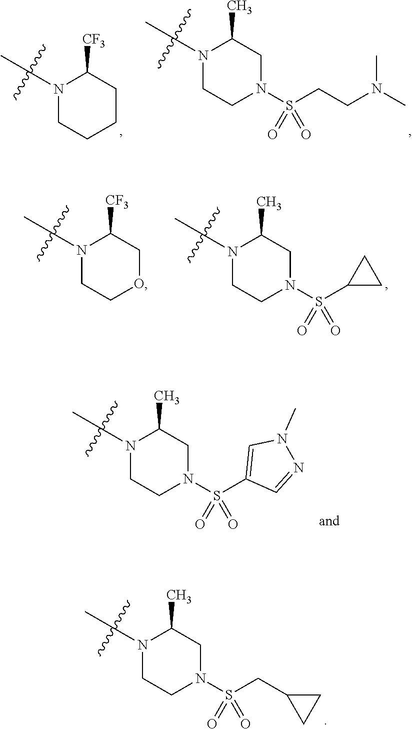

In some embodiments, provided herein is a compound selected from the group consisting of:

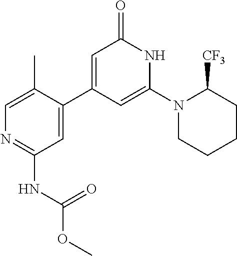 [SMILES: COC(=O)Nc1cc(-c2cc(N3CCCC[C@@H]3C(F)(F)F)[nH]c(=O)c2)c(C)cn1]

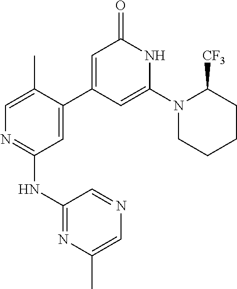 [SMILES: Cc1cncc(Nc2cc(-c3cc(N4CCCC[C@@H]4C(F)(F)F)[nH]c(=O)c3)c(C)cn2)n1]

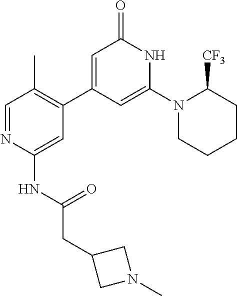 [SMILES: Cc1cnc(NC(=O)CC2CN(C)C2)cc1-c1cc(N2CCCC[C@@H]2C(F)(F)F)[nH]c(=O)c1]

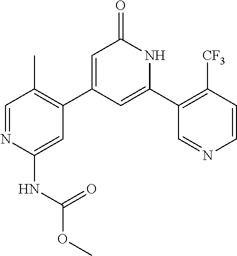 [SMILES: COC(=O)Nc1cc(-c2cc(-c3cnccc3C(F)(F)F)[nH]c(=O)c2)c(C)cn1]

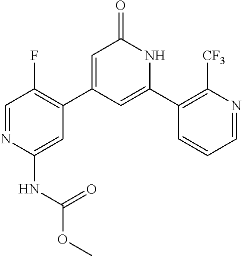 [SMILES: COC(=O)Nc1cc(-c2cc(-c3cccnc3C(F)(F)F)[nH]c(=O)c2)c(F)cn1]

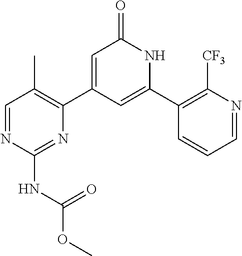 [SMILES: COC(=O)Nc1ncc(C)c(-c2cc(-c3cccnc3C(F)(F)F)[nH]c(=O)c2)n1]

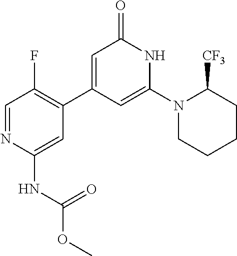 [SMILES: COC(=O)Nc1cc(-c2cc(N3CCCC[C@@H]3C(F)(F)F)[nH]c(=O)c2)c(F)cn1]

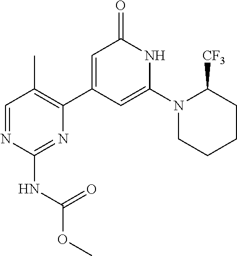 [SMILES: COC(=O)Nc1ncc(C)c(-c2cc(N3CCCC[C@@H]3C(F)(F)F)[nH]c(=O)c2)n1]

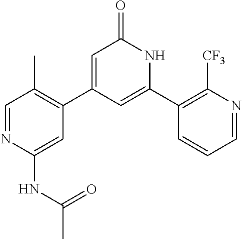 [SMILES: CC(=O)Nc1cc(-c2cc(-c3cccnc3C(F)(F)F)[nH]c(=O)c2)c(C)cn1]

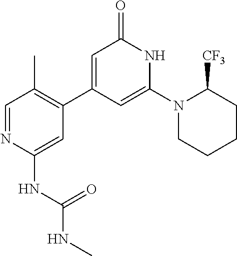 [SMILES: CNC(=O)Nc1cc(-c2cc(N3CCCC[C@@H]3C(F)(F)F)[nH]c(=O)c2)c(C)cn1]

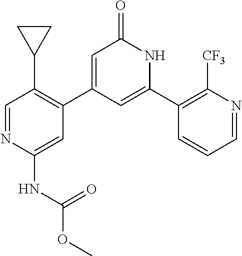 [SMILES: COC(=O)Nc1cc(-c2cc(-c3cccnc3C(F)(F)F)[nH]c(=O)c2)c(C2CC2)cn1]

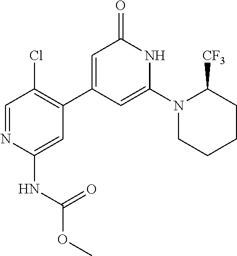 [SMILES: COC(=O)Nc1cc(-c2cc(N3CCCC[C@@H]3C(F)(F)F)[nH]c(=O)c2)c(Cl)cn1]

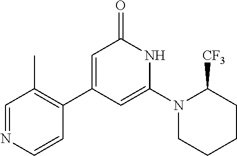 [SMILES: Cc1cnccc1-c1cc(N2CCCC[C@@H]2C(F)(F)F)[nH]c(=O)c1]

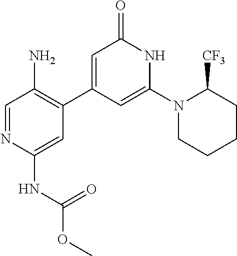 [SMILES: COC(=O)Nc1cc(-c2cc(N3CCCC[C@@H]3C(F)(F)F)[nH]c(=O)c2)c(N)cn1]

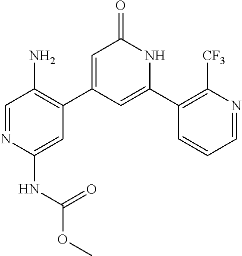 [SMILES: COC(=O)Nc1cc(-c2cc(-c3cccnc3C(F)(F)F)[nH]c(=O)c2)c(N)cn1]

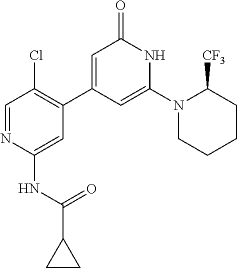 [SMILES: O=C(Nc1cc(-c2cc(N3CCCC[C@@H]3C(F)(F)F)[nH]c(=O)c2)c(Cl)cn1)C1CC1]

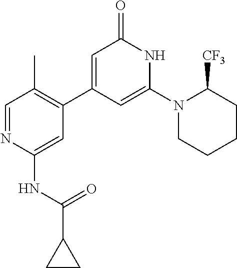 [SMILES: Cc1cnc(NC(=O)C2CC2)cc1-c1cc(N2CCCC[C@@H]2C(F)(F)F)[nH]c(=O)c1]

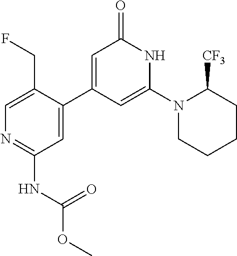 [SMILES: COC(=O)Nc1cc(-c2cc(N3CCCC[C@@H]3C(F)(F)F)[nH]c(=O)c2)c(CF)cn1]

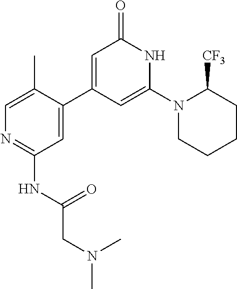 [SMILES: Cc1cnc(NC(=O)CN(C)C)cc1-c1cc(N2CCCC[C@@H]2C(F)(F)F)[nH]c(=O)c1]

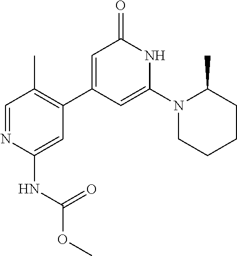 [SMILES: COC(=O)Nc1cc(-c2cc(N3CCCC[C@@H]3C)[nH]c(=O)c2)c(C)cn1]

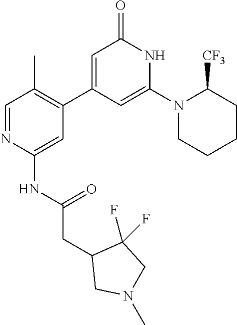 [SMILES: Cc1cnc(NC(=O)CC2CN(C)CC2(F)F)cc1-c1cc(N2CCCC[C@@H]2C(F)(F)F)[nH]c(=O)c1]

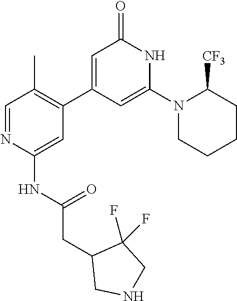 [SMILES: Cc1cnc(NC(=O)CC2CNCC2(F)F)cc1-c1cc(N2CCCC[C@@H]2C(F)(F)F)[nH]c(=O)c1]

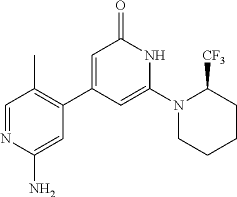 [SMILES: Cc1cnc(N)cc1-c1cc(N2CCCC[C@@H]2C(F)(F)F)[nH]c(=O)c1]

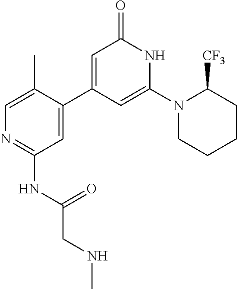 [SMILES: CNCC(=O)Nc1cc(-c2cc(N3CCCC[C@@H]3C(F)(F)F)[nH]c(=O)c2)c(C)cn1]

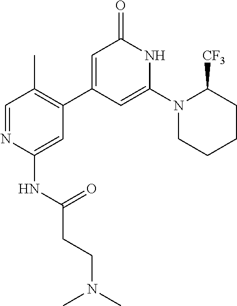 [SMILES: Cc1cnc(NC(=O)CCN(C)C)cc1-c1cc(N2CCCC[C@@H]2C(F)(F)F)[nH]c(=O)c1]

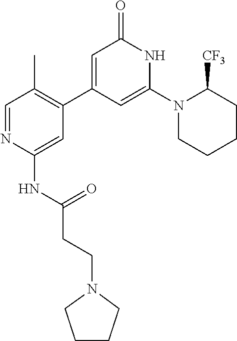 [SMILES: Cc1cnc(NC(=O)CCN2CCCC2)cc1-c1cc(N2CCCC[C@@H]2C(F)(F)F)[nH]c(=O)c1]

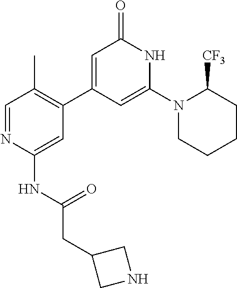 [SMILES: Cc1cnc(NC(=O)CC2CNC2)cc1-c1cc(N2CCCC[C@@H]2C(F)(F)F)[nH]c(=O)c1]

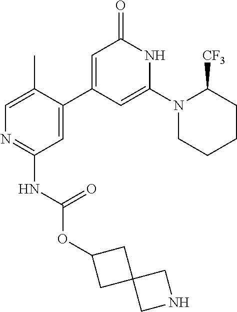 [SMILES: Cc1cnc(NC(=O)OC2CC3(CNC3)C2)cc1-c1cc(N2CCCC[C@@H]2C(F)(F)F)[nH]c(=O)c1]

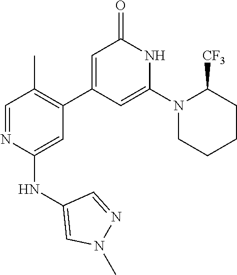 [SMILES: Cc1cnc(Nc2cnn(C)c2)cc1-c1cc(N2CCCC[C@@H]2C(F)(F)F)[nH]c(=O)c1]

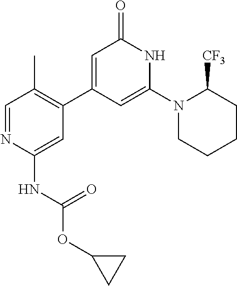 [SMILES: Cc1cnc(NC(=O)OC2CC2)cc1-c1cc(N2CCCC[C@@H]2C(F)(F)F)[nH]c(=O)c1]

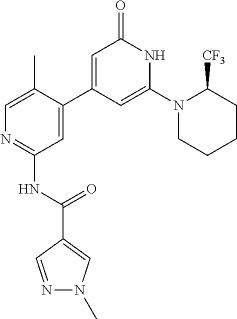 [SMILES: Cc1cnc(NC(=O)c2cnn(C)c2)cc1-c1cc(N2CCCC[C@@H]2C(F)(F)F)[nH]c(=O)c1]

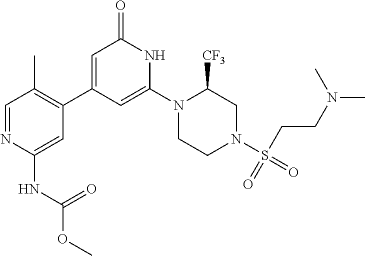 [SMILES: COC(=O)Nc1cc(-c2cc(N3CCN(S(=O)(=O)CCN(C)C)C[C@@H]3C(F)(F)F)[nH]c(=O)c2)c(C)cn1]

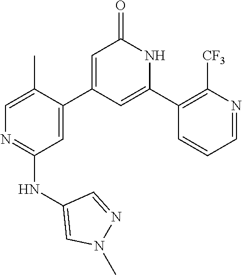 [SMILES: Cc1cnc(Nc2cnn(C)c2)cc1-c1cc(-c2cccnc2C(F)(F)F)[nH]c(=O)c1]

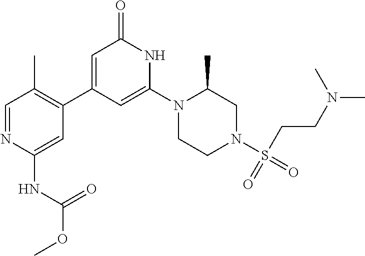 [SMILES: COC(=O)Nc1cc(-c2cc(N3CCN(S(=O)(=O)CCN(C)C)C[C@@H]3C)[nH]c(=O)c2)c(C)cn1]

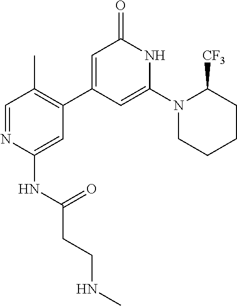 [SMILES: CNCCC(=O)Nc1cc(-c2cc(N3CCCC[C@@H]3C(F)(F)F)[nH]c(=O)c2)c(C)cn1]

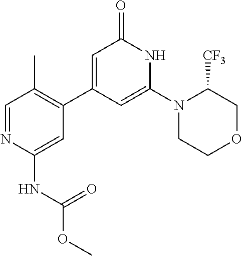 [SMILES: COC(=O)Nc1cc(-c2cc(N3CCOC[C@H]3C(F)(F)F)[nH]c(=O)c2)c(C)cn1]

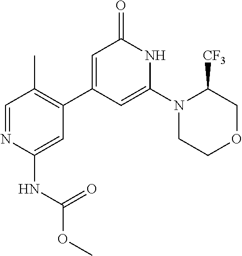 [SMILES: COC(=O)Nc1cc(-c2cc(N3CCOC[C@@H]3C(F)(F)F)[nH]c(=O)c2)c(C)cn1]

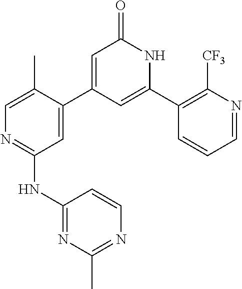 [SMILES: Cc1nccc(Nc2cc(-c3cc(-c4cccnc4C(F)(F)F)[nH]c(=O)c3)c(C)cn2)n1]

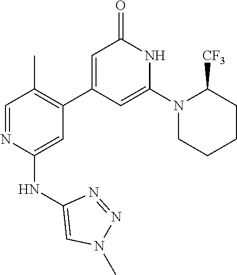 [SMILES: Cc1cnc(Nc2cn(C)nn2)cc1-c1cc(N2CCCC[C@@H]2C(F)(F)F)[nH]c(=O)c1]

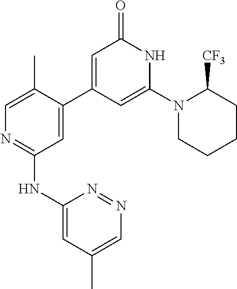 [SMILES: Cc1cnnc(Nc2cc(-c3cc(N4CCCC[C@@H]4C(F)(F)F)[nH]c(=O)c3)c(C)cn2)c1]

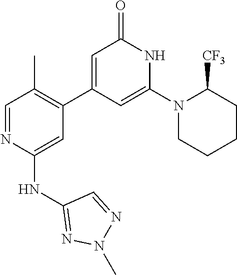 [SMILES: Cc1cnc(Nc2cnn(C)n2)cc1-c1cc(N2CCCC[C@@H]2C(F)(F)F)[nH]c(=O)c1]

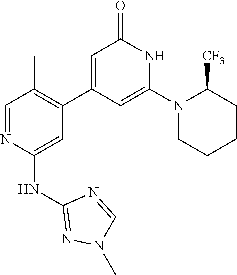 [SMILES: Cc1cnc(Nc2ncn(C)n2)cc1-c1cc(N2CCCC[C@@H]2C(F)(F)F)[nH]c(=O)c1]

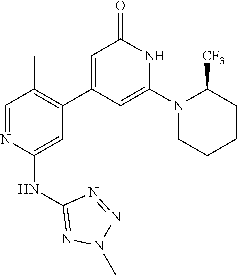 [SMILES: Cc1cnc(Nc2nnn(C)n2)cc1-c1cc(N2CCCC[C@@H]2C(F)(F)F)[nH]c(=O)c1]

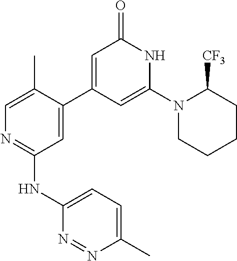 [SMILES: Cc1ccc(Nc2cc(-c3cc(N4CCCC[C@@H]4C(F)(F)F)[nH]c(=O)c3)c(C)cn2)nn1]

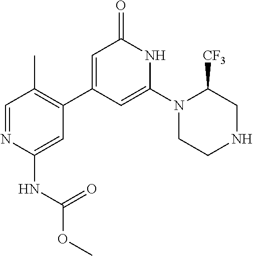 [SMILES: COC(=O)Nc1cc(-c2cc(N3CCNC[C@@H]3C(F)(F)F)[nH]c(=O)c2)c(C)cn1]

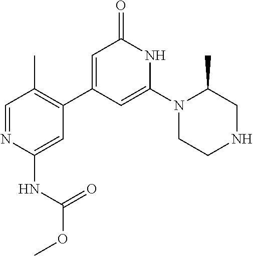 [SMILES: COC(=O)Nc1cc(-c2cc(N3CCNC[C@@H]3C)[nH]c(=O)c2)c(C)cn1]

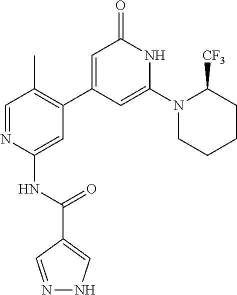 [SMILES: Cc1cnc(NC(=O)c2cn[nH]c2)cc1-c1cc(N2CCCC[C@@H]2C(F)(F)F)[nH]c(=O)c1]

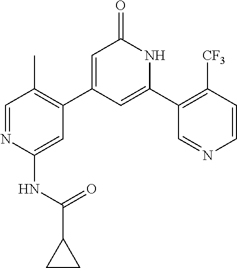 [SMILES: Cc1cnc(NC(=O)C2CC2)cc1-c1cc(-c2cnccc2C(F)(F)F)[nH]c(=O)c1]

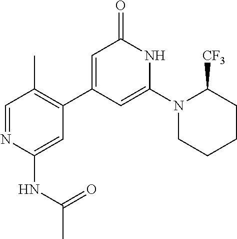 [SMILES: CC(=O)Nc1cc(-c2cc(N3CCCC[C@@H]3C(F)(F)F)[nH]c(=O)c2)c(C)cn1]

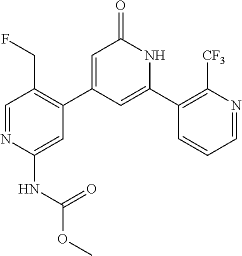 [SMILES: COC(=O)Nc1cc(-c2cc(-c3cccnc3C(F)(F)F)[nH]c(=O)c2)c(CF)cn1]

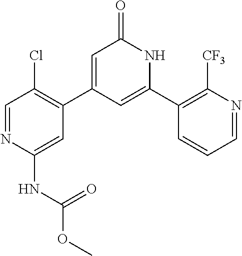 [SMILES: COC(=O)Nc1cc(-c2cc(-c3cccnc3C(F)(F)F)[nH]c(=O)c2)c(Cl)cn1]

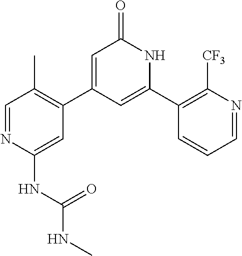 [SMILES: CNC(=O)Nc1cc(-c2cc(-c3cccnc3C(F)(F)F)[nH]c(=O)c2)c(C)cn1]

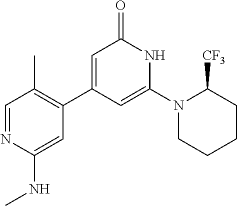 [SMILES: CNc1cc(-c2cc(N3CCCC[C@@H]3C(F)(F)F)[nH]c(=O)c2)c(C)cn1]

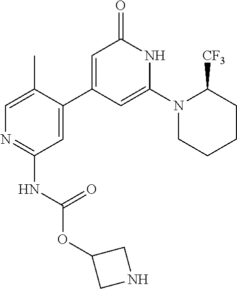 [SMILES: Cc1cnc(NC(=O)OC2CNC2)cc1-c1cc(N2CCCC[C@@H]2C(F)(F)F)[nH]c(=O)c1]

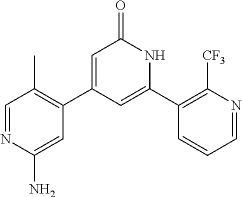 [SMILES: Cc1cnc(N)cc1-c1cc(-c2cccnc2C(F)(F)F)[nH]c(=O)c1]

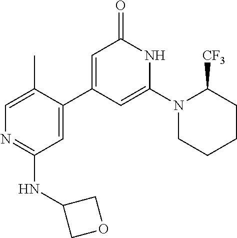 [SMILES: Cc1cnc(NC2COC2)cc1-c1cc(N2CCCC[C@@H]2C(F)(F)F)[nH]c(=O)c1]

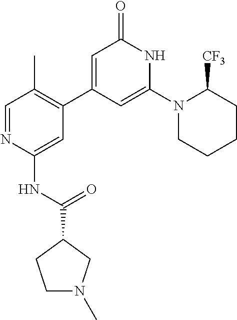 [SMILES: Cc1cnc(NC(=O)[C@H]2CCN(C)C2)cc1-c1cc(N2CCCC[C@@H]2C(F)(F)F)[nH]c(=O)c1]

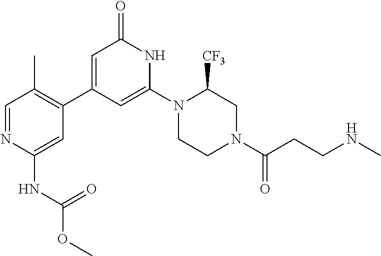 [SMILES: CNCCC(=O)N1CCN(c2cc(-c3cc(NC(=O)OC)ncc3C)cc(=O)[nH]2)[C@@H](C(F)(F)F)C1]

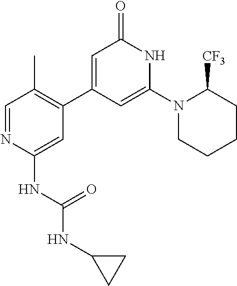 [SMILES: Cc1cnc(NC(=O)NC2CC2)cc1-c1cc(N2CCCC[C@@H]2C(F)(F)F)[nH]c(=O)c1]

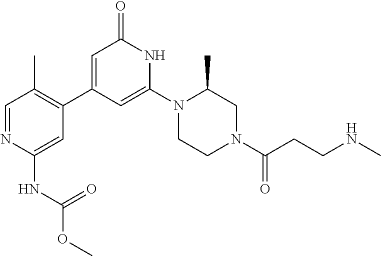 [SMILES: CNCCC(=O)N1CCN(c2cc(-c3cc(NC(=O)OC)ncc3C)cc(=O)[nH]2)[C@@H](C)C1]

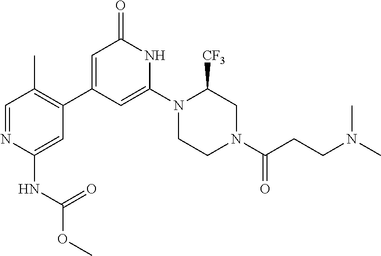 [SMILES: COC(=O)Nc1cc(-c2cc(N3CCN(C(=O)CCN(C)C)C[C@@H]3C(F)(F)F)[nH]c(=O)c2)c(C)cn1]

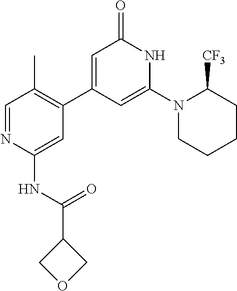 [SMILES: Cc1cnc(NC(=O)C2COC2)cc1-c1cc(N2CCCC[C@@H]2C(F)(F)F)[nH]c(=O)c1]

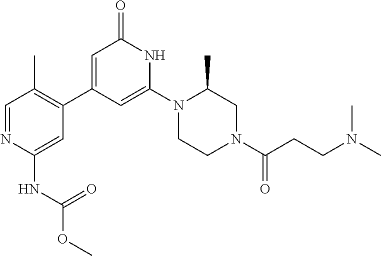 [SMILES: COC(=O)Nc1cc(-c2cc(N3CCN(C(=O)CCN(C)C)C[C@@H]3C)[nH]c(=O)c2)c(C)cn1]

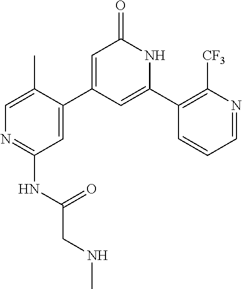 [SMILES: CNCC(=O)Nc1cc(-c2cc(-c3cccnc3C(F)(F)F)[nH]c(=O)c2)c(C)cn1]

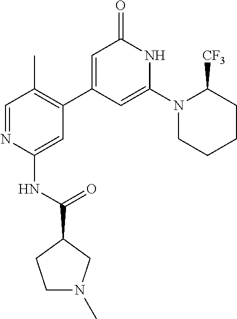 [SMILES: Cc1cnc(NC(=O)[C@@H]2CCN(C)C2)cc1-c1cc(N2CCCC[C@@H]2C(F)(F)F)[nH]c(=O)c1]

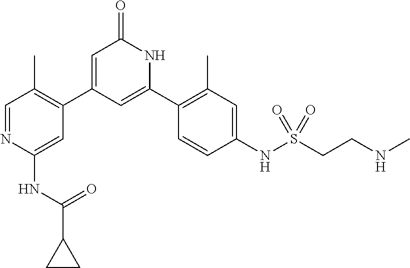 [SMILES: CNCCS(=O)(=O)Nc1ccc(-c2cc(-c3cc(NC(=O)C4CC4)ncc3C)cc(=O)[nH]2)c(C)c1]

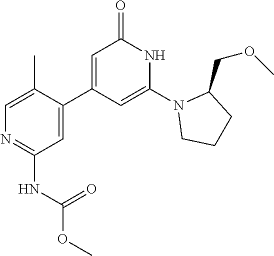 [SMILES: COC[C@H]1CCCN1c1cc(-c2cc(NC(=O)OC)ncc2C)cc(=O)[nH]1]

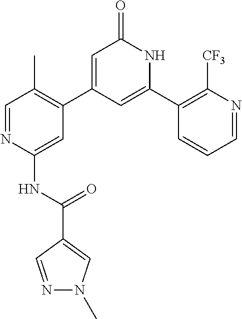 [SMILES: Cc1cnc(NC(=O)c2cnn(C)c2)cc1-c1cc(-c2cccnc2C(F)(F)F)[nH]c(=O)c1]

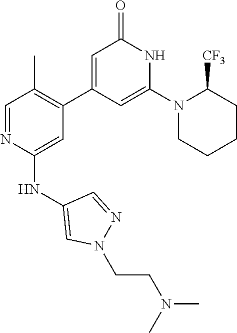 [SMILES: Cc1cnc(Nc2cnn(CCN(C)C)c2)cc1-c1cc(N2CCCC[C@@H]2C(F)(F)F)[nH]c(=O)c1]

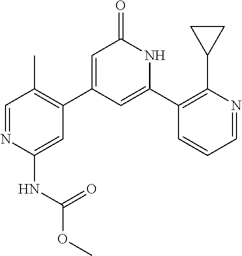 [SMILES: COC(=O)Nc1cc(-c2cc(-c3cccnc3C3CC3)[nH]c(=O)c2)c(C)cn1]

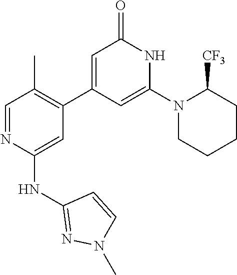 [SMILES: Cc1cnc(Nc2ccn(C)n2)cc1-c1cc(N2CCCC[C@@H]2C(F)(F)F)[nH]c(=O)c1]

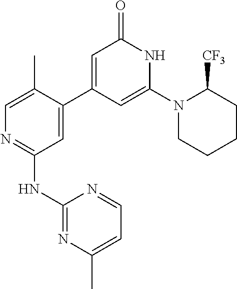 [SMILES: Cc1ccnc(Nc2cc(-c3cc(N4CCCC[C@@H]4C(F)(F)F)[nH]c(=O)c3)c(C)cn2)n1]

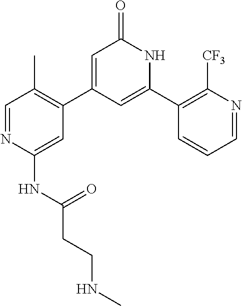 [SMILES: CNCCC(=O)Nc1cc(-c2cc(-c3cccnc3C(F)(F)F)[nH]c(=O)c2)c(C)cn1]

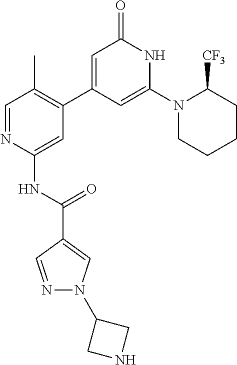 [SMILES: Cc1cnc(NC(=O)c2cnn(C3CNC3)c2)cc1-c1cc(N2CCCC[C@@H]2C(F)(F)F)[nH]c(=O)c1]

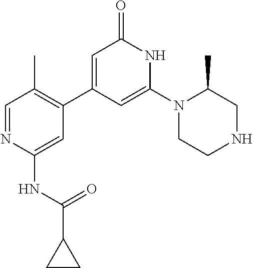 [SMILES: Cc1cnc(NC(=O)C2CC2)cc1-c1cc(N2CCNC[C@@H]2C)[nH]c(=O)c1]

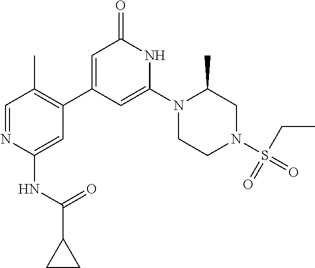 [SMILES: CCS(=O)(=O)N1CCN(c2cc(-c3cc(NC(=O)C4CC4)ncc3C)cc(=O)[nH]2)[C@@H](C)C1]

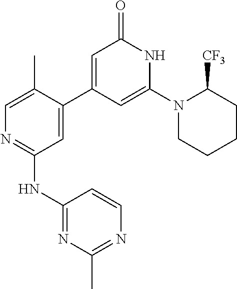 [SMILES: Cc1nccc(Nc2cc(-c3cc(N4CCCC[C@@H]4C(F)(F)F)[nH]c(=O)c3)c(C)cn2)n1]

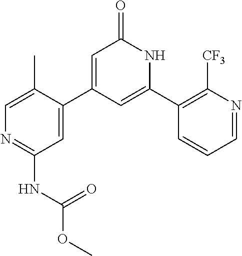 [SMILES: COC(=O)Nc1cc(-c2cc(-c3cccnc3C(F)(F)F)[nH]c(=O)c2)c(C)cn1]

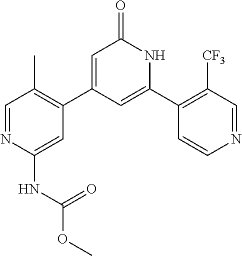 [SMILES: COC(=O)Nc1cc(-c2cc(-c3ccncc3C(F)(F)F)[nH]c(=O)c2)c(C)cn1]

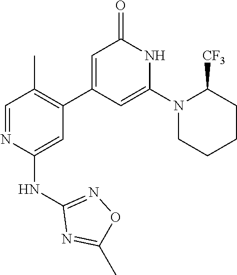 [SMILES: Cc1nc(Nc2cc(-c3cc(N4CCCC[C@@H]4C(F)(F)F)[nH]c(=O)c3)c(C)cn2)no1]

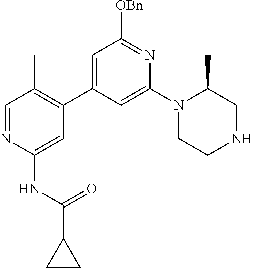 [SMILES: Cc1cnc(NC(=O)C2CC2)cc1-c1cc(OCc2ccccc2)nc(N2CCNC[C@@H]2C)c1]

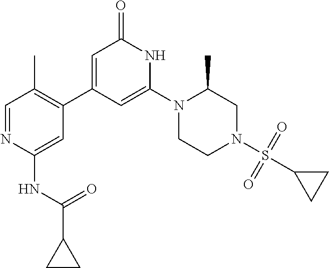 [SMILES: Cc1cnc(NC(=O)C2CC2)cc1-c1cc(N2CCN(S(=O)(=O)C3CC3)C[C@@H]2C)[nH]c(=O)c1]

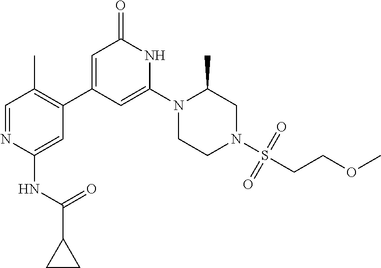 [SMILES: COCCS(=O)(=O)N1CCN(c2cc(-c3cc(NC(=O)C4CC4)ncc3C)cc(=O)[nH]2)[C@@H](C)C1]

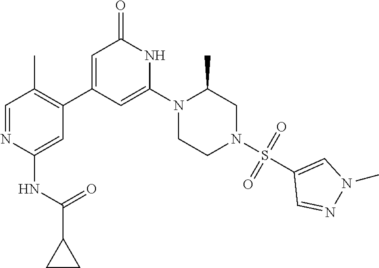 [SMILES: Cc1cnc(NC(=O)C2CC2)cc1-c1cc(N2CCN(S(=O)(=O)c3cnn(C)c3)C[C@@H]2C)[nH]c(=O)c1]

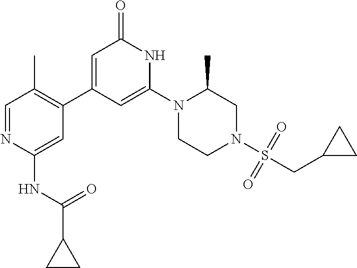 [SMILES: Cc1cnc(NC(=O)C2CC2)cc1-c1cc(N2CCN(S(=O)(=O)CC3CC3)C[C@@H]2C)[nH]c(=O)c1]

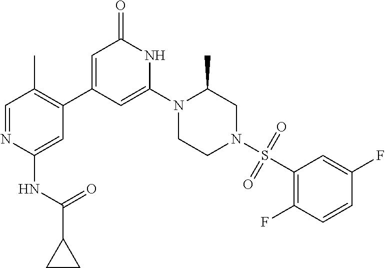 [SMILES: Cc1cnc(NC(=O)C2CC2)cc1-c1cc(N2CCN(S(=O)(=O)c3cc(F)ccc3F)C[C@@H]2C)[nH]c(=O)c1]

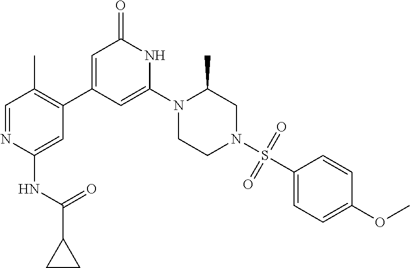 [SMILES: COc1ccc(S(=O)(=O)N2CCN(c3cc(-c4cc(NC(=O)C5CC5)ncc4C)cc(=O)[nH]3)[C@@H](C)C2)cc1]

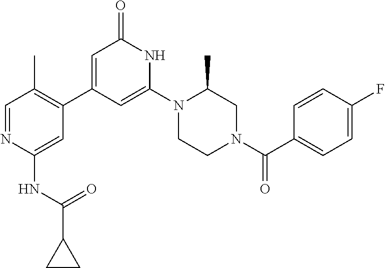 [SMILES: Cc1cnc(NC(=O)C2CC2)cc1-c1cc(N2CCN(C(=O)c3ccc(F)cc3)C[C@@H]2C)[nH]c(=O)c1]

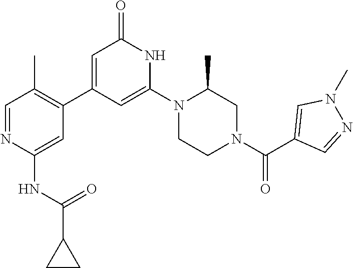 [SMILES: Cc1cnc(NC(=O)C2CC2)cc1-c1cc(N2CCN(C(=O)c3cnn(C)c3)C[C@@H]2C)[nH]c(=O)c1]

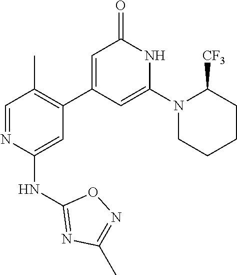 [SMILES: Cc1noc(Nc2cc(-c3cc(N4CCCC[C@@H]4C(F)(F)F)[nH]c(=O)c3)c(C)cn2)n1]

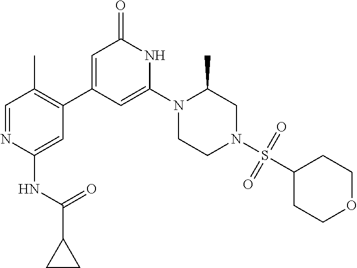 [SMILES: Cc1cnc(NC(=O)C2CC2)cc1-c1cc(N2CCN(S(=O)(=O)C3CCOCC3)C[C@@H]2C)[nH]c(=O)c1]

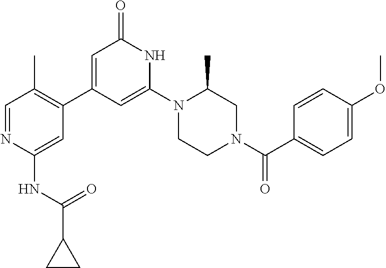 [SMILES: COc1ccc(C(=O)N2CCN(c3cc(-c4cc(NC(=O)C5CC5)ncc4C)cc(=O)[nH]3)[C@@H](C)C2)cc1]

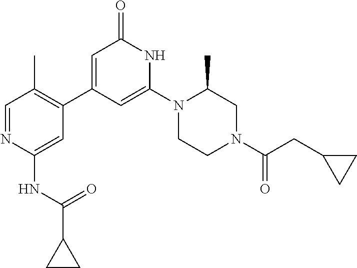 [SMILES: Cc1cnc(NC(=O)C2CC2)cc1-c1cc(N2CCN(C(=O)CC3CC3)C[C@@H]2C)[nH]c(=O)c1]

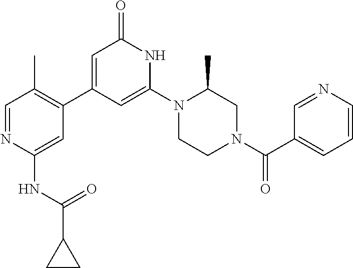 [SMILES: Cc1cnc(NC(=O)C2CC2)cc1-c1cc(N2CCN(C(=O)c3cccnc3)C[C@@H]2C)[nH]c(=O)c1]

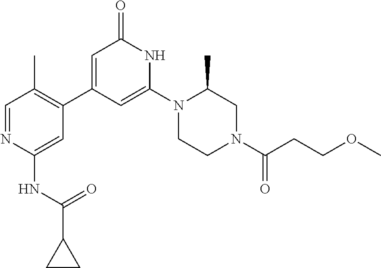 [SMILES: COCCC(=O)N1CCN(c2cc(-c3cc(NC(=O)C4CC4)ncc3C)cc(=O)[nH]2)[C@@H](C)C1]

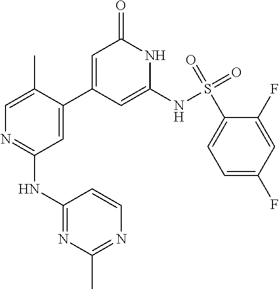 [SMILES: Cc1nccc(Nc2cc(-c3cc(NS(=O)(=O)c4ccc(F)cc4F)[nH]c(=O)c3)c(C)cn2)n1]

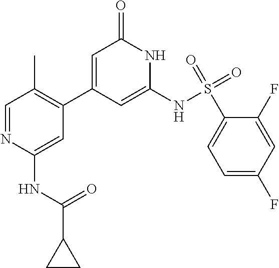 [SMILES: Cc1cnc(NC(=O)C2CC2)cc1-c1cc(NS(=O)(=O)c2ccc(F)cc2F)[nH]c(=O)c1]

and pharmaceutically acceptable salts, enantiomers, stereoisomers, and tautomers thereof.

### Pharmaceutical Compositions and Kits

Another aspect of this disclosure provides pharmaceutical compositions comprising compounds as disclosed herein, or pharmaceutically acceptable salts, enantiomers, stereoisomers, or tautomers thereof, formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid, or liquid form, which contains one or more of the compounds described herein, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound provided herein, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills, and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills, and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous, or organic solvents, or mixtures thereof, and powders. Liquid dosage forms for oral administration include pharmaceutically acceptable emulsions, microemulsions, solutions, suspensions, syrups, and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof. Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax, or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of the present disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another embodiment, provided are enteral pharmaceutical formulations including a disclosed compound, an enteric material, and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5.

Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives described herein.

Advantageously, provided herein are kits for use by a e.g., a consumer in need of treatment of cancer. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### Combination Therapy

Compounds described herein, e.g., a compound of Formula I, Formula I-A, Formula I-B, Formula I-C, Formula I-D, Formula I-E, Formula I-F, Formula I-G, Formula I-H, Formula I-I, or Formula I-J as defined herein, can be administered in combination with one or more additional therapeutic agents (e.g., one or more other additional agents described herein) to treat a disorder described herein, such as a cancer described herein. For example, provided in the present disclosure is a pharmaceutical composition comprising a compound described herein, e.g., a compound of Formula I, Formula I-A, Formula I-B, Formula I-C, Formula I-D, Formula I-E, Formula I-F, Formula I-G, Formula I-H, Formula I-I, or Formula I-J as defined herein, one or more additional therapeutic agents, and a pharmaceutically acceptable excipient. In some embodiments, a compound of Formula I, Formula I-A, Formula I-B, Formula I-C, Formula I-D, Formula I-E, Formula I-F, Formula I-G, Formula I-H, Formula I-I, or Formula I-J as defined herein and one additional therapeutic agent is administered. In some embodiments, a compound of Formula I, Formula I-A, Formula I-B, Formula I-C, Formula I-D, Formula I-E, Formula I-F, Formula I-G, Formula I-H, Formula I-I, or Formula I-J as defined herein and two additional therapeutic agents are administered. In some embodiments, a compound of Formula I, Formula I-A, Formula I-B, Formula I-C, Formula I-D, Formula I-E, Formula I-F, Formula I-G, Formula I-H, Formula I-I, or Formula I-J as defined herein and three additional therapeutic agents are administered. Combination therapy can be achieved by administering two or more therapeutic agents, each of which is formulated and administered separately. For example, a compound of Formula I, Formula I-A, Formula I-B, Formula I-C, Formula I-D, Formula I-E, Formula I-F, Formula I-G, Formula I-H, Formula I-I, or Formula I-J as defined herein and an additional therapeutic agent can be formulated and administered separately. Combination therapy can also be achieved by administering two or more therapeutic agents in a single formulation, for example a pharmaceutical composition comprising a compound of Formula I, Formula I-A, Formula I-B, Formula I-C, Formula I-D, Formula I-E, Formula I-F, Formula I-G, Formula I-H, Formula I-I, or Formula I-J as one therapeutic agent and one or more additional therapeutic agents such as an antibiotic, a viral protease inhibitor, or an anti-viral nucleoside anti-metabolite.

For example, a compound of Formula I, Formula I-A, Formula I-B, Formula I-C, Formula I-D, Formula I-E, Formula I-F, Formula I-G, Formula I-H, Formula I-I, or Formula I-J as defined herein and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc.

Examples of additional therapeutics agents that can be used in combination with a VPS34 inhibitor such as a compound of Formula I, Formula I-A, Formula I-B, Formula I-C, Formula I-D, Formula I-E, Formula I-F, Formula I-G, Formula I-H, Formula I-I, or Formula I-J as defined herein, includes, but not limited to, a STING agonist (e.g., DMXAA, ADU-S100, or pharmaceutically acceptable salt thereof), an anti-PD-1 therapeutic, an anti PD-L1 therapeutic, or a CTLA4 inhibitor.

### Methods of Use

Provided herein, in some embodiments, are methods of treating cancer in a patient in need thereof, comprising administering to the patient a therapeutically effective amount of the compound disclosed herein, or pharmaceutically acceptable salts, enantiomers, stereoisomers, or tautomers thereof, or of the pharmaceutical composition disclosed herein.

In some embodiments, the cancer is selected from the group consisting of breast cancer, such as triple negative breast cancer, bladder cancer, liver cancer, cervical cancer, pancreatic cancer, leukemia, lymphoma, renal cancer, colon cancer, glioma, prostate cancer, ovarian cancer, melanoma and lung cancer, gastrointestinal stromal tumors, esophageal cancer, gastric cancer, glioma, glioblastoma, ovarian cancer, head cancer, neck cancer, urothelial cancer, uterine cancer, prostate cancer, hepatic cancer, an osteosarcoma, a sarcoma, multiple myeloma, a cancer that is metastatic to bone, and a papillary thyroid carcinoma, as well as hypoxic tumors.

In some embodiments, the method further comprises radiation therapy.

Also provided herein, in some embodiments, are methods of treating type II diabetes in a patient in need thereof, comprising administering to the patient a therapeutically effective amount of the compound disclosed herein, or pharmaceutically acceptable salts, enantiomers, stereoisomers, or tautomers thereof, or of the pharmaceutical composition disclosed herein.

Provided herein, in some embodiments, are methods of treating a disease selected from inflammatory diseases, neurodegenerative disorders, autoimmune diseases and viral infections, comprising administering to the patient a therapeutically effective amount of the compound disclosed herein, or pharmaceutically acceptable salts, enantiomers, stereoisomers, or tautomers thereof, or of the pharmaceutical composition disclosed herein.

In some embodiments, provided herein are methods of treating a viral infection, comprising administering to the patient a therapeutically effect amount of the compound disclosed herein, or pharmaceutically acceptable salts, enantiomers, stereoisomers, or tautomers thereof, or of the pharmaceutical composition disclosed herein.

In some embodiments, the viral infection is a caused by a coronavirus. In some embodiments, the viral infection is caused by a virus selected from the group consisting of a coronavirus, a rhinovirus and a flavivirus. In some embodiments, the viral infection is caused by a rhinovirus. In some embodiments, the viral infection is caused by a flavivirus.

In some embodiments, the viral infection is caused by a coronavirus selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2. In some embodiments, the viral infection is caused by SARS. In some embodiments, the viral infection is caused by SARS-CoV. In some embodiments, the viral infection is caused by SARS-CoV-2. In some embodiments, the viral infection is caused by MERS-CoV. In some embodiments, the viral infection is COVID-19.

In some embodiments, the viral infection is caused by a positive RNA virus. In some embodiments, the virus is a positive-sense RNA virus. In some embodiments, the virus is a sense RNA virus. In some embodiments, the virus is a sense-strand RNA virus. In some embodiments, the virus a positive-strand RNA virus. In some embodiments, the virus is a positive (+) RNA virus. In some embodiments, the virus is a positive-sense single-stranded RNA virus. In some embodiments, the positive RNA virus is selected from the group consisting of a virus of the Coronaviridae family, a virus of the Flaviviridae family, and a virus of the Picornaviridae family. In some embodiments, the positive RNA virus is selected from the group consisting of a rhinovirus, a flavivirus, a picornavirus, and a coronavirus. In some embodiments, the positive RNA virus is a picornavirus. In some embodiments, the positive RNA virus is a rhinovirus. In some embodiments, the positive RNA virus is a human rhinovirus. In some embodiments, the positive RNA virus is a flavivirus. In some embodiments, the positive RNA virus is coronavirus. In some embodiments, the positive RNA virus is selected from the group consisting of SARS CoV-1, SARS CoV-2, MERS, hepatitis C (HCV), rhinovirus, Dengue virus, Zika virus, and West Nile virus. In some embodiments, the positive RNA virus is a coronavirus. In some embodiments, the coronavirus is selected from the group consisting of SARS CoV-1, SARS CoV-2 and MERS. In some embodiments, the coronavirus is SARS CoV-1. In some embodiments, the coronavirus is SARS-CoV-2. In some embodiments, the positive RNA virus (e.g., coronavirus) is of any variant resulting from mutation or novel variants emerging from other species (e.g., species of mammals, e.g., a mink). In some embodiments, the positive RNA virus is MERS. In some embodiments, the positive RNA virus is hepatitis C. In some embodiments, the positive RNA virus is Zika virus. In some embodiments, the positive RNA virus is Dengue virus. In some embodiments, the positive RNA virus is West Nile virus.

In some embodiments, the viral infection is a respiratory viral infection. In some embodiments, the viral infection is an upper respiratory viral infection or a lower respiratory viral infection.

In some embodiments, the method further comprises administering a therapeutically effective amount of one or more other agents or compositions to the patient.

In some embodiments, the one or more other additional agents is selected from the group consisting of ribavirin, favipiravir, ST-193, oseltamivir, zanamivir, peramivir, danoprevir, ritonavir, and remdesivir.

In some embodiments, the one or more other additional agents is selected from the group consisting of protease inhibitors, fusion inhibitors, M2 proton channel blockers, polymerase inhibitors, 6-endonuclease inhibitors, neuraminidase inhibitors, reverse transcriptase inhibitor, aciclovir, acyclovir, protease inhibitors, arbidol, atazanavir, atripla, boceprevir, cidofovir, combivir, darunavir, docosanol, edoxudine, entry inhibitors, entecavir, famciclovir, fomivirsen, fosamprenavir, foscarnet, fosfonet, ganciclovir, ibacitabine, immunovir, idoxuridine, imiquimod, inosine, integrase inhibitor, interferons, lopinavir, loviride, moroxydine, nexavir, nucleoside analogues, penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine.

In some embodiments, the one or more other additional agents is selected from the group consisting of lamivudine, an interferon alpha, a VAP anti-idiotypic antibody, enfuvirtide, amantadine, rimantadine, pleconaril, aciclovir, zidovudine, fomivirsen, a protease inhibitor, double-stranded RNA activated caspase oligomerizer (DRACO), rifampicin, zanamivir, oseltamivir, danoprevir, ritonavir, and remdesivir.

In some embodiments, the one or more other additional agents is selected from the group consisting of quinine (optionally in combination with clindamycin), chloroquine, amodiaquine, artemisinin and its derivatives, doxycycline, pyrimethamine, mefloquine, halofantrine, hydroxychloroquine, eflornithine, nitazoxanide, ornidazole, paromomycin, pentamidine, primaquine, pyrimethamine, proguanil (optionally in combination with atovaquone), a sulfonamide, tafenoquine, tinidazole and a PPT1 inhibitor.

In some embodiments, the one or more other additional agents is an RNA polymerase inhibitor. In some embodiments, the RNA polymerase inhibitor is selected from the group consisting of remdesivir, sofosbuvir, 7-deaza-2-CMA, galidesvir, and AT-527. In some embodiments, the RNA polymerase inhibitor is remdesivir.

In some embodiments, the one or more other additional agents is selected from the group consisting of a TMPRSS protease inhibitor, a lysosomal blocking agent (e.g., hydroxychloroquine), a PIKfyve inhibitor (e.g., apilimod), an anti-SARSCOV-2 antibody, a cocktail of anti-SARSCOV-2 antibodies, an anti-inflammatory agent, an anti-TNF agent (e.g., adalimumab, infliximab, etanercept, golimumab, or certolizumab), a histimine H1/H2 blocker (e.g., famotidine, nizatidine, ranitidine, and cimetidine), a steroid, an anti-coagulant, a complement targeting agent, a statin, and an ACE inhibitor. In some embodiments, TMPRSS protease inhibitor is selected from the group consisting of a TMPRSS4 inhibitor, a TMPRSS11A inhibitor, a TMPRSS11D inhibitor, TMPRSS11E1 inhibitor, and a TMPRSS2 inhibitor. In some embodiments, the TMPRSS protease inhibitor is a TMRSS2 protease inhibitor. In some embodiments, the TMRESS-2 protease inhibitor is selected from camostat and nafamostat. In some embodiments, the anti-SARSCOV-2 antibody is selected from LY-CoV555 (bamlanivimab) and LY-CoVO16 (etesevimab). In some embodiments, the cocktail of anti-SARSCOV-2 antibodies is REGN-COV2. In some embodiments, the anti-inflammatory agent is an IL-6 antagonist (e.g., siltuximab, sarilumab, olokizumab, BMS-945429, sirukumab, and clazakizumab). In some embodiments, the steroid is dexamethasone. In some embodiments, the anti-coagulant is low-molecular weight heparin. In some embodiments, the complement targeting agent is eculizumab. In some embodiments, the statin is selected from the group consisting of atorvastatin, fluvastatin, lovastatin, pitavastatin, pravastatin, rosuvastatin, and simvastatin.

In some embodiments, the ACE inhibitor is selected from the group consisting of benazepril, captopril enalapril/enalaprilat, fosinopril, lisinopril moexipril, perindopril quinapril, and ramipril.

In some embodiments, the one or more other additional agents is selected from the group consisting of remdesivir, camostat, nafamostat, hydroxychloroquine, chloroquine, apilimod, LY-CoV555 (bamlanivimab), LY-CoVO16 (etesevimab), REGN-COV2, tocilizumab, siltuximab, sarilumab, olokizumab, BMS-945429, sirukumab, clazakizumab, adalimumab, infliximab, etanercept, golimumab, certolizumab, famotidine, nizatidine, ranitidine, cimetidine, dexamethasone, low molecular weight heparin, eculizumab, atorvastatin, fluvastatin, lovastatin, pitavastatin, pravastatin, rosuvastatin, simvastatin, benazepril, captopril enalapril/enalaprilat, fosinopril, lisinopril moexipril, perindopril quinapril, and ramipril.

In some embodiments, the method comprises administering one or more one or more other additional agents selected from the group consisting of remdesivir, sofosbuvir, 7-deaza-2-CMA, galidesvir, AT-527, temoporfin, novobiocin, curcumin, voxilaprevir, grazopevir, glecaprevir,camostat, nafamostat, hydroxychloroquine, chloroquine, apilimod, imatinib, dasatinib, ponatinib, velpatasvir, ledipasvir, elbasivir, pibrentasvir, NITD008, LY-CoV555 (bamlanivimab), LY-CoVO16 (etesevimab), REGN-COV2, tocilizumab, siltuximab, sarilumab, olokizumab, BMS-945429, sirukumab, clazakizumab, adalimumab, infliximab, etanercept, golimumab, certolizumab, famotidine, nizatidine, ranitidine, cimetidine, dexamethasone, low molecular weight heparin, eculizumab, atorvastatin, fluvastatin, lovastatin, pitavastatin, pravastatin, rosuvastatin, simvastatin, benazepril, captopril enalapril/enalaprilat, fosinopril, lisinopril moexipril, perindopril quinapril, ramipril, and adoptive NK cell therapy.

In some embodiments, the one or more other additional agents is selected from the group consisting of a ABL inhibitor and a JAK inhibitor.

In some embodiments, the one or more other additional agents is an ABL inhibitor (e.g., imatinib, dasatinib, or ponatinib). In some embodiments, the ABL inhibitor is selected from the group consisting of imatinib, dasatinib, and ponatinib. In some embodiments, the ABL inhibitor is imatinib. In some embodiments, the ABL inhibitor is dasatinib. In some embodiments, the ABL inhibitor is ponatinib.

In some embodiments, the one or more other additional agents is a JAK inhibitor.

In some embodiments, the JAK inhibitor is selected from the group consisting of baricitinib, ruxolitinib, tofacitinib, and upadacitinib. In some embodiments, the JAK inhibitor is baricitinib. In some embodiments, the JAK inhibitor is ruxolitinib. In some embodiments, the JAK inhibitor is tofacitinib. In some embodiments, the JAK inhibitor is upadacitinib.

In some embodiments, the one or more other additional agents is a protease inhibitor. In embodiments, the protease inhibitor is selected from the group consisting of temoporfin, novobiocin, curcumin, voxilaprevir, grazopevir, and glecaprevir.

In some embodiments, the one or more other additional agents is an NS5A inhibitor. In embodiments, the NS5A inhibitor is selected from the group consisting of velpatasvir, ledipasvir, elbasivir, and pibrentasvir.

In some embodiments, the one or more other additional agents is a pyrimidine synthesis inhibitor. In some embodiments, the pyrimidine synthesis inhibitor is NITD008.

In some embodiments, the one or more other additional agents is an adoptive natural killer (NK) cell therapy. In some embodiments, the additional therapeutic agent is a vaccine. In some embodiments, the vaccine is a coronavirus vaccine. In some embodiments, the vaccine is selected from the group consisting of BNT162b2, mRNA-1273, AZD1222, and Ad26.COV2.S. In some embodiments, the vaccine is a protein-based vaccine. In some embodiments, the vaccine is an RNA-based vaccine. In some embodiments, the vaccine is an attenuated virus vaccine. In some embodiments, the vaccine is an inactivated virus vaccine. In some embodiments, the vaccine is a non-replicating viral vector vaccine.

In some embodiments, the compound described herein is orally administered to the patient. In some embodiments, the compound described herein is parenterally administered to the patient.

In some embodiments, provided herein is a compound disclosed herein, or pharmaceutically acceptable salts, enantiomers, stereoisomers, or tautomers thereof, or of the pharmaceutical composition disclosed herein for use in the treatment or prophylaxis of a disease.

In some embodiments, provided herein is a compound disclosed herein, or pharmaceutically acceptable salts, enantiomers, stereoisomers, or tautomers thereof, or a pharmaceutical composition disclosed herein, for use in treating cancer.

In some embodiments, the cancer is selected from the group consisting of breast cancer, such as triple negative breast cancer, bladder cancer, liver cancer, cervical cancer, pancreatic cancer, leukemia, lymphoma, renal cancer, colon cancer, glioma, prostate cancer, ovarian cancer, melanoma and lung cancer, gastrointestinal stromal tumors, esophageal cancer, gastric cancer, glioma, glioblastoma, ovarian cancer, head cancer, neck cancer, urothelial cancer, uterine cancer, prostate cancer, hepatic cancer, an osteosarcoma, a sarcoma, multiple myeloma, a cancer that is metastatic to bone, and a papillary thyroid carcinoma, as well as hypoxic tumors.

Also provided herein, in some embodiments, is a compound disclosed herein, or pharmaceutically acceptable salts, enantiomers, stereoisomers, or tautomers thereof, or a pharmaceutical composition disclosed herein, for use in treating cancer, wherein said cancer treatment further comprises radiation therapy.

Also provided herein, in some embodiments, is a compound disclosed herein, or pharmaceutically acceptable salts, enantiomers, stereoisomers, or tautomers thereof, or a pharmaceutical composition disclosed herein, for use in treating type II diabetes.

Provided herein, in some embodiments, is a compound disclosed herein, or pharmaceutically acceptable salts, enantiomers, stereoisomers, or tautomers thereof, or a pharmaceutical composition disclosed herein, for use in treating a disease selected from inflammatory diseases, neurodegenerative disorders, cardiovascular disorders, autoimmune diseases and viral infections.

In some embodiments, provided herein is a compound disclosed herein, or pharmaceutically acceptable salts, enantiomers, stereoisomers, or tautomers thereof, or a pharmaceutical composition disclosed herein, for use in treating a viral infection.

In some embodiments, the viral infection is a caused by a coronavirus. In some embodiments, the viral infection is caused by a virus selected from the group consisting of a coronavirus, a rhinovirus and a flavivirus. In some embodiments, the viral infection is caused by a rhinovirus. In some embodiments, the viral infection is caused by a flavivirus.

In some embodiments, the viral infection is caused by a coronavirus selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2. In some embodiments, the viral infection is caused by SARS. In some embodiments, the viral infection is caused by SARS-CoV. In some embodiments, the viral infection is caused by SARS-CoV-2. In some embodiments, the viral infection is caused by MERS-CoV. In some embodiments, the viral infection is COVID-19.

In some embodiments, the viral infection is caused by a positive RNA virus. In some embodiments, the virus is a positive-sense RNA virus. In some embodiments, the virus is a sense RNA virus. In some embodiments, the virus is a sense-strand RNA virus. In some embodiments, the virus a positive-strand RNA virus. In some embodiments, the virus is a positive (+) RNA virus. In some embodiments, the virus is a positive-sense single-stranded RNA virus. In some embodiments, the positive RNA virus is selected from the group consisting of a virus of the Coronaviridae family, a virus of the Flaviviridae family, and a virus of the Picornaviridae family. In some embodiments, the positive RNA virus is selected from the group consisting of a rhinovirus, a flavivirus, a picornavirus, and a coronavirus. In some embodiments, the positive RNA virus is a picornavirus. In some embodiments, the positive RNA virus is a rhinovirus. In some embodiments, the positive RNA virus is a human rhinovirus. In some embodiments, the positive RNA virus is a flavivirus. In some embodiments, the positive RNA virus is coronavirus. In some embodiments, the positive RNA virus is selected from the group consisting of SARS CoV-1, SARS CoV-2, MERS, hepatitis C (HCV), rhinovirus, Dengue virus, Zika virus, and West Nile virus. In some embodiments, the positive RNA virus is a coronavirus. In some embodiments, the coronavirus is selected from the group consisting of SARS CoV-1, SARS CoV-2 and MERS. In some embodiments, the coronavirus is SARS CoV-1. In some embodiments, the coronavirus is SARS-CoV-2. In some embodiments, the positive RNA virus (e.g., coronavirus) is of any variant resulting from mutation or novel variants emerging from other species (e.g., species of mammals, e.g., a mink). In some embodiments, the positive RNA virus is MERS. In some embodiments, the positive RNA virus is hepatitis C. In some embodiments, the positive RNA virus is Zika virus. In some embodiments, the positive RNA virus is Dengue virus. In some embodiments, the positive RNA virus is West Nile virus.

In some embodiments, the viral infection is a respiratory viral infection. In some embodiments, the viral infection is an upper respiratory viral infection or a lower respiratory viral infection.

In some embodiments, the use further comprises administering a therapeutically effective amount of one or more other agents or compositions to the patient.

In some embodiments, the one or more other additional agents is selected from the group consisting of ribavirin, favipiravir, ST-193, oseltamivir, zanamivir, peramivir, danoprevir, ritonavir, and remdesivir.

In some embodiments, the one or more other additional agents is selected from the group consisting of protease inhibitors, fusion inhibitors, M2 proton channel blockers, polymerase inhibitors, 6-endonuclease inhibitors, neuraminidase inhibitors, reverse transcriptase inhibitor, aciclovir, acyclovir, protease inhibitors, arbidol, atazanavir, atripla, boceprevir, cidofovir, combivir, darunavir, docosanol, edoxudine, entry inhibitors, entecavir, famciclovir, fomivirsen, fosamprenavir, foscarnet, fosfonet, ganciclovir, ibacitabine, immunovir, idoxuridine, imiquimod, inosine, integrase inhibitor, interferons, lopinavir, loviride, moroxydine, nexavir, nucleoside analogues, penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine.

In some embodiments, the one or more other additional agents is selected from the group consisting of lamivudine, an interferon alpha, a VAP anti-idiotypic antibody, enfuvirtide, amantadine, rimantadine, pleconaril, aciclovir, zidovudine, fomivirsen, a protease inhibitor, double-stranded RNA activated caspase oligomerizer (DRACO), rifampicin, zanamivir, oseltamivir, danoprevir, ritonavir, and remdesivir.

In some embodiments, the one or more other additional agents is selected from the group consisting of quinine (optionally in combination with clindamycin), chloroquine, amodiaquine, artemisinin and its derivatives, doxycycline, pyrimethamine, mefloquine, halofantrine, hydroxychloroquine, eflornithine, nitazoxanide, ornidazole, paromomycin, pentamidine, primaquine, pyrimethamine, proguanil (optionally in combination with atovaquone), a sulfonamide, tafenoquine, tinidazole and a PPT1 inhibitor.

In some embodiments, the one or more other additional agents is an RNA polymerase inhibitor. In some embodiments, the RNA polymerase inhibitor is selected from the group consisting of remdesivir, sofosbuvir, 7-deaza-2-CMA, galidesvir, and AT-527. In some embodiments, the RNA polymerase inhibitor is remdesivir.

In some embodiments, the one or more other additional agents is selected from the group consisting of a TMPRSS protease inhibitor, a lysosomal blocking agent (e.g., hydroxychloroquine), a PIKfyve inhibitor (e.g., apilimod), an anti-SARSCOV-2 antibody, a cocktail of anti-SARSCOV-2 antibodies, an anti-inflammatory agent, an anti-TNF agent (e.g., adalimumab, infliximab, etanercept, golimumab, or certolizumab), a histimine H1/H2 blocker (e.g., famotidine, nizatidine, ranitidine, and cimetidine), a steroid, an anti-coagulant, a complement targeting agent, a statin, and an ACE inhibitor. In some embodiments, TMPRSS protease inhibitor is selected from the group consisting of a TMPRSS4 inhibitor, a TMPRSS11A inhibitor, a TMPRSS11D inhibitor, TMPRSS11E1 inhibitor, and a TMPRSS2 inhibitor. In some embodiments, the TMPRSS protease inhibitor is a TMRSS2 protease inhibitor. In some embodiments, the TMRESS-2 protease inhibitor is selected from camostat and nafamostat. In some embodiments, the anti-SARSCOV-2 antibody is selected from LY-CoV555 (bamlanivimab) and LY-CoVO16 (etesevimab). In some embodiments, the cocktail of anti-SARSCOV-2 antibodies is REGN-COV2. In some embodiments, the anti-inflammatory agent is an IL-6 antagonist (e.g., siltuximab, sarilumab, olokizumab, BMS-945429, sirukumab, and clazakizumab). In some embodiments, the steroid is dexamethasone. In some embodiments, the anti-coagulant is low-molecular weight heparin. In some embodiments, the complement targeting agent is eculizumab. In some embodiments, the statin is selected from the group consisting of atorvastatin, fluvastatin, lovastatin, pitavastatin, pravastatin, rosuvastatin, and simvastatin.

In some embodiments, the ACE inhibitor is selected from the group consisting of benazepril, captopril enalapril/enalaprilat, fosinopril, lisinopril moexipril, perindopril quinapril, and ramipril.

In some embodiments, the one or more other additional agents is selected from the group consisting of remdesivir, camostat, nafamostat, hydroxychloroquine, chloroquine, apilimod, LY-CoV555 (bamlanivimab), LY-CoVO16 (etesevimab), REGN-COV2, tocilizumab, siltuximab, sarilumab, olokizumab, BMS-945429, sirukumab, clazakizumab, adalimumab, infliximab, etanercept, golimumab, certolizumab, famotidine, nizatidine, ranitidine, cimetidine, dexamethasone, low molecular weight heparin, eculizumab, atorvastatin, fluvastatin, lovastatin, pitavastatin, pravastatin, rosuvastatin, simvastatin, benazepril, captopril enalapril/enalaprilat, fosinopril, lisinopril moexipril, perindopril quinapril, and ramipril.

In some embodiments, the use comprises administering one or more one or more other additional agents selected from the group consisting of remdesivir, sofosbuvir, 7-deaza-2-CMA, galidesvir, AT-527, temoporfin, novobiocin, curcumin, voxilaprevir, grazopevir, glecaprevir,camostat, nafamostat, hydroxychloroquine, chloroquine, apilimod, imatinib, dasatinib, ponatinib, velpatasvir,ledipasvir, elbasivir, pibrentasvir, NITD008, LY-CoV555 (bamlanivimab), LY-CoVO16 (etesevimab), REGN-COV2, tocilizumab, siltuximab, sarilumab, olokizumab, BMS-945429, sirukumab, clazakizumab, adalimumab, infliximab, etanercept, golimumab, certolizumab, famotidine, nizatidine, ranitidine, cimetidine, dexamethasone, low molecular weight heparin, eculizumab, atorvastatin, fluvastatin, lovastatin, pitavastatin, pravastatin, rosuvastatin, simvastatin, benazepril, captopril enalapril/enalaprilat, fosinopril, lisinopril moexipril, perindopril quinapril, ramipril, and adoptive NK cell therapy.

In some embodiments, the one or more other additional agents is selected from the group consisting of a ABL inhibitor and a JAK inhibitor.

In some embodiments, the one or more other additional agents is an ABL inhibitor (e.g., imatinib, dasatinib, or ponatinib). In some embodiments, the ABL inhibitor is selected from the group consisting of imatinib, dasatinib, and ponatinib. In some embodiments, the ABL inhibitor is imatinib. In some embodiments, the ABL inhibitor is dasatinib. In some embodiments, the ABL inhibitor is ponatinib.

In some embodiments, the one or more other additional agents is a JAK inhibitor.

In some embodiments, the JAK inhibitor is selected from the group consisting of baricitinib, ruxolitinib, tofacitinib, and upadacitinib. In some embodiments, the JAK inhibitor is baricitinib. In some embodiments, the JAK inhibitor is ruxolitinib. In some embodiments, the JAK inhibitor is tofacitinib. In some embodiments, the JAK inhibitor is upadacitinib.

In some embodiments, the one or more other additional agents is a protease inhibitor. In embodiments, the protease inhibitor is selected from the group consisting of temoporfin, novobiocin, curcumin, voxilaprevir, grazopevir, and glecaprevir.

In some embodiments, the one or more other additional agents is an NS5A inhibitor. In embodiments, the NS5A inhibitor is selected from the group consisting of velpatasvir, ledipasvir, elbasivir, and pibrentasvir.

In some embodiments, the one or more other additional agents is a pyrimidine synthesis inhibitor. In some embodiments, the pyrimidine synthesis inhibitor is NITD008.

In some embodiments, the one or more other additional agents is an adoptive natural killer (NK) cell therapy. In some embodiments, the additional therapeutic agent is a vaccine. In some embodiments, the vaccine is a coronavirus vaccine. In some embodiments, the vaccine is selected from the group consisting of BNT162b2, mRNA-1273, AZD1222, and Ad26.COV2.S. In some embodiments, the vaccine is a protein-based vaccine. In some embodiments, the vaccine is an RNA-based vaccine. In some embodiments, the vaccine is an attenuated virus vaccine. In some embodiments, the vaccine is an inactivated virus vaccine. In some embodiments, the vaccine is a non-replicating viral vector vaccine.

In some embodiments, the compound described herein is orally administered to the patient. In some embodiments, the compound described herein is parenterally administered to the patient.

In some embodiments, provided herein is use of a compound disclosed herein, or pharmaceutically acceptable salts, enantiomers, stereoisomers, or tautomers thereof, or a pharmaceutical composition disclosed herein, in the preparation of a medicament for treating cancer.

In some embodiments, the cancer is selected from the group consisting of breast cancer, such as triple negative breast cancer, bladder cancer, liver cancer, cervical cancer, pancreatic cancer, leukemia, lymphoma, renal cancer, colon cancer, glioma, prostate cancer, ovarian cancer, melanoma and lung cancer, gastrointestinal stromal tumors, esophageal cancer, gastric cancer, glioma, glioblastoma, ovarian cancer, head cancer, neck cancer, urothelial cancer, uterine cancer, prostate cancer, hepatic cancer, an osteosarcoma, a sarcoma, multiple myeloma, a cancer that is metastatic to bone, and a papillary thyroid carcinoma, as well as hypoxic tumors.

In some embodiments, the use further comprises administering radiation therapy.

In some embodiments, provided herein is use of a compound disclosed herein, or pharmaceutically acceptable salts, enantiomers, stereoisomers, or tautomers thereof, or a pharmaceutical composition disclosed herein, in the preparation of a medicament for treating type II diabetes.

Also provided herein, in some embodiments, is use of a compound disclosed herein, or pharmaceutically acceptable salts, enantiomers, stereoisomers, or tautomers thereof, or a pharmaceutical composition disclosed herein, in the preparation of a medicament for treating a disease selected from inflammatory diseases, neurodegenerative disorders, cardiovascular disorders, autoimmune diseases and viral infections.

In some embodiments, provided herein is use of a compound disclosed herein, or pharmaceutically acceptable salts, enantiomers, stereoisomers, or tautomers thereof, or a pharmaceutical composition disclosed herein, in the preparation of a medicament for treating a viral infection.

In some embodiments, the viral infection is a caused by a coronavirus. In some embodiments, the viral infection is caused by a virus selected from the group consisting of a coronavirus, a rhinovirus and a flavivirus. In some embodiments, the viral infection is caused by a rhinovirus. In some embodiments, the viral infection is caused by a flavivirus.

In some embodiments, the viral infection is caused by a coronavirus selected from the group consisting of: 229E alpha coronavirus, NL63 alpha coronavirus, OC43 beta coronavirus, HKU1 beta coronavirus, Middle East Respiratory Syndrome (MERS) coronavirus (MERS-CoV), severe acute respiratory syndrome (SARS) coronavirus (SARS-CoV), and SARS-CoV-2. In some embodiments, the viral infection is caused by SARS. In some embodiments, the viral infection is caused by SARS-CoV. In some embodiments, the viral infection is caused by SARS-CoV-2. In some embodiments, the viral infection is caused by MERS-CoV. In some embodiments, the viral infection is COVID-19.

In some embodiments, the viral infection is caused by a positive RNA virus. In some embodiments, the virus is a positive-sense RNA virus. In some embodiments, the virus is a sense RNA virus. In some embodiments, the virus is a sense-strand RNA virus. In some embodiments, the virus a positive-strand RNA virus. In some embodiments, the virus is a positive (+) RNA virus. In some embodiments, the virus is a positive-sense single-stranded RNA virus. In some embodiments, the positive RNA virus is selected from the group consisting of a virus of the Coronaviridae family, a virus of the Flaviviridae family, and a virus of the Picornaviridae family. In some embodiments, the positive RNA virus is selected from the group consisting of a rhinovirus, a flavivirus, a picornavirus, and a coronavirus. In some embodiments, the positive RNA virus is a picornavirus. In some embodiments, the positive RNA virus is a rhinovirus. In some embodiments, the positive RNA virus is a human rhinovirus. In some embodiments, the positive RNA virus is a flavivirus. In some embodiments, the positive RNA virus is coronavirus. In some embodiments, the positive RNA virus is selected from the group consisting of SARS CoV-1, SARS CoV-2, MERS, hepatitis C (HCV), rhinovirus, Dengue virus, Zika virus, and West Nile virus. In some embodiments, the positive RNA virus is a coronavirus. In some embodiments, the coronavirus is selected from the group consisting of SARS CoV-1, SARS CoV-2 and MERS. In some embodiments, the coronavirus is SARS CoV-1. In some embodiments, the coronavirus is SARS-CoV-2. In some embodiments, the positive RNA virus (e.g., coronavirus) is of any variant resulting from mutation or novel variants emerging from other species (e.g., species of mammals, e.g., a mink). In some embodiments, the positive RNA virus is MERS. In some embodiments, the positive RNA virus is hepatitis C. In some embodiments, the positive RNA virus is Zika virus. In some embodiments, the positive RNA virus is Dengue virus. In some embodiments, the positive RNA virus is West Nile virus.

In some embodiments, the viral infection is a respiratory viral infection. In some embodiments, the viral infection is an upper respiratory viral infection or a lower respiratory viral infection.

In some embodiments, the use further comprises administering a therapeutically effective amount of one or more other agents or compositions to the patient.

In some embodiments, the one or more other additional agents is selected from the group consisting of ribavirin, favipiravir, ST-193, oseltamivir, zanamivir, peramivir, danoprevir, ritonavir, and remdesivir.

In some embodiments, the one or more other additional agents is selected from the group consisting of protease inhibitors, fusion inhibitors, M2 proton channel blockers, polymerase inhibitors, 6-endonuclease inhibitors, neuraminidase inhibitors, reverse transcriptase inhibitor, aciclovir, acyclovir, protease inhibitors, arbidol, atazanavir, atripla, boceprevir, cidofovir, combivir, darunavir, docosanol, edoxudine, entry inhibitors, entecavir, famciclovir, fomivirsen, fosamprenavir, foscarnet, fosfonet, ganciclovir, ibacitabine, immunovir, idoxuridine, imiquimod, inosine, integrase inhibitor, interferons, lopinavir, loviride, moroxydine, nexavir, nucleoside analogues, penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine.

In some embodiments, the one or more other additional agents is selected from the group consisting of lamivudine, an interferon alpha, a VAP anti-idiotypic antibody, enfuvirtide, amantadine, rimantadine, pleconaril, aciclovir, zidovudine, fomivirsen, a protease inhibitor, double-stranded RNA activated caspase oligomerizer (DRACO), rifampicin, zanamivir, oseltamivir, danoprevir, ritonavir, and remdesivir.

In some embodiments, the one or more other additional agents is selected from the group consisting of quinine (optionally in combination with clindamycin), chloroquine, amodiaquine, artemisinin and its derivatives, doxycycline, pyrimethamine, mefloquine, halofantrine, hydroxychloroquine, eflornithine, nitazoxanide, ornidazole, paromomycin, pentamidine, primaquine, pyrimethamine, proguanil (optionally in combination with atovaquone), a sulfonamide, tafenoquine, tinidazole and a PPT1 inhibitor.

In some embodiments, the one or more other additional agents is an RNA polymerase inhibitor. In some embodiments, the RNA polymerase inhibitor is selected from the group consisting of remdesivir, sofosbuvir, 7-deaza-2-CMA, galidesvir, and AT-527. In some embodiments, the RNA polymerase inhibitor is remdesivir.

In some embodiments, the one or more other additional agents is selected from the group consisting of a TMPRSS protease inhibitor, a lysosomal blocking agent (e.g., hydroxychloroquine), a PIKfyve inhibitor (e.g., apilimod), an anti-SARSCOV-2 antibody, a cocktail of anti-SARSCOV-2 antibodies, an anti-inflammatory agent, an anti-TNF agent (e.g., adalimumab, infliximab, etanercept, golimumab, or certolizumab), a histimine H1/H2 blocker (e.g., famotidine, nizatidine, ranitidine, and cimetidine), a steroid, an anti-coagulant, a complement targeting agent, a statin, and an ACE inhibitor. In some embodiments, TMPRSS protease inhibitor is selected from the group consisting of a TMPRSS4 inhibitor, a TMPRSS11A inhibitor, a TMPRSS11D inhibitor, TMPRSS11E1 inhibitor, and a TMPRSS2 inhibitor. In some embodiments, the TMPRSS protease inhibitor is a TMRSS2 protease inhibitor. In some embodiments, the TMRESS-2 protease inhibitor is selected from camostat and nafamostat. In some embodiments, the anti-SARSCOV-2 antibody is selected from LY-CoV555 (bamlanivimab) and LY-CoVO16 (etesevimab). In some embodiments, the cocktail of anti-SARSCOV-2 antibodies is REGN-COV2. In some embodiments, the anti-inflammatory agent is an IL-6 antagonist (e.g., siltuximab, sarilumab, olokizumab, BMS-945429, sirukumab, and clazakizumab). In some embodiments, the steroid is dexamethasone. In some embodiments, the anti-coagulant is low-molecular weight heparin. In some embodiments, the complement targeting agent is eculizumab. In some embodiments, the statin is selected from the group consisting of atorvastatin, fluvastatin, lovastatin, pitavastatin, pravastatin, rosuvastatin, and simvastatin.

In some embodiments, the ACE inhibitor is selected from the group consisting of benazepril, captopril enalapril/enalaprilat, fosinopril, lisinopril moexipril, perindopril quinapril, and ramipril.

In some embodiments, the one or more other additional agents is selected from the group consisting of remdesivir, camostat, nafamostat, hydroxychloroquine, chloroquine, apilimod, LY-CoV555 (bamlanivimab), LY-CoVO16 (etesevimab), REGN-COV2, tocilizumab, siltuximab, sarilumab, olokizumab, BMS-945429, sirukumab, clazakizumab, adalimumab, infliximab, etanercept, golimumab, certolizumab, famotidine, nizatidine, ranitidine, cimetidine, dexamethasone, low molecular weight heparin, eculizumab, atorvastatin, fluvastatin, lovastatin, pitavastatin, pravastatin, rosuvastatin, simvastatin, benazepril, captopril enalapril/enalaprilat, fosinopril, lisinopril moexipril, perindopril quinapril, and ramipril.

In some embodiments, the use comprises administering one or more one or more other additional agents selected from the group consisting of remdesivir, sofosbuvir, 7-deaza-2-CMA, galidesvir, AT-527, temoporfin, novobiocin, curcumin, voxilaprevir, grazopevir, glecaprevir,camostat, nafamostat, hydroxychloroquine, chloroquine, apilimod, imatinib, dasatinib, ponatinib, velpatasvir,ledipasvir, elbasivir, pibrentasvir, NITD008, LY-CoV555 (bamlanivimab), LY-CoVO16 (etesevimab), REGN-COV2, tocilizumab, siltuximab, sarilumab, olokizumab, BMS-945429, sirukumab, clazakizumab, adalimumab, infliximab, etanercept, golimumab, certolizumab, famotidine, nizatidine, ranitidine, cimetidine, dexamethasone, low molecular weight heparin, eculizumab, atorvastatin, fluvastatin, lovastatin, pitavastatin, pravastatin, rosuvastatin, simvastatin, benazepril, captopril enalapril/enalaprilat, fosinopril, lisinopril moexipril, perindopril quinapril, ramipril, and adoptive NK cell therapy.

In some embodiments, the one or more other additional agents is selected from the group consisting of a ABL inhibitor and a JAK inhibitor.

In some embodiments, the one or more other additional agents is an ABL inhibitor (e.g., imatinib, dasatinib, or ponatinib). In some embodiments, the ABL inhibitor is selected from the group consisting of imatinib, dasatinib, and ponatinib. In some embodiments, the ABL inhibitor is imatinib. In some embodiments, the ABL inhibitor is dasatinib. In some embodiments, the ABL inhibitor is ponatinib.

In some embodiments, the one or more other additional agents is a JAK inhibitor. In some embodiments, the JAK inhibitor is selected from the group consisting of baricitinib, ruxolitinib, tofacitinib, and upadacitinib. In some embodiments, the JAK inhibitor is baricitinib. In some embodiments, the JAK inhibitor is ruxolitinib. In some embodiments, the JAK inhibitor is tofacitinib. In some embodiments, the JAK inhibitor is upadacitinib.

In some embodiments, the one or more other additional agents is a protease inhibitor. In embodiments, the protease inhibitor is selected from the group consisting of temoporfin, novobiocin, curcumin, voxilaprevir, grazopevir, and glecaprevir.

In some embodiments, the one or more other additional agents is an NS5A inhibitor. In embodiments, the NS5A inhibitor is selected from the group consisting of velpatasvir, ledipasvir, elbasivir, and pibrentasvir.

In some embodiments, the one or more other additional agents is a pyrimidine synthesis inhibitor. In some embodiments, the pyrimidine synthesis inhibitor is NITD008.

In some embodiments, the one or more other additional agents is an adoptive natural killer (NK) cell therapy. In some embodiments, the additional therapeutic agent is a vaccine. In some embodiments, the vaccine is a coronavirus vaccine. In some embodiments, the vaccine is selected from the group consisting of BNT162b2, mRNA-1273, AZD1222, and Ad26.COV2.S. In some embodiments, the vaccine is a protein-based vaccine. In some embodiments, the vaccine is an RNA-based vaccine. In some embodiments, the vaccine is an attenuated virus vaccine. In some embodiments, the vaccine is an inactivated virus vaccine. In some embodiments, the vaccine is a non-replicating viral vector vaccine.

In some embodiments, the compound described herein is orally administered to the patient. In some embodiments, the compound described herein is parenterally administered to the patient.

In some embodiments, provided herein are methods of treating cancer in a patient in need thereof comprising (i) administering to the patient a therapeutically effective amount of the compound disclosed herein, or pharmaceutically acceptable salts, enantiomers, stereoisomers, or tautomers thereof; and (ii) administering to the patient a therapeutically effective amount of a STING agonist; wherein administering the therapeutically effective amount of the STING agonist and the compound results in an increased expression level of at least one chemokine in the patient as compared to any increase in the expression level of the at least one chemokine resulting from administering the compound alone to the patient.

In some embodiments, provided herein are methods of upregulating at least one chemokine in a cell comprising contacting the cell sample with (i) the compound disclosed herein, or pharmaceutically acceptable salts, enantiomers, stereoisomers, or tautomers thereof; and (ii) a STING agonist in an amount sufficient to increase the expression level of the at least one chemokine in the cell.

In some embodiments, the cancer is selected from the group consisting of gastrointestinal stromal tumors, a esophageal cancer, a gastric cancer, a melanoma, a glioma, a glioblastoma, an ovarian cancer, a bladder cancer, a head cancer, a neck cancer, a urothelial cancer, a uterine cancer, a pancreatic cancer, a prostate cancer, a lung cancer, a breast cancer, a renal cancer, a hepatic cancer, an osteosarcoma, a sarcoma, a multiple myeloma, a cervical carcinoma, a cancer that is metastatic to bone, a papillary thyroid carcinoma, a non-small cell lung cancer, a lymphoma, a leukemia, and a colorectal cancer.

In some embodiments, the STING agonist is selected from the group consisting of 5,6-dimethylxanthenone-4-acetic acid (DMXAA), ADU-S100, MK-1454, MK-2118, BMS-986301, GSK3745417, SB-11285, BI1387446 (BI-STING), E7766, TAK-676, SNX281, SYNB1891, JNJ-67544412, JNJ-′6196, GSK532, TTI-10001, ALG-031048, MSA-1, MSA-2, CRD-5500, MV-626, SR-8314, SR-8291, SR8541A, SR-717, STING antibody-drug conjugates (ADC), and IMSA-101, and pharmaceutically acceptable salts thereof.

In some embodiments, the STING agonist is selected from the group consisting of ADU-S100, MK-1454, MK-2118, BMS-986301, GSK3745417, SB-11285, BI1387446 (BI-STING), E7766, TAK-676, SNX281, SYNB1891, and IMSA-101, and pharmaceutically acceptable salts thereof.

In some embodiments, the STING agonist is selected from the group consisting of ADU-S100 and MSA-2, and pharmaceutically acceptable salts thereof.

### EXAMPLES

The compounds described herein can be prepared in a number of ways based on the teachings contained herein and synthetic procedures known in the art. In the description of the synthetic methods described below it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

The following abbreviation are used in this disclosure and have the following definitions: “ADP” is adenosine diphosphate, “aq” is aqueous, “ATP” is adenosine triphosphate, “Ar” is argon gas, “Bn” is benzyl, “BPD” is bis(pinacolato)diboron, “Boc” is t-butylcarbonate, “BSA” is bovin serium albumin, “tert-BuOK” is potassium tert-butoxide, “tert-BuONa” is sodium tert-butoxide, “conc” is concentrated, “CaCl₂)” is calcium chloride, “CDCl₃” is chloroform-deuterium, “Cs₂CO₃” is cesium carbonate, “DAST” is diethylaminosulfur trifluoride, “DCM” is dichloromethane, “DIEA” is N,N-diisopropylethylamine, “DMB” is dimenthylbenzylamine, “DMF” is N,N-dimethylformamide, “dppf” is 1,1′-bis(diphenylphosphino)ferrocene, “DMSO-d₆” is dimethylsulfoxide-deuterium, “DSC” is N,N′-disuccinimidyl carbonate, “DTT” is dithiothreitol, “EDC” is 1-ethyl-3-(3-dimethylaminopropyl)carbodiimide, “ESI” is electrospray ionization, “EtOAc” is ethyl acetate, “EtOH” is ethanol, “GST” is glutathione S-transferase, “h” is hour or hours, “HATU” is hexafluorophosphate azabenzotriazole tetramethyl uronium, “H₂” is hydrogen gas, “HCl” is hydrochloric acid, “H₂O” is water, “IC₅₀” is half maximal inhibitory concentration, “LDA” is lithium diisopropylamide, “LiHMDS” is lithium hexamethyldisilazide, “MeCN” is acetonitrile, “MeOD” is methanol-deuterium, “MeOH” is methanol, “MgSO₄” is magnesium sulfate, “MHz” is megahertz, “min” is minute or minutes, “MS” is mass spectrometry, “MTBE” is methyl tert-butyl ether, “m/z” is mass/charge number, “NaCN” is sodium cyanide, “NADH” is nicotinamide adenine dinucleotide, “NaH” is sodium hydride, “NaHCO₃” is sodium bicarbonate, “Na₂SO₄” is sodium sulfate, “NMI” is N-methylimidazole, “NMR” is nuclear magnetic resonance, “PBS” is phosphate buffered saline, “Pd” is palladium, “Pd/C” is palladium on carbon, “Pd(dppf)Cl₂ is “bis(diphenylphosphino)ferrocene dichloropalladium”, “Pd(PPh₃)₄” is Palladium-tetrakis(triphenylphosphine), “PEPPSI-IPr” is [1,3-bis(2,6-diisopropylphenyl)imidazol-2-ylidene](3-chloropyridyl)palladium(II) dichloride, “pet-ether” is petroleum ether, “PMB” is pare-methoxybenzyl, “RLU” is relative luminescence unit, “rt” is room temperature which is also known as “ambient temp,” which will be understood to consist of a range of normal laboratory temperatures ranging from 15-25° C., “sat'd.” is saturated, “SFC” is supercritical fluid chromatography, “SM” is starting material, “S_{N}Ar” is nucleophilic aromatic substitution, “T3P” is 1-propanephosphonic acid anhydride, “TBAF” is tetrabutyl ammonium fluoride, “TBDMS” is tert-butyldimethylsilyl, “TCFH” is chloro-N,N,N′,N′-tetramethylchloroformamidinium hexafluorophosphate, “TEA” is triethylamine, “TFA” is trifluoroacetic acid, “Tf₂O” is trifluoromethanesulfonic anhydride, “THF” is tetrahydrofuran, “tris” is tris(hydroxymethyl)aminomethane, and “TMS” is trimethylsilyl.

### General Chemistry

Exemplary compounds described herein are available by the general synthetic methods illustrated in the Schemes below, intermediate preparations, and the accompanying Examples.

### Synthetic Schemes

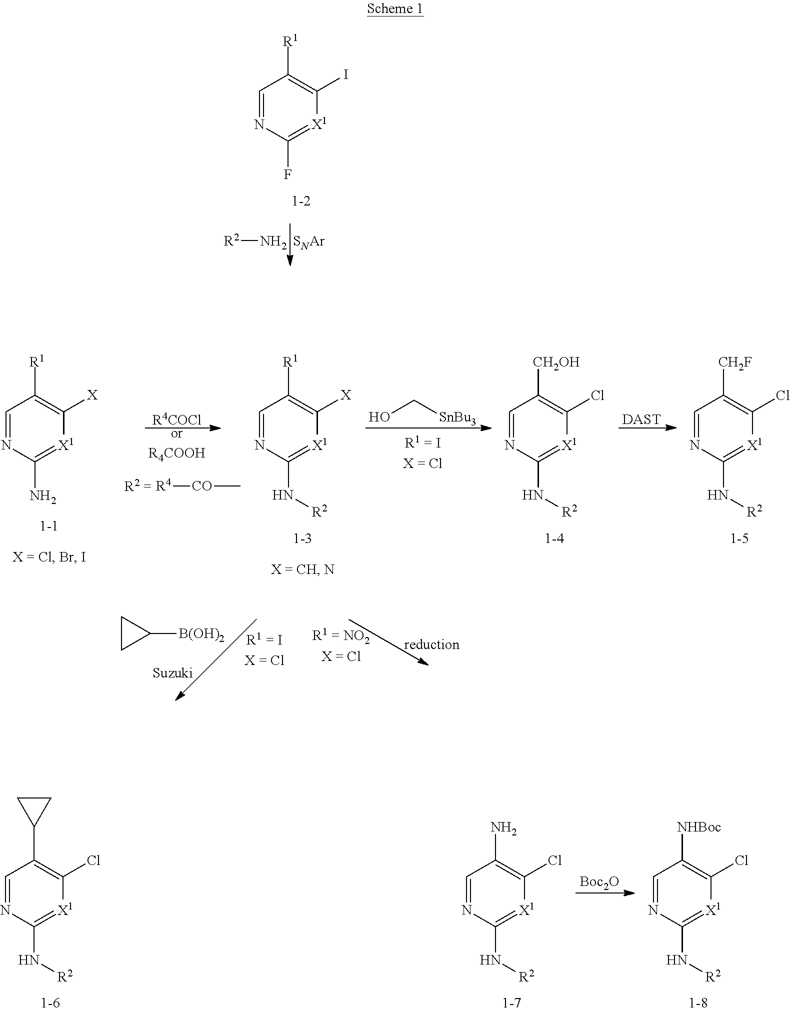

Scheme 1 illustrates an exemplary preparation of intermediates 1-3, 1-5, 1-6, and 1-8. Amines 1-1 (commercially available or synthesized by those skilled in the art) react with R⁴COCl (commercially available or synthesized by those skilled in the art) by acylation in the presence of base such as pyridine, TEA, and tert-BuOK under ambient or elevated temperature to afford intermediates 1-3. In another embodiment, amines 1-1 react with R⁴COOH (commercially available or synthesized by those skilled in the art) by a typical amide coupling reaction in the presence of coupling reagent such as HATU, T3P, or TCFH to afford intermediates 1-3. Alternatively, intermediate 1-3 can be prepared from 1-2 (commercially available or synthesized by those skilled in the art) with R²—NH₂ by S_{N}Ar reaction in a presence of base such as LDA, or Cs₂CO₃. Compounds 1-3 (when R¹═I) can be converted to 1-5 and 1-6: (1) Stille coupling reaction with SnBu₃CH₂OH followed by fluorination reaction with DAST and (2) Suzuki reaction with cyclopropylboronic acid. In another embodiment, compounds 1-3 (when R¹ ═NO₂) can be reduced to amines and then N-Boc protection to afford 1-8.

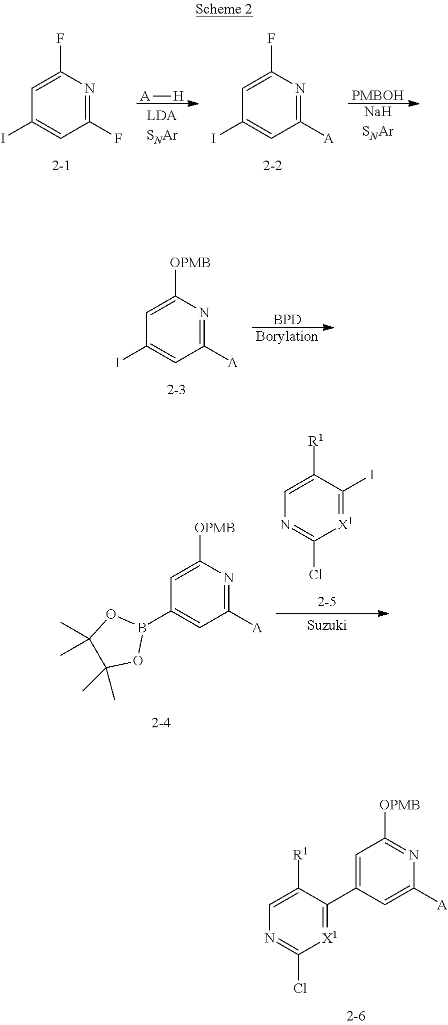

Scheme 2 illustrates an exemplary preparation of intermediate 2-6. Compound 2,6-difluoro-4-iodopyridine, 2-1 reacts with amines A-H (commercially available or synthesized by those skilled in the art) in the presence of base such as LDA to afford 2-2. S_{N}Ar reaction of 2-2 with PMBOH in the presence of base such as NaH affords 2-3. Compounds 2-3 upon borylation provide boronates 2-4. Borylation is a well-documented reaction to those skilled in the art (for example, Pd (0) catalyzed reaction with BPD). A typical Suzuki reaction of boronates 2-4 with iodides 2-5 (commercially available or synthesized by those skilled in the art) affords intermediates 2-6.

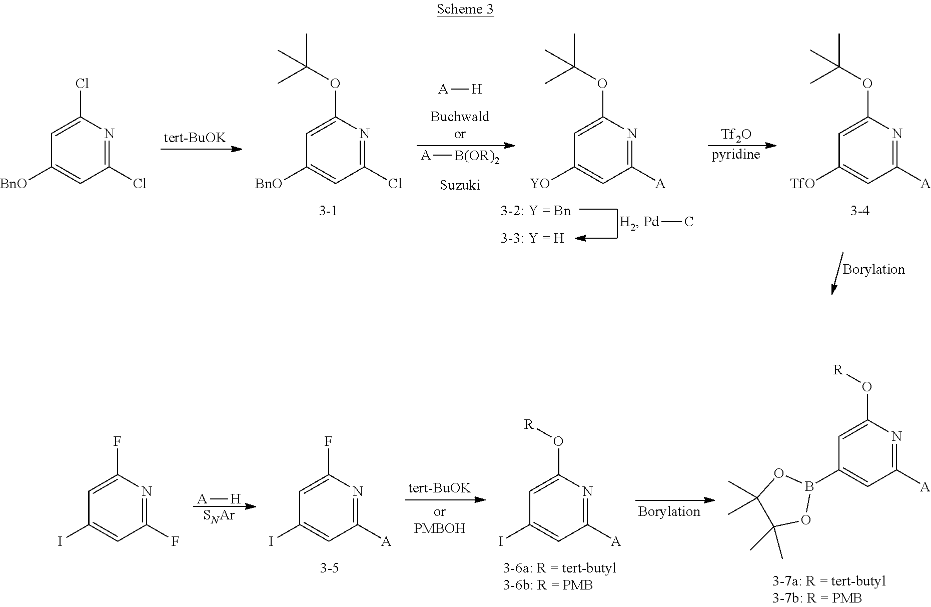

Scheme 3 illustrates an exemplary preparation of intermediates 3-7a (R=tert-butyl) and 3-7b (R=PMB). S_{N}Ar reaction of 4-(benzyloxy)-2,6-dichloropyridine with tert-BuOK affords 3-1. 4-(Benzyloxy)-2-(tert-butoxy)-6-chloropyridine 3-1 reacts with amines A-H (commercially available or synthesized by those skilled in the art) under Buchwald-Hartwig amination conditions (PEPPSI-IPr as a catalyst) or boronates or boronic acids A-B(OR)₂ (commercially available or synthesized by those skilled in the art) under Suzuki reaction conditions (Pd(dppf)Cl₂ as a catalyst) to afford the “A” substituted pyridines 3-2 (Y=Bn). Deprotection of 3-2 (Y=Bn) under Pd catalyzed hydrogenation affords intermediates 3-3 (Y═H). Treatment of 3-3 with Tf₂O in the presence of a base, such as pyridine affords triflates 3-4. Triflates 3-4 can be converted to boronates 3-7a (R=tert-butyl) by borylation under Pd (0) conditions. In another embodiment, intermediates 3-7a (R=tert-butyl) and 3-7b (R=PMB) can be prepared from 2,6-difluoro-4-iodopyridine. First step: S_{N}Ar reaction of 2,6-difluoro-4-iodopyridine with amines A-H affords 3-5. Second step: compounds 3-5 react with tert-BuOK or PMBOH in the presence of base such as NaH by S_{N}Ar reaction to afford 3-6a (R=tert-butyl) and 3-6b (R=PMB). Third step: iodides 3-6a and 3-6b can be converted to borated 7-7a and 3-7b respectively by borylation with BPD under Pd (0) conditions.

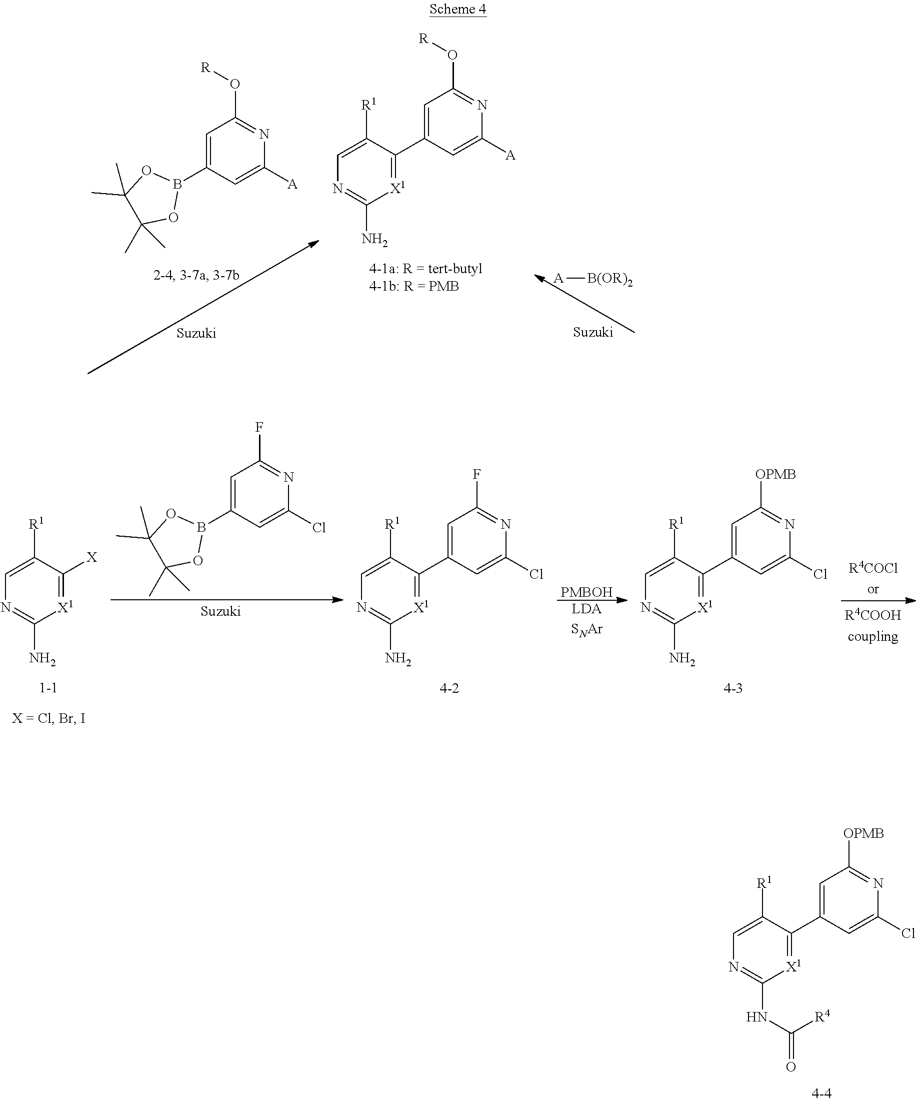

Scheme 4 illustrates an exemplary preparation of intermediates 4-la, 4-1b, and 4-4. Suzuki reaction of 1-1 (commercially available or synthesized by those skilled in the art) with boronates 2-4, 3-7a and 3-7b (scheme 3) in the presence of Pd catalyst such as Pd(dppf)Cl₂ to afford 4-la (R=tert-butyl) and 4-1b (R=PMB) respectively. In another embodiment, compounds 1-1 (X═Cl, Br, I) react with 2-chloro-6-fluoro-4-(4,4,5,5-tetramethyl-1,3,2-dioxaborolan-2-yl)pyridine under Suzuki reaction conditions to afford 4-2. S_{N}Ar reaction of 4-2 with PMBOH in the presence of base, such as LDA affords 4-3. Compounds 4-3 react with R⁴COCl (commercially available or synthesized by those skilled in the art) by acylation in the presence of base such as pyridine, TEA, and tert-BuOK under ambient or elevated temperature or R⁴COOH (commercially available or synthesized by those skilled in the art) by a typical amide coupling reaction in the presence of coupling reagent, such as HATU, T3P, or TCFH to afford intermediates 4-4. In another embodiment, Suzuki reaction of 4-3 with boronic acids or boronate A-B(OR)₂ affords 4-1b (R=PMB).

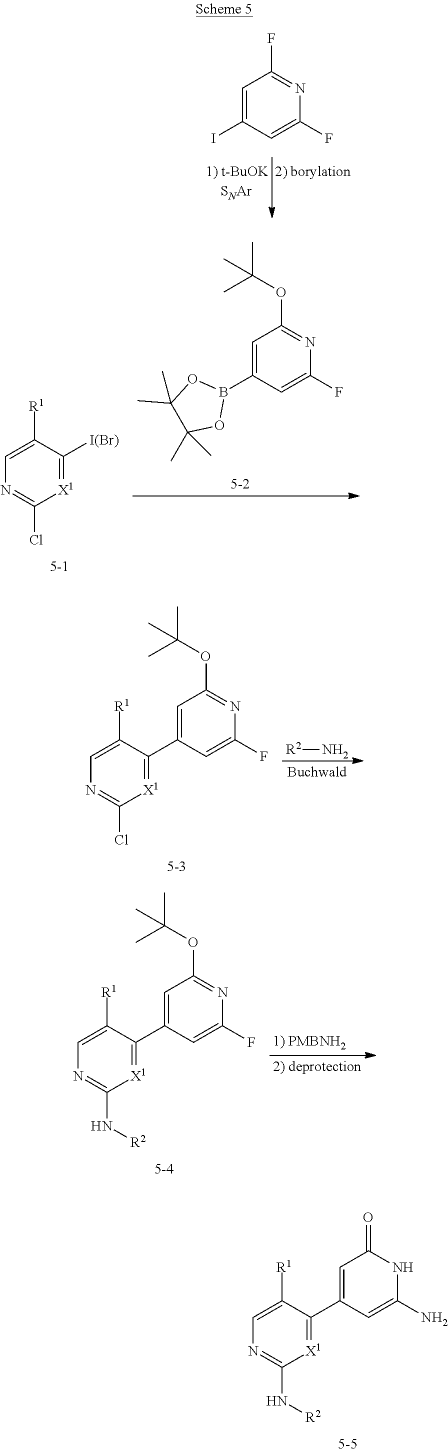

Scheme 5 illustrates an exemplary preparation of intermediates 5-5. Compounds 5-1 (commercially available or synthesized by those skilled in the art) react with boronates 5-2 (prepared by S_{N}Ar reaction of 2,6-difluoro-4-iodopyridine with tert-BuOK, followed by a typical borylation with BPD under Pd (0) conditions) under Suzuki reaction conditions to afford 5-3. Compounds 5-3 can be reacted with amines R²—NH₂ (commercially available or synthesized by those skilled in the art) by Buchwald-Hartwig cross coupling reaction (for example: Pd(dppf)Cl₂, tert-BuONa in an elevated temperature) to obtain 5-4. S_{N}Ar reaction of 5-4 with PMBNH₂, followed by deprotection with TFA/Et₃SiH affords intermediates 5-5.

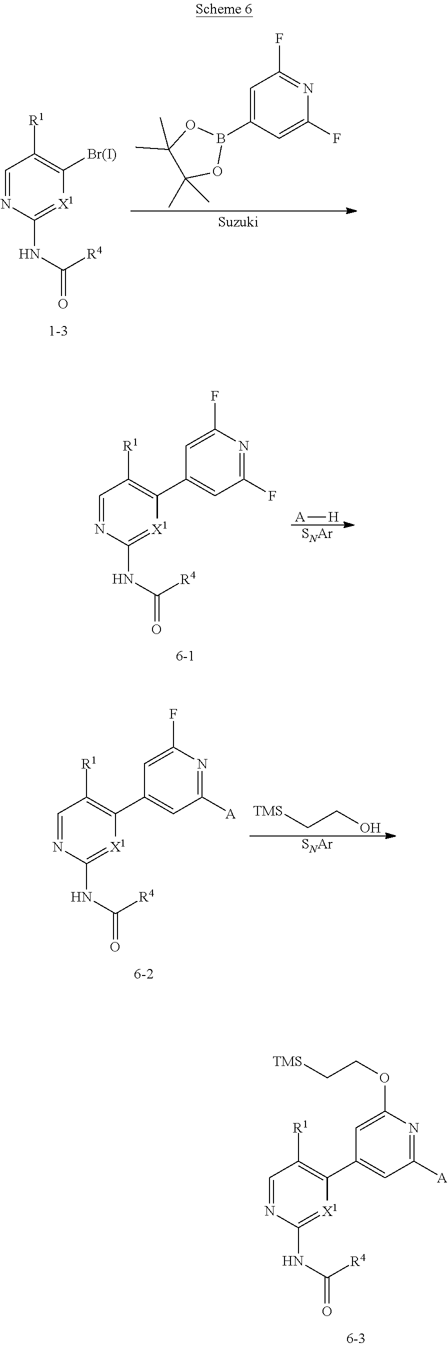

Scheme 6 illustrates an exemplary preparation of intermediates 6-3. Suzuki reaction of 1-3 with 2,6-difluoro-4-(4,4,5,5-tetramethyl-1,3,2-dioxaborolan-2-yl)pyridine affords 6-1. Compounds 6-1 react with amines A-H (commercially available or synthesized by those skilled in the art) in the presence of base such as DIEA to afford 6-2. S_{N}Ar reaction of 6-2 with 2-(trimethylsilyl)ethan-1-ol in the presence of base such as NaH affords intermediates 6-3.

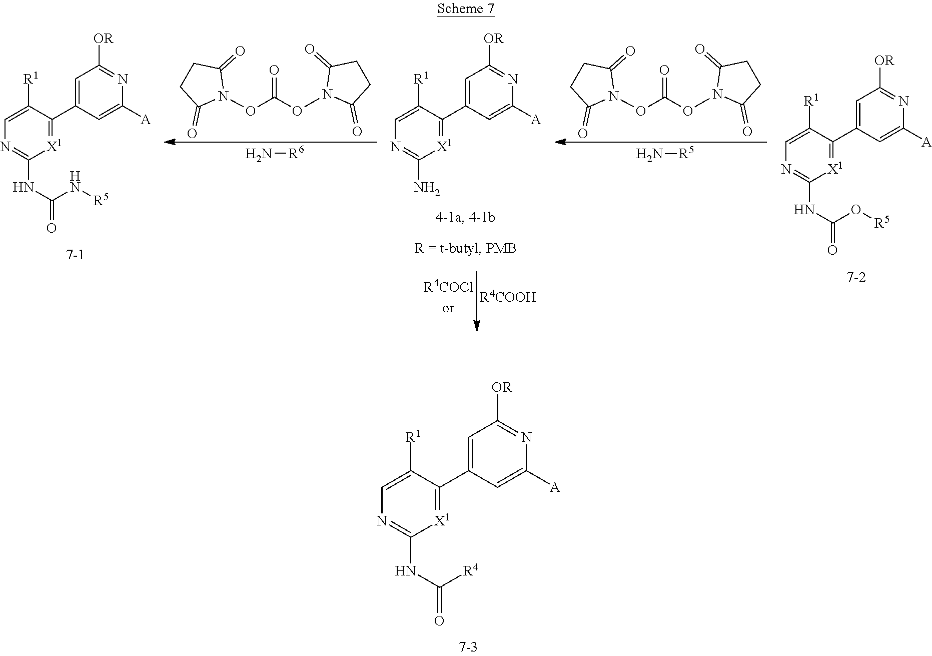

Scheme 7 illustrates an exemplary preparation of intermediates 7-1, 7-2, and 7-3. Urea formation of 4-la or 4-1b with amines R⁶—NH₂ (commercially available or synthesized by those skilled in the art) in the presence of N,N′-DSC affords ureas 7-1. In a similar manner, 4-la or 4-1b can be reacted with alcohols R⁵—OH in the presence of N,N′-DSC to afford carbamates 7-2. Compounds 4-la or 4-1b react with R⁴COCl (commercially available or synthesized by those skilled in the art) in the presence of base such as pyridine, TEA, and tert-BuOK under ambient or elevated temperature or R⁴COOH (commercially available or synthesized by those skilled in the art) in the presence of coupling reagents such as HATU, T3P, or TCFH to afford amides 7-3.

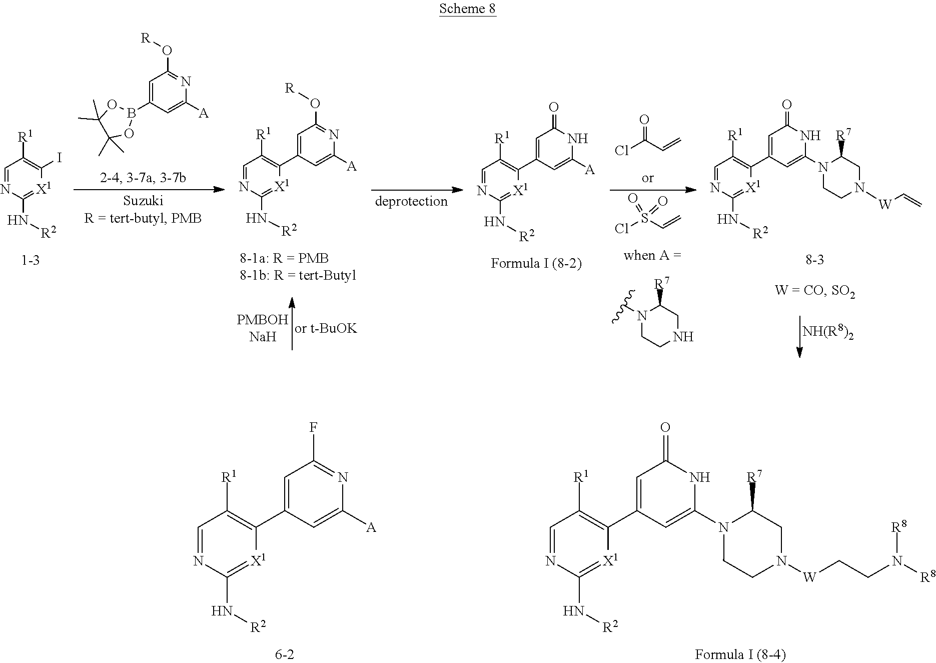

Scheme 8 illustrates an exemplary preparation of compounds of Formula I (8-2 and 8-4). Suzuki reaction of 1-3 with boronates (2-4, 3-7a, 3-7b) affords 8-la and 8-1b (R=tert-butyl, PMB). In another embodiment, S_{N}Ar reaction of 6-2 with PMBOH in the presence of base such as NaH or tert-BuOK affords 8-1b (R=PMB) and 8-la (R=tert-butyl) respectively. Compounds 8-la (R=tert-butyl) and 8-1b (R=PMB) can be deprotected with either TFA or HCl to afford compounds of Formula I (8-2). Compounds 8-2 react with acryloyl chloride or ethenesulfonyl chloride in the presence of base such as TEA or DIEA to afford 8-3 (W═CO, SO₂). Michael reaction of 8-3 with amines NH(R⁸)₂ (commercially available or synthesized by those skilled in the art) affords compounds of Formula I (8-4).

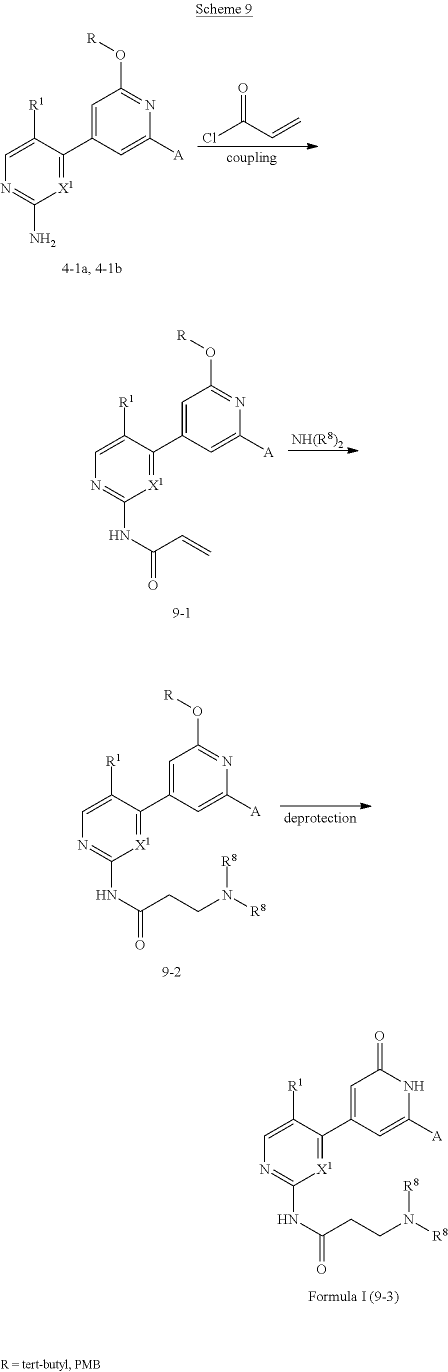

Scheme 9 illustrates an exemplary preparation of compounds of Formula I (9-3). Compounds 4-la and 4-1b react with acryloyl chloride in the presence of base such as pyridine to afford 9-1. Michael reaction of 9-1 with amines NH(R⁸)₂ (commercially available or synthesized by those skilled in the art) affords 9-2. Deprotection of 9-2 under acidic conditions (TFA or HCl) affords compounds of Formula I (9-3).

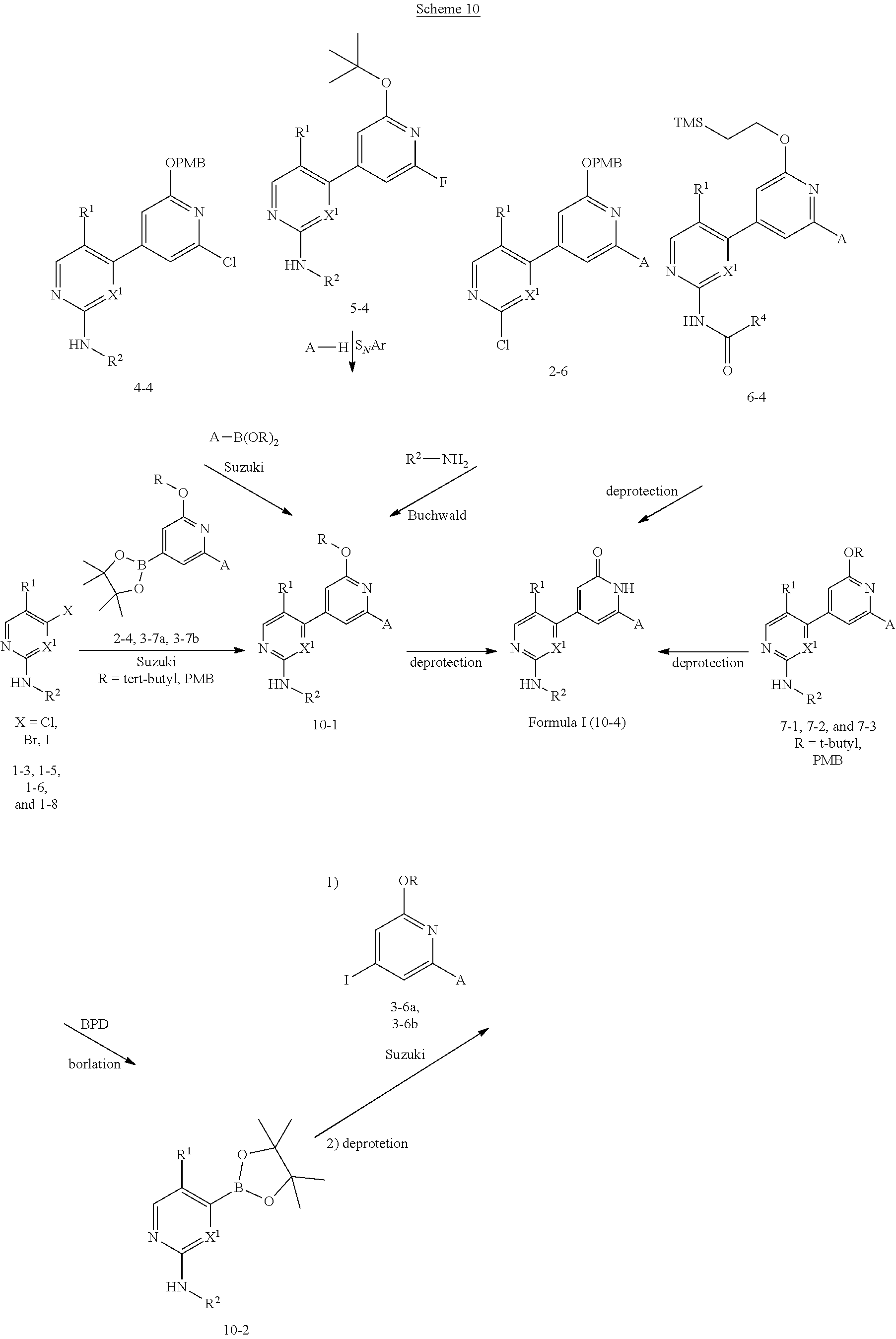

Scheme 10 illustrates an exemplary preparation of compounds of Formula I (10-4). Intermediates 1-3, 1-5, 1-6, and 1-8 (X═Cl, Br, I)) react with boronates 2-4, 3-7a, or 3-7b under Suzuki condition to afford intermediates 10-1. Alternatively, intermediates 10-1 can be prepared from (1) chlorides 2-6 with amines R²—NH₂ (commercially available or synthesized by those skilled in the art) by Buchwald-Hartwig amination reaction conditions such as using PEPPSI-IPr as a catalyst (2) chlorides 4-4 with boronates or boronic acid A-B(OR)₂ (commercially available or synthesized by those skilled in the art) by Suzuki reaction and (3) fluorides 5-4 with amines A-H by S_{N}Ar reaction. Deprotection of 10-1 under acidic conditions such as HCl or TFA affords compounds of Formula I (10-4). In another embodiment, compounds 1-3, 1-5, 1-6, and 1-8 upon borylation provide boronates 10-2. Borylation is a well-documented reaction to those skilled in the art (for example, Pd (0) catalyzed reaction with BPD). Suzuki reaction of 10-2 with bromides or iodides 3-6a or 3-6b, followed by deprotection with TFA or HCl affords compounds of Formula I (10-4). In a similar manner, deprotection of compounds 6-4, 7-1, 7-2, and 7-3 under acidic conditions such as TFA or HCl affords compounds of Formula I (10-4).

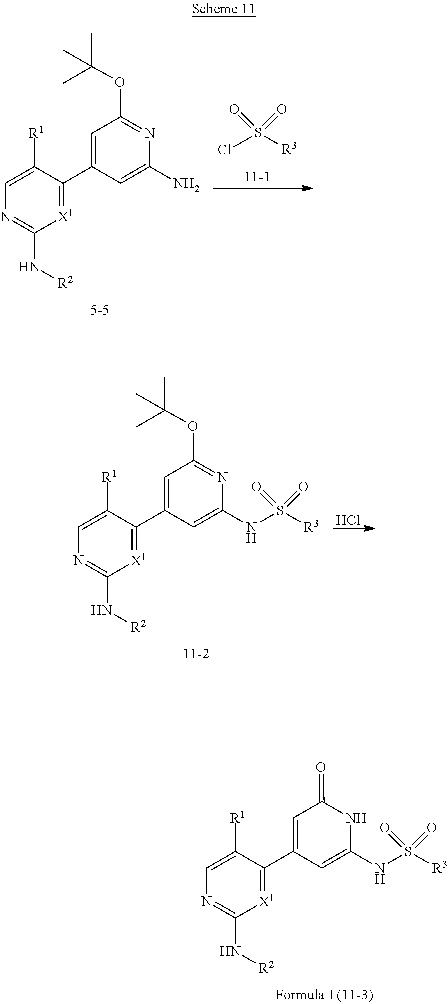

Scheme 11 illustrates an exemplary preparation of compounds of Formula I (11-3). Compounds 5-5 react with sulfonyl chlorides (11-1, commercially available or synthesized by those skilled in the art) to afford 11-2. Deprotection of 11-2 under acidic conditions such as HCl affords compounds Formula I (11-3).

### Preparation of Intermediates and Final Compounds.

Using the synthetic procedures and methods described herein and methods known to those skilled in the art, the following compounds were made:

### Preparation of Intermediate A1: methyl (4-bromo-5-methylpyridin-2-yl)carbamate

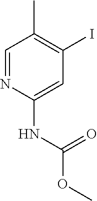

A solution of 4-iodo-5-methyl-pyridin-2-amine (25.0 g, 107 mmol) in pyridine (250 mL) was treated with methyl carbonochloridate (25.2 g, 267 mmol, 20.6 mL) dropwise under an ice-water bath. The mixture was stirred at 0° C. for 2 h, then the reaction mixture was warmed to rt for 10 h. The reaction mixture was quenched with MeOH (50 mL) and concentrated under reduced pressure. The crude product was triturated with 50% NaHCO₃(aq, 100 mL) at 20° C. for 30 min. The solid was filtered, washed with water, and dried under high vacuum to afford methyl N-(4-iodo-5-methyl-2-pyridyl)carbamate (A1, 30.0 g, 91%) as a white solid. ¹H NMR (400 MHz, DMSO-d₆): δ 10.23 (s, 1H), 8.33 (s, 1H), 8.10 (s, 1H), 3.66 (s, 3H), 2.50 (s, 3H); MS (ESI) m/z: 292.8 (M+H⁺).

### Preparation of Intermediate A2: N-(4-iodo-5-methylpyridin-2-yl)-2-methylpyrimidin-4-amine N-(4-iodo-5-methylpyridin-2-yl)-2-methylpyrimidin-4-amine

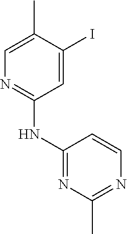 [SMILES: Cc1nccc(Nc2cc(I)c(C)cn2)n1]

A solution of 2-fluoro-4-iodo-5-methyl-pyridine (5.0 g, 21 mmol) and 2-methylpyrimidin-4-amine (2.8 g, 25 mmol) in DMF (80 mL) was treated with Cs₂CO₃ (21 g, 63 mmol). The mixture was heated at 100° C. for 24 h and then cooled to rt. The reaction mixture was diluted with water and extracted with EtOAc (3×). The combined organics were washed with brine, dried over anhydrous Na₂SO₄, filtered, and concentrated under reduced pressure. The resulting crude was purified by silica gel column chromatography (0 to 30% EtOAc/pet-ether) to give N-(4-iodo-5-methylpyridin-2-yl)-2-methylpyrimidin-4-amine (A2, 2.0 g, 29%) as a yellow solid. ¹H NMR (400 MHz, CDCl₃): δ 8.26 (d, J=5.8 Hz, 1H), 8.16 (s, 1H), 7.97 (s, 1H), 7.16 (d, J=5.6 Hz, 1H), 2.55 (s, 3H), 2.32 (s, 3H); MS (ESI) m/z: 327.0 (M+H⁺).

The following compounds are prepared essentially by method of preparation A1 or A2.

| Ex | | | Yield | | MS (m/z: |
|---|---|---|---|---|---|
| No | SM | Product | (%) | ¹H NMR (400 MHz, DMSO-d₆): δ | M + H⁺) |
| A3 | 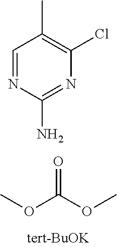 | 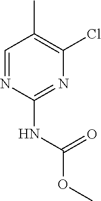 | 71 | 8.40 (s, 1H), 3.78 (s, 3H), 2.28 (s, 3H). | 202.1 |
| A4 | 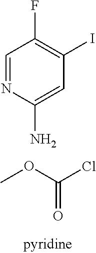 | 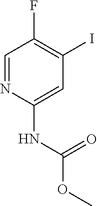 | 75 | 10.40 (s, 1H). 8.30 (d, J = 4.4 Hz, 1H), 8.21 (s, 1H), 3.67 (s, 3H). | 297.0 |
| A5 | 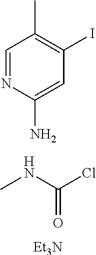 | 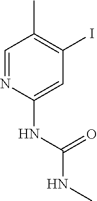 | 87 | 9.13 (s, 1H), 8.01 (s, 1H), 7.97 (s, 1H), 7.61 (s, 1H), 2.69 (s, 3H), 2.23 (s, 3H). | 292.2 |
| A6 | 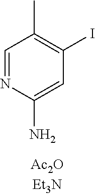 | 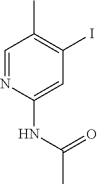 | 74 | | 277.0 |
| A7 | 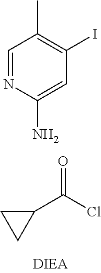 | 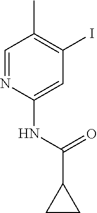 | 96 | 10.80 (s, 1H), 8.60 (s, 1H), 8.15 (s, 1H), 2.28 (s, 3H), 1.96 (m, 1H), 0.78-0.83 (m, 4H). | 303.0 |
| A8 | 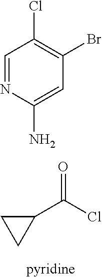 | 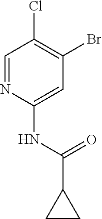 [SMILES: O=C(Nc1cc(Br)c(Cl)cn1)C1CC1] | 94 | | 276.9 |
| A9 | 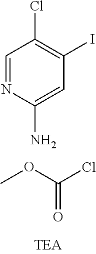 | 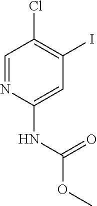 | 83 | | 313.1 |
| A10 | 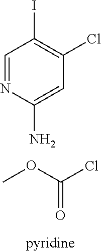 | 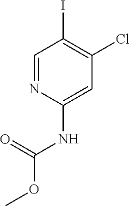 | 99 | 8.99 (s, 1H), 8.58 (s, 1H), 8.25 (s, 1H), 3.85 (m, 3H). | 313.1 |
| A11 | 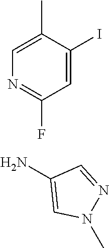 | 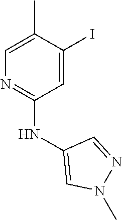 | 62 | | 315.0 |
| | LDA | | | | |
| A12 | 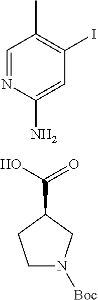 | 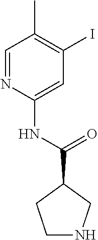 [SMILES: Cc1cnc(NC(=O)[C@@H]2CCNC2)cc1I] | 95 | | 332.1 |
| | HATU | | | | |
| | HCl | | | | |
| A13 | 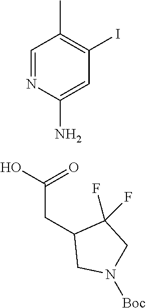 | 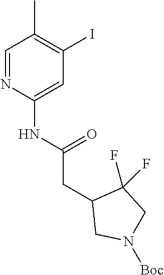 [SMILES: Cc1cnc(NC(=O)CC2CN(C(=O)OC(C)(C)C)CC2(F)F)cc1I] | 72 | 8.70 (s, 1H), 8.08 (s, 1H), 8.03 (s, 1H), 3.98 (dd, J = 8.4, 10.8 Hz, 1H), 3.75 (m, 2H), 3.20 (t, J = 10.0 Hz, 1H), 2.81 (s, 2H), 2.50 (m, 1H), 2.36 (s, 3H), 1.47 (s, 9H). | 482.1 |
| | TCFH | | | | |
| A14 | 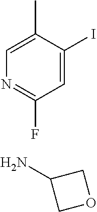 | 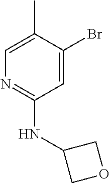 | 33 | | 291.2 |
| | LDA | | | | |
| A15 | 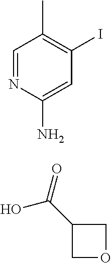 | 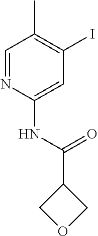 | 52 | 7.73 (s, 1H), 7.63 (s, 1H), 4.29-4.52 (m, 2H), 3.75 (m, 2H), 2.86 (m, 1H), 2.30 (s, 3H), in D20. | 319.2 |
| | TCFH | | | | |
| A16 | 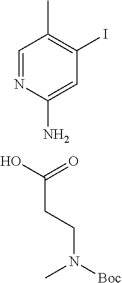 | 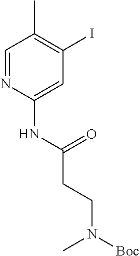 [SMILES: Cc1cnc(NC(=O)CCN(C)C(=O)OC(C)(C)C)cc1I] | 81 | | 364.0 |
| | TCFH | | | | |
| A17 | 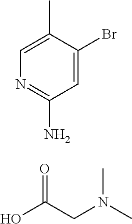 | 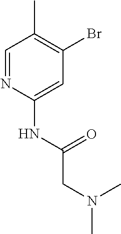 | 53 | | 272.2 |
| | HATU | | | | |
| A18 | 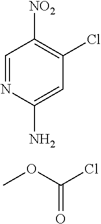 | 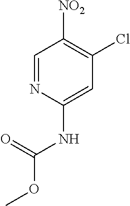 | 21 | | 232.1 |
| | TEA | | | | |
| | DMAP | | | | |

### Preparation of Intermediate A19: methyl (4-chloro-5-cyclopropylpyridin-2-yl)carbamate

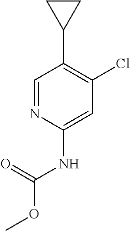

A mixture of methyl N-(4-chloro-5-iodo-2-pyridyl)carbamate (A10, 0.50 g, 1.6 mmol), cyclopropylboronic acid (0.27 g, 3.2 mmol), and Na₂CO₃ (0.34 g, 3.2 mmol) in a mixture of 1,4-dioxane (25 mL) and H₂O (5 mL) was degassed and purged with N₂ for 3 min. Pd(dppf)Cl₂ (0.12 g, 0.1 eq) was added and then the mixture was heated at 80° C. for 12 h under N₂ atmosphere. The mixture was cooled to rt and then diluted with H₂O (20 mL). The mixture was filtered through a pad of celite and the filtrate was extracted with EtOAc (3×). The combined organic layers were dried over anhydrous Na₂SO₄, filtered, and concentrated under reduced pressure. The residue was purified by silica gel column chromatography (0 to 6% EtOAc/pet-ether) to obtain N-(4-chloro-5-cyclopropyl-2-pyridyl)carbamate (A19, 0.16 g 44%) as a white solid. ¹H NMR (400 MHz, DMSO-d₆): δ 8.05 (s, 1H), 7.91 (s, 1H), 3.82 (s, 3H), 2.01 (m, 1H), 0.95-1.04 (m, 2H), 0.68-0.72 (m, 2H).

### Preparation of intermediate A20: methyl (4-chloro-5-(fluoromethyl)pyridin-2-yl)carbamate

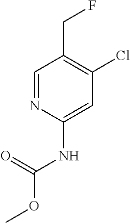

A solution of methyl N-(4-chloro-5-iodo-2-pyridyl)carbamate (A10, 1.0 g, 3.2 mmol), Pd(PPh₃)₄ (0.37 g, 0.32 mmol) and tributylstannyl methanol (3.08 g, 9.60 mmol) in 1,4-dioxane (20 mL) was heated at 100° C. for 8 h. The reaction mixture was cooled to rt and then concentrated under reduced pressure. The crude product was triturated with MTBE (5 mL) and the resulting solid was filtered to obtain methyl (4-chloro-5-(hydroxymethyl)pyridin-2-yl)carbamate (0.26 g, 38%) as a green solid. (ESI) m/z: 217.2 (M+H⁺).

A solution of methyl (4-chloro-5-(hydroxymethyl)pyridin-2-yl)carbamate (0.23 g, 1.06 mmol) in DCM (4 mL) was added to DAST (0.86 g, 5.31 mmol) dropwise. The reaction mixture was stirred at −70° C. for 3 h. The reaction mixture was quenched with MeOH (1 mL), then concentrated under reduced pressure. The residue was treated with water (10 mL) and then extracted with EtOAc (2×). The combined organic layers were dried over anhydrous Na₂SO₄, filtered, and concentrated under reduced pressure. The residue was triturated with MeOH (5 mL) and the precipitates was collected by filtration to give methyl (4-chloro-5-(fluoromethyl)pyridin-2-yl)carbamate (A20, 0.11 g, 47%) as a brown solid. MS (ESI) m/z: 219.1 (M+H⁺).

### Preparation of Intermediate A21: (R)-N-(4-iodo-5-methylpyridin-2-yl)-1-methylpyrrolidine-3-carboxamide

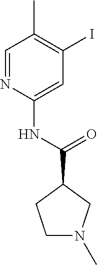 [SMILES: Cc1cnc(NC(=O)[C@@H]2CCN(C)C2)cc1I]

A mixture of (R)-N-(4-iodo-5-methylpyridin-2-yl)pyrrolidine-3-carboxamide (A12, 0.90 g, 2.45 mmol), formaldehyde (0.40 g, 4.90 mmol), AcOH (one drop) and NaBH₃CN (0.46 g, 7.34 mmol) in MeOH (18 mL) was stirred at 0° C. for 1 h under N₂ atmosphere. This reaction mixture was concentrated under reduced pressure, then water (10 ml) was added. The solution was extracted with EtOAc (3×) and the combined organic layers were dried over anhydrous Na₂SO₄, filtered, and concentrated under reduced pressure. The residue was purified by silica gel column chromatography (0 to 20% DCM/MeOH) to afford (R)-N-(4-iodo-5-methylpyridin-2-yl)-1-methylpyrrolidine-3-carboxamide (0.75 g, 89%). MS (ESI) m/z: 346.2 (M+H⁺).

The following compounds are prepared essentially by method of preparation of intermediate A21.

| Ex | | | Yield | | MS (m/z: |
|---|---|---|---|---|---|
| No | SM | Product | (%) | ¹H NMR (400 MHz, CDCl₃): δ | M + H⁺) |
| A22 | A13 CH₂O | 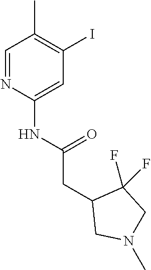 [SMILES: Cc1cnc(NC(=O)CC2CN(C)CC2(F)F)cc1I] | 83 | 8.75 (s, 1H), 8.45 (s, 1H), 8.00 (s, 1H), 2.93-3.23 (m, 4H), 2.48 (m, 2H), 2.38 (s, 3H), 2.36 (s, 3H), 2.12 (s, 1H). | 396.1 |

### Preparation of Intermediate A23: methyl (5-amino-4-chloropyridin-2-yl)carbamate

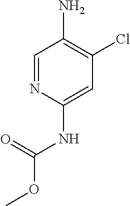

A solution of methyl N-(4-chloro-5-nitro-2-pyridyl)carbamate (A18, 0.84 g, 3.63 mmol) in a mixture of EtOH (20 mL) and H₂O (4 mL) was treated with Fe (0.81 g, 14.5 mmol), NH₄Cl (0.78 g, 14.5 mmol). The mixture was stirred at 80° C. for 8 h and then the hot solution was filter through a pad of celite and washed with hot EtOH (3×). The filtrate was concentrated under reduced pressure to obtain methyl (5-amino-4-chloropyridin-2-yl)carbamate (A23, 1.2 g, 98%) as a reddish brown solid. ¹H NMR (400 MHz, DMSO-d₆): δ 9.85 (br d, J=2.4 Hz, 1H), 7.82 (br s, 1H), 7.54-7.72 (m, 1H), 3.63 (br s, 3H); MS (ESI) m/z: 202.2 (M+H⁺).

### Preparation of Intermediate A24: N-(4-iodo-5-methylpyridin-2-yl)-5-methyl-1,2,4-oxadiazol-3-amine

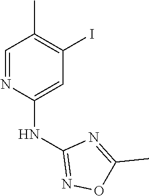

A mixture of 4-iodo-5-methyl-pyridin-2-amine (2.0 g, 8.55 mmol) and NaHCO₃ (0.72 g, 8.55 mmol) in DCM (15 mL) and water (15 mL) was treated with a solution of thiocarbonyl dichloride (0.72 mL, 9.40 mmol) in DCM (15 mL) at 0° C. The reaction mixture was stirred at 0° C. for 2 h under N₂ atmosphere. The reaction mixture was extracted with EtOAc (3×) and the combined organic layers were washed with brine, dried over anhydrous Na₂SO₄, filtered, and concentrated under reduced pressure. The residue was purified by silica gel column chromatography (5 to 10% EtOAc/pet-ether) to obtain 4-iodo-2-isothiocyanato-5-methylpyridine (0.53 g, 121%) as a yellow solid. MS (ESI) m/z: 277.1 (M+H⁺).

A solution of 4-iodo-2-isothiocyanato-5-methylpyridine (0.53 g, 1.92 mmol) and N,N-diethylacetamidine (0.22 g, 1.92 mmol) in DMF (20 mL) was stirred at rt for 1 h. Et₃N (0.67 mL, 4.80 mmol) was added to the mixture followed by slow addition of NH₂OH·HCl (0.13 g, 1.92 mmol) at rt with stirring. A fine powder of AgNO₃ (0.42 g, 2.47 mmol) was added slowly to this suspension and the mixture was stirred at rt for 4 h. The mixture was filtered through a pad of celite and washed with DCM. The filtrate was concentrated under reduced pressure. The residue was purified by C-18 prep-HPLC (5 to 45% H₂O (0.2% FA)/MeCN) to obtain N-(4-iodo-5-methylpyridin-2-yl)-5-methyl-1,2,4-oxadiazol-3-amine (A24, 0.05 g, 8%) as a white solid. MS (ESI) m/z: 317.1 (M+H⁺).

### Preparation of Intermediate BI: 2′-chloro-6′-fluoro-5-methyl-[4,4′-bipyridin]-2-amine

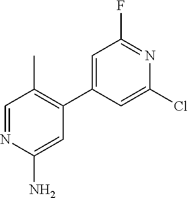 [SMILES: Cc1cnc(N)cc1-c1cc(F)nc(Cl)c1]

Solution of 4-bromo-5-methylpyridin-2-amine (4.0 g, 21.4 mmol), 2-chloro-6-fluoro-4-(4,4,5,5-tetramethyl-1,3,2-dioxaborolan-2-yl)pyridine (6.6 g, 25.7 mmol) in a mixture of 1,4-dioxane (75 mL) and water (11 mL) was treated with Cs₂CO₃ (16.0 g, 49.2 mmol). The mixture was sparged with Ar for 2 min and then Pd(dppf)Cl₂ (0.94 g, 1.28 mmol) was added. The reaction mixture was heated at 80° C. for 3 h and then cooled to rt. The mixture was diluted with DCM and then filtered through a pad of celite. The filtrate was treated with sat'd NaHCO₃ (aq) and then the solution was extracted with DCM (2×). The combined organics were dried over anhydrous MgSO₄, filtered, and concentrated under reduced pressure. The crude was purified by reverse phase column chromatography (0 to 100% H₂O (0.1% FA)/MeCN) to obtain 2′-chloro-6′-fluoro-5-methyl-[4,4′-bipyridin]-2-amine (B1, 1.55 g, 30%) as a white solid. ¹H NMR (400 MHz, DMSO-d₆): δ 7.89 (s, 1H), 7.58 (s, 1H), 7.34 (d, J=1.4 Hz, 1H), 6.44 (d, J=1.7 Hz, 1H), 6.26 (s, 2H), 2.03 (s, 3H); MS (ESI) m/z: 238.0 (M+H⁺).

The following compounds are prepared essentially by method of preparation of intermediate B1.

| Ex | | | Yield | | MS (m/z: |
|---|---|---|---|---|---|
| No | SM | Product | (%) | ¹H NMR (400 MHz, DMSO-d₆): δ | M + H⁺) |
| B2 | 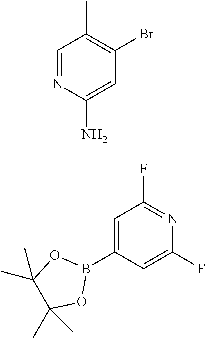 | 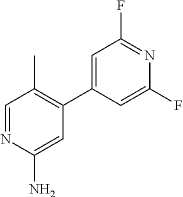 [SMILES: Cc1cnc(N)cc1-c1cc(F)nc(F)c1] | 75 | 8.06 (s, 1H), 6.77 (s, 2H), 6.38 (s, 1H), 4.50 (s, 2H), 2.11 (s, 3H), in CDCl₃. | 222.1 |

| | | | | | | |
|---|---|---|---|---|---|---|
| B3 | 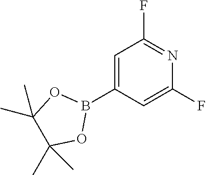 [SMILES: CC1(C)OB(c2cc(F)nc(F)c2)OC1(C)C] | A1 | 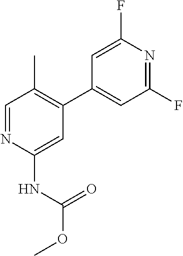 [SMILES: COC(=O)Nc1cc(-c2cc(F)nc(F)c2)c(C)cn1] | 37 | 10.32 (s, 1H), 8.26 (s, 1H), 7.75 (s, 1H), 7.32 (s, 2H), 3.66 (s, 3H), 2.18 (s, 3H). | 280.0 |

| | | | | | |
|---|---|---|---|---|---|
| B4 | 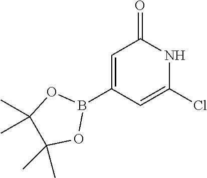 | A16 | 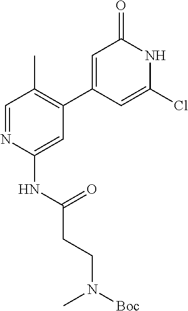 [SMILES: Cc1cnc(NC(=O)CCN(C)C(=O)OC(C)(C)C)cc1-c1cc(Cl)[nH]c(=O)c1] | crude | 421.2 |

| | | | | | |
|---|---|---|---|---|---|
| B5 | 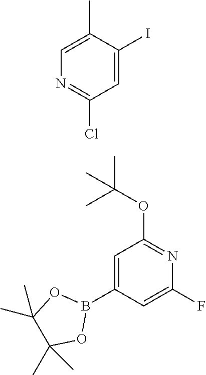 | 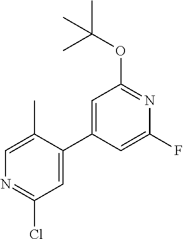 | 75 | 8.38 (s, 1H), 7.45 (s, 1H), 6.80 (s, 1H), 6.71 (s, 1H), 2.21 (s, 3H), 1.57 (s, 9H). | 295.3 |

### Preparation of Intermediate C1: 4-(benzyloxy)-2-(tert-butoxy)-6-chloropyridine

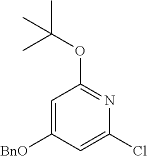 [SMILES: CC(C)(C)Oc1cc(OCc2ccccc2)cc(Cl)n1]

A suspension of 4-(benzyloxy)-2,6-dichloropyridine (5.0 g, 20 mmol) in 2-methyltetrahydrofuran (30 mL) was added tert-BuOK (2.5 g, 22 mmol). The reaction mixture was heated at 70° C. for 2 h and then cooled to rt. The precipitate was filtered and the solid was dissolved in DCM. The solution was dried over anhydrous Na₂SO₄, filtered, and concentrated under reduced pressure to provide 4-(benzyloxy)-2-(tert-butoxy)-6-chloropyridine (4.1 g, 71%) as colorless liquid. MS (ESI) m/z: 236.2 (M+H⁺).

### Preparation of Intermediate C2: (R)-2-fluoro-4-iodo-6-(2-(trifluoromethyl)piperidin-1-yl)pyridine

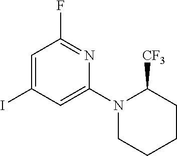 [SMILES: Fc1cc(I)cc(N2CCCC[C@@H]2C(F)(F)F)n1]

A solution of (2R)-2-(trifluoromethyl)piperidine (7.0 g, 46 mmol) in THF (100 mL) was treated with 2.0 M LDA (23 mL, 46 mmol) at −40° C. and stirred for 15 min. 2,6-Difluoro-4-iodo-pyridine (10 g, 46 mmol) was added under the same conditions and then stirred at 0° C. for 1 h. The reaction mixture was quenched with saturated NH₄Cl (100 mL) and the aqueous phase was extracted with EtOAc (3×). The combined organics were washed with brine (100 mL), dried over anhydrous MgSO₄, filtered, and concentrated under reduced pressure. The residue was purified by silica gel column chromatography (0 to 20% EtOAc/pet-ether) to afford (C2, 28 g, 45%) as a pale-yellow oil. ¹H NMR (400 MHz, DMSO-d₆): δ 7.22 (s, 1H), 6.80 (d, J=3.2 Hz, 1H), 5.29 (m, 1H), 4.13 (d, J=12.4 Hz, 1H), 2.96 (t, J=12.8 Hz, 1H), 1.95 (s, 1H), 1.69-1.79 (m, 2H), 1.64 (d, J=4.0 Hz, 2H), 1.44 (m, 1H).

The following compounds are prepared essentially by method of preparation C1 and C2.

| Ex | | | Yield | | MS (m/z: |
|---|---|---|---|---|---|
| No | SM | Product | (%) | 1H NMR (400 MHz, CDCl₃): δ | M + H⁺) |
| C3 | 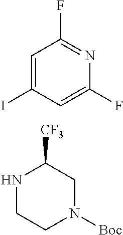 | 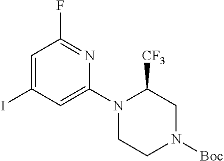 [SMILES: CC(C)(C)OC(=O)N1CCN(c2cc(I)cc(F)n2)[C@@H](C(F)(F)F)C1] | 64 | 6.83 (s, 1H), 6.70 (d, J = 2.8 Hz, 1H), 5.17 (m, 1H), 4.49 (d, J = 14.0 Hz, 1H), 4.29 (d, J = 12.0 Hz, 1H), 3.71 (m, 1H), 3.39 (t, J = 11.2 Hz, 1H), 3.23 (dd, J = 2.8, 14.8 Hz, 1H), 2.82-2.96 (m, 1H), 1.49 (s, 9H). | 420.0 |
| C4 | 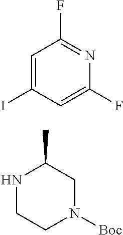 | 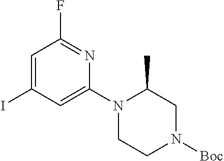 [SMILES: C[C@H]1CN(C(=O)OC(C)(C)C)CCN1c1cc(I)cc(F)n1] | 77 | | 366.2 |

### Preparation of Intermediate D1: (R)-4-(Benzyloxy)-2-(Tert-Butoxy)-6-(2-(trifluoromethyl)piperidin-1-yl)pyridine

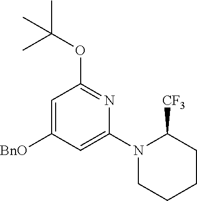 [SMILES: CC(C)(C)Oc1cc(OCc2ccccc2)cc(N2CCCC[C@@H]2C(F)(F)F)n1]

A suspension of 4-(benzyloxy)-2-(tert-butoxy)-6-chloropyridine (C1, 4.0 g, 14 mmol) and (R)-2-trifluoromethyl piperidine (2.5 g, 16 mmol) in 1,4-dioxane (30 mL) was treated with sodium butan-1-olate (3.9 g, 41 mmol) followed by PEPSI-iPr (0.1 g, 0.15 mmol). The resulting reaction mixture was heated at 90° C. for 2 h and then the mixture concentrated under reduced pressure. The residue was treated with EtOAc (30 mL) and the solution was washed with brine. The organic layer was dried over anhydrous Na₂SO₄, filtered, and concentrated under reduced pressure. The residue was dissolved in DCM and then filtered through a pad silica gel. The filtrate was concentrated under reduced pressure to provide (R)-4-(benzyloxy)-2-(tert-butoxy)-6-(2-(trifluoromethyl)piperidin-1-yl)pyridine (D1, 3.8 g, 68%) as a brown solid. ¹H NMR (400 MHz, DMSO-d6): δ 7.23-7.48 (m, 5H), 5.99 (s, 1H), 5.67 (s, 1H), 5.27-5.42 (m, 1H), 5.08 (s, 2H), 3.94 (br d, J=12.6 Hz, 1H), 2.97 (m, 1H), 1.98 (br d, J=13.2 Hz, 1H), 1.64 (br s, 5H), 1.48 (s, 9H); MS (ESI) m/z: 409.2 (M+H⁺).

### Preparation of Intermediate D2: (R)-4-iodo-2-((4-methoxybenzyl)oxy)-6-(2-(trifluoromethyl)piperidin-1-yl)pyridine

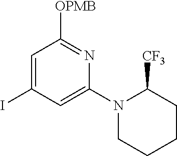

A solution of NaH (2.8 g, 70.2 mmol, 60% in mineral oil) in THF (200 mL) was stirred under N₂ atmosphere. (4-Methoxyphenyl)methanol (6.4 mL, 51.4 mmol) was added dropwise at 0° C. and the mixture was stirred at 0° C. for 20 min. 2-Fluoro-4-iodo-6-[(2R)-2-(trifluoromethyl)-1-piperidyl]pyridine (17.5 g, 46.8 mmol) was added and the resulting mixture was stirred at 55° C. for 12 h. The reaction mixture was quenched with NH₄Cl (aq, 200 mL), and diluted with EtOAc (200 mL). The organic layer was washed with brine, dried over anhydrous MgSO₄, filtered, and concentrated under reduced pressure. The residue was purified by silica gel column chromatography (0 to 5% EtOAc/pet-ether) to afford (R)-4-iodo-2-((4-methoxybenzyl)oxy)-6-(2-(trifluoromethyl)piperidin-1-yl)pyridine (20.0 g, 83%) as a yellow oil. ¹H NMR (400 MHz, DMSO-d₆): δ 7.31 (d, J=8.8 Hz, 2H), 6.90 (d, J=8.4 Hz, 2H), 6.79 (s, 1H), 6.52 (s, 1H), 5.39 (m, 1H), 5.17 (s, 2H), 4.11 (m, 1H), 3.74 (s, 3H), 2.96 (br t, J=12.4 Hz, 1H), 1.95 (br s, 1H), 1.71 (br d, J=12.8 Hz, 2H), 1.63 (br s, 2H), 1.43 (m, 1H); MS (ESI) m/z: 493.2 (M+H⁺).

The following compounds are prepared essentially by method of preparation of intermediates D1 and D2.

| Ex | | | Yield | | MS (m/z: |
|---|---|---|---|---|---|
| No | SM | Product | (%) | ¹H NMR (400 MHz, DMSO-d₆): δ | M + H⁺) |
| D3 | C1 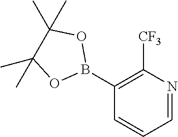 Suzuki | 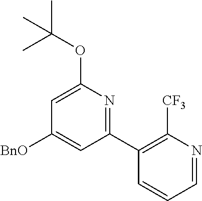 [SMILES: CC(C)(C)Oc1cc(OCc2ccccc2)cc(-c2cccnc2C(F)(F)F)n1] | 85 | 8.80 (d, J = 4.0 Hz, 1H), 8.00 (d, J = 7.6 Hz, 1H), 7.79 (dd, J = 4.4, 7.6 Hz, 1H), 7.30-7.50 (m, 5H), 6.75 (d, J = 1.6 Hz, 1H), 6.38 (d, J = 1.6 Hz, 1H), 5.19 (s, 2H), 1.49 (s, 9H). | 403.0 |
| D4 | 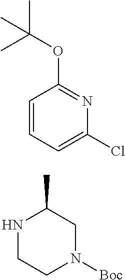 | 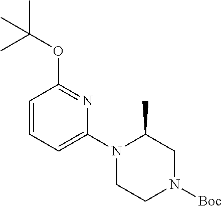 [SMILES: C[C@H]1CN(C(=O)OC(C)(C)C)CCN1c1cccc(OC(C)(C)C)n1] | 65 | 7.36 (t, J = 8.0 Hz, 1H), 6.08 (d, J = 8.0 Hz, 1H), 6.02 (d, J = 8.0 Hz, 1H), 4.43 (s, 1H), 3.91-4.21 (m, 2H), 3.80 (m, 1H), 3.00-3.22 (m, 3H), 1.57 (s, 9H), 1.50 (s, 9H), 1.12 (d, J = 6.4 Hz, 3H), in CDCl₃. | 350.2 |
| D5 | 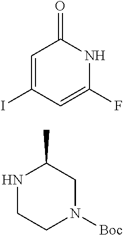 | 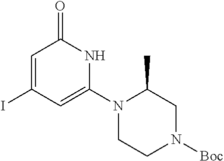 [SMILES: C[C@H]1CN(C(=O)OC(C)(C)C)CCN1c1cc(I)cc(=O)[nH]1] | 50 | | 420.0 |
| D6 | 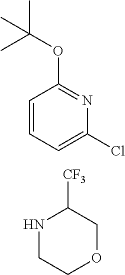 | 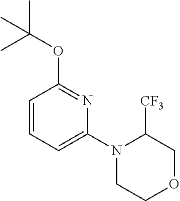 [SMILES: CC(C)(C)Oc1cccc(N2CCOCC2C(F)(F)F)n1] | 95 | 7.41 (t, J = 8.0 Hz, 1H), 6.11 (dd, J = 1.6, 8.0 Hz, 2H), 5.05 (m, 1H), 4.33 (d, J = 12.4 Hz, 1H), 4.03 (dd, J = 3.2, 10.8 Hz, 1H), 3.78 (m, 1H), 3.44-3.68 (m, 3H), 1.55 (s, 9H), in CDCl₃. | 305.3 |
| D7 | C3 PMBOH NaH | 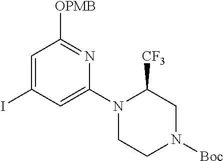 | 21 | 7.32 (d, J = 8.8 Hz, 2H), 6.90 (d, J = 8.8 Hz, 2H), 6.63 (s, 1H), 6.52 (s, 1H), 5.18 (s, 2H), 4.47 (m, 1H), 4.25 (m, 1H), 3.91 (s, 1H), 3.82 (s, 3H), 3.71 (d, J = 12.4 Hz, 1H), 3.37 (m, 1H), 3.21 (m, 1H), 2.83-3.11 (m, 1H), 1.49 (s, 9H) in CDCl₃. | 616.1 |
| D8 | 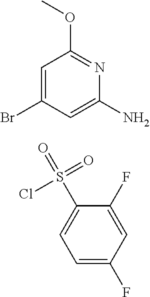 | 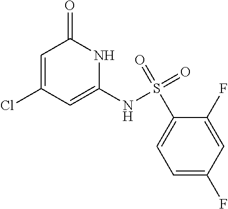 [SMILES: O=c1cc(Cl)cc(NS(=O)(=O)c2ccc(F)cc2F)[nH]1] | 41 | | 367.0 |
| | BBr₃ | | | | |
| D9 | 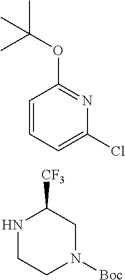 | 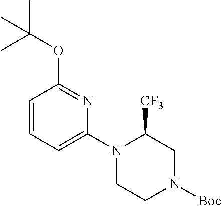 [SMILES: CC(C)(C)OC(=O)N1CCN(c2cccc(OC(C)(C)C)n2)[C@@H](C(F)(F)F)C1] | 65 | 7.41 (m, 1H), 6.14 (d, J = 7.6 Hz, 1H), 6.11 (d, J = 7.6 Hz, 1H), 5.19 (br d, J = 2.4 Hz, 1H), 4.49 (d, J = 14.4 Hz, 1H), 4.25 (d, J = 12.4 Hz, 1H), 3.63 (m, 1H), 3.20-3.48 (m, 2H), 2.98 (d, J = 11.4 Hz, 1H), 1.55 (s, 9H), 1.49 (s, 9H) in CDCl₃. | 404.4 |
| D10 | C4 PMBOH NaH | 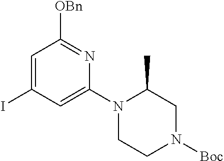 [SMILES: C[C@H]1CN(C(=O)OC(C)(C)C)CCN1c1cc(I)cc(OCc2ccccc2)n1] | 91 | 7.29-7.43 (m, 5H), 6.55 (s, 1H), 6.46 (s, 1H), 5.23-5.53 (m, 2H), 4.40 (m, 1H), 4.18 (m, 1H), 3.94 (m, 1H), 3.84 (br d, J = 12.4 Hz, 1H), 2.92-3.16 (m, 3H), 1.49 (s, 9H), 1.09 (d, J = 6.8 Hz, 3H) in CDCl₃. | 510.2 |

### Preparation of Intermediate E1: (R)-2-(tert-butoxy)-6-(2-(trifluoromnethyl)piperidin-1-yl)pyridin-4-ol

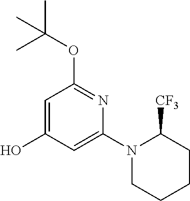 [SMILES: CC(C)(C)Oc1cc(O)cc(N2CCCC[C@@H]2C(F)(F)F)n1]

A solution of (R)-4-(benzyloxy)-2-(tert-butoxy)-6-(2-(trifluoromnethyl)piperidin-1-yl)pyridine (D1, 3.5 g, 8.6 mmol) in MeOH (30 mL) was treated with palladium on charcoal (10% wet) (0.9 g, 8.6 mmol). The reaction mixture was subjected to hydrogenation on Parr shaker at 40 psi for 2 h. The reaction mixture was filtered through a pad of celite, washed with MeOH. The filtrate was dried over anhydrous Na₂SO₄, filtered, and concentrated under reduced pressure to provide (R)-2-(tert-butoxy)-6-(2-(trifluoromethyl)piperidin-1-yl)pyridin-4-ol (E1, 2.6 g, 95%) as a brown solid. ¹H NMR (400 MHz, DMSO-d₆): δ 10.00 (s, 1H), 5.73 (s, 1H), 5.43 (s, 1H), 5.20-5.35 (m, 1H), 3.81 (m, 1H), 2.96 (br t, J=12.8 Hz, 1H), 1.99 (m, 1H), 1.61-1.77 (m, 4H), 1.50 (m, 1H), 1.45 (s, 9H); MS (ESI) m/z: 319.2 (M+H⁺).

The following compounds are prepared essentially by method of preparation E1.

| Ex | | | Yield | | MS (m/z: |
|---|---|---|---|---|---|
| No | SM | Product | (%) | ¹H NMR (400 MHz, DMSO-d₆): δ | M + H⁺) |
| E2 | D3 | 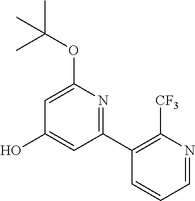 [SMILES: CC(C)(C)Oc1cc(O)cc(-c2cccnc2C(F)(F)F)n1] | 60 | 11.23 (s, 1H), 9.30 (d, J = 4.8 Hz, 1H), 8.50 (d, J = 8.0 Hz, 1H), 8.29 (dd, J = 4.4, 7.6 Hz, 1H), 7.01 (d, J = 1.2 Hz, 1H), 6.58 (d, J = 1.2 Hz, 1H), 1.98 (s, 9H). | 313.1 |

### Preparation of Intermediate F1: (R)-2-(tert-butoxy)-6-(2-(trifluoromethyl)piperidin-1-yl)pyridin-4-yl trifluoromethanesulfonate

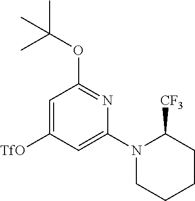 [SMILES: CC(C)(C)Oc1cc(OS(=O)(=O)C(F)(F)F)cc(N2CCCC[C@@H]2C(F)(F)F)n1]

A solution of (R)-2-(tert-butoxy)-6-(2-(trifluoromethyl)piperidin-1-yl)pyridin-4-ol (E1, 2.6 g, 8.2 mmol) in DCM (30 mL) and Et₃N (2.0 mL, 15 mmol) was cooled to 0° C. Trifluoromethanesulfonic anhydride (1.5 mL, 9.2 mmol) was added under the same conditions and then the reaction mixture was warmed to rt and stirred for 2 h. The reaction mixture quenched with sat'd NaHCO₃ (aq) solution and the mixture was extracted with DCM (2×). The combined organics were dried over anhydrous Na₂SO₄, filtered, and concentrated under reduced pressure to provide (R)-2-(tert-butoxy)-6-(2-(trifluoromethyl)piperidin-1-yl)pyridin-4-yl trifluoromethanesulfonate (F1, 2.8 g, 76%) as a red viscous mass. ¹H NMR (400 MHz, CDCl₃): δ 5.99 (s, 1H), 5.94 (s, 1H), 5.12-5.37 (m, 1H), 3.81 (br d, J=13.2 Hz, 1H), 3.20 (br t, J=14.0 Hz, 1H), 2.13 (m, 1H), 1.70-1.89 (m, 4H), 1.56-1.59 (m, 1H), 1.55 (s, 9H); MS (ESI) m/z: 450.9 (M+H⁺).

The following compounds are prepared essentially by method of preparation F1.

| Ex | | | Yield | | MS (m/z: |
|---|---|---|---|---|---|
| No | SM | Product | (%) | ¹H NMR (400 MHz, CDCl₃): δ | M + H⁺) |
| F2 | E2 | 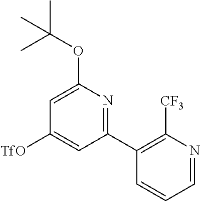 [SMILES: CC(C)(C)Oc1cc(OS(=O)(=O)C(F)(F)F)cc(-c2cccnc2C(F)(F)F)n1] | 76 | 8.80 (d, J = 3.6 Hz, 1H), 7.88 (d, J = 7.8 Hz, 1H), 7.61 (dd, J = 4.8, 7.8 Hz, 1H), 6.90 (d, J = 2.0 Hz, 1H), 6.65 (d, J = 2.0 Hz, 1H), 1.58 (s, 9H). | 445.0 |

### Preparation of Intermediate G1: (R)-2-(tert-butoxy)-4-(4,4,5,5-tetramethyl-1,3,2-dioxaborolan-2-yl)-6-(2-(trifluoromethyl)piperidin-1-yl)pyridine

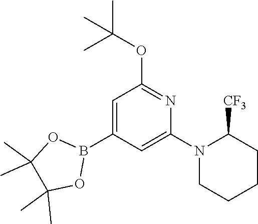 [SMILES: CC(C)(C)Oc1cc(B2OC(C)(C)C(C)(C)O2)cc(N2CCCC[C@@H]2C(F)(F)F)n1]

A mixture of (R)-2-(tert-butoxy)-6-(2-(trifluoromethyl)piperidin-1-yl)pyridin-4-yl trifluoromethanesulfonate (F1, 1.0 g, 2.5 mmol) and BPD (0.7 g, 2.8 mmol) in 1,4-dioxane (30 mL) was treated with KOAc (0.7 g, 7.1 mmol) followed by Pd(dppf)Cl₂ (0.1 g, 0.12 mmol). The reaction mixture stirred at 110° C. for 3 h and then cooled to rt. The mixture was filtered through a pad of celite and washed with DCM. The filtrate was concentrated under reduced pressure. The residue was filter through a short silica gel column with EtOAc (100 mL). The filtrate was concentrated under reduced pressure to provide (R)-2-(tert-butoxy)-4-(4,4,5,5-tetramethyl-1,3,2-dioxaborolan-2-yl)-6-(2-(trifluoromethyl)piperidin-1-yl)pyridine (G1, 0.70 g, 65%) as a dark colored semi solid. ¹H NMR (400 MHz, DMSO-d₆): δ 8.81 (d, J=4.4 Hz, 1H), 8.02 (d, J=7.6 Hz, 1H), 7.79 (dd, J=4.8, 8.0 Hz, 1H), 7.16 (s, 1H), 6.92 (s, 1H), 1.49 (s, 9H), 1.29 (s, 12H); MS (ESI) m/z: 429.0 (M+H⁺).

The following compounds are prepared essentially by method of preparation G1.

| Ex | | | Yield | | MS (m/z: |
|---|---|---|---|---|---|
| No | SM | Product | (%) | ¹H NMR (400 MHz, CDCl₃): δ | M + H+) |
| G2 | F2 | 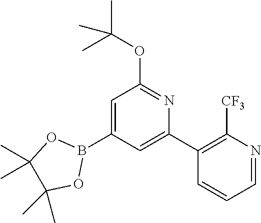 [SMILES: CC(C)(C)Oc1cc(B2OC(C)(C)C(C)(C)O2)cc(-c2cccnc2C(F)(F)F)n1] | 86 | 8.81 (d, J = 4.4 Hz, 1H), 8.02 (d, J = 7.6 Hz, 1H), 7.79 (dd, J = 8.0, 4.8 Hz, 1H), 7.16 (s, 1H), 6.92 (s, 1H), 1.49 (s, 9H), 1.29 (s, 12H). | 422.9 |
| G3 | D2 iPrMgCl iPrOBPin | 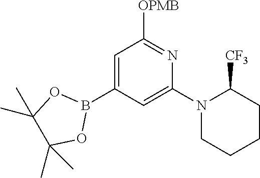 | 57 | 7.31 (d, J = 8.4 Hz, 2H), 6.89 (d, J = 8.4 Hz, 2H), 6.51 (s, 1H), 6.30 (s, 1H), 5.43 (m, 1H), 5.19 (d, J = 1.6 Hz, 2H), 3.98 (m, 1H), 3.73 (s, 3H), 3.01 (t, J = 12.4 Hz, 1H), 1.95 (s, 1H), 1.72 (d, J = 13.2 Hz, 2H), 1.63 (s, 2H), 1.44 (m, 1H) 1.26-1.29 (m, 12H). | 493.5 |
| G4 | D9 | 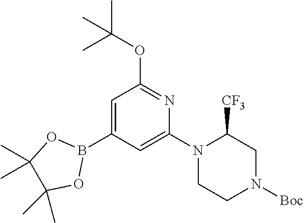 [SMILES: CC(C)(C)OC(=O)N1CCN(c2cc(B3OC(C)(C)C(C)(C)O3)cc(OC(C)(C)C)n2)[C@@H](C(F)(F)F)C1] | 91 | 6.51 (d, J = 4.4 Hz, 2H), 5.31 (s, 1H), 5.23 (m, 1H), 4.49 (br d, J = 14.8 Hz, 1H), 4.25 (m, 1H), 3.80 (br d, J = 11.4 Hz, 1H), 3.39 (br t, J = 11.6 Hz, 1H), 3.24 (br d, J = 12.8 Hz, 1H), 3.01 (m, 1H), 1.52 (s, 9H), 1.48 (s, 9H), 1.33 (s, 12H) in CDCl₃. | 530.5 |
| G5 | A7 | 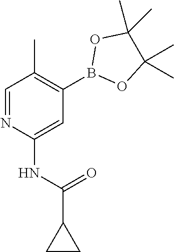 [SMILES: Cc1cnc(NC(=O)C2CC2)cc1B1OC(C)(C)C(C)(C)O1] | crude | | 303.4 |

### Preparation of Intermediate G6: 4-(6-(tert-butoxy)-4-(4,4,5,5-tetramethyl-1,3,2-dioxaborolan-2-yl)pyridin-2-yl)-3-(trifluorodimethyl)morpholine

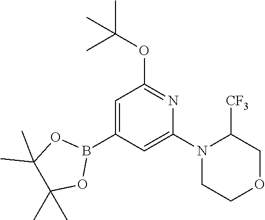 [SMILES: CC(C)(C)Oc1cc(B2OC(C)(C)C(C)(C)O2)cc(N2CCOCC2C(F)(F)F)n1]

A mixture of 4-(6-tert-butoxy-2-pyridyl)-3-(trifluoromethyl)morpholine (0.10 g, 0.33 mmol), BPD (0.42 g, 1.64 mmol), [Ir(OMe)(COD)]₂ (22 mg, 0.1 eq), and 4-tert-butyl-2-(4-tert-butyl-2-pyridyl)pyridine (18 mg, 0.2 eq) in MTBE (10 mL) was degassed and purged with N₂ for 3 min. The mixture was heated at 80° C. for 2 h under microwave. The reaction mixture was filtered through a pad of celite and the filtrate was concentrated under reduced pressure. The residue was purified by silica gel column chromatography (0 to 20% EtOAc/pet-ether) to afford 4-[6-tert-butoxy-4-(4,4,5,5-tetramethyl-1,3,2-dioxaborolan-2-yl)-2-pyridyl]-3-(trifluoromethyl)morpholine (G6, 0.35 g, crude) as a brown oil. ¹H NMR (400 MHz, CDCl₃): δ 6.52 (s, 1H), 6.48 (s, 1H), 5.12 (m, 1H), 4.32 (d, J=12.4 Hz, 1H), 4.02 (dd, J=2.8, 10.4 Hz, 1H), 3.77 (m, 1H), 3.47-3.69 (m, 3H), 1.52 (s, 9H), 1.33 (s, 12H)

The following compounds are prepared essentially by method of preparation of intermediate G6.

| | | | | MS |
|---|---|---|---|---|
| | | | | (m/z: |
| Ex | | | Yield | M + |
| No | SM | Product | (%) | H⁺) |
| G7 | D4 | 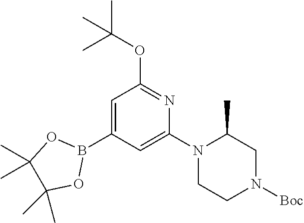 [SMILES: C[C@H]1CN(C(=O)OC(C)(C)C)CCN1c1cc(B2OC(C)(C)C(C)(C)O2)cc(OC(C)(C)C)n1] | 98 | 394.2 |

### Preparation of Intermediate H1: 2′-chloro-6′-((4-methoxybenzyl)oxy)-5-methyl-[4,4′-bipyridin]-2-amine

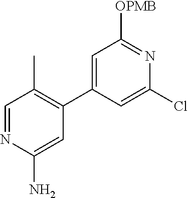

A solution of (4-methoxyphenyl)methanol (0.78 g, 5.6 mmol) in 1,4-dioxane (25 mL) was treated with LiHMDS (9.3 mL, 9.3 mmol) at rt. The reaction mixture was stirred at rt for 10 min and then 2′-chloro-6′-fluoro-5-methyl-[4,4′-bipyridin]-2-amine (B1, 1.7 g, 7.2 mmol) was added. The reaction was heated at 100° C. for 24 h and then cooled to rt. The reaction mixture was diluted with DCM and sat'd NaHCO₃ (aq). The mixture was extracted with DCM (2×) and then the combined organics were dried over anhydrous MgSO₄, filtered, and concentrated under reduced pressure. The crude was purified silica gel column chromatography (0 to 100% EtOAc/hexanes) to obtain 2′-chloro-6′-((4-methoxybenzyl)oxy)-5-methyl-[4,4′-bipyridin]-2-amine (H1, 2.22 g, 116%) as an amber oil. MS (ESI) m/z: 356.2 (M+H⁺).

The following compounds are prepared essentially by method of preparation of intermediate H1.

| Ex | | | Yield | MS (m/z: |
|---|---|---|---|---|
| No | SM | Product | (%) | M + H⁺) |
| H2 | B2 PMBOH LiHMDS | 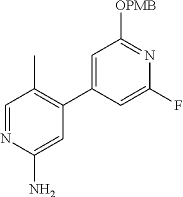 | 96 | 340.2 |

### Preparation of Intermediate H3: N-(2′-chloro-6′-((4-methoxybenzyl)oxy)-5-methyl-[4,4′-bipyridin]-2-yl)cyclopropanecarboxamide

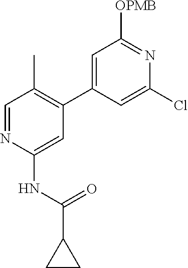

A solution of 2′-chloro-6′-((4-methoxybenzyl)oxy)-5-methyl-[4,4′-bipyridin]-2-amine (H1, 1.0 g, 2.8 mmol) in DCM (10 mL) was treated with pyridine (0.36 mL, 4.5 mmol). The mixture was stirred at rt and then cyclopropanecarbonyl chloride (0.46 mL, 5.06 mmol) was added dropwise. The reaction mixture was stirred at rt for 1 h and then concentrated under reduced pressure. The residue was diluted with water (30 mL) and the solution was extracted with DCM (3×). The combined organics were dried over anhydrous Na₂SO₄, filtered, and concentrated under reduced pressure. The residue was purified by silica gel column chromatography (0 to 20% EtOAc/pet-ether) to give N-(2′-chloro-6′-((4-methoxybenzyl)oxy)-5-methyl-[4,4′-bipyridin]-2-yl)cyclopropanecarboxamide (1.1 g, 74%) as a yellow solid. ¹H NMR (400 MHz, DMSO-d₆): δ 10.86 (s, 1H), 8.26 (s, 1H), 7.95 (s, 1H), 7.42 (d, J=8.4 Hz, 2H), 7.15 (d, J=0.8 Hz, 1H), 6.95 (d, J=8.4 Hz, 1H), 6.88 (d, J=0.8 Hz, 1H), 5.29 (s, 2H), 3.76 (s, 3H), 2.17 (s, 3H), 1.99 (m, 1H), 0.80 (s, 2H), 0.78 (s, 2H); MS (ESI) m/z: 424.2 (M+H⁺).

### Preparation of Intermediate H4: 2′-(tert-butoxy)-6′-fluoro-5-methyl-N-(2-methylpyrimidin-4-yl)-[4,4′-bipyridin]-2-amine

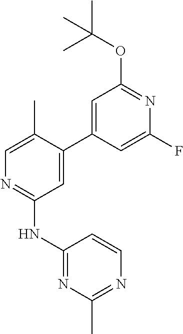 [SMILES: Cc1nccc(Nc2cc(-c3cc(F)nc(OC(C)(C)C)c3)c(C)cn2)n1]

A mixture of 2-tert-butoxy-4-(2-chloro-5-methyl-4-pyridyl)-6-fluoro-pyridine (B5, 1.24 g, 4.21 mmol) and 2-methylpyrimidin-4-amine (1.38 g, 12.6 mmol) in 2-methylbutan-2-ol (15 mL) was treated with Cs₂CO₃ (4.11 g, 12.6 mmol). The reaction mixture was degassed and purged with N₂ gas for 3 min. BINAP (0.26 g, 0.42 mmol) and rac-BINAP-Pd-G3 (0.42 g, 0.42 mmol) were added and then the mixture was heated at 100° C. for 3 h under N₂ atmosphere. After cooling to rt, the reaction mixture was concentrated under reduced pressure. The residue was diluted with H₂O (50 mL) and extracted with EtOAc (3×). The combined organic layers were dried over anhydrous Na₂SO₄, filtered, and concentrated under reduced pressure. The residue was purified by silica gel column chromatography (0 to 30% EtOAc/pet-ether) to give 2′-(tert-butoxy)-6′-fluoro-5-methyl-N-(2-methylpyrimidin-4-yl)-[4, 4′-bipyridin]-2-amine (H4, 1.23 g, 68%) as a yellow solid. ¹H NMR (400 MHz, DMSO-d₆): δ 10.15 (s, 1H), 8.32 (d, J=5.6 Hz, 1H), 8.24 (s, 1H), 7.66 (d, J=6.0 Hz, 1H), 7.48 (s, 1H), 6.76 (s, 1H), 6.65 (s, 1H), 2.45 (s, 3H), 2.16 (s, 3H), 1.58 (s, 9H); MS (ESI) m/z: 368.4 (M+H⁺).

### Preparation of Intermediate I1: 6′-(tert-butoxy)-2″-chloro-5′-methyl-2-(trifluoromethyl)-3,2′:4′,4″-terpyridine

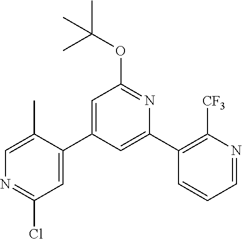 [SMILES: Cc1cnc(Cl)cc1-c1cc(OC(C)(C)C)nc(-c2cccnc2C(F)(F)F)c1]

A solution of 6-(tert-butoxy)-4-(4,4,5,5-tetramethyl-1,3,2-dioxaborolan-2-yl)-2′-(trifluoromethyl)-2,3′-bipyridine (G2, 8.0 g, 18.9 mmol) and 2-chloro-4-iodo-5-methyl-pyridine (4.8 g, 18.9 mmol) in a mixture of 1,4-dioxane (100 mL) and H₂O (5 mL) was treated with Na₂CO₃ (4.0 g, 37.8 mmol). The reaction mixture was degassed with N₂ for 3 min and then Pd(dppf)Cl₂ (1.4 g, 1.89 mmol) was added. The reaction mixture was heated at 90° C. for 3 h under N₂ atmosphere. The reaction mixture was cooled to rt and diluted with water (200 mL). The mixture was extracted with EtOAc (3×). The combined organic layers were dried over anhydrous MgSO₄, filtered, and concentrated under reduced pressure. The residue was purified by silica gel column chromatography (0 to 30% EtOAc/pet-ether) to afford 6′-(tert-butoxy)-2″-chloro-5′-methyl-2-(trifluoromethyl)-3,2′:4′,4″-terpyridine (7.8 g, 83%) as a yellow solid. ¹H NMR (400 MHz, CDCl₃): δ 8.78 (d, J=3.6 Hz, 1H), 8.31 (s, 1H), 7.92 (d, J=7.2 Hz, 1H), 7.60 (dd, J=4.4, 8.0 Hz, 1H), 7.22 (s, 1H), 6.88 (s, 1H), 6.64 (d, J=1.2 Hz, 1H), 2.28 (s, 3H), 1.61 (s, 9H).

The following compounds are prepared essentially by method of preparation of intermediate I1.

| Ex | | | | Yield | | MS (m/z: |
|---|---|---|---|---|---|---|
| No | SM | | Product | (%) | ¹H NMR (400 MHz, DMSO-d₆): δ | M + H⁺) |
| I2 | 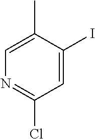 | G3 | 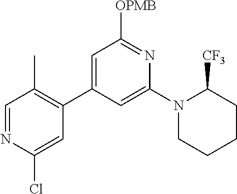 | 68 | 8.34 (s, 1H), 7.38-7.35 (m, 3H), 6.92 (d, J = 8.8 Hz, 2H), 6.44 (s, 1H), 6.14 (s, 1H), 5.47 (m, 1H), 5.23 (s, 2H), 4.23 (d, J = 13.6 Hz, 1H), 3.75 (s, 3H), 3.01 (t, J = 12.8 Hz, 1H), 2.20 (s, 3H), 2.01 (s, 1H), 1.62-1.79 (m, 4H), 1.47 (m, 1H). | 492.3 |

### Preparation of Intermediate J1: 6′-(tert-butoxy)-5′-methyl-2-(trifluoromethyl)-[3,4′:2′,4″-terpyridin]-2″-amine

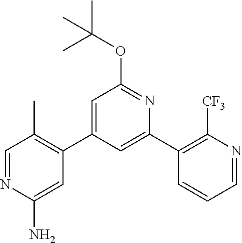 [SMILES: Cc1cnc(N)cc1-c1cc(OC(C)(C)C)nc(-c2cccnc2C(F)(F)F)c1]

A suspension of 2′-(tert-butoxy)-6′-(4,4,5,5-tetramethyl-1,3,2-dioxaborolan-2-yl)-2-(trifluoromethyl)-3,4′-bipyridine (G2, 3.5 g, 8.3 mmol) and 4-iodo-5-methylpyridin-2-amine (2.5 g, 11 mmol) in a mixture of EtOH (45 mL) and water (5 mL) was treated with K₂CO₃ (2.5 g, 18.1 mmol). The reaction mixture was degassed and purged with N₂ gas for 3 min and then XPhos-Pd-G2 (0.040 g, 0.051 mmol) was added. The reaction mixture was heated at 100° C. for 2 h and then cooled to rt. The mixture was concentrated under reduced pressure and the residue was treated with DCM (200 mL). The solid was filtered off and the filtrate was concentrated under reduced pressure. The residue was purified by silica gel column chromatography (0 to 20% MeOH/DCM) to give 6′-(tert-butoxy)-5′-methyl-2-(trifluoromethyl)-[3,4′:2′,4″-terpyridin]-2″-amine (2.6 g, 78%) as a greenish foamy solid. MS (ESI) m/z: 403.2 (M+H⁺).

The following compounds are prepared essentially by method of preparation of intermediate J1.

| Ex | | | | Yield | | MS (m/z: |
|---|---|---|---|---|---|---|
| No | SM | | Product | (%) | ¹H NMR (400 MHz, DMSO-d₆): δ | M + H⁺) |
| J2 | 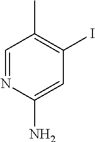 | G3 | 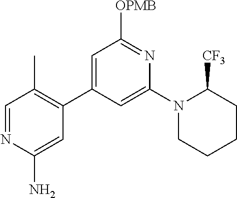 | 60 | 7.80 (s, 1H), 7.36 (d, J = 8.4 Hz, 2H), 6.92 (d, J = 8.4 Hz, 2H), 6.31 (d, J = 5.6 Hz, 2H), 6.02 (s, 1H), 5.75 (s, 2H), 5.45 (m, 1H), 5.22 (s, 2H), 4.18 (br d, J = 12.0 Hz, 1H), 3.75 (s, 3H), 3.01 (br t, J = 12.8 Hz, 1H), 1.99 (s, 3H), 1.96 (m, 1H), 1.72 (br d, J = 12.0 Hz, 2H), 1.65 (s, 2H), 1.51 (s, 1H). | 473.2 |
| J3 | 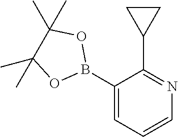 | H1 | 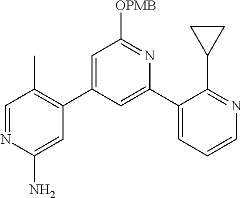 | 72 | | 439.2 |
| J4 | 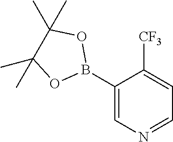 | H1 | | 69 | | 467.2 |
| J5 | 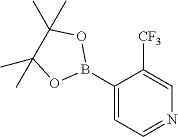 | H1 | | 73 | | 467.2 |
| J6 | 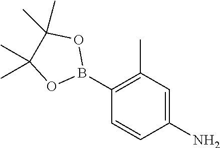 | H3 | | 76 | 10.81 (s, 1H), 8.26 (s, 1H), 8.00 (s, 1H), 7.40 (d, J = 8.8 Hz, 2H), 7.23 (d, J = 8.8 Hz, 1H), 6.97 (d, J = 1.2 Hz, 1H), 6.93 (d, J = 8.4 Hz, 2H), 6.65 (d, J = 1.2 Hz, 1H), 6.47 (dd, J = 2.4, 4.4 Hz, 2H), 5.33 (s, 2H), 5.24 (s, 2H), 3.75 (s, 3H), 2.34 (s, 3H), 2.22 (s, 3H), 2.00 (m, 1H), 0.79 (d, J = 6.0 Hz, 4H). | 295.3 |

### Preparation of Intermediate J7: (R)-2′-((4-methoxybenzyl)oxy)-6′-(2-(methoxymethyl)pyrrolidin-1-yl)-5-methyl-[4,4′-bipyridin]-2-amine

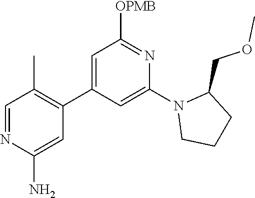

A solution of (R)-2-(methoxymethyl)pyrrolidine (0.22 mL, 1.8 mmol) in 1,4-dioxane (4.4 mL) was treated with LiHMDS (3.5 mL, 3.5 mmol). The reaction mixture was stirred for 5 min at r. 2′-Fluoro-6′-((4-methoxybenzyl)oxy)-5-methyl-[4,4′-bipyridin]-2-amine (H2, 0.3 g, 0.88 mmol) was added and the reaction stirred 165° C. for 45 min. The reaction was cooled to rt and then quenched with sat'd NaHCO₃ (aq). The mixture was extracted with DCM (2×) and the combined organics were dried over anhydrous MgSO₄, filtered, and concentrated under reduced pressure. The crude was purified by silica gel column chromatography (0 to 100% EtOAc/hexanes) to obtain (R)-2′-((4-methoxybenzyl)oxy)-6′-(2-(methoxymethyl)pyrrolidin-1-yl)-5-methyl-[4,4′-bipyridin]-2-amine (0.20 g, 53%) as a clear amber oil. MS (ESI) m/z: 435.2 (M+H⁺).

### Preparation of Intermediate J8: 6-amino-5′-methyl-2′-((2-methylpyrimidin-4-yl)amino)-[4,4′-bipyridin]-2(1H)-one

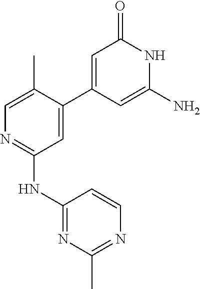 [SMILES: Cc1nccc(Nc2cc(-c3cc(N)[nH]c(=O)c3)c(C)cn2)n1]

A solution of 2′-(tert-butoxy)-6′-fluoro-5-methyl-N-(2-methylpyrimidin-4-yl)-[4,4′-bipyridin]-2-amine (0.50 g, 1.36 mmol) in (4-methoxyphenyl)methanamine (5 mL) was heated at 110° C. for 36 h. The reaction mixture was cooled to rt, then quenched with 1.0 M HCl (adjust pH=6). The solution was extracted with EtOAc (3×) and the combined organics were dried over anhydrous Na₂SO₄, filtered, and concentrated under reduced pressure. The residue was purified by silica gel column chromatography (0 to 50% EtOAc/pet-ether) to give 6′-(tert-butoxy)-N²′-(4-methoxybenzyl)-5-methyl-N²-(2-methylpyrimidin-4-yl)-[4,4′-bipyridine]-2,2′-diamine (0.68 g 79%) as a yellow solid. ¹H NMR (400 MHz, CDCl₃): δ 8.35 (d, J=6.0 Hz, 1H), 8.16 (s, 1H), 7.52 (d, J=6.0 Hz, 1H), 7.30 (d, J=8.4 Hz, 2H), 7.25 (br s, 1H), 7.22 (d, J=8.4 Hz, 1H), 6.90 (d, J=8.4 Hz, 2H), 5.95 (s, 1H), 5.85 (s, 1H), 4.70 (br t, J=4.4 Hz, 1H), 4.45 (d, J=5.6 Hz, 2H), 3.82 (s, 3H), 2.61 (s, 3H), 2.19 (s, 3H), 1.59 (s, 9H).

A solution of 6′-(tert-butoxy)-N²-(4-methoxybenzyl)-5-methyl-N²-(2-methylpyrimidin-4-yl)-[4,4′-bipyridine]-2,2′-diamine (0.44 g, 0.91 mmol) in TFA (5 mL) was added Et₃SiH (0.29 mL, 2 eq). The mixture was stirred at 40° C. for 3 h and then concentration under reduced pressure. The crude was triturated with EtOAc and the solid was filtered to give 6-amino-5′-methyl-2′-((2-methylpyrimidin-4-yl)amino)-[4,4′-bipyridin]-2(11H)-one TFA salt (0.38 g, 77%) as a pale green solid. ¹H NMR (400 MHz, DMSO-d₆): δ 11.49 (br s, 1H), 8.49 (d, J=6.8 Hz, 1H), 8.33 (s, 1H), 7.91 (br m, 1H), 7.43 (br m, 1H), 6.11-6.41 (m, 2H), 5.38 (s, 1H), 5.33 (s, 1H), 2.60 (s, 3H), 2.22 (s, 3H), one NH is under solvents; MS (ESI) m/z: 309.3 (M+H⁺).

### Preparation of Intermediate K1: methyl (S)-(2′-fluoro-5-methyl-6′-(2-methylpiperidin-1-yl)-[4,4′-bipyridin]-2-yl)carbamate

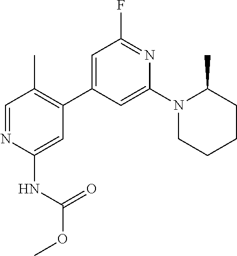 [SMILES: COC(=O)Nc1cc(-c2cc(F)nc(N3CCCC[C@@H]3C)c2)c(C)cn1]

A solution of (2S)-2-methylpiperidine (0.17 mL, 1.43 mmol) in DMSO (10 mL) was treated with DIEA (0.50 mL, 2.86 mmol) at 0° C. Methyl N-[4-(2,6-difluoro-4-pyridyl)-5-methyl-2-pyridyl]carbamate (B3, 0.40 g, 1.43 mmol) was added then the mixture was heated at 80° C. for 5 h. The reaction mixture was cooled and then quenched with water. The resulting solid was filtered to obtain methyl (S)-(2′-fluoro-5-methyl-6′-(2-methylpiperidin-1-yl)-[4,4′-bipyridin]-2-yl)carbamate (0.50 g, crude) as a gray solid. MS (ESI) m/z: 359.2 (M+H⁺).

The following compounds are prepared essentially by method of preparation K1.

| Ex | | | Yield | ¹H NMR (400 MHz, | MS (m/z: |
|---|---|---|---|---|---|
| No | SM | Product | (%) | CDCl₃): δ | M + H⁺) |
| K2 | 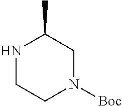 | 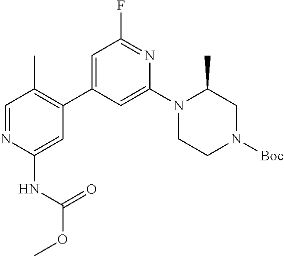 [SMILES: COC(=O)Nc1cc(-c2cc(F)nc(N3CCN(C(=O)OC(C)(C)C)C[C@@H]3C)c2)c(C)cn1] | 52 | 8.17 (s, 1H), 8.09 (s, 1H), 7.87 (s, 1H), 6.28 (s, 1H), 6.14 (d, J = 1.6 Hz, 1H), 4.47 (s, 1H), 4.28-3.87 (m, 3H), 3.81 (s, 3H), 3.20 (dt, J = 3.6, 12.4 Hz, 2H), 3.01 (m, 1H), 2.21 (s, 3H), 1.50 (s, 9H), 1.19 (d, J = 6.4 Hz, 3H). | 460.4 |

### Preparation of Example L1: methyl (R)-(5-methyl-2′-oxo-6′-(2-(trifluoromethyl)-4-(vinylsulfonyl)piperazin-1-yl)-1′,2′-dihydro-[4,4′-bipyridin]-2-yl)carbamate

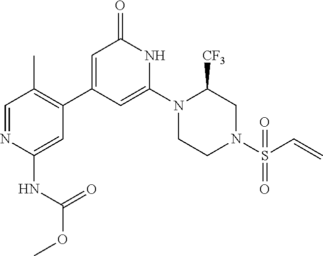 [SMILES: C=CS(=O)(=O)N1CCN(c2cc(-c3cc(NC(=O)OC)ncc3C)cc(=O)[nH]2)[C@@H](C(F)(F)F)C1]

A solution of methyl N-[5-methyl-4-[2-[(2S)-2-methylpiperazin-1-yl]-6-oxo-1H-pyridin-4-yl]-2-pyridyl]carbamate (45, 0.1 g, 0.25 mmol) in DCM (1 mL) was added DMF (a drop) and Et₃N (0.07 mL). Ethenesulfonyl chloride (0.03 g, 0.25 mmol) in DCM (1 mL) was added at 0° C. and stirred at rt for 0.5 h. The reaction mixture was concentrated under reduced pressure to obtain methyl (R)-(5-methyl-2′-oxo-6′-(2-(trifluoromethyl)-4-(vinyl sulfonyl)piperazin-1-yl)-1,2′-dihydro-[4,4′-bipyridin]-2-yl)carbamate (L1, crude) which was used for the next step without further purification. MS (ESI) m/z: 448.6 (M+H⁺).

| Ex | | | | Yield | | MS (m/z: |
|---|---|---|---|---|---|---|
| No | SM | | Product | (%) | ¹H NMR (400 MHz, DMSO-d₆): δ | M + H⁺) |
| L2 | 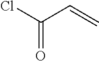 | 46 | 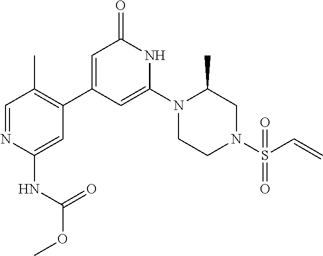 [SMILES: C=CS(=O)(=O)N1CCN(c2cc(-c3cc(NC(=O)OC)ncc3C)cc(=O)[nH]2)[C@@H](C)C1] | crude | | 448.6 |
| L3 | 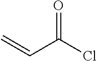 | J2 | 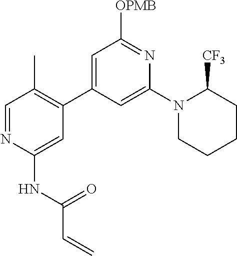 | crude | | 527.4 |
| L4 | 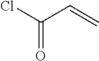 | 45 | 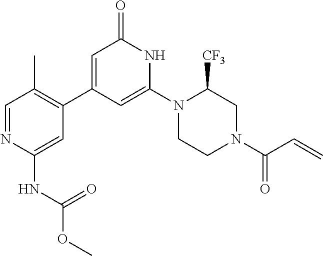 [SMILES: C=CC(=O)N1CCN(c2cc(-c3cc(NC(=O)OC)ncc3C)cc(=O)[nH]2)[C@@H](C(F)(F)F)C1] | crude | | 466.2 |
| L5 | 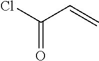 | 46 | 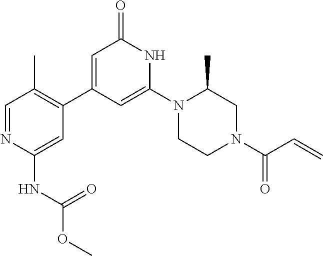 [SMILES: C=CC(=O)N1CCN(c2cc(-c3cc(NC(=O)OC)ncc3C)cc(=O)[nH]2)[C@@H](C)C1] | crude | | 412.4 |
| L6 | 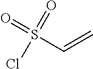 | J6 | 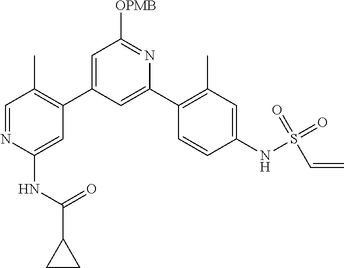 | 32 | 10.83 (s, 1H), 10.11 (s, 1H), 8.27 (s, 1H), 8.01 (s, 1H), 7.44 (d, J = 8.0 Hz, 1H), 7.40 (d, J = 8.0 Hz, 1H), 7.10 (s, 1H), 7.08 (s, 2H), 6.94 (d, J = 8.8 Hz, 2H), 6.84 (m, 1H), 6.79 (d, J = 1.2 Hz, 1H), 6.17 (d, J = 16.4 Hz, 1H), 6.07 (d, J = 10.0 Hz, 1H), 5.33 (s, 2H), 3.75 (s, 3H), 2.35-2.39 (m, 3H), 2.23 (s, 3H), 1.98(m, 1H), 0.79 (d, J = 6.4 Hz, 4H). | 585.4 |

### Preparation of Example 1: methyl (R)-(5-methyl-2′-oxo-6′-(2-(trifluoromethyl)piperidin-1-yl)-1′,2′-dihydro-[4,4′-bipyridin]-2-yl)carbamate 12221

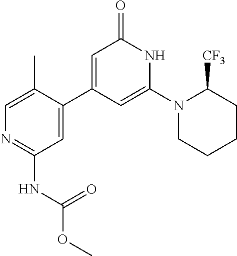 [SMILES: COC(=O)Nc1cc(-c2cc(N3CCCC[C@@H]3C(F)(F)F)[nH]c(=O)c2)c(C)cn1]

A mixture of methyl (4-iodo-5-methylpyridin-2-yl)carbamate (A1, 2.04 g, 6.98 mmol) and (R)-2-(tert-butoxy)-4-(4,4,5,5-tetramethyl-1,3,2-dioxaborolan-2-yl)-6-(2-(trifluoromethyl)piperidin-1-yl)pyridine (G3, 4.27 g, 6.98 mmol) in a mixture of 1,4-dioxane (75 mL) and H₂O (15 mL) was treated with Na₂CO₃ (1.48 g, 13.96 mmol). The reaction mixture was degassed with N₂ for 3 min and then Pd(dppf)Cl₂ (0.50 g, 0.70 mmol). The mixture was heated at 80° C. for 3 h under N₂ atmosphere and then cooled to rt. The reaction mixture was diluted with water (200 mL) and the aqueous phase was extracted with EtOAc (3×). The combined organic layers were dried over anhydrous MgSO₄, filtered, and concentrated under reduced pressure. The residue was purified by silica gel column chromatography (0 to 20% EtOAc/pet-ether) to obtain methyl (R)-(2′-(tert-butoxy)-5-methyl-6′-(2-(trifluoromethyl)piperidin-1-yl)-[4,4′-bipyridin]-2-yl)carbamate (2.9 g, 79%) as a yellow solid. ¹H NMR: (400 MHz, CDCl₃): δ 8.42 (s, 1H), 8.15 (s, 1H), 7.86 (s, 1H), 6.06 (s, 1H), 6.00 (d, J=0.8 Hz, 1H), 5.34 (m, 1H), 3.92 (m, 1H), 3.80 (m, 3H), 3.19 (br t, J=12.8 Hz, 1H), 2.21 (s, 3H), 2.12 (m, 1H), 1.71-1.86 (m, 4H), 1.61 (m, 1H), 1.58 (s, 9H): MS (ESI) m/z: 467.1 (M+H⁺).

Methyl (R)-(2′-(tert-butoxy)-5-methyl-6′-(2-(trifluoromethyl)piperidin-1-yl)-[4,4′-bipyridin]-2-yl)carbamate (1.82 g, 3.90 mmol) was treated with HCl/EtOAc (20 mL). The mixture was stirred at 25° C. for 2 h and then concentrated under reduced pressure. The residue was dissolved in MeOH (20 mL) and NaHCO₃ (0.07 mL, 1.72 mmol) was added and then the mixture was stirred at rt for 0.5 h. The reaction mixture was filtered and the filtrated was concentrated under reduced pressure. The residue was triturated with MeCN and stirred at rt for 20 min to obtain methyl (R)-(5-methyl-2′-oxo-6′-(2-(trifluoromethyl)piperidin-1-yl)-1′,2′-dihydro-[4,4′-bipyridin]-2-yl)carbamate (1, 0.40 g, 62%) as a white solid. ¹H NMR: (400 MHz, DMSO-d6): δ 10.16 (br s, 1H), 8.17 (s, 1H), 7.66 (s, 1H), 6.24 (s, 1H), 5.87 (s, 1H), 5.48 (m, 1H), 4.11 (br d, J=12.4 Hz, 1H), 3.65 (s, 3H), 2.98 (t, J=12.6 Hz, 1H), 2.15 (s, 3H), 1.97 (br d, J=13.6 Hz, 1H), 1.59-1.83 (m, 4H), 1.45 (m, 1H), one NH is under solvents; MS (ESI) m/z: 411.1 (M+H⁺).

### Preparation of Example 2: (R)-5′-methyl-2′-((6-methylpyrazin-2-yl)amino)-6-(2-(trifluoromethyl)piperidin-1-yl)-[4,4′-bipyridin]-2(1H)-one 13741

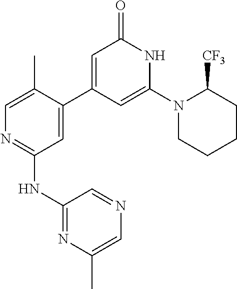 [SMILES: Cc1cncc(Nc2cc(-c3cc(N4CCCC[C@@H]4C(F)(F)F)[nH]c(=O)c3)c(C)cn2)n1]

A mixture of 6-methylpyrazin-2-amine (0.037 g, 0.31 mmol) and (R)-2-chloro-2′-((4-methoxybenzyl)oxy)-5-methyl-6′-(2-(trifluoromethyl)piperidin-1-yl)-4,4′-bipyridine (I2, 0.15 g, 0.31 mmol) in DMF (15 mL) was treated with Cs₂CO₃ (0.20 g, 0.61 mmol). The mixture was degassed and purged with N₂ gas for 3 min and then Pd₂(dba)₃ (0.028 g, 0.1 eq) and Xantphos (0.35 g, 0.2 eq) were added. The reaction mixture was stirred at 100° C. for 6 h and then quenched by NH₄Cl (20 mL)/water (50 mL) at rt. The solution was extracted with EA (3×) and the combined organic layers were washed with brine, dried over anhydrous Na₂SO₄, filtered, and concentrated under reduced pressure. The residue was purified by silica gel column chromatography (0 to 20% EtOAc/pet-ether) to obtain (R)-2′-((4-methoxybenzyl)oxy)-5-methyl-N-(6-methylpyrazin-2-yl)-6′-(2-(trifluoromethyl)piperidin-1-yl)-[4,4′-bipyridin]-2-amine (0.18 g, 84%) as a gray solid.

A solution of (R)-2′-((4-methoxybenzyl)oxy)-5-methyl-N-(6-methylpyrazin-2-yl)-6′-(2-(trifluoromethyl)piperidin-1-yl)-[4,4′-bipyridin]-2-amine (0.18 g, 0.32 mmol) in HCl/EtOAc (6 mL) was stirred at rt for 1 h. The reaction mixture was concentrated under reduced pressure and the residue was purified by C-18 prep-HPLC (25 to 55% H₂O (0.05% NH₃H₂O+10 mM NH₄HCO₃)/MeCN) to obtain (0.067 g, 47%) as a white solid. ¹H NMR: (400 MHz, DMSO-d6): δ 10.40 (br s, 1H), 9.91 (s, 1H), 8.94 (s, 1H), 8.17 (s, 1H), 7.96 (s, 1H), 7.50 (s, 1H), 6.26 (s, 1H), 5.90 (s, 1H), 5.48 (m, 1H), 4.14 (br d, J=11.2 Hz, 1H), 2.99 (br t, J=12.8 Hz, 1H), 2.36 (s, 3H), 2.15 (s, 3H), 1.97 (br d, J=13.2 Hz, 1H), 1.62-1.80 (m, 4H), 1.46 (m, 1H); MS (ESI) m/z: 445.1 (M+H⁺).

### Preparation of Example 3: (R)-N-(5-methyl-2′-oxo-6′-(2-(trifluoromethyl)piperidin-1-yl)-1′,2′-dihydro-[4,4′-bipyridin]-2-yl)-2-(1-methylazetidin-3-yl)acetamide 13692

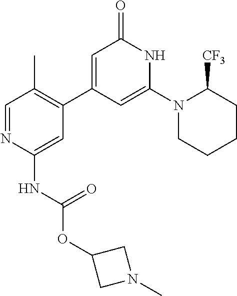 [SMILES: Cc1cnc(NC(=O)OC2CN(C)C2)cc1-c1cc(N2CCCC[C@@H]2C(F)(F)F)[nH]c(=O)c1]

A solution of 2-(1-tert-butoxycarbonylazetidine-3-yl)acetic acid (0.23 g, 1.06 mmol) in DMF (5 mL) was treated NMI (0.25 mL, 3.17 mmol) and TCFH (0.44 g, 1.59 mmol), then 4-[2-[(4-methoxyphenyl)methoxy]-6-[(2R)-2-(trifluoromethyl)-1-piperidyl]-4-pyridyl]-5-methyl-pyridin-2-amine (0.5 g, 1.06 mmol) was added and stirred at rt for 2 h. The reaction mixture was filtered through a pad of celite and the filtrate was concentrated under reduced pressure. The residue was purified by silica gel chromatography (0 to 45% EtOAc/pet-ether) to afford tert-butyl (R)-3-(2-((2′-((4-methoxybenzyl)oxy)-5-methyl-6′-(2-(trifluoromethyl)piperidin-1-yl)-[4,4′-bipyridin]-2-yl)amino)-2-oxoethyl)azetidine-1-carboxylate (0.60 g, 85%) as a white solid. MS (ESI) m/z: 670.4 (M+H⁺).

To a solution tert-butyl (R)-3-(2-((2′-((4-methoxybenzyl)oxy)-5-methyl-6′-(2-(trifluoromethyl)piperidin-1-yl)-[4,4′-bipyridin]-2-yl)amino)-2-oxoethyl)azetidine-1-carboxylate (0.40 g, 0.60 mmol) in DCM (15 mL) was added [tert-butyl(dimethyl)silyl]trifluoromethanesulfonate (0.14 mL, 0.63 mmol) slowly and stirred at rt for 1 h. The reaction mixture was concentrated under reduced pressure to give (R)-2-(azetidin-3-yl)-N-(2′-(4-methoxyphenoxy)-5-methyl-6′-(2-(trifluoromethyl)piperidin-1-yl)-[4,4′-bipyridin]-2-yl)acetamide (0.37 g. crude) as a white solid. MS (ESI) m/z: 570.4 (M+H⁺).

To a solution of (R)-2-(azetidin-3-yl)-N-(2′-(4-methoxyphenoxy)-5-methyl-6′-(2-(trifluoromethyl)piperidin-1-yl)-[4,4′-bipyridin]-2-yl)acetamide (0.34 g, 0.60 mmol) in DCE (5 mL) was added HCHO (0.13 mL, 1.79 mmol, 37% purity, 3.0 eq) and stirred at rt for 0.5 h. NaBH₃CN (0.056 g, 1.5 eq) was added and stirred for 0.5 h. The reaction mixture was concentrated under reduced pressure, then water (10 mL) was added. The mixture was extracted with EtOAc (3×), and then the combined organics were dried over anhydrous Na₂SO₄, filtered, and concentrated under reduced pressure to afford N-[4-[2-[(4-methoxyphenyl)methoxy]-6-[(2R)-2-(trifluoromethyl)-1-piperidyl]-4-pyridyl]-5-methyl-2-pyridyl]-2-(1-methylazetidin-3-yl)acetamide (0.55 g, crude) as a brown solid. MS (ESI) m/z: 584.3 (M+H⁺).

To a solution of N-[4-[2-[(4-methoxyphenyl)methoxy]-6-[(2R)-2-(trifluoromethyl)-1-piperidyl]-4-pyridyl]-5-methyl-2-pyridyl]-2-(1-methylazetidin-3-yl)acetamide (0.50 g, 0.86 mmol) in DCM (10 mL) was treated with TFA (0.5 mL). The reaction mixture was stirred at rt for 0.5 h and then concentrated under reduced pressure. The residue was purified by C-18 prep-HPLC (20 to 50% H₂O (10 mM NH₄HCO₃)/MeCN) to afford (R)-N-(5-methyl-2′-oxo-6′-(2-(trifluoromethyl)piperidin-1-yl)-1′,2′-dihydro-[4,4′-bipyridin]-2-yl)-2-(1-methylazetidin-3-yl)acetamide (77, 0.053 g, 13%) as a white solid. ¹H NMR (400 MHz, DMSO-d₆): δ 10.46 (s, 1H), 10.01 (br s, 1H), 8.20 (s, 1H), 7.90 (s, 1H), 6.22 (s, 1H), 5.85 (s, 1H), 5.48 (m, 1H), 4.10 (br d, J=12.8 Hz, 1H), 3.27-3.30 (m, 2H), 2.98 (br t, J=12.8 Hz, 1H), 2.74-2.79 (m, 2H), 2.60-2.67 (m, 3H), 2.16 (s, 3H), 2.15 (s, 3H), 1.97 (m, 1H), 1.60-1.81 (m, 4H), 1.46 (m, 1H); MS (ESI) m/z: 464.1 (M+H⁺).

### Preparation of Example 4: methyl (5′-methyl-6′-oxo-4-(trifluoromethyl)-1′,6′-dihydro-[3,2′:4′,4″-terpyridin]-2″-yl)carbamate 13821

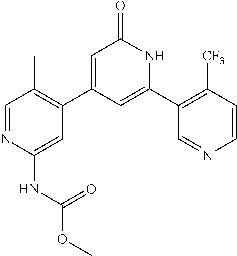 [SMILES: COC(=O)Nc1cc(-c2cc(-c3cnccc3C(F)(F)F)[nH]c(=O)c2)c(C)cn1]

A solution of 6′-((4-methoxybenzyl)oxy)-5″-methyl-4-(trifluoromethyl)-[3,2′:4′,4″-terpyridin]-2″-amine (J4, 0.27 g, 0.58 mmol) in pyridine (5 mL) and cooled to 0° C. Methyl chloroformate (0.18 mL, 2.3 mmol) was added dropwise and the reaction mixture was allowed to warm to rt overnight. The reaction was concentrated under reduced pressure. The crude was purified by silica gel column chromatography (0 to 100% EtOAc/hexanes) to obtain methyl (6′-((4-methoxybenzyl)oxy)-5″-methyl-4-(trifluoromethyl)-[3,2′:4′,4″-terpyridin]-2″-yl)carbamate (0.31 g, 100%) as a clear amber solid.

A solution of methyl (6′-((4-methoxybenzyl)oxy)-5″-methyl-4-(trifluoromethyl)-[3,2′:4′,4″-terpyridin]-2″-yl)carbamate (0.31 g, 0.58 mmol) in MeOH (5 mL) was treated with TFA (3.5 mL, 45 mmol). The reaction mixture was heated to 60° C. for 16 h and then concentrated under reduced pressure. The residue was treated with MeCN (5 mL) and sonicated for 10 min. The resulting solid was filtered and washed with MeCN to obtain methyl (5″-methyl-6′-oxo-4-(trifluoromethyl)-1′,6′-dihydro-[3,2′:4′,4″-terpyridin]-2″-yl)carbamate (78, 0.11 g, 44%) as a white solid. ¹H NMR (400 MHz, DMSO-d₆): δ 11.99 (br s, 1H), 10.23 (s, 1H), 8.87-8.99 (m, 2H), 8.20 (s, 1H), 7.90 (d, J=5.2 Hz, 1H), 7.74 (s, 1H), 6.48 (s, 2H), 3.66 (s, 3H), 2.20 (s, 3H); MS (ESI) m/z: 405.0 (M+H⁺).

The following compounds are prepared essentially by method of preparation examples 1, 2, 3, and 4.

| Ex | | | Yield | | MS (m/z: |
|---|---|---|---|---|---|
| No | SM | Product | (%) | ¹H NMR (400 MHz, DMSO-d₆): δ | M + H⁺) |
| 5 125 99 | A4 G2 HCl | 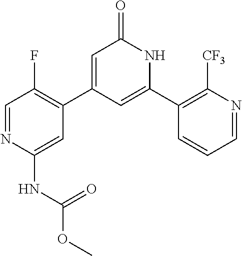 [SMILES: COC(=O)Nc1cc(-c2cc(-c3cccnc3C(F)(F)F)[nH]c(=O)c2)c(F)cn1] | 50 | 10.46 (s, 1H), 8.86 (d, J = 3.9 Hz, 1H), 8.41 (d, J = 1.8 Hz, 1H), 8.17 (d, J = 7.3 Hz, 1H), 7.99 (d, J = 5.6 Hz, 1H), 7.85 (dd, J = 4.8, 7.9 Hz, 1H), 6.73 (s, 1H), 6.62 (br s, 1H), 3.68 (s, 3H). | 409.0 |
| | | HCl salt | | | |
| 6 126 33 | A3 G2 HCl | 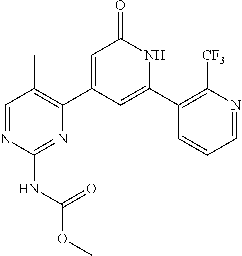 [SMILES: COC(=O)Nc1ncc(C)c(-c2cc(-c3cccnc3C(F)(F)F)[nH]c(=O)c2)n1] | 41 | 10.41 (s, 1H), 8.86 (d, J = 4.1 Hz, 1H), 8.59 (s, 1H), 8.13 (d, J = 7.8 Hz, 1H), 7.84 (dd, J = 4.8, 7.9 Hz, 1H), 6.67 (br s, 1H), 3.64 (s, 3H), 2.27 (s, 3H). | 406.2 |
| 7 126 34 | A4 G1 HCl | 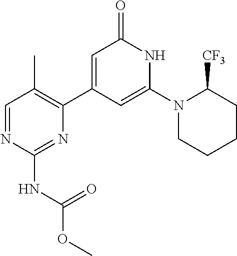 [SMILES: COC(=O)Nc1ncc(C)c(-c2cc(N3CCCC[C@@H]3C(F)(F)F)[nH]c(=O)c2)n1] | 39 | 10.59 (br s, 1H), 10.38 (s, 1H), 8.36 (m, 1H), 7.93 (d, J = 5.6 Hz, 1H), 6.45 (s, 1H), 6.09 (s, 1H), 5.52 (br s, 1H), 4.10 (m, 1H), 3.69 (s, 3H), 3.02 (t, J = 12.4 Hz, 1H), 1.99 (m, 1H), 1.63-1.78 (m, 4H), 1.49 (m, 1H). | 415.1 |
| 8 126 63 | A3 G1 HCl | 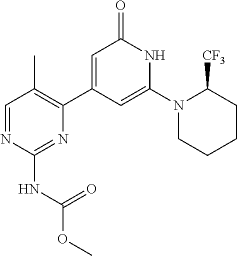 [SMILES: COC(=O)Nc1ncc(C)c(-c2cc(N3CCCC[C@@H]3C(F)(F)F)[nH]c(=O)c2)n1] | 22 | 10.42 (br s, 1H), 10.36 (s, 1H), 8.55 (s, 1H), 6.44 (br s, 1H), 6.07 (s, 1H), 5.44- 5.61 (m, 1H), 4.01-4.14 (m, 1H), 3.64 (s, 3H), 2.95-3.06 (m, 1H), 2.21 (s, 3H), 1.94-2.05 (m, 1H), 1.60-1.82 (m, 4H), 1.46-1.52 (m, 1H) | 412.2 |
| 9 126 96 | A6 G2 HCl | 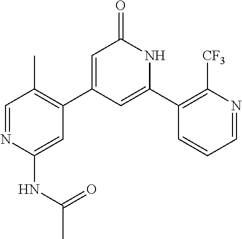 [SMILES: CC(=O)Nc1cc(-c2cc(-c3cccnc3C(F)(F)F)[nH]c(=O)c2)c(C)cn1] | 41 | 10.53 (s, 1H), 8.85 (d, J = 4.8 Hz, 1H), 8.24 (s, 1H), 8.17 (d, J = 8.0 Hz, 1H), 7.99 (s, 1H), 7.84 (dd, J = 7.9, 4.8 Hz, 1H), 6.35-6.45 (m, 2H), 2.21 (s, 3H), 2.09 (s, 3H). | 389.1 |
| 10 127 41 | A5 G1 HCl | 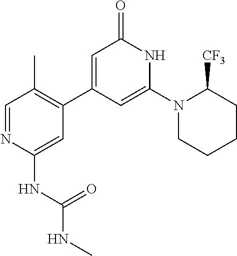 [SMILES: CNC(=O)Nc1cc(-c2cc(N3CCCC[C@@H]3C(F)(F)F)[nH]c(=O)c2)c(C)cn1] | 13 | 8.12 (s, 1H), 7.14 (s, 1H), 6.25 (s, 1H), 5.91 (s, 1H), 5.42 (m, 1H), 4.03 (br d, J = 11.9 Hz, 1H), 2.96 (br t, J = 12.5 Hz, 1H), 2.71 (s, 3H), 2.14 (s, 3H), 1.96 (br d, J = 13.2 Hz, 1H), 1.52-1.75 (m, 4H), 1.42 (m, 1H), 3 NHs are under solvents, in D₂O. | 410.0 |
| 11 127 61 | A19 G2 HCl | 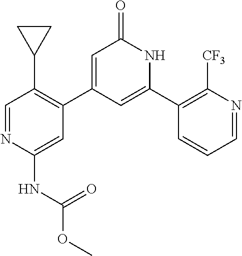 [SMILES: COC(=O)Nc1cc(-c2cc(-c3cccnc3C(F)(F)F)[nH]c(=O)c2)c(C2CC2)cn1] | 40 | 10.47 (s, 1H), 8.89 (d, J = 4.0 Hz, 1H), 8.20 (d, J = 8.0 Hz, 1H), 8.09 (s, 1H), 7.88 (dd, J = 4.8, 8.0 Hz, 1H), 7.74 (s, 1H), 6.57 (s, 1H), 6.53 (s, 1H), 4.40 (br s, 1H), 3.72 (s, 3H), 1.88 (m, 1H), 0.84- 0.92 (m, 2H), 0.68-0.76 (m, 2H). | 431.0 |
| 12 128 24 | A9 G1 HCl | 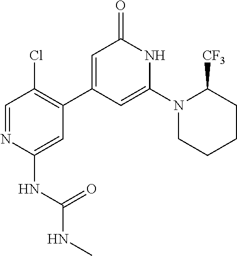 [SMILES: CNC(=O)Nc1cc(-c2cc(N3CCCC[C@@H]3C(F)(F)F)[nH]c(=O)c2)c(Cl)cn1] | 54 | 10.58 (s, 2H), 8.46 (s, 1H), 7.90 (s, 1H), 6.39 (s, 1H), 6.02 (s, 1H), 5.55 (m, 1H), 4.15 (br d, J = 12.0 Hz, 1H), 3.74 (s, 3H), 3.06 (br t, J = 12.8 Hz, 1H), 2.04 (br d, J = 13.6 Hz, 1H), 1.64-1.90 (m, 4H), 1.52 (m, 1H). | 431.1 |
| 13 129 53 | 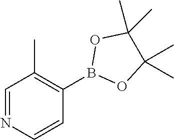 F1 | 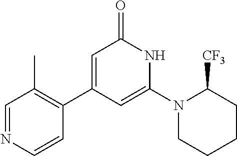 [SMILES: Cc1cnccc1-c1cc(N2CCCC[C@@H]2C(F)(F)F)[nH]c(=O)c1] | 44 | δ 10.38 (s, 1H), 8.48 (s, 1H), 8.42 (d, J = 4.8 Hz, 1H), 7.21 (m, 1H), 6.25 (s, 1H), 5.87 (s, 1H), 5.48 (m,1H), 4.12 (br d, J = 13.5 Hz, 1H), 2.96 (t, J = 13.0 Hz, 1H), 2.22 (s, 3H), 1.95 (m, 1H), 1.68 (m, 4H), 1.44 (m, 1H). | 338.2 |
| | HCl | | | | |
| 14 129 56 | A23 Boc₂O G1 HCl | 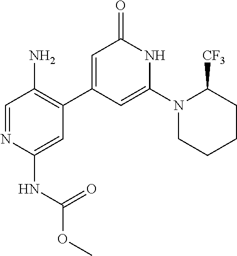 [SMILES: COC(=O)Nc1cc(-c2cc(N3CCCC[C@@H]3C(F)(F)F)[nH]c(=O)c2)c(N)cn1] | 44 | 10.37 (s, 1H), 9.69 (s, 1H), 7.81 (s, 1H), 7.45 (s, 1H), 6.37 (s, 1H), 5.98 (s, 1H), 5.51 (m, 1H), 4.84 (s, 2H), 4.09 (m, 1H), 3.62 (s, 3H), 3.00(t, J = 12.4 Hz, 1H), 1.98 (br d, J = 13.6 Hz, 1H), 1.66- 1.76 (m, 4H), 1.47 (m, 1H). | 412.2 |
| 15 129 85 | A23 G2 Boc₂O HCl | 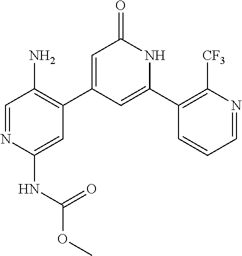 [SMILES: COC(=O)Nc1cc(-c2cc(-c3cccnc3C(F)(F)F)[nH]c(=O)c2)c(N)cn1] | 43 | 12.10 (br s, 1H), 9.81 (s, 1H), 8.86 (d, J = 4.0 Hz, 1H), 8.17 (d, J = 7.2 Hz, 1H), 7.83-7.89 (m, 2H), 7.52 (s, 1H), 6.56 (br s, 1H), 6.35 (br s, 1H), 4.62- 5.41 (br s, 2H), 3.63 (s, 3H). | 406.1 |
| 16 130 06 | A8 G1 HCl | 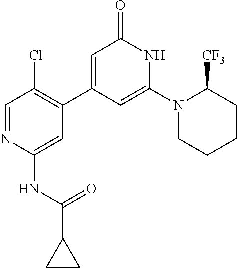 [SMILES: O=C(Nc1cc(-c2cc(N3CCCC[C@@H]3C(F)(F)F)[nH]c(=O)c2)c(Cl)cn1)C1CC1] | 44 | 11.11 (s, 1H), 10.53 (br s 1H), 8.50 (s, 1H), 8.15 (s, 1H), 6.36 (s, 1H), 5.98 (s, 1H), 5.54 (br m, 1H), 4.11 (br m, 1H), 3.04 (br t, J = 13.2 Hz, 1H), 1.98-2.09 (m, 2H), 1.65-1.88 (m, 4H), 1.51 (m, 1H), 0.82-0.92 (m, 4H). | 441.1 |
| 17 130 07 | A7 G1 HCl | 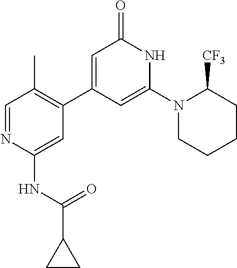 [SMILES: Cc1cnc(NC(=O)C2CC2)cc1-c1cc(N2CCCC[C@@H]2C(F)(F)F)[nH]c(=O)c1] | 64 | 10.78 (s, 1H), 10.39 (br s, 1H), 8.21 (s, 1H), 7.92 (s, 1H), 6.23 (s, 1H), 5.85 (s, 1H), 5.49 (br m, 1H), 4.10 (br m, 1H), 2.98 (br t, J = 13.2 Hz, 1H), 2.16 (s, 3H), 1.93-2.01 (m, 2H), 1.62-1.81 (m, 4H), 1.46 (m, 1H), 0.78-0.80 (m, 4H). | 421.2 |
| 18 131 06 | A20 G1 HCl | 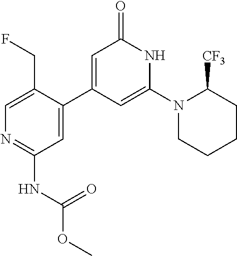 [SMILES: COC(=O)Nc1cc(-c2cc(N3CCCC[C@@H]3C(F)(F)F)[nH]c(=O)c2)c(CF)cn1] | 30 | 10.5 (br s, 1H), 10.49 (s, 1H), 8.47 (d, J = 2.4 Hz, 1H), 7.83 (s, 1H), 6.31 (br s, 1H), 5.96 (s, 1H), 5.50 (m, 1H), 5.32 (d, J = 48.0 Hz, 2H), 4.10 (m, 1H), 3.69 (s, 3H), 3.01 (br t, J = 12.6 Hz, 1H), 1.99 (m, 1H), 1.61-1.85 (m, 4H), 1.43- 1.56 (m, 1H). | 429.1 |
| 19 133 68 | A17 G3 TFA | 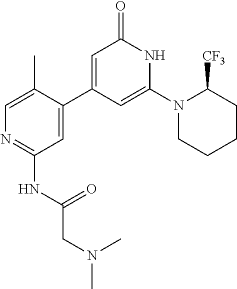 [SMILES: Cc1cnc(NC(=O)CN(C)C)cc1-c1cc(N2CCCC[C@@H]2C(F)(F)F)[nH]c(=O)c1] | 41 | 10.78 (br s, 1H), 10.49 (br s, 1H), 8.27 (s, 1H), 7.92 (s, 1H), 6.25 (s, 1H), 5.87 (s, 1H), 5.48 (br m, 1H), 4.11 (br m, 1H), 3.85 (br m, 2H), 2.98 (br t, J = 13.2 Hz, 1H), 2.69 (s, 6H), 2.19 (s, 3H), 1.97 (br m, 1H), 1.60-1.82 (m, 4H), 1.39-1.51 (m, 1H). | 438.2 |
| 20 134 39 | K1 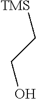 TFA | 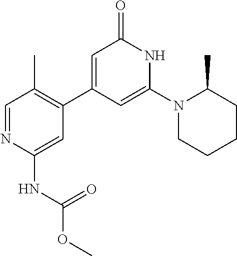 [SMILES: COC(=O)Nc1cc(-c2cc(N3CCCC[C@@H]3C)[nH]c(=O)c2)c(C)cn1] | 16 | 10.15 (s, 1H), 8.15 (s, 1H), 7.65 (s, 1H), 5.92 (br m, 1H), 5.73 (s, 1H), 5.70 (s, 1H), 4.52 (br m, 1H), 3.99 (br m, 1H), 3.65 (s, 3H), 2.84 (br t, J = 12.0 Hz, 1H), 2.16 (s, 3H), 1.50-1.71 (m, 5H), 1.40 (m, 1H), 1.08 (d, J = 6.8 Hz, 3H). | 357.2 |
| 21 134 40 | A22 G3 HCl | 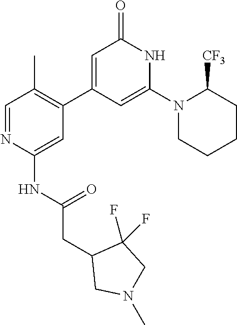 [SMILES: Cc1cnc(NC(=O)CC2CN(C)CC2(F)F)cc1-c1cc(N2CCCC[C@@H]2C(F)(F)F)[nH]c(=O)c1] | 50 | 10.57 (s, 1H), 8.22 (s, 1H), 7.91 (s, 1H), 6.23 (s, 1H), 5.86 (s, 1H), 5.50 (m, 1H), 4.12 (br m, 1H), 2.97-3.13 (m, 3H), 2.72-2.96 (m, 2H), 2.58-2.72 (m, 2H), 2.34-2.55 (m, 1H), 2.25 (s, 3H), 2.24 (m, 1H), 2.16 (s, 3H), 1.97 (m, 1H), 1.57-1.85 (br m, 4H), 1.46 (br m, 1H). | 514.2 |
| 22 134 41 | A13 G3 HCl | 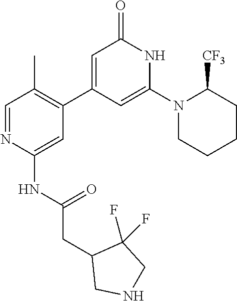 [SMILES: Cc1cnc(NC(=O)CC2CNCC2(F)F)cc1-c1cc(N2CCCC[C@@H]2C(F)(F)F)[nH]c(=O)c1] | 48 | 10.57 (s, 1H), 8.23 (s, 1H), 7.92 (s, 1H), 6.24 (s, 1H), 5.87 (s, 1H), 5.49 (m, 1H), 4.12 (br d, J = 12.0 Hz, 1H), 3.11-3.27 (m, 3H), 2.92-3.04 (m, 2H), 2.54-2.77 (m, 3H), 2.47-2.49 (m, 1H), 2.17 (s, 3H), 1.98 (br d, J = 13.2 Hz, 1H), 1.60- 1.84 (br m, 4H), 1.47 (br m, 1H), one NH is under solvents. | 500.2 |
| 23 126 99 | J2 TFA | 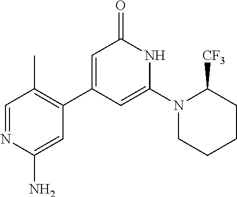 [SMILES: Cc1cnc(N)cc1-c1cc(N2CCCC[C@@H]2C(F)(F)F)[nH]c(=O)c1] | 22 | 10.34 (br s, 1H), 7.79 (m, 1H), 6.29 (s, 1H), 6.18 (br s, 1H), 5.82 (s, 1H), 5.73 (s, 2H), 5.46 (br s, 1H), 4.11 (br d, J = 12.9 Hz, 1H), 2.98 (br t, J = 12.5 Hz, 1H), 1.99 (s, 3H), 1.95 (m, 1H), 1.58- 1.82 (m, 4H), 1.47 (br m, 1H). | 353.1 |
| 24 134 98 | 23 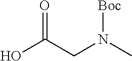 HATU HCl | 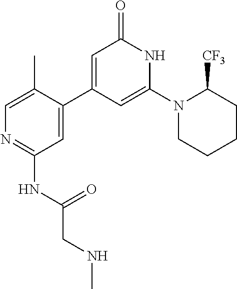 [SMILES: CNCC(=O)Nc1cc(-c2cc(N3CCCC[C@@H]3C(F)(F)F)[nH]c(=O)c2)c(C)cn1] | 44 | 11.19 (s, 1H), 9.17 (br m, 2H), 8.28 (s, 1H), 7.88 (s, 1H), 6.26 (s, 1H), 5.92 (s, 1H), 5.46 (m, 1H), 4.12 (br d, J = 13.2 Hz, 1H), 3.94-3.98 (br m, 2H), 2.98 (br t, J = 12.8 Hz, 1H), 2.59 (t, J = 5.6 Hz, 3H), 2.19 (s, 3H), 1.97 (m, 1H), 1.62- 1.82 (br m, 4H), 1.45 (br m, 1H). | 424.1 |
| 25 134 99 | L3 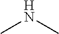 TEA TFA | 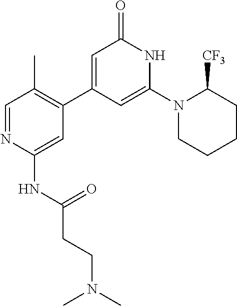 [SMILES: Cc1cnc(NC(=O)CCN(C)C)cc1-c1cc(N2CCCC[C@@H]2C(F)(F)F)[nH]c(=O)c1] | 44 | 10.67 (s, 1H), 8.21 (s, 1H), 7.94 (s, 1H), 6.23 (s, 1H), 5.86 (s, 1H), 5.45 (br m, 1H), 4.10 (br d, J = 12.4 Hz, 1H), 2.98 (br t, J = 12.6 Hz, 1H), 2.62-2.71 (m, 2H), 2.53-2.58 (m, 2H), 2.26 (s, 6H), 2.16 (s, 3H), 1.97 (m, 1H), 1.56-1.84 (br m, 4H), 1.47 (br m, 1H). | 452.2 |
| 26 135 00 | L3 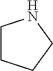 TEA TFA | 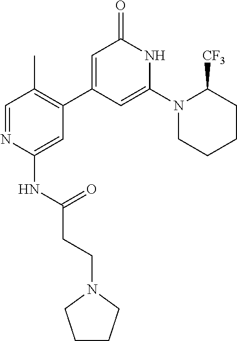 [SMILES: Cc1cnc(NC(=O)CCN2CCCC2)cc1-c1cc(N2CCCC[C@@H]2C(F)(F)F)[nH]c(=O)c1] | 32 | 10.69 (s, 1H), 8.22 (s, 1H), 8.19 (s, 1H), 7.96 (s, 1H), 6.24 (s, 1H), 5.87 (s, 1H), 5.51 (m, 1H), 4.11 (br d, J = 10.8 Hz, 1H), 2.99 (br t, J = 12.4 Hz, 1H), 2.74 (t, J = 7.2 Hz, 2H), 2.56 (t, J = 7.4 Hz, 2H), 2.17 (s, 3H), 1.98 (m, 1H), 1.59- 1.85 (m, 8H), 1.48 (m, 1H), 6 Hs are under solvents. | 478.2 |
| 27 135 01 | 23 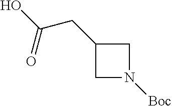 HATU TFA | 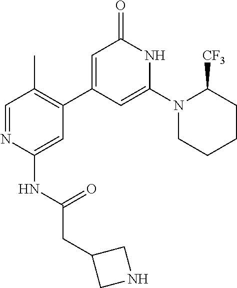 [SMILES: Cc1cnc(NC(=O)CC2CNC2)cc1-c1cc(N2CCCC[C@@H]2C(F)(F)F)[nH]c(=O)c1] | 37 | 10.62 (s, 1H), 8.30 (s, 0.13H), 8.22 (s, 1H), 7.89 (s, 1H), 6.21 (s, 1H), 5.84 (s, 1H), 5.48 (m, 1H), 4.09 (d, J = 12.8 Hz, 1H), 3.95-4.02 (m, 2H), 3.74-3.77 (m, 2H), 2.98 (t, J = 12.8 Hz, 1H), 2.78 (d, J = 7.6 Hz, 2H), 2.16 (s, 3H), 1.97 (m, 1H), 1.60-1.81 (m, 4H), 1.38-1.52 (m, 1H) 2 Hs are under solvents. | 450.2 |
| 28 135 46 | J2 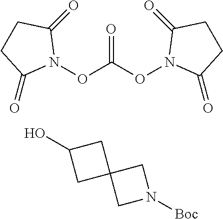 TFA | 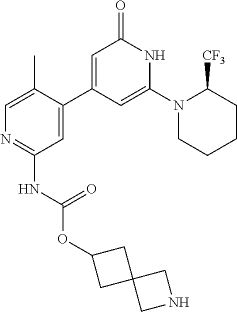 [SMILES: Cc1cnc(NC(=O)OC2CC3(CNC3)C2)cc1-c1cc(N2CCCC[C@@H]2C(F)(F)F)[nH]c(=O)c1] | 18 | 10.09 (s, 1H), 8.16 (s, 1H), 7.62 (s, 1H), 6.22 (s, 1H), 5.85 (s, 1H), 5.49 (d, J = 8.7 Hz, 1H), 4.77 (m, 1H), 4.11 (d, J = 13.5 Hz, 1H), 3.54 (d, J = 20.3 Hz, 4H), 2.98 (t, J = 13.0 Hz, 1H), 2.52-2.60 (m, 2H), 2.14 (s, 5H), 1.97 (d, J = 14.2 Hz, 1H), 1.58-1.87 (m, 4H), 1.46 (br s, 1H). | 492.2 |
| 29 135 73 | A11 G3 TFA | 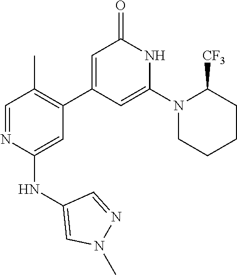 [SMILES: Cc1cnc(Nc2cnn(C)c2)cc1-c1cc(N2CCCC[C@@H]2C(F)(F)F)[nH]c(=O)c1] | 16 | 10.36 (br s, 1H), 8.64 (s, 1H), 7.98 (s, 1H), 7.91 (s, 1H), 7.37 (s, 1H), 6.48 (s, 1H), 6.23 (br s, 1H), 5.86 (s, 1H), 5.47 (m, 1H), 4.14 (m, 1H), 3.79 (s, 3H), 2.98 (br t, J = 12.4 Hz, 1H), 2.06 (s, 3H), 1.97 (m, 1H), 1.63-1.78 (m, 4H), 1.45 (m, 1H). | 433.2 |
| 30 135 97 | J2 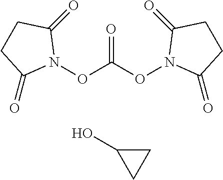 TFA | 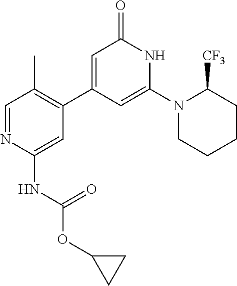 [SMILES: Cc1cnc(NC(=O)OC2CC2)cc1-c1cc(N2CCCC[C@@H]2C(F)(F)F)[nH]c(=O)c1] | 28 | 10.41 (br s, 1H), 10.11 (s, 1H), 8.16 (s, 1H), 7.66 (s, 1H), 6.24 (s, 1H), 5.87 (s, 1H), 5.49 (s, 1H), 4.12 (d, J = 13.4 Hz, 1H), 4.03 (m, 1H), 2.99 (t, J = 13.0 Hz, 1H), 2.15 (s, 3H), 1.98 (d, J = 13.8 Hz, 1H), 1.60-1.83 (m, 4H), 1.47 (s, 1H), 0.62-0.73 (m, 4H). | 437.2 |
| 31 136 00 | J2 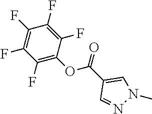 TCFH TFA | 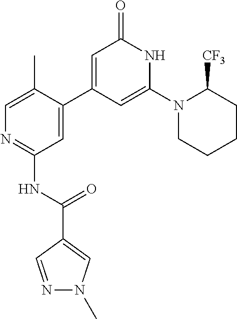 [SMILES: Cc1cnc(NC(=O)c2cnn(C)c2)cc1-c1cc(N2CCCC[C@@H]2C(F)(F)F)[nH]c(=O)c1] | 12 | 10.50 (s, 1H), 10.42 (br s, 1H), 8.41 (s, 1H), 8.27 (s, 1H), 8.12 (s, 1H), 8.04 (s, 1H), 6.28 (s, 1H), 5.91 (s, 1H), 5.51 (br m, 1H), 4.12 (m, 1H), 3.88 (s, 3H), 3.00 (t, J = 13.2 Hz, 1H), 2.20 (s, 3H), 1.98 (m, 1H), 1.57-1.85 (m, 4H), 1.47 (m, 1H). | 431.2 |
| 32 136 20 | L1 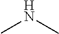 DIEA | 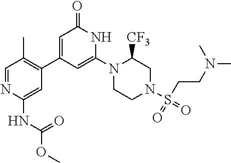 [SMILES: COC(=O)Nc1cc(-c2cc(N3CCN(S(=O)(=O)CCN(C)C)C[C@@H]3C(F)(F)F)[nH]c(=O)c2)c(C)cn1] | 8 | 10.59 (br s, 1H), 10.17 (s, 1H), 8.18 (s, 1H), 7.67 (s, 1H), 6.34 (s, 1H), 5.95 (s, 1H), 5.56 (br m, 1H), 4.26 (m, 1H), 3.90 (m, 1H), 3.65 (s, 3H), 3.62 (m, 1H), 3.22-3.27 (m, 3H), 3.17 (m, 1H), 2.96 (m, 1H), 2.60 (t, J = 6.8 Hz, 2H), 2.15 (s, 6H), 2.14 (s, 3H). | 547.2 |
| 33 136 49 | A11 G2 HCl | 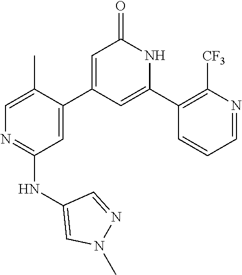 [SMILES: Cc1cnc(Nc2cnn(C)c2)cc1-c1cc(-c2cccnc2C(F)(F)F)[nH]c(=O)c1] | 10 | 12.11 (s, 1H), 8.83 (d, J = 4.7 Hz, 1H), 8.74 (s, 1H), 8.12 (d, J = 7.8 Hz, 1H), 7.98 (s, 1H), 7.89 (s, 1H), 7.82 (dd, J = 4.7, 7.9 Hz, 1H), 7.37 (s, 1H), 6.52 (s, 1H), 6.40 (s, 2H), 3.78 (s, 3H), 2.08 (s, 3H). | 427.2 |
| 34 136 64 | L2 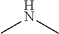 | 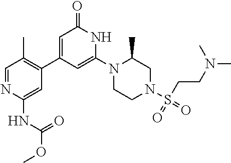 [SMILES: COC(=O)Nc1cc(-c2cc(N3CCN(S(=O)(=O)CCN(C)C)C[C@@H]3C)[nH]c(=O)c2)c(C)cn1] | 3 | 10.39 (br s, 1H), 10.16 (s, 1H), 8.18 (s, 1H), 7.67 (s, 1H), 6.17 (br s, 1H), 5.84 (m, 1H), 4.61 (br m, 1H), 4.14 (br m, 1H), 3.65 (s, 3H), 3.61 (m, 1H), 3.43 (br d, J = 11.2 Hz, 1H), 3.21 (t, J = 7.2 Hz, 2H), 3.08 (m, 1H), 3.00 (m, 1H), 2.91 (m, 1H), 2.61 (m, 2H), 2.16 (s, 6H), 2.15 (s, 3H), 1.14 (d, J = 6.0 Hz, 3H). | 493.2 |
| 35 136 91 | L3 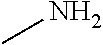 Et₃N HCl | 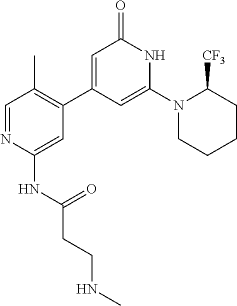 [SMILES: CNCCC(=O)Nc1cc(-c2cc(N3CCCC[C@@H]3C(F)(F)F)[nH]c(=O)c2)c(C)cn1] | 7 | 10.77 (br s, 1H), 8.20 (s, 1H), 7.94 (s, 1H), 6.23 (s, 1H), 5.86 (s, 1H), 5.48 (m, 1H), 4.09 (br d, J = 12.8 Hz, 1H), 2.98 (br t, J = 12.8 Hz, 1H), 2.74 (t, J = 6.4 Hz, 2H), 2.48 (t, J = 6.4 Hz, 2H), 2.29 (s, 3H), 2.15 (s, 3H), 1.97 (m, 1H), 1.62-1.81 (m, 4H), 1.45 (m, 1H), 2 NHs are under solvents. | 438.2 |
| 36 137 17 | G6 A1 SFC separation | 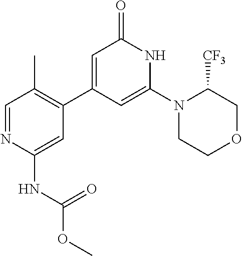 [SMILES: COC(=O)Nc1cc(-c2cc(N3CCOC[C@H]3C(F)(F)F)[nH]c(=O)c2)c(C)cn1] | 35 | 10.40-10.75 (br s, 1H), 10.18 (s, 1H), 8.17 (s, 1H), 7.67 (s, 1H), 6.25 (s, 1H), 5.93 (s, 1H), 5.23 (m, 1H), 4.16 (d, J = 12.8 Hz, 1H), 3.93 (dd, J = 3.2, 11.6 Hz, 2H), 3.71 (br d, J = 11.6 Hz, 1H), 3.65 (s, 3H), 3.51 (dt, J = 2.0, 11.6 Hz, 1H), 3.22 (br t, J = 12.0 Hz, 1H), 2.14 (s, 3H). | 413.1 |
| 37 137 16 | G6 A1 SFC separation | 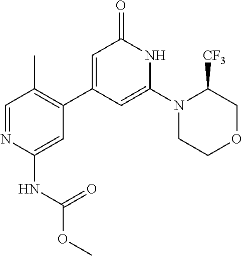 [SMILES: COC(=O)Nc1cc(-c2cc(N3CCOC[C@@H]3C(F)(F)F)[nH]c(=O)c2)c(C)cn1] | 32 | 10.17 (s, 1H), 8.17 (s, 1H), 7.66 (s, 1H), 6.25 (s, 1H), 5.92 (s, 1H), 5.23 (m, 1H), 4.16 (d, J = 12.4 Hz, 1H), 3.93 (dd, J = 2.8, 11.2 Hz, 2H), 3.72 (br d, J = 11.2 Hz, 1H), 3.65 (s, 3H), 3.51 (td, J = 2.0, 11.6 Hz, 1H), 3.22 (br t, J = 11.6 Hz, 1H), 2.14 (s, 3H), one NH is under solvents. | 413.0 |
| 38 121 57 | A2 G2 HCl | 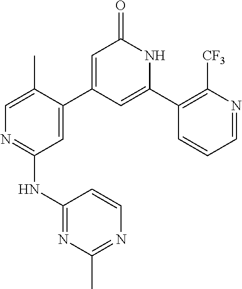 [SMILES: Cc1nccc(Nc2cc(-c3cc(-c4cccnc4C(F)(F)F)[nH]c(=O)c3)c(C)cn2)n1] | 76 | 11.54 (br s, 1H), 8.86 (d, J = 4.6 Hz, 1H), 8.49 (d, J = 7.0 Hz, 1H), 8.38 (s, 1H), 8.16 (br d, J = 7.8 Hz, 1H), 7.85 (dd, J = 4.8, 7.8 Hz, 2H), 7.61 (br s, 1H), 6.49 (br s, 1H), 2.58-2.62 (m, 4H), 2.26 (s, 3H). | 439.1 |
| 39 137 65 | I2 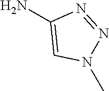 TFA | 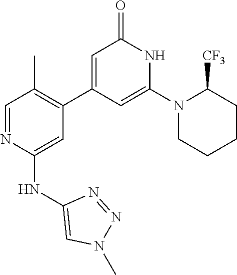 [SMILES: Cc1cnc(Nc2cn(C)nn2)cc1-c1cc(N2CCCC[C@@H]2C(F)(F)F)[nH]c(=O)c1] | 31 | 10.37 (br s, 1H), 9.61 (s, 1H), 8.12 (s, 1H), 8.04 (s, 1H), 6.76 (s, 1H), 6.23 (s, 1H), 5.85 (s, 1H), 5.48 (br m, 1H), 4.23 (br m, 1H), 4.02 (s, 3H), 2.98 (br t, J = 12.8 Hz, 1H), 2.08 (s, 3H), 1.97 (br d, J = 13.6 Hz, 1H), 1.62-1.80 (m, 4H), 1.46 (m, 1H). | 434.1 |
| 40 137 68 | I2 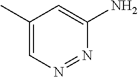 TFA | 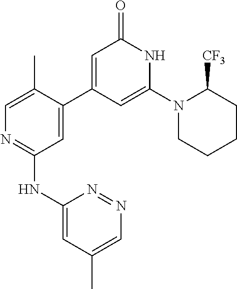 [SMILES: Cc1cnnc(Nc2cc(-c3cc(N4CCCC[C@@H]4C(F)(F)F)[nH]c(=O)c3)c(C)cn2)c1] | 26 | 10.40 (br s, 1H), 9.96 (s, 1H), 8.61 (d, J = 1.6 Hz, 1H), 8.16 (s, 1H), 7.84 (s, 1H), 7.60 (s, 1H), 6.27 (br s, 1H), 5.89 (s, 1H), 5.49 (br m, 1H), 4.13 (br m, 1H), 2.99 (br t, J = 12.4 Hz, 1H), 2.28 (s, 3H), 2.14 (s, 3H), 1.97 (d, J = 12.8 Hz, 1H), 1.62-1.82 (m, 4H), 1.47 (m, 1H). | 445.0 |
| 41 137 69 | I2 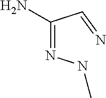 TFA | 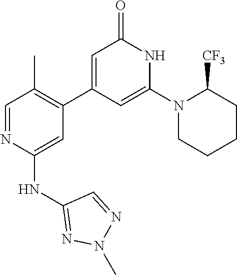 [SMILES: Cc1cnc(Nc2cnn(C)n2)cc1-c1cc(N2CCCC[C@@H]2C(F)(F)F)[nH]c(=O)c1] | 36 | 10.36 (br s, 1H), 9.57 (s, 1H), 8.07 (s, 1H), 7.86 (s, 1H), 6.86 (s, 1H), 6.23 (s, 1H), 5.86 (s, 1H), 5.47 (m, 1H), 4.12 (br d, J = 12.8 Hz, 1H), 4.02 (s, 3H), 2.98 (br t, J = 12.8 Hz, 1H), 2.09 (s, 3H), 1.97 (br d, J = 13.6 Hz, 1H), 1.59- 1.80 (m, 4H), 1.48 (m, 1H). | 434.2 |
| 42 137 70 | I2 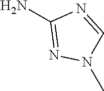 TFA | 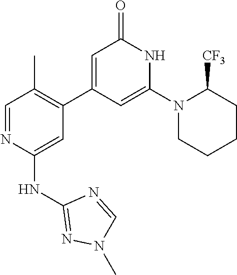 [SMILES: Cc1cnc(Nc2ncn(C)n2)cc1-c1cc(N2CCCC[C@@H]2C(F)(F)F)[nH]c(=O)c1] | 35 | 10.40 (br s, 1H), 9.45 (s, 1H), 8.20 (s, 1H), 8.07 (s, 1H), 7.71 (s, 1H), 6.25 (br s, 1H), 5.87 (s, 1H), 5.49 (br m, 1H), 4.15 (br m, 1H), 3.75 (s, 3H), 2.99 (br t, J = 13.2 Hz, 1H), 2.11 (s, 3H), 1.98 (br d, J = 14.8 Hz, 1H), 1.57-1.85 (m, 4H), 1.47 (m, 1H). | 434.2 |
| 43 138 00 | I2 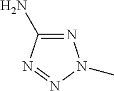 TFA | 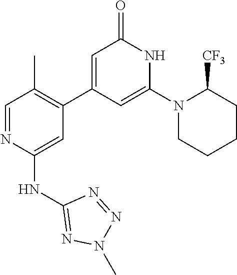 [SMILES: Cc1cnc(Nc2nnn(C)n2)cc1-c1cc(N2CCCC[C@@H]2C(F)(F)F)[nH]c(=O)c1] | 7 | 10.23 (s, 1H), 8.13 (s, 1H), 7.51 (s, 1H), 6.23 (s, 1H), 5.87 (s, 1H), 5.48 (br m, 1H), 4.24 (s, 3H), 4.13 (br d, J = 12.8 Hz, 1H), 2.98 (br t, J = 13.2 Hz, 1H), 2.12 (s, 3H), 1.97 (br d, J = 14.4 Hz, 1H), 1.61-1.80 (m, 4H), 1.46 (m, 1H), one NH is under solvents. | 435.2 |
| 44 138 02 | 12 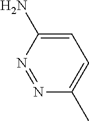 TFA | 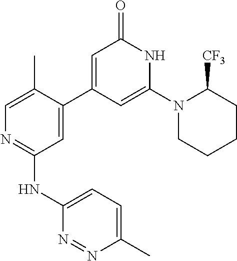 [SMILES: Cc1ccc(Nc2cc(-c3cc(N4CCCC[C@@H]4C(F)(F)F)[nH]c(=O)c3)c(C)cn2)nn1] | 40 | 10.38 (br s, 1H), 9.94 (s, 1H), 8.13 (s, 1H), 7.95 (d, J = 9.2 Hz, 1H), 7.56 (s, 1H), 7.41 (d, J = 9.2 Hz, 1H), 6.25 (s, 1H), 5.88 (s, 1H), 5.49 (br m, 1H), 4.11 (br d, J = 9.6 Hz, 1H), 2.98 (br t, J = 13.6 Hz, 1H), 2.49 (s, 3H), 2.13 (s, 3H), 1.97 (br d, J = 13.6 Hz, 1H), 1.58-1.86 (m, 4H), 1.46 (m, 1H). | 445.2 |
| 45 | A1 G4 HCl | 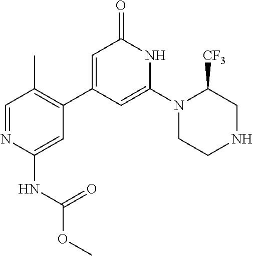 [SMILES: COC(=O)Nc1cc(-c2cc(N3CCNC[C@@H]3C(F)(F)F)[nH]c(=O)c2)c(C)cn1] | 83 | 10.36 (s, 1H), 8.21 (s, 1H), 7.66 (s, 1H), 6.39 (s, 1H), 6.03 (s, 1H), 4.39 (d, J = 13.6 Hz, 1H), 3.67 (s, 3H), 3.39-3.50 (m, 2H), 3.23-3.33 (m, 3H), 2.97-3.16 (m, 1H), 2.16 (s, 3H), 1.98 (s, 1H), 1.91 (s, 1H). | 568.4 |
| 46 | K2 PMBOH NaH TFA or A1 G7 HCl | 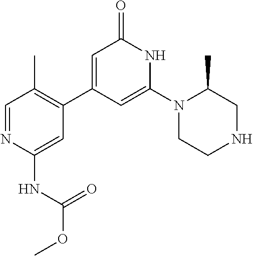 [SMILES: COC(=O)Nc1cc(-c2cc(N3CCNC[C@@H]3C)[nH]c(=O)c2)c(C)cn1] | 24 | | 358.3 |
| 47 139 30 | J2 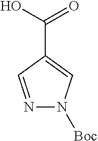 TFA | 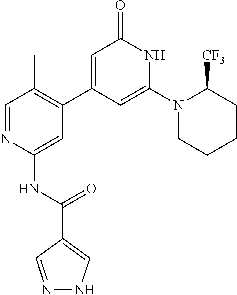 [SMILES: Cc1cnc(NC(=O)c2cn[nH]c2)cc1-c1cc(N2CCCC[C@@H]2C(F)(F)F)[nH]c(=O)c1] | 15 | 10.47 (s, 1H), 8.32 (s, 2H), 8.26 (s, 1H), 8.05 (s, 1H), 6.22 (s, 1H), 5.88 (s, 1H), 5.50 (m, 1H), 4.11 (m, 1H), 2.99 (br t, J = 12.8 Hz, 1H), 2.19 (s, 3H), 1.97 (m, 1H), 1.63-1.81 (m, 4H), 1.46 (m, 1H), 2 NH are under solvents. | 447.2 |
| 48 139 67 | J4 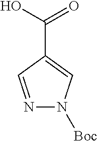 TFA | 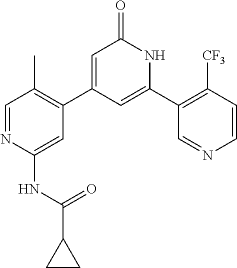 [SMILES: Cc1cnc(NC(=O)C2CC2)cc1-c1cc(-c2cnccc2C(F)(F)F)[nH]c(=O)c1] | 23 | 12.04 (br s, 1H), 10.84 (s, 1H), 8.95 (d, J = 5.2 Hz, 1H), 8.89 (s, 1H), 8.25 (s, 1H), 8.00 (s, 1H), 7.89 (d, J = 5.2 Hz, 1H), 6.46 (s, 1H), 2.21 (s, 3H), 1.99 (m, 1H), 0.80 (d, J = 6.1 Hz, 4H). | 415.2 |
| 49 126 98 | A6 G1 HCl | 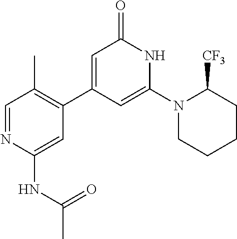 [SMILES: CC(=O)Nc1cc(-c2cc(N3CCCC[C@@H]3C(F)(F)F)[nH]c(=O)c2)c(C)cn1] | 32 | 11.87 (br s, 1H), 8.29 (s, 1H), 7.78 (s, 1H), 7.22-7.55 (br s, 1H), 6.31 (s, 1H), 5.94 (s, 1H), 5.46 (m, 1H), 4.13 (br d, J = 12.3 Hz, 1H), 2.99 (br t, J = 12.9 Hz, 1H), 2.22 (s, 3H), 2.20 (s, 3H), 1.91-2.08 (m, 1H), 1.55-1.88 (m, 4H), 1.37-1.54 (m, 1H). | 359.2 |
| 50 130 51 | A20 G2 HCl | 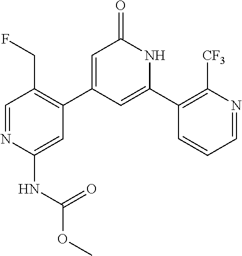 [SMILES: COC(=O)Nc1cc(-c2cc(-c3cccnc3C(F)(F)F)[nH]c(=O)c2)c(CF)cn1] | 38 | 12.24 (br s, 1H), 10.57 (s, 1H), 8.86 (d, J = 4.0 Hz, 1H), 8.49 (d, J = 2.0 Hz, 1H), 8.17 (d, J = 7.2 Hz, 1H), 7.89 (s, 1H), 7.84 (dd, J = 4.8, 8.0 Hz, 1H), 6.51 (br s, 1H), 6.30 (br s, 1H), 5.37 (d, J = 48.4 Hz, 2H), 3.69 (s, 3H). | 423.1 |
| 51 126 30 | A9 G2 HCl | 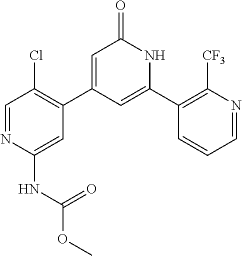 [SMILES: COC(=O)Nc1cc(-c2cc(-c3cccnc3C(F)(F)F)[nH]c(=O)c2)c(Cl)cn1] | 96 | 10.57 (s, 1H), 8.85 (d, J = 4.4 Hz, 1H), 8.44 (s, 1H), 8.16 (d, J = 7.8 Hz, 1H), 7.91 (s, 1H), 7.84 (dd, J = 4.7, 7.8 Hz, 1H), 6.57 (br s, 1H), 3.68 (s, 3H). | 425.0 |
| 52 127 01 | A5 G2 HCl | 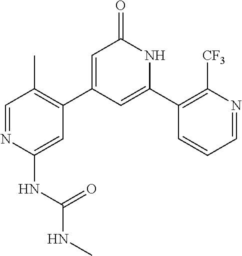 [SMILES: CNC(=O)Nc1cc(-c2cc(-c3cccnc3C(F)(F)F)[nH]c(=O)c2)c(C)cn1] | 10 | 8.89 (m, 1H), 8.19-8.24 (m, 2H), 7.87 (dd, J = 4.9, 7.9 Hz, 1H), 7.38 (s, 1H), 7.06 (d, J = 1.2 Hz, 1H), 7.01(d, J = 1.2 Hz, 1H), 2.86 (s, 3H), 2.32 (s, 3H), 3 NHs are under solvents, in MeOD | 404.1 |
| 53 128 25 | 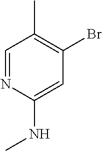 G1 HCl | 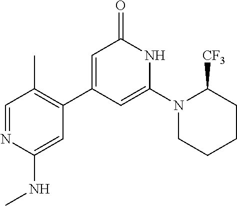 [SMILES: CNc1cc(-c2cc(N3CCCC[C@@H]3C(F)(F)F)[nH]c(=O)c2)c(C)cn1] | 50 | 10.38 (br s, 1H), 7.93 (s, 1H), 6.32-6.35 (m, 2H), 6.26 (br s, 1H), 5.89 (s, 1H), 5.48 (m, 1H), 4.11 (br m, 1H), 3.03 (br t, J = 13.2 Hz, 1H), 2.81 (d, J = 4.8 Hz, 3H), 2.07 (s, 3H), 2.02 (m, 1H), 1.67- 1.89 (m, 4H), 1.44 (m, 1H). | 367.2 |
| 54 134 72 | J2 triphosgene 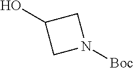 TFA | 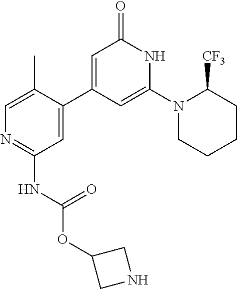 [SMILES: Cc1cnc(NC(=O)OC2CNC2)cc1-c1cc(N2CCCC[C@@H]2C(F)(F)F)[nH]c(=O)c1] | 26 | 10.41 (s, 1H), 8.20 (s, 1H), 7.61 (s, 1H), 6.23 (s, 1H), 5.86 (s, 1H), 5.48 (m, 1H), 5.18 (m, 1H), 4.10 (br d, J = 12.4 Hz, 1H), 4.02-4.07 (m, 2H), 3.77-3.84 (m, 2H), 2.98 (br t, J = 12.8 Hz, 2H), 2.16 (s, 3H), 1.98 (br d, J = 14.4 Hz, 1H), 1.61-1.80 (m, 4H), 1.46 (m, 1H), one NH is under solvents. | 452.1 |
| 55 126 67 | J1 HCl | 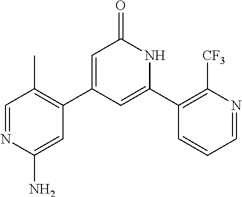 [SMILES: Cc1cnc(N)cc1-c1cc(-c2cccnc2C(F)(F)F)[nH]c(=O)c1] | 57 | 8.84 (d, J = 3.8 Hz, 1H), 8.14 (m, 1H), 7.72-7.87 (m, 2H), 6.41 (br m, 1H), 6.33 (s, 1H), 5.85 (s, 2H), 2.01 (s, 3H), 2 Hs are under solvents. | 347.0 |
| 56 134 73 | A14 G3 TFA | 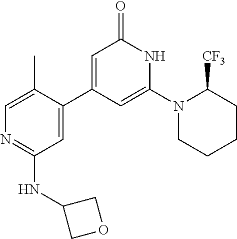 [SMILES: Cc1cnc(NC2COC2)cc1-c1cc(N2CCCC[C@@H]2C(F)(F)F)[nH]c(=O)c1] | 24 | 7.99 (s, 1H), 6.84 (s, 1H), 6.12 (s, 1H), 5.90 (s, 1H), 5.43 (br m, 1H), 4.77 (m, 1H), 4.55 (dd, J = 2.4, 12.4 Hz, 1H), 4.12 (m, 1H), 3.98 (m, 1H), 3.75 (dd, J = 3.6, 11.3 Hz, 1H), 3.68 (dd, J = 3.6, 12.4 Hz 1H), 3.15 (br t, J = 12.8 Hz, 1H), 2.13 (s, 3H), 2.08 (m, 1H), 1.65- 1.90 (m, 4H), 1.55 (m, 1H), 2 NHs are under solvents, in MeOD | 409.2 |
| 57 135 30 | 23 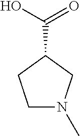 TCFH | 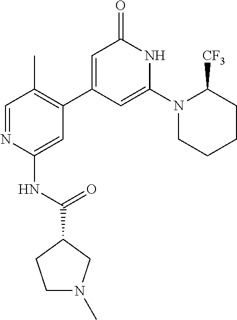 [SMILES: Cc1cnc(NC(=O)[C@H]2CCN(C)C2)cc1-c1cc(N2CCCC[C@@H]2C(F)(F)F)[nH]c(=O)c1] | 37 | 10.43 (m, 2H), 8.21 (s, 1H), 7.94 (s, 1H), 6.23 (s, 1H), 5.86 (s, 1H), 5.49 (m, 1H), 4.10 (br d, J = 13.4 Hz, 1H), 3.15 (m, 1H), 2.98 (t, J = 13.0 Hz, 1H), 2.76 (t, J = 8.5 Hz, 1H), 2.54 (m, 1H), 2.46 (m, 1H), 2.39 (m, 1H), 2.24 (s, 3H), 2.16 (s, 3H), 1.92-2.02 (m, 3H), 1.52- 1.78 (br m, 4H), 1.46 (br m, 1H). | 464.2 |
| 58 136 01 | 45 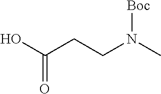 HCl | 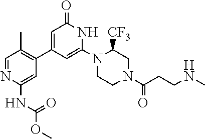 [SMILES: CNCCC(=O)N1CCN(c2cc(-c3cc(NC(=O)OC)ncc3C)cc(=O)[nH]2)[C@@H](C(F)(F)F)C1] | 21 | 10.17 (br s, 1H), 8.18 (s, 1H), 7.67 (s, 1H), 6.30 (s, 1H), 5.94 (s, 1H), 5.44 (br m, 1H), 4.67 (d, J = 14.0 Hz, 0.6H), 4.31 (d, J = 13.6 Hz, 0.4H), 4.20 (d, J = 14.0 Hz, 0.4H), 4.12 (d, J = 12.0 Hz, 1H), 3.93 (d, J = 14.0 Hz, 0.6H), 3.65 (s, 3H), 3.59 (m, 1H), 3.10-3.25 (br m, 3H), 2.85 (m, 1H), 2.62-2.71 (m, 2H), 2.57 (m, 1H), 2.41 (m, 1H), 2.26 (s, 3H), 2.15 (s, 3H). | 497.1 |
| 59 136 10 | J2 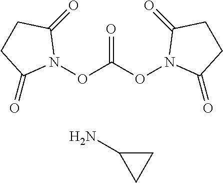 TFA | 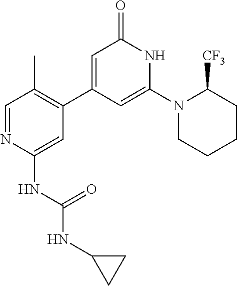 [SMILES: Cc1cnc(NC(=O)NC2CC2)cc1-c1cc(N2CCCC[C@@H]2C(F)(F)F)[nH]c(=O)c1] | 43 | 10.42 (br s, 1H), 9.01 (s, 1H), 8.07 (s, 1H), 7.98 (s, 1H), 7.28 (s, 1H), 6.22 (s, 1H), 5.84 (s, 1H), 5.48 (s, 1H), 4.11 (d, J = 13.5 Hz, 1H), 2.98 (t, J = 13.0 Hz, 1H), 2.57 (m, 1H), 2.10 (s, 3H), 1.97 (d, J = 14.2 Hz, 1H), 1.56-1.82 (m, 4H), 1.45 (br m, 1H), 0.62-0.69 (m, 2H), 0.40-0.45 (m, 2H). | 436.2 |
| 60 135 96 | 46 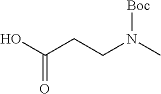 TCFH HCl | 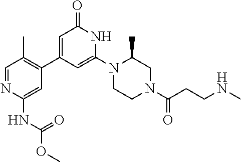 [SMILES: CNCCC(=O)N1CCN(c2cc(-c3cc(NC(=O)OC)ncc3C)cc(=O)[nH]2)[C@@H](C)C1] | 32 | 9.72 (br s, 1H), 8.16 (s, 1H), 7.63 (s, 1H), 6.03 (s, 1H), 5.80 (s, 1H), 4.46 (m, 1H), 4.19 (m, 1H), 3.96 (m, 1H), 3.68 (s, 1H), 3.65 (s, 3H), 2.73 (t, J = 6.4 Hz, 2H), 2.30 (s, 3H), 2.17 (s, 3H), 1.07 (d, J = 4.4 Hz, 3H), 6Hs are under solvents, at high temp. | 443.2 |
| 61 136 16 | L4 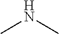 | 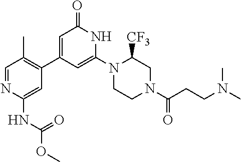 [SMILES: COC(=O)Nc1cc(-c2cc(N3CCN(C(=O)CCN(C)C)C[C@@H]3C(F)(F)F)[nH]c(=O)c2)c(C)cn1] | 20 | 10.4-10.8 (br s, 1H), 10.17 (s, 1H), 8.18 (s, 1H), 7.67 (s, 1H), 6.30 (s, 1H), 5.95 (s, 1H), 5.44 (br m, 1H), 4.67 (br d, J = 14.0 Hz, 1H), 4.26 (m, 1H), 4.11 (d, J = 11.6 Hz, 1H), 3.96 (br d, J = 11.6 Hz, 1H), 3.65 (s, 3H), 3.60 (m, 1H), 3.22 (m, 1H), 3.12 (m, 1H), 2.88 (m, 1H), 2.46 (m, 2H), 2.15 (s, 3H), 2.13 (s, 6H). | 511.1 |
| 62 136 17 | A15 G3 TFA | 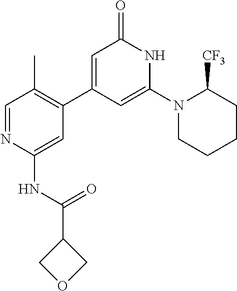 [SMILES: Cc1cnc(NC(=O)C2COC2)cc1-c1cc(N2CCCC[C@@H]2C(F)(F)F)[nH]c(=O)c1] | 27 | 10.45 (br s, 1H), 7.87 (s, 1H), 6.59 (s, 1H), 6.28 (s, 1H), 5.89 (s, 1H), 5.45 (br m, 1H), 4.81 (t, J = 4.4 Hz, 1H), 4.39 (dd, J = 6.0, 14.0 Hz, 1H), 4.14-4.22 (m, 2H), 3.71 (m, 1H), 2.97 (br t, J = 12.0 Hz, 2H), 2.02 (s, 3H), 1.97 (m, 1H), 1.63-1.78 (m, 4H), 1.45 (m, 1H), one NH is under solvents. | 437.2 |
| 63 136 19 | L5 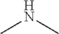 | 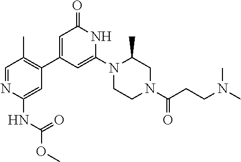 [SMILES: COC(=O)Nc1cc(-c2cc(N3CCN(C(=O)CCN(C)C)C[C@@H]3C)[nH]c(=O)c2)c(C)cn1] | 17 | 10.38 (br s, 1H), 10.16 (s, 1H), 8.16 (s, 1H), 7.66 (s, 1H), 6.03 (br s, 1H), 5.80 (s, 1H), 4.47 (br m, 1H), 4.33 (m, 0.5H), 4.22 (m, 0.5H), 3.90-4.02 (m, 1.5H), 3.80 (m, 0.5H), 3.65 (s, 3H), 3.21 (m, 1H), 3.08 (m, 1H), 2.93 (m, 1H), 2.78 (m, 1H), 2.16 (s, 3H), 2.15 (s, 3H), 2.14 (s, 3H), 1.06 (d, J = 6.4 Hz, 1.5H), 0.98 (d, J = 6.4 Hz, 1.5H), 4Hs are under solvents. | 457.2 |
| 64 136 42 | J1 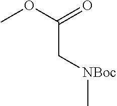 LiHMDS HCl | 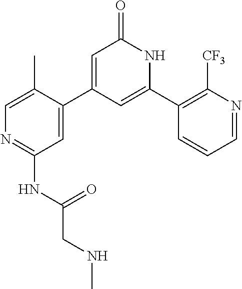 [SMILES: CNCC(=O)Nc1cc(-c2cc(-c3cccnc3C(F)(F)F)[nH]c(=O)c2)c(C)cn1] | 6 | 8.81 (m, 1H), 8.23 (s, 1H), 8.15 (d, J = 7.8 Hz, 1H), 8.01 (s, 1H), 7.82 (dd, J = 4.8, 7.9 Hz, 1H), 6.41 (d, J = 13.2 Hz, 2H), 3.24 (s, 2H), 2.29 (s, 3H), 2.19 (s, 3H). 3 NHs are under solvents. | 418.2 |
| 65 136 63 | A21 G3 TFA | 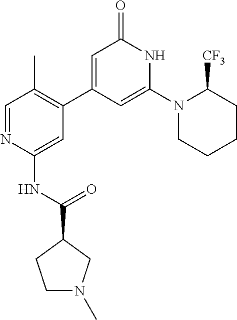 [SMILES: Cc1cnc(NC(=O)[C@@H]2CCN(C)C2)cc1-c1cc(N2CCCC[C@@H]2C(F)(F)F)[nH]c(=O)c1] | 35 | 10.43 (s, 1H), 10.2-10.6 (br s, 1H), 8.21 (s, 1H), 7.93 (s, 1H), 6.22 (s, 1H), 5.86 (s, 1H), 5.49 (m, 1H), 4.10 (br d, J = 11.6 Hz, 1H), 3.15 (m, 1H), 2.98 (br t, J = 13.2 Hz, 1H), 2.75 (t, J = 8.8 Hz, 1H), 2.56 (m, 1H), 2.45 (m, 1H), 2.38 (m, 1H), 2.23 (s, 3H), 2.16 (s, 3H), 1.91-2.02 (m, 3H), 1.62-1.80 (m, 4H), 1.46 (m, 1H). | 464.2 |
| 66 140 18 | L6 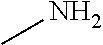 TEA TFA | 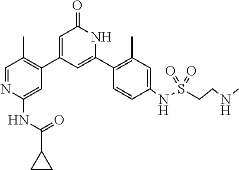 [SMILES: CNCCS(=O)(=O)Nc1ccc(-c2cc(-c3cc(NC(=O)C4CC4)ncc3C)cc(=O)[nH]2)c(C)c1] | 35 | 10.83 (s, 1H), 8.24 (s, 1H), 7.99 (s, 1H), 7.31 (d, J = 8.8 Hz, 1H), 7.10 (s, 1H), 7.08 (m, 1H), 6.25 (d, J = 1.2 Hz, 1H), 6.12 (s, 1H), 3.26 (t, J = 7.2 Hz, 2H), 2.87 (t, J = 7.2 Hz, 2H), 2.29 (s, 3H), 2.25 (s, 3H), 2.24 (s, 3H), 1.99 (m, 1H), 0.79 (d, J = 6.0 Hz, 4H), 3 NHs are under solvents. | 496.2 |
| 67 137 07 | J7 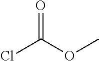 TFA | 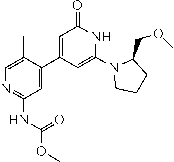 [SMILES: COC[C@H]1CCCN1c1cc(-c2cc(NC(=O)OC)ncc2C)cc(=O)[nH]1] | 38 | 10.28 (br s, 1H), 10.15 (s, 1H), 8.16 (s, 1H), 7.67 (s, 1H), 5.66 (s, 2H), 4.13 (s, 1H), 3.65 (s, 3H), 3.48 (m, 1H), 3.41 (t, J = 8.5 Hz, 1H), 3.26-3.32 (m, 4H), 3.21 (m, 1H), 2.17 (s, 3H), 1.86-2.03 (m, 4H). | 373.2 |
| 68 136 88 | J1 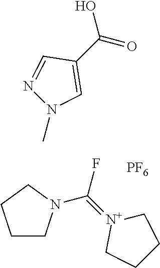 HCl | 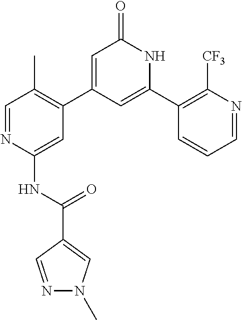 [SMILES: Cc1cnc(NC(=O)c2cnn(C)c2)cc1-c1cc(-c2cccnc2C(F)(F)F)[nH]c(=O)c1] | 11 | 12.04 (s, 1H), 10.54 (s, 1H), 8.83 (d, J = 4.7 Hz, 1H), 8.40 (s, 1H), 8.28 (s, 1H), 8.15 (d, J = 7.9 Hz, 1H), 8.09 (d, J = 8.3 Hz, 2H), 7.82 (dd, J = 4.8, 7.9 Hz, 1H), 6.44 (m, 2H), 3.86 (s, 3H), 2.21 (s, 3H). | 455.2 |
| 69 137 71 | 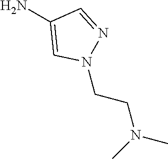 I2 TFA | 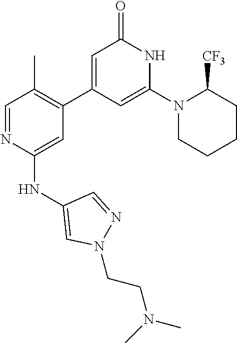 [SMILES: Cc1cnc(Nc2cnn(CCN(C)C)c2)cc1-c1cc(N2CCCC[C@@H]2C(F)(F)F)[nH]c(=O)c1] | 31 | 10.30 (br s, 1H), 8.61 (s, 1H), 7.98 (s, 1H), 7.92 (s, 1H), 7.40 (s, 1H), 6.48 (s, 1H), 6.22 (s, 1H), 5.85 (s, 1H), 5.47 (m, 1H), 4.12 (t, J = 6.4 Hz, 2H), 4.10 (br m, 1H), 2.98 (br t, J = 12.8 Hz, 1H), 2.61 (t, J = 6.8 Hz, 2H), 2.15 (s, 6H), 2.06 (s, 3H), 1.97 (m, 1H), 1.62-1.78 (m, 4H), 1.45 (m, 1H). | 490.1 |
| 70 138 16 | J3 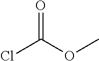 TFA | 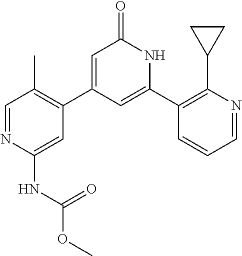 [SMILES: COC(=O)Nc1cc(-c2cc(-c3cccnc3C3CC3)[nH]c(=O)c2)c(C)cn1] | 34 | 12.13 (s, 1H), 10.22 (s, 1H), 8.52-8.47 (m, 1H), 8.20 (s, 1H), 7.71-7.78 (m, 2H), 7.24 (dd, J = 4.8, 7.7 Hz, 1H), 6.37 (s, 1H), 3.66 (s, 3H), 2.23 (s, 3H), 2.14- 2.05 (m, 1H), 1.06-0.90 (m, 4H). | 377.2 |
| 71 138 01 | I2 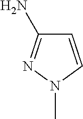 TFA | 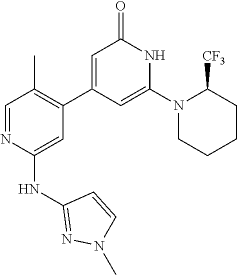 [SMILES: Cc1cnc(Nc2ccn(C)n2)cc1-c1cc(N2CCCC[C@@H]2C(F)(F)F)[nH]c(=O)c1] | 15 | 9.08 (s, 1H), 8.00 (s, 1H), 7.48 (d, J = 2.0 Hz, 1H), 7.10 (s, 1H), 6.26 (d, J = 2.4 Hz, 1H), 6.22 (s, 1H), 5.86 (s, 1H), 5.47 (br m, 1H), 4.13 (br d, J = 12.8 Hz, 1H), 3.71 (s, 3H), 2.99 (br t, J = 12.8 Hz, 1H), 2.07 (s, 3H), 1.98 (br d, J = 13.6 Hz, 1H), 1.64-1.80 (m, 4H), 1.46 (m, 1H), one NH is under solvents. | 433.2 |
| 72 137 67 | I2 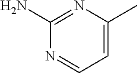 HCl | 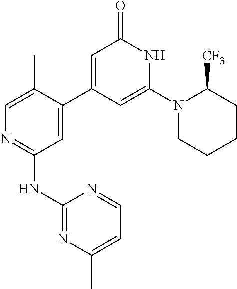 [SMILES: Cc1ccnc(Nc2cc(-c3cc(N4CCCC[C@@H]4C(F)(F)F)[nH]c(=O)c3)c(C)cn2)n1] | 45 | 10.35 (br s, 1H), 9.61 (s, 1H), 8.36 (d, J = 4.8 Hz, 1H), 8.17 (s, 2H), 6.80 (d, J = 5.2 Hz, 1H), 6.28 (br s, 1H), 5.91 (s, 1H), 5.48 (br m, 1H), 4.15 (br d, J = 11.2 Hz, 1H), 2.98 (br t, J = 12.8 Hz, 1H), 2.35 (s, 3H), 2.16 (s, 3H), 1.97 (br d, J = 14.0 Hz, 1H), 1.62-1.82 (m, 4H), 1.47 (m, 1H). | 445.8 |
| 73 139 19 | B4 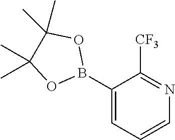 HCl | 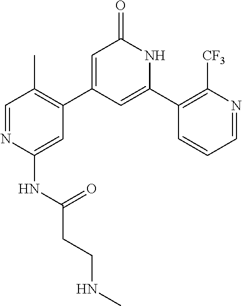 [SMILES: CNCCC(=O)Nc1cc(-c2cc(-c3cccnc3C(F)(F)F)[nH]c(=O)c2)c(C)cn1] | 8 | 10.88 (s, 1H), 8.83 (dd, J = 1.5, 4.8 Hz, 1H), 8.74 (br s, 2H), 8.26 (s, 1H), 8.14 (m, 1H), 7.97 (s, 1H), 7.82 (dd, J = 4.7, 7.9 Hz, 1H), 6.41 (m, 2H), 3.14 (m, 2H), 2.85 (t, J = 6.8 Hz, 2H), 2.53 (t, J = 5.4 Hz, 3H), 2.20 (s, 3H). | 432.0 |
| 74 139 71 | J2 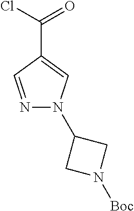 TFA Et₃SiH | 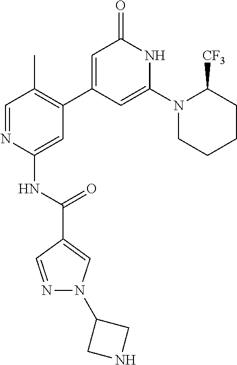 [SMILES: Cc1cnc(NC(=O)c2cnn(C3CNC3)c2)cc1-c1cc(N2CCCC[C@@H]2C(F)(F)F)[nH]c(=O)c1] | 9 | 10.53 (s, 1H), 8.64 (s, 1H), 8.27 (s, 1H), 8.18 (s, 1H), 8.04 (s, 1H), 6.27 (s, 1H), 5.90 (s, 1H), 5.50 (m, 1H), 5.21 (m, 1H), 4.13 (br d, J = 12.4 Hz, 1H), 3.88 (br t, J = 6.0 Hz, 2H), 3.75 (br t, J = 7.2 Hz, 2H), 2.99 (br t, J = 13.2 Hz, 1H), 2.19 (s, 3H), 1.98 (br d, J = 14.4 Hz, 1H), 1.60-1.84 (m, 5H), 1.47 (m, 1H), one NH is under solvents. | 502.2 |
| 75 | G5 D5 HCl | 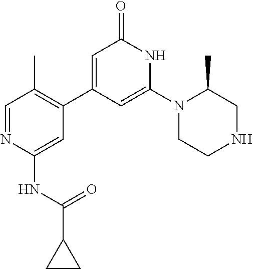 [SMILES: Cc1cnc(NC(=O)C2CC2)cc1-c1cc(N2CCNC[C@@H]2C)[nH]c(=O)c1] | 102 | | 368.2 |
| | | HCl salt | | | |
| 76 139 98 | 75 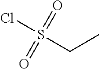 pyridine | 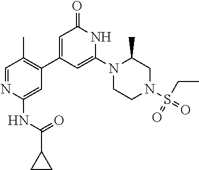 [SMILES: CCS(=O)(=O)N1CCN(c2cc(-c3cc(NC(=O)C4CC4)ncc3C)cc(=O)[nH]2)[C@@H](C)C1] | 2 | 10.76 (s, 1H), 10.35 (br s, 1H), 8.20 (s, 1H), 7.91 (s, 1H), 6.07 (br s, 1H), 5.80 (s, 1H), 4.58 (br s, 1H), 4.09 (br s, 1H), 3.59 (d, J = 11.3 Hz, 1H), 3.42 (d, J = 11.3 Hz, 1H), 3.08 (m, 2H), 3.01 (m, 2H), 2.88 (m, 1H), 2.15 (s, 3H), 1.97 (m, 1H), 1.21 (t, J = 7.4 Hz, 3H), 1.10 (d, J = 6.5 Hz, 3H), 0.77 (m, 4H). | 460.2 |
| 77 122 06 | A2 G1 | 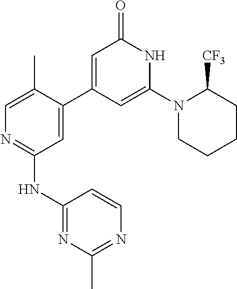 [SMILES: Cc1nccc(Nc2cc(-c3cc(N4CCCC[C@@H]4C(F)(F)F)[nH]c(=O)c3)c(C)cn2)n1] | 21 | 10.41 (br s, 1H), 10.12 (s, 1H), 8.31 (d, J = 6.0 Hz, 1H), 8.20 (s, 1H), 7.63 (d, J = 5.6 Hz, 1H), 7.51 (s, 1H), 6.29 (s, 1H), 5.90 (s, 1H), 5.48 (br m, 1H), 4.15 (br m, 1H), 2.99 (br t, J = 12.4 Hz, 1H), 2.44 (s, 3H), 2.16 (s, 3H), 1.97 (m, 1H), 1.66-1.78 (m, 4H), 1.47 (m, 1H). | 445.3 |
| 78 122 36 | A1 G2 HCl | 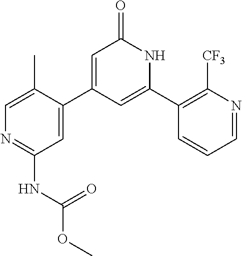 [SMILES: COC(=O)Nc1cc(-c2cc(-c3cccnc3C(F)(F)F)[nH]c(=O)c2)c(C)cn1] | 97 | 11.15 (s, 1H), 8.86 (d, J = 3.9 Hz, 1H), 8.53 (br s, 1H), 8.29 (m, 1H), 8.18 (d, J = 7.4 Hz, 1H), 7.85 (dd, J = 4.8, 8.0 Hz, 1H), 7.74 (s, 1H), 6.55 (d, J = 1.2 Hz, 1H), 6.50 (s, 1H), 3.69-3.79 (m, 3H), 2.23 (s, 3H). | 405.0 |
| | | HCl salt | | | |
| 79 138 22 | J5 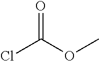 TFA | 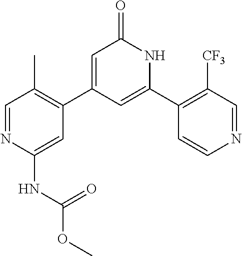 [SMILES: COC(=O)Nc1cc(-c2cc(-c3ccncc3C(F)(F)F)[nH]c(=O)c2)c(C)cn1] | 63 | 11.92 (br s, 1H), 10.23 (s, 1H), 9.08 (s, 1H), 8.97 (d, J = 5.0 Hz, 1H), 8.20 (s, 1H), 7.74 (d, J = 4.3 Hz, 2H), 6.52 (s, 2H), 3.66 (s, 3H), 2.19 (s, 3H). | 405.0 |
| 80 141 23 | A24 G3 HCl | 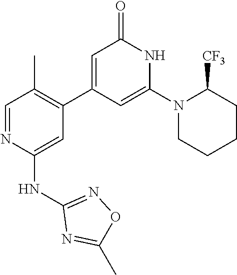 [SMILES: Cc1nc(Nc2cc(-c3cc(N4CCCC[C@@H]4C(F)(F)F)[nH]c(=O)c3)c(C)cn2)no1] | 41 | 10.78 (br s, 1H), 8.81 (s, 1H), 7.51 (s, 1H), 6.34 (s, 1H), 5.95 (s, 1H), 5.51 (br m, 1H), 4.10-4.22 (m, 2H), 2.99 (br t, J = 12.0 Hz, 1H), 2.22 (s, 3H), 2.14 (br s, 3H), 1.98 (br m, 1H), 1.59-1.82 (m, 4H), 145 (br m, 1H), one NH is under solvents. | 435.1 |
| 81 | D10 G5 HCl | 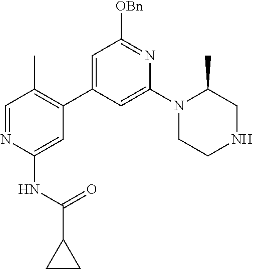 [SMILES: Cc1cnc(NC(=O)C2CC2)cc1-c1cc(OCc2ccccc2)nc(N2CCNC[C@@H]2C)c1] | 86 | | 458.2 |
| 82 | 81 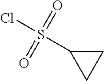 DIEA H₂, Pd-C | 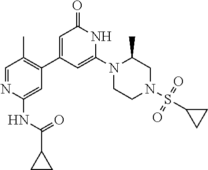 [SMILES: Cc1cnc(NC(=O)C2CC2)cc1-c1cc(N2CCN(S(=O)(=O)C3CC3)C[C@@H]2C)[nH]c(=O)c1] | 29 | 10.78 (s, 1H), 8.21 (s, 1H), 7.92 (s, 1H), 6.06 (br s, 1H), 5.81 (s, 1H), 4.62 (br m, 1H), 4.12 (m, 1H), 3.60 (br d, J = 10.8 Hz, 1H), 3.43 (br d, J = 11.2 Hz, 1H), 2.60 (m, 1H), 2.16 (s, 3H), 1.99 (m, 1H), 1.12 (d, J = 6.6 Hz, 3H), 0.89- 1.02 (m, 4H), 0.79 (d, J = 6.0 Hz, 4H), one NH is under solvents. | 472.2 |
| 83 141 58 | 81 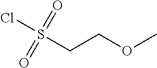 DIEA H₂, Pd-C | 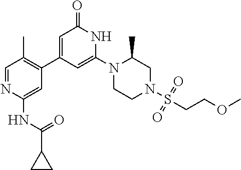 [SMILES: COCCS(=O)(=O)N1CCN(c2cc(-c3cc(NC(=O)C4CC4)ncc3C)cc(=O)[nH]2)[C@@H](C)C1] | 22 | 10.78 (s, 1H), 10.47 (br m, 1H), 8.21 (s, 1H), 7.92 (s, 1H), 6.07 (br s, 1H), 5.81 (s, 1H), 4.59 (br s, 1H), 4.09 (d, J = 11.6 Hz, 1H), 3.66 (t, J = 6.0 Hz, 2H), 3.58 (br d, J = 11.6 Hz, 1H), 3.40 (br d, J = 12.0 Hz, 1H), 3.35-3.37 (m, 2H), 3.26 (s, 3H), 2.97-3.07 (m, 2H), 2.87 (m, 1H), 2.16 (s, 3H), 1.99 (m, 1H), 1.11 (d, J = 6.4 Hz, 3H), 0.73-0.82 (m, 4H). | 490.1 |
| 84 141 59 | 81 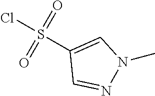 DIEA H₂, Pd-C | 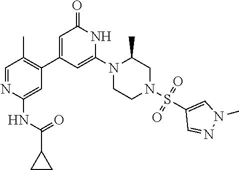 [SMILES: Cc1cnc(NC(=O)C2CC2)cc1-c1cc(N2CCN(S(=O)(=O)c3cnn(C)c3)C[C@@H]2C)[nH]c(=O)c1] | 38 | 10.77 (s, 1H), 10.02-10.60 (m, 1H), 8.33 (s, 1H), 8.20 (s, 1H), 7.90 (s, 1H), 7.78 (s, 1H), 6.02 (br s, 1H), 5.78 (s, 1H), 4.59 (br m, 1H), 4.09 (br m, 1H), 3.89 (s, 3H), 3.56 (br d, J = 11.2 Hz, 1H), 3.39 (m, 1H), 3.07 (m, 1H), 2.39 (dd, J = 3.6, 11.2 Hz, 1H), 2.25 (m, 1H), 2.14 (s, 3H), 1.98 (m, 1H), 1.15 (d, J = 6.4 Hz, 3H), 0.78 (d, J = 6.0 Hz, 4H). | 512.2 |
| 85 141 60 | 81 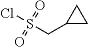 DIEA H₂, Pd-C | 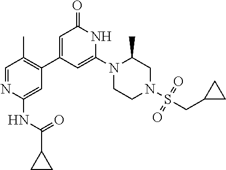 [SMILES: Cc1cnc(NC(=O)C2CC2)cc1-c1cc(N2CCN(S(=O)(=O)CC3CC3)C[C@@H]2C)[nH]c(=O)c1] | 20 | 10.78 (s, 1H), 10.35 (br s, 1H), 8.21 (s, 1H), 7.92 (s, 1H), 6.08 (br m, 1H), 5.81 (s, 1H), 4.59 (br m, 1H), 4.10 (br m, 1H), 3.63 (br d, J = 10.0 Hz, 1H), 3.45 (br d, J = 11.6 Hz, 1H), 3.09 (d, J = 2.8 Hz, 1H), 3.04 (d, J = 7.2 Hz, 2H), 2.96- 3.02 (m, 1H), 2.90 (m, 1H), 2.16 (s, 3H), 1.99 (m, 1H), 1.11 (d, J = 6.4 Hz, 3H), 1.00 (m, 1H), 0.79 (d, J = 6.4 Hz, 4H), 0.56-0.62 (m, 2H), 0.30-0.38 (m, 2H). | 486.2 |
| 86 141 61 | 81 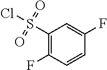 DIEA H₂, Pd-C | 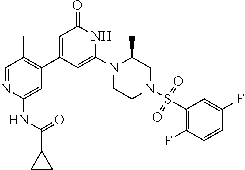 [SMILES: Cc1cnc(NC(=O)C2CC2)cc1-c1cc(N2CCN(S(=O)(=O)c3cc(F)ccc3F)C[C@@H]2C)[nH]c(=O)c1] | 39 | 10.77 (s, 1H), 10.12-10.60 (br s, 1H), 8.20 (s, 1H), 7.90 (s, 1H), 7.54-7.70 (m, 3H), 6.02 (s, 1H), 5.79 (s, 1H), 4.59 (br m, 1H), 4.10 (br d, J = 13.2 Hz, 1H), 3.73 (br d, J = 11.6 Hz, 1H), 3.54 (br d, J = 12.0 Hz, 1H), 3.06 (dt, J = 3.2, 13.2 Hz, 1H), 2.81 (br d, J = 10.0 Hz, 1H), 2.66 (br t, J = 10.4 Hz, 1H), 2.14 (s, 3H), 1.98 (m, 1H), 1.10 (d, J = 6.6 Hz, 3H), 0.78 (d, J = 6.0 Hz, 4H). | 544.2 |
| 87 141 62 | 81 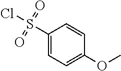 DIEA H₂, Pd-C | 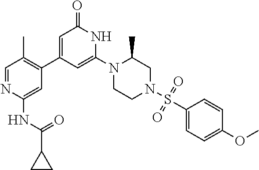 [SMILES: COc1ccc(S(=O)(=O)N2CCN(c3cc(-c4cc(NC(=O)C5CC5)ncc4C)cc(=O)[nH]3)[C@@H](C)C2)cc1] | 19 | 10.76 (s, 1H), 10.30-10.60 (br s, 1H), 8.19 (s, 1H), 7.89 (s, 1H), 7.68 (d, J = 8.8 Hz, 2H), 7.16 (d, J = 8.8 Hz, 2H), 6.00 (s, 1H), 5.78 (s, 1H), 4.56 (br m, 1H), 4.05 (br d, J = 12.0 Hz, 1H), 3.85 (s, 3H), 3.63 (br d, J = 10.4 Hz, 1H), 3.45 (br d, J = 11.6 Hz, 1H), 3.06 (td, J = 2.8, 12.8 Hz, 1H), 2.35 (dd, J = 3.6, 12.0 Hz, 1H), 2.21 (td, J = 3.2, 11.6 Hz, 1H), 2.12 (s, 3H), 1.98 (m, 1H), 1.13 (d, J = 6.6 Hz, 3H), 0.78 (d, J = 6.0 Hz, 4H). | 538.2 |
| 88 141 63 | 81 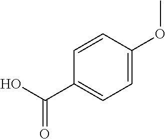 | 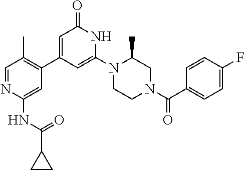 [SMILES: Cc1cnc(NC(=O)C2CC2)cc1-c1cc(N2CCN(C(=O)c3ccc(F)cc3)C[C@@H]2C)[nH]c(=O)c1] | 29 | 10.76 (s, 1H), 8.19 (s, 1H), 7.91 (s, 1H), 7.51 (br s, 2H), 7.29 (t, J = 8.8 Hz, 2H), 5.95 (br m, 1H), 5.76 (s, 1H), 4.36-4.86 (br m, 1H), 3.90-3.42 (br m, 1H), 3.37- 3.88 (br m, 3H), 2.93-3.14 (br m, 2H), 2.15 (s, 3H), 1.98 (br m, 1H), 0.85-1.33 (br m, 3H), 0.78 (d, J = 6.0 Hz, 4H), one NH is under solvents. | 490.2 |
| | TCFH | | | | |
| | H₂, Pd-C | | | | |
| 89 141 64 | 81 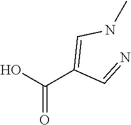 TCFH | 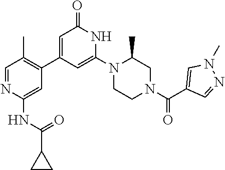 [SMILES: Cc1cnc(NC(=O)C2CC2)cc1-c1cc(N2CCN(C(=O)c3cnn(C)c3)C[C@@H]2C)[nH]c(=O)c1] | 32 | 10.77 (s, 1H), 8.20 (s, 1H), 8.09 (s, 1H), 7.92 (s, 1H), 7.69 (s, 1H), 5.96 (br m, 1H), 5.77 (s, 1H), 4.49 (br m, 1H), 4.01-4.41 (br m, 2H), 3.95 (br m, 1H), 3.86 (s, 3H), 3.39-3.63 (br m, 1H), 3.03-3.18 (br m, 2H), 2.17 (s, 3H), 1.99 (m, 1H), 1.03 (d, J = 6.6 Hz, 3H), 0.78 (d, J = 6.0 Hz, 4H), one NH is under solvents. | 476.2 |
| | H₂, Pd-C | | | | |
| 90 141 84 | I2 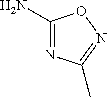 TFA | 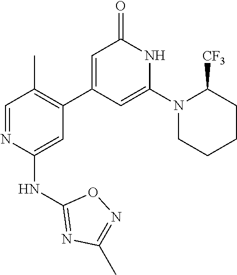 [SMILES: Cc1noc(Nc2cc(-c3cc(N4CCCC[C@@H]4C(F)(F)F)[nH]c(=O)c3)c(C)cn2)n1] | 6 | 11.42 (br m, 1H), 10.44 (br m, 1H), 8.24 (s, 1H), 7.74 (s, 1H), 6.29 (br s, 1H), 5.88 (s, 1H), 5.48 (br m, 1H), 4.15 (m, 1H), 2.98 (t, J = 13.6 Hz, 1H), 2.21 (s, 3H), 2.16 (s, 3H), 1.96 (m, 1H), 1.63-1.81 (br m, 4H), 1.46 (br m, 1H). | 435.1 |
| 91 141 85 | 81 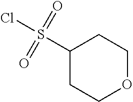 DIEA H₂, Pd-C | 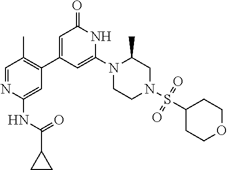 [SMILES: Cc1cnc(NC(=O)C2CC2)cc1-c1cc(N2CCN(S(=O)(=O)C3CCOCC3)C[C@@H]2C)[nH]c(=O)c1] | 41 | 10.78 (s, 1H), 10.40 (br m, 1H), 8.21 (s, 1H), 7.92 (s, 1H), 6.06 (br s, 1H), 5.81 (s, 1H), 4.56 (br s, 1H), 4.07 (br m, 1H), 3.92 (dd, J = 3.6, 11.2 Hz, 2H), 3.66 (br d, J = 9.2 Hz, 1H), 3.43-3.54 (m, 2H), 3.34-3.38 (m, 2H), 3.20 (dd, J = 2.8, 12.0 Hz, 1H), 2.94-3.08 (m, 2H), 2.17 (s, 3H), 1.99 (m, 1H), 1.87 (br d, J = 12.4 Hz, 2H), 1.57-1.70 (m, 2H), 1.10 (d, J = 6.8 Hz, 3H), 0.79 (br d, J = 6.4 Hz, 4H). | 516.2 |
| 92 141 38 | 81 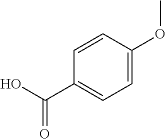 | 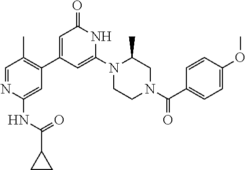 [SMILES: COc1ccc(C(=O)N2CCN(c3cc(-c4cc(NC(=O)C5CC5)ncc4C)cc(=O)[nH]3)[C@@H](C)C2)cc1] | 18 | 1H NMR (400 MHz, DMSO-d6): δ 10.77 (s, 1H), 10.21 (br s, 1H), 8.21 (s, 1H), 7.92 (s, 1H), 7.40 (d, J = 8.4 Hz, 2H), 7.00 (d, J = 8.4 Hz, 2H), 6.02 (br s, 1H), 5.80 (s, 1H), 4.52 (br s, 1H), 3.91 (br m, 1H), 3.80 (s, 3H), 3.00-3.17 (br m, 2H), 2.16 (s, 3H), 1.98 (m, 1H), 1.02 (br s, 3H), 0.78 (m, 4H), 3Hs are under solvents. | 502.2 |
| | TCFH | | | | |
| | H₂, Pd-C | | | | |
| 93 141 37 | 81 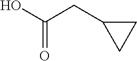 TCFH H₂, Pd-C | 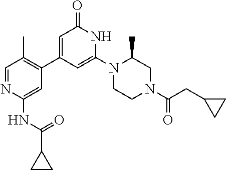 [SMILES: Cc1cnc(NC(=O)C2CC2)cc1-c1cc(N2CCN(C(=O)CC3CC3)C[C@@H]2C)[nH]c(=O)c1] | 31 | 10.45 (s, 1H), 9.72 (br s, 1H), 8.19 (s, 1H), 7.90 (s, 1H), 5.99 (s, 1H), 5.80 (s, 1H), 4.47 (m, 1H), 3.95 (m, 1H), 3.68- 4.47 (br m, 2H), 3.25-3.51 (br m, 2H), 2.55 (m, 1H), 2.24-2.39 (m, 2H), 2.18 (s, 3H), 2.01 (m, 1H), 1.06 (m, 3H), 0.99 (m, 1H), 0.72-0.89 (m, 4H), 0.43- 0.52 (m, 2H), 0.12-0.20 (m, 2H), at high temp. | 450.2 |
| 94 141 39 | 81 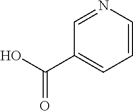 TCFH H₂, Pd-C | 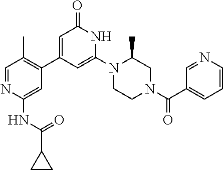 [SMILES: Cc1cnc(NC(=O)C2CC2)cc1-c1cc(N2CCN(C(=O)c3cccnc3)C[C@@H]2C)[nH]c(=O)c1] | 20 | 10.77 (s, 1H), 8.67 (d, J = 4.0 Hz, 1H), 8.63 (m, 1H), 8.20 (s, 1H), 7.92 (s, 1H), 7.87 (m, 1H), 7.50 (dd, J = 4.8, 8.0 Hz, 1H), 5.97 (br s, 1H), 5.77 (br s, 1H), 44.65 (br m, 0.5H), 4.30-4.50 (br m, 1.5H), 4.12 (m, 0.5H), 3.87 (m, 0.5H), 3.41-3.67 (m, 2H), 3.01-3.17 (m, 2H), 2.16 (s, 3H), 1.98 (m, 1H), 1.12 (br m, 1.5H), 0.98 (br m, 1.5H), 0.78 (m, 4H), one NH is under solvents. | 473.2 |
| 95 141 40 | 81 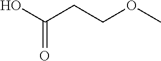 TCFH H₂, Pd-C | 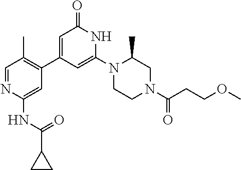 [SMILES: COCCC(=O)N1CCN(c2cc(-c3cc(NC(=O)C4CC4)ncc3C)cc(=O)[nH]2)[C@@H](C)C1] | 21 | 10.77 (s, 1H), 10.43 (br m, 1H), 8.21 (s, 1H), 7.92 (s, 1H), 6.02 (s, 1H), 5.79 (s, 1H), 4.47 (br m, 1H), 4.32 (d, J = 12.0 Hz, 0.5H), 4.22 (d, J = 11.5Hz, 0.5H), 3.95 (br m, 1.5H), 3.83 (m, 0.5H), 3.57 (t, J = 6.0 Hz, 2H), 3.23 (d, J = 3.2 Hz, 3H), 3.18 (m, 0.5H), 3.05 (m, 0.5H), 2.88-3.00 (brm, 1H), 2.63-2.80 (br m, 1H), 2.57 (m, 1H), 2.17 (s, 3H), 1.99 (m, 1H), 1.04 (d, J = 6.4 Hz, 1.5H), 0.97 (d, J = 6.4 Hz, 1.5H), 0.75-0.83 | 454.1 |
| | | | | (m, 4H), one NH is under solvents. | |

### Preparation of Example 96: 2,4-difluoro-N-(5′-methyl-2′-((2-methylpyrimidin-4-yl)amino)-6-oxo-1,6-dihydro-[4,4′-bipyridin]-2-yl)benzenesulfonamide 14016

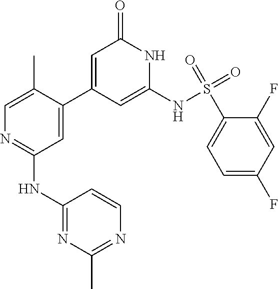 [SMILES: Cc1nccc(Nc2cc(-c3cc(NS(=O)(=O)c4ccc(F)cc4F)[nH]c(=O)c3)c(C)cn2)n1]

A solution of 6-amino-4-[5-methyl-2-[(2-methylpyrimidin-4-yl)amino]-4-pyridyl]-1H-pyridin-2-one TFA salt (J8, 0.20 g, 0.47 mmol) in pyridine (3 mL) was added 2,4-difluorobenzenesulfonyl chloride (0.06 mL, 0.47 mmol) and stirred at rt for 1 h. The reaction was quenched with water (10 mL) and the solution was extracted with EtOAc (3×), the combined organics were dried over anhydrous Na₂SO₄, filtered, and concentrated under reduced pressure. The residue was purified by C-18 prep-HPLC (30 to 60% H₂O (0.05% NH₃H₂O-10 mM NH₄HCO₃)/MeCN) to give 2,4-difluoro-N-(5′-methyl-2′-((2-methylpyrimidin-4-yl)amino)-6-oxo-1,6-dihydro-[4,4′-bipyridin]-2-yl)benzenesulfonamide (81, 0.050 g, 22%) as a white solid. ¹H NMR (400 MHz, DMSO-d₆): δ 9.85 (s, 1H), 8.30 (d, J=6.0 Hz, 1H), 8.21 (s, 1H), 8.04 (m, 1H), 7.59 (m, 1H), 7.55 (m, 2H), 7.33 (m, 1H), 6.40 (d, J=1.2 Hz, 1H), 6.25 (br s, 2H), 6.22 (d, J=0.8 Hz, 1H), 2.46 (s, 3H), 2.12 (s, 3H); MS (ESI) m/z: 485.1 (M+H⁺).

The following compounds are prepared essentially by method of preparation example 96.

| Ex | | | Yield | | MS (m/z: |
|---|---|---|---|---|---|
| No | SM | Product | (%) | 1H NMR (400 MHz, DMSO-d6): d | M + H+) |
| 97 140 93 | G5 D8 | 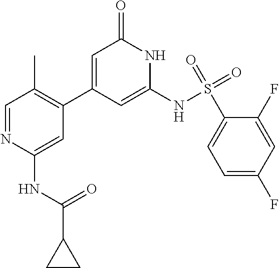 [SMILES: Cc1cnc(NC(=O)C2CC2)cc1-c1cc(NS(=O)(=O)c2ccc(F)cc2F)[nH]c(=O)c1] | 10 | 10.71 (s, 1H), 10.40 (br m, 1H), 8.12 (s, 1H), 7 79 (s, 1H), 7.65-7.89 (m, 1H), 7.27 (m, 1H), 7.09 (m, 1H), 7.05-7.15 (br s, 1H), 5.44 (br s, 1H), 5.30 (br s, 1H), 1.99 (s, 3H), 1.92-2.02 (m, 1H), 0.74-0.85 (m, 4H). | 461.1 |

### Biochemical Assay for VPS34

Activity of VPS34 kinase was determined using an ADP-Glo kinase activity assay (Promega Corp). Assays were conducted in 384-well plates (5 μL assay volume) using 25 nM VPS34 (ThermoFisher Scientific), 100 μg/μL PI:3PS Lipid Kinase Substrate (Promega Corp.), and 1 mM ATP in kinase buffer. Inhibition of VPS34 was measured by adding serial diluted test compound (final assay concentration of 0.1% DMSO) followed by a 1-hour incubation at 37° C. ADP-Glo reagent was then added (5 mL) followed by a 40 min incubation at rt. Kinase detection reagent was then added (10 mL) and followed by a 60 min incubation at rt. Luminescence was then detected. The RLU at each compound concentration of was converted to percent inhibition using controls (i.e., reaction with no test compound and reaction with a known inhibitor) and IC₅₀ values were calculated by fitting a four-parameter sigmoidal curve to the data using GeneData Screener software.

VPS34 Protein Sequence (Full Length with GST Tag)

(SEQ ID NO: 1)
MAPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLVPRHNQTSLYKKAGTMGEAEKFHY
IYSCDLDINVQLKIGSLEGKREQKSYKAVLEDPMLKFSGLYQETCSDLY
VTCQVFAEGKPLALPVRTSYKAFSTRWNWNEWLKLPVKYPDLPRNAQVA
LTIWDVYGPGKAVPVGGTTVSLFGKYGMFRQGMHDLKVWPNVEADGSEP
TKTPGRTSSTLSEDQMSRLAKLTKAHRQGHMVKVDWLDRLTFREIEMIN
ESEKRSSNFMYLMVEFRCVKCDDKEYGIVYYEKDGDESSPILTSFELVK
VPDPQMSMENLVESKHHKLARSLRSGPSDHDLKPNAATRDQLNIIVSYP
PTKQLTYEEQDLVWKFRYYLTNQEKALTKFLKCVNWDLPQEAKQALELL
GKWKPMDVEDSLELLSSHYTNPTVRRYAVARLRQADDEDLLMYLLQLVQ
ALKYENFDDIKNGLEPTKKDSQSSVSENVSNSGINSAEIDSSQIITSPL
PSVSSPPPASKTKEVPDGENLEQDLCTFLISRACKNSTLANYLYWYVIV
ECEDQDTQQRDPKTHEMYLNVMRRFSQALLKGDKSVRVMRSLLAAQQTF
VDRLVHLMKAVQRESGNRKKKNERLQALLGDNEKMNLSDVELIPLPLEP
QVKIRGIIPETATLFKSALMPAQLFFKTEDGGKYPVIFKHGDDLRQDQL
ILQIISLMDKLLRKENLDLKLTPYKVLATSTKHGFMQFIQSVPVAEVLD
TEGSIQNFFRKYAPSENGPNGISAEVMDTYVKSCAGYCVITYILGVGDR
HLDNLLLTKTGKLFHIDFGYILGRDPKPLPPPMKLNKEMVEGMGGTQSE
QYQEFRKQCYTAFLHLRRYSNLILNLFSLMVDANIPDIALEPDKTVKKV
QDKFRLDLSDEEAVHYMQSLIDESVHALFAAVVEQIHKFAQYWRK

TABLE 1
Inhibition of biochemical activity of VPS34
by exemplary compounds (“Ex. No.”).

| Ex No | VPS34 |
|---|---|
| 1 | ++++ |
| 2 | ++++ |
| 3 | +++ |
| 4 | ++++ |
| 5 | +++ |
| 6 | ++ |
| 7 | +++ |
| 8 | +++ |
| 9 | +++ |
| 10 | +++ |
| 11 | + |
| 12 | +++ |
| 13 | + |
| 14 | ++++ |
| 15 | +++ |
| 16 | +++ |
| 17 | ++++ |
| 18 | +++ |
| 19 | + |
| 20 | +++ |
| 21 | ++ |
| 22 | +++ |
| 23 | + |
| 24 | +++ |
| 25 | +++ |
| 26 | +++ |
| 27 | +++ |
| 28 | +++ |
| 29 | ++ |
| 30 | +++ |
| 31 | ++ |
| 32 | +++ |
| 33 | ++ |
| 34 | ++++ |
| 35 | ++ |
| 36 | + |
| 37 | ++++ |
| 38 | ++++ |
| 39 | ++ |
| 40 | ++ |
| 41 | +++ |
| 42 | ++++ |
| 43 | +++ |
| 44 | ++++ |
| 47 | ++ |
| 48 | +++ |
| 49 | ++++ |
| 50 | + |
| 51 | + |
| 52 | + |
| 53 | + |
| 54 | + |
| 55 | + |
| 56 | + |
| 57 | + |
| 58 | + |
| 59 | + |
| 60 | + |
| 61 | + |
| 62 | + |
| 63 | + |
| 64 | + |
| 65 | + |
| 66 | +++ |
| 67 | + |
| 68 | + |
| 69 | + |
| 70 | + |
| 71 | + |
| 72 | ++++ |
| 73 | + |
| 74 | + |
| 76 | +++ |
| 77 | ++++ |
| 78 | +++ |
| 79 | +++ |
| 80 | + |
| 82 | ++++ |
| 83 | +++ |
| 84 | ++++ |
| 85 | ++++ |
| 86 | +++ |
| 87 | +++ |
| 88 | + |
| 89 | ++ |
| 92 | + |
| 93 | + |
| 94 | + |
| 95 | + |
| 96 | +++ |
| 97 | +++ |

For Table 1, “++++” refers to an IC₅₀ less than or equal to 100 nM; “+++” refers to an IC₅₀ greater than 100 nM and less than or equal to 500 nM; “++” refers to an IC₅₀ greater than 500 nM and less than or equal to 1000 nM; and “+” refers to an IC₅₀ greater than 1000 nM and less than or equal to 10000 nM.

### Lipid Kinase Selectivity Panel

The compounds of the present invention were tested in a standard lipid kinase selectivity panel:

16 other lipid kinases (Reaction Biology, Malvern, PA, USA), under conditions of non-disclosure, non-dissemination, and non-use. In brief, compounds (see Table 2) were tested at in single dose duplicates at 1 μM against lipid kinases, including PI3Kα, PI3Kβ, and PI3Kδ, and reactions were carried out at 10 μM ATP.

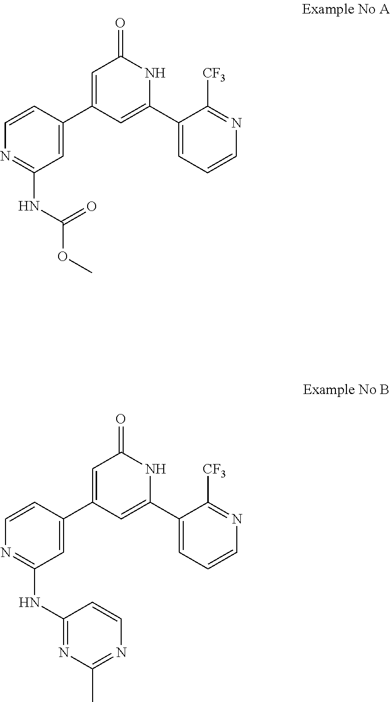 [SMILES: COC(=O)Nc1cc(-c2cc(-c3cccnc3C(F)(F)F)[nH]c(=O)c2)ccn1 ; Cc1nccc(Nc2cc(-c3cc(-c4cccnc4C(F)(F)F)[nH]c(=O)c3)ccn2)n1]

TABLE 2
Percent inhibition relative to DMSO control of PI3K family members.

| | | | | | | PI3K | PI3K | PI3K | |
|---|---|---|---|---|---|---|---|---|---|
| | PI3Kα | PI3Kb | PI3Kd | | PI3K | (p110α | (p110α | (p110α | |
| Ex | (p110α/ | (p110β/ | (p110δ/ | PI3Kg | (p110α/ | (E542K)/ | (E545K)/ | (H1047R)/ | |
| No | p85α) | p85α) | p85α) | (p110γ) | p65α) | p85α) | p85α) | p85α) | PI3KC2α |
| A | 87 | 47 | 96 | 82 | 90 | 88 | 87 | 89 | 1 |
| B | 101 | 99 | 102 | 113 | 101 | 10 | 100 | 101 | 15 |
| 1 | 22 | 13 | 9 | 12 | 5 | 4 | 15 | 10 | −1 |
| 5 | 17 | −6 | 58 | 23 | 15 | 32 | 32 | 35 | 3 |
| 6 | −2 | −14 | −2 | 5 | −26 | 4 | 3 | 19 | −3 |
| 7 | 25 | −7 | 42 | 41 | 33 | 30 | 36 | 44 | 2 |
| 10 | −3 | −12 | 1 | −16 | −23 | 0 | 3 | −3 | 0 |
| 12 | −10 | −6 | 3 | −2 | 8 | −9 | 4 | −2 | 2 |
| 14 | −3 | −9 | −2 | 11 | −7 | −5 | 3 | 0 | −1 |
| 15 | −19 | −4 | −5 | 16 | −12 | −11 | −2 | 10 | −26 |
| 16 | −24 | −5 | −2 | 29 | −24 | −2 | −8 | 14 | −11 |
| 17 | −15 | −10 | 014 | 7 | −17 | −3 | −5 | 6 | −21 |
| 18 | −5 | −3 | 13 | −13 | −2 | −1 | 2 | 16 | −18 |
| 20 | −2 | −9 | −4 | −10 | −11 | −5 | −3 | −11 | 5 |
| 22 | 3 | −3 | 4 | −8 | −4 | −4 | −1 | −4 | 2 |
| 24 | −1 | −9 | −9 | −7 | −6 | −4 | −2 | −17 | 2 |
| 25 | 4 | −7 | −7 | −4 | 0 | 1 | −1 | −7 | −5 |
| 26 | −2 | −10 | −5 | −5 | −4 | −2 | −1 | −8 | 4 |
| 27 | 4 | −1 | 2 | −8 | 7 | 1 | 1 | −11 | 0 |
| 28 | −31 | 0 | 6 | −10 | −3 | −20 | −5 | −3 | 3 |
| 38 | 24 | 18 | 52 | 66 | 19 | 16 | 24 | 23 | 4 |
| 49 | 0 | −8 | −7 | 15 | 3 | 4 | −4 | 8 | 0 |
| 77 | 30 | 11 | 17 | 65 | 24 | 33 | 28 | 22 | 2 |
| 78 | 20 | 14 | 10 | 30 | 15 | 14 | 12 | 8 | −3 |

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

1. A compound represented by Formula (I): 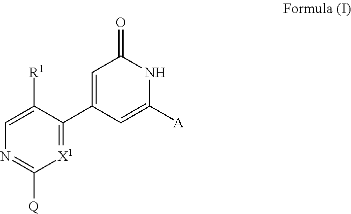
or a pharmaceutically acceptable salt, enantiomer, stereoisomer, or tautomer thereof, wherein:
X¹ is selected from the group consisting of CH and N;
A is selected from the group consisting of cycloalkyl, heterocyclyl, aryl, heteroaryl, and —NHSO₂R³;
Q is selected from the group consisting of H, —NHR², and —NHC(O)LR⁴;
R¹ is selected from the group consisting of alkyl, haloalkyl, halide, cycloalkyl, and amino;
R² is selected from the group consisting of H, alkyl, cycloalkyl, heterocyclyl, aryl, and heteroaryl;
R³ is selected from the group consisting of alkyl, amino, cycloalkyl, heterocyclyl, aryl and heteroaryl;
R⁴ is selected from the group consisting of H, alkyl, cycloalkyl, heterocyclyl, aryl, heteroaryl, alkoxy, amino, O-cycloalkyl, O-heterocyclyl, O-aryl, O-heteroaryl, NH-cycloalkyl, NH-heterocyclyl, NH-aryl, and NH-heteroaryl; and
L is selected from the group consisting of a bond and alkyl,
wherein the cycloalkyl, heterocyclyl, aryl, or heteroaryl, at each occurrence, is optionally substituted with one or more independent occurrences of halide, alkyl, haloalkyl, cycloalkyl, cycloalkylalkyl, alkoxy, haloalkoxy, alkoxyalkyl, haloalkoxyalkyl, hydroxy, hydroxyalkyl, cyano, cyanoalkyl, oxo, amino, alkylamino, amide, acyl, carbamoyl, sulfone, sulfonamide, heterocyclyl, or heteroaryl.

2. (canceled)

3. (canceled)

4. (canceled)

5. (canceled)

6. (canceled)

7. The compound of claim 1, wherein X¹ is CH.

8. The compound of claim 1, wherein X¹ is N.

9. The compound of claim 1, wherein R¹ is selected from the group consisting of alkyl, halide and amino.

10. The compound of n claim 1, wherein R¹ is CH₃.

11. (canceled)

12. (canceled)

13. The compound of claim 1, wherein Q is —NHR².

14. The compound of claim 1, wherein R² is heteroaryl.

15. (canceled)

16. The compound of claim 1, wherein R¹¹, R¹², R¹³, R¹⁴, R²¹, R²², R²³, and R²⁴ are each independently selected from the group consisting of H, C₁-C₆alkyl, and C₁-C₆alkylamino.

17. (canceled)

18. The compound of claim 1, wherein R² is selected from the group consisting of

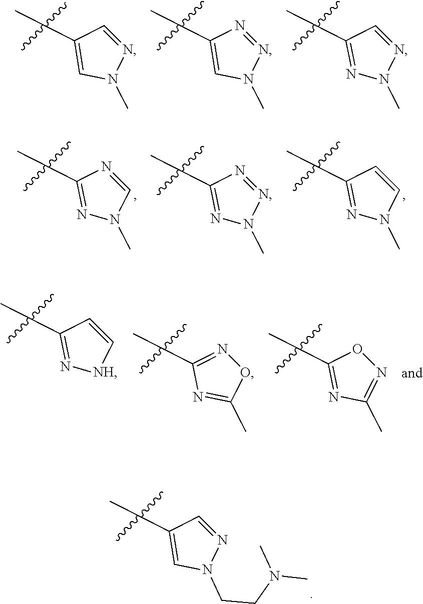

19. (canceled)

20. (canceled)

21. (canceled)

22. The compound of claim 1, wherein R² is selected from the group consisting of

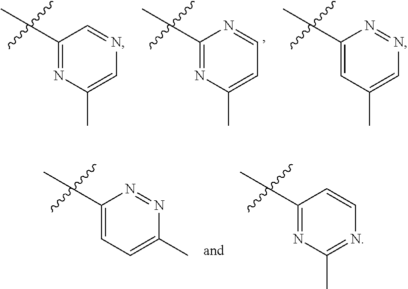

23. (canceled)

24. (canceled)

25. The compound of claim 1, wherein Q is NHC(O)LR⁴.

26. The compound of claim 1, wherein R⁴ is selected from the group consisting of alkyl, alkoxy, amino, alkylamino cycloalkyl, heterocyclyl, aryl, heteroaryl, and O-heterocyclyl.

27. (canceled)

28. The compound of claim 1, wherein R⁴ is selected from the group consisting of alkyl, alkoxy, amino, cycloalkyl, heterocyclyl, heteroaryl, and O-heterocyclyl, wherein each of heterocyclyl, heteroaryl, and O-heterocyclyl is optionally substituted with one or more occurrences of a substituent independently selected from the group consisting of alkyl and halogen.

29. The compound of claim 1, wherein LR⁴ is selected from the group consisting of methyl, methoxy, —N(H)CH₃,

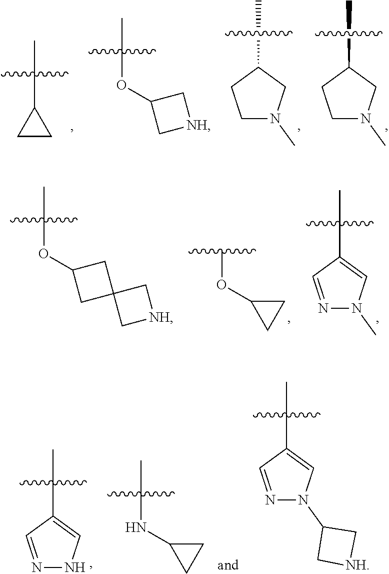

30. (canceled)

31. The compound of claim 1, wherein L is alkyl.

32. The compound of claim 1, wherein R⁴ is selected from the group consisting of alkoxy, amino, cycloalkyl, heterocyclyl, aryl, and heteroaryl, wherein heterocyclyl is optionally substituted with one or more occurrences of a substituent independently selected from the group consisting of alkyl and halogen.

33. (canceled)

34. The compound of claim 1, wherein LR⁴ is selected from the group consisting of

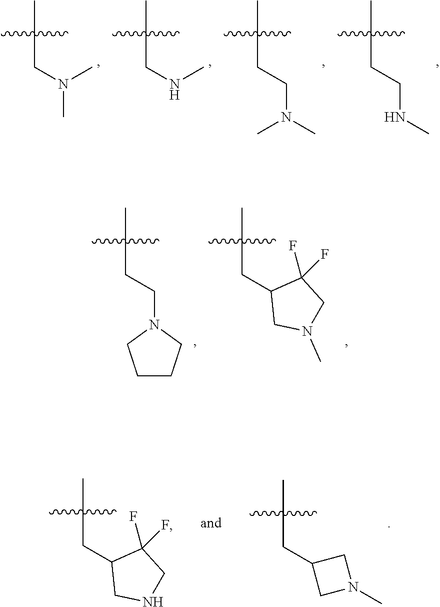

35. (canceled)

36. The compound of a claim 1, wherein A is —NHSO₂R³, and wherein R³ is phenyl optionally substituted with one or more occurrences of R³³, wherein each occurrence of R³³ is independently selected from the group consisting of H, amino, and halogen.

37. The compound of claim 1, wherein A is selected from the group consisting of aryl and heteroaryl.

38. (canceled)

39. (canceled)

40. (canceled)

41. (canceled)

42. (canceled)

43. (canceled)

44. (canceled)

45. The compound of claim 1, wherein A is selected from the group consisting of

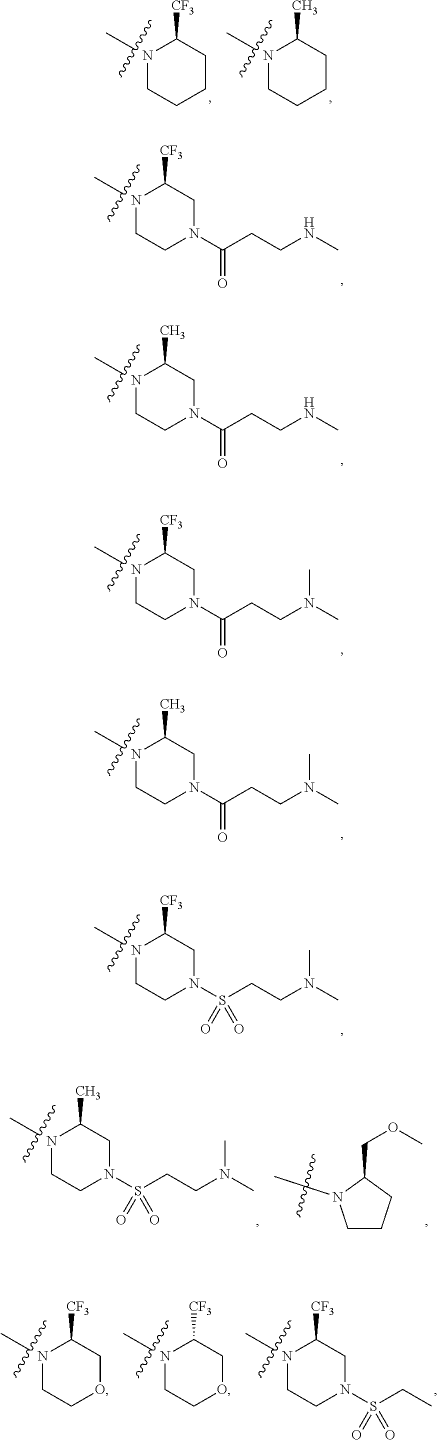

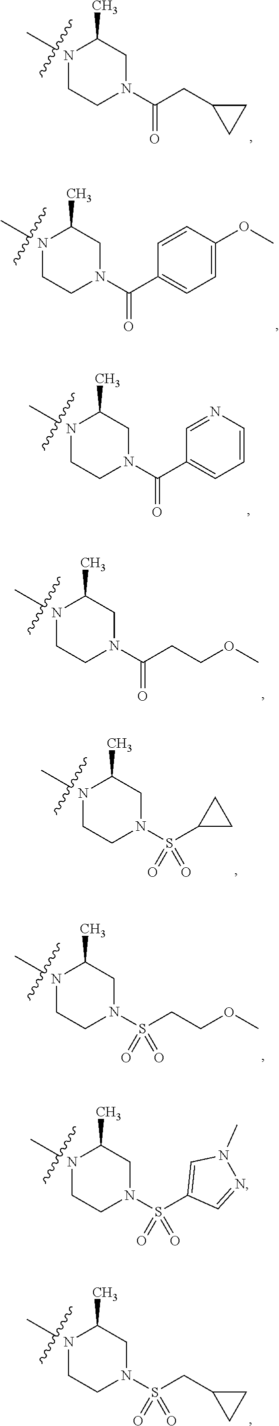

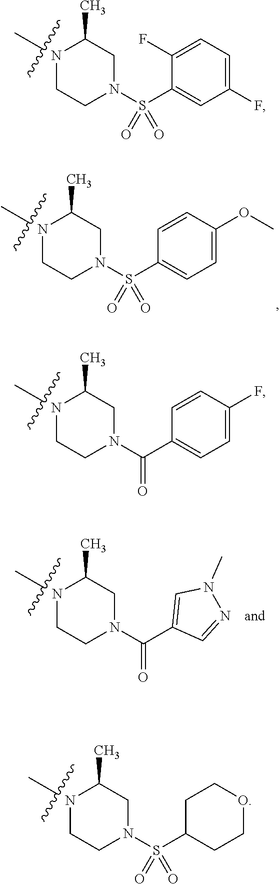

46. (canceled)

47. A compound selected from the group consisting of: 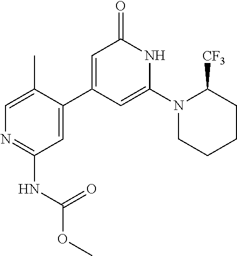 [SMILES: COC(=O)Nc1cc(-c2cc(N3CCCC[C@@H]3C(F)(F)F)[nH]c(=O)c2)c(C)cn1] 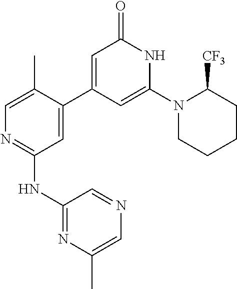 [SMILES: Cc1cncc(Nc2cc(-c3cc(N4CCCC[C@@H]4C(F)(F)F)[nH]c(=O)c3)c(C)cn2)n1] 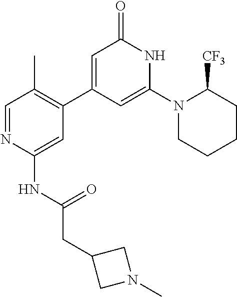 [SMILES: Cc1cnc(NC(=O)CC2CN(C)C2)cc1-c1cc(N2CCCC[C@@H]2C(F)(F)F)[nH]c(=O)c1] 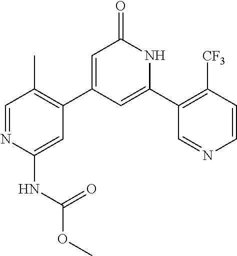 [SMILES: COC(=O)Nc1cc(-c2cc(-c3cnccc3C(F)(F)F)[nH]c(=O)c2)c(C)cn1] 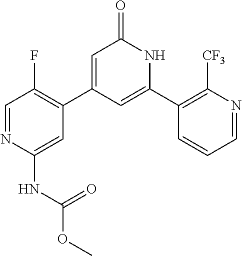 [SMILES: COC(=O)Nc1cc(-c2cc(-c3cccnc3C(F)(F)F)[nH]c(=O)c2)c(F)cn1] 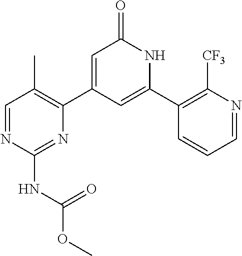 [SMILES: COC(=O)Nc1ncc(C)c(-c2cc(-c3cccnc3C(F)(F)F)[nH]c(=O)c2)n1] 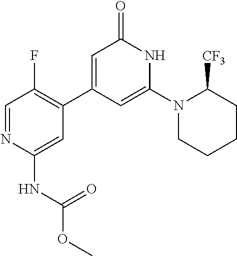 [SMILES: COC(=O)Nc1cc(-c2cc(N3CCCC[C@@H]3C(F)(F)F)[nH]c(=O)c2)c(F)cn1] 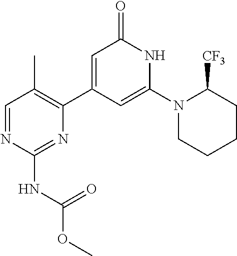 [SMILES: COC(=O)Nc1ncc(C)c(-c2cc(N3CCCC[C@@H]3C(F)(F)F)[nH]c(=O)c2)n1] 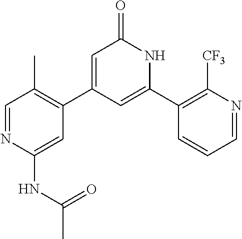 [SMILES: CC(=O)Nc1cc(-c2cc(-c3cccnc3C(F)(F)F)[nH]c(=O)c2)c(C)cn1] 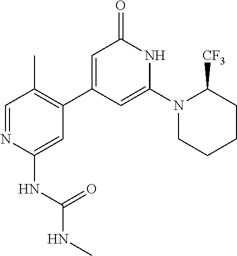 [SMILES: CNC(=O)Nc1cc(-c2cc(N3CCCC[C@@H]3C(F)(F)F)[nH]c(=O)c2)c(C)cn1] 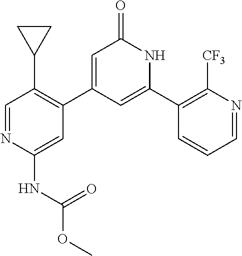 [SMILES: COC(=O)Nc1cc(-c2cc(-c3cccnc3C(F)(F)F)[nH]c(=O)c2)c(C2CC2)cn1] 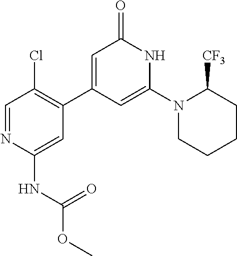 [SMILES: COC(=O)Nc1cc(-c2cc(N3CCCC[C@@H]3C(F)(F)F)[nH]c(=O)c2)c(Cl)cn1] 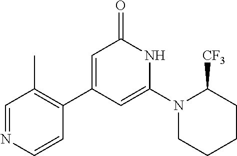 [SMILES: Cc1cnccc1-c1cc(N2CCCC[C@@H]2C(F)(F)F)[nH]c(=O)c1] 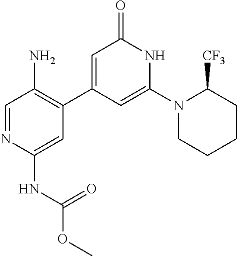 [SMILES: COC(=O)Nc1cc(-c2cc(N3CCCC[C@@H]3C(F)(F)F)[nH]c(=O)c2)c(N)cn1] 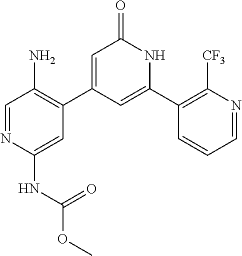 [SMILES: COC(=O)Nc1cc(-c2cc(-c3cccnc3C(F)(F)F)[nH]c(=O)c2)c(N)cn1] 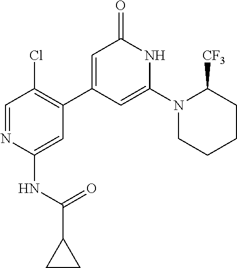 [SMILES: O=C(Nc1cc(-c2cc(N3CCCC[C@@H]3C(F)(F)F)[nH]c(=O)c2)c(Cl)cn1)C1CC1] 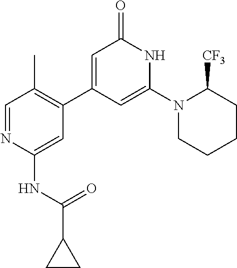 [SMILES: Cc1cnc(NC(=O)C2CC2)cc1-c1cc(N2CCCC[C@@H]2C(F)(F)F)[nH]c(=O)c1] 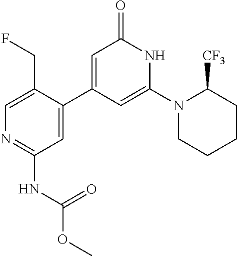 [SMILES: COC(=O)Nc1cc(-c2cc(N3CCCC[C@@H]3C(F)(F)F)[nH]c(=O)c2)c(CF)cn1] 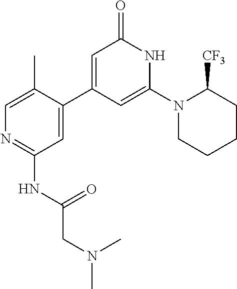 [SMILES: Cc1cnc(NC(=O)CN(C)C)cc1-c1cc(N2CCCC[C@@H]2C(F)(F)F)[nH]c(=O)c1] 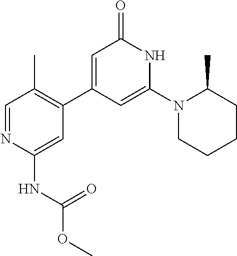 [SMILES: COC(=O)Nc1cc(-c2cc(N3CCCC[C@@H]3C)[nH]c(=O)c2)c(C)cn1] 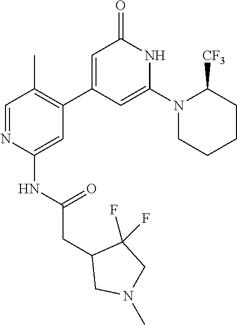 [SMILES: Cc1cnc(NC(=O)CC2CN(C)CC2(F)F)cc1-c1cc(N2CCCC[C@@H]2C(F)(F)F)[nH]c(=O)c1] 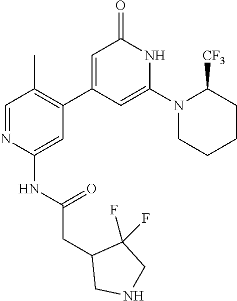 [SMILES: Cc1cnc(NC(=O)CC2CNCC2(F)F)cc1-c1cc(N2CCCC[C@@H]2C(F)(F)F)[nH]c(=O)c1] 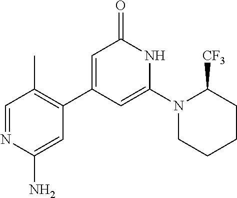 [SMILES: Cc1cnc(N)cc1-c1cc(N2CCCC[C@@H]2C(F)(F)F)[nH]c(=O)c1] 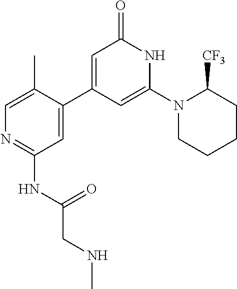 [SMILES: CNCC(=O)Nc1cc(-c2cc(N3CCCC[C@@H]3C(F)(F)F)[nH]c(=O)c2)c(C)cn1] 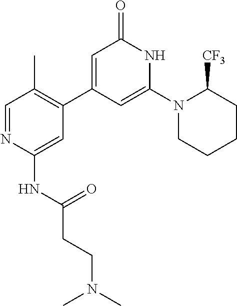 [SMILES: Cc1cnc(NC(=O)CCN(C)C)cc1-c1cc(N2CCCC[C@@H]2C(F)(F)F)[nH]c(=O)c1] 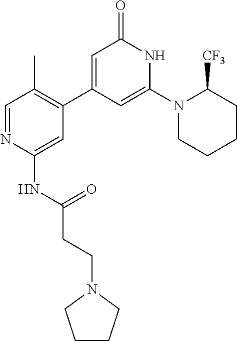 [SMILES: Cc1cnc(NC(=O)CCN2CCCC2)cc1-c1cc(N2CCCC[C@@H]2C(F)(F)F)[nH]c(=O)c1] 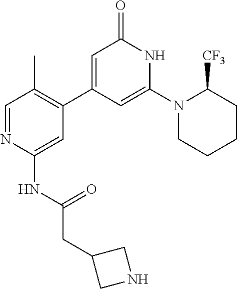 [SMILES: Cc1cnc(NC(=O)CC2CNC2)cc1-c1cc(N2CCCC[C@@H]2C(F)(F)F)[nH]c(=O)c1] 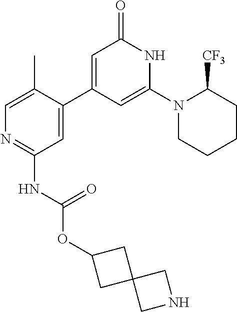 [SMILES: Cc1cnc(NC(=O)OC2CC3(CNC3)C2)cc1-c1cc(N2CCCC[C@@H]2C(F)(F)F)[nH]c(=O)c1] 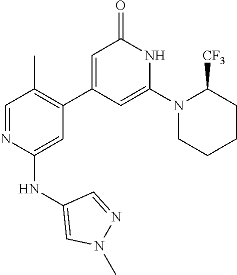 [SMILES: Cc1cnc(Nc2cnn(C)c2)cc1-c1cc(N2CCCC[C@@H]2C(F)(F)F)[nH]c(=O)c1] 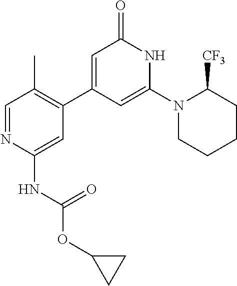 [SMILES: Cc1cnc(NC(=O)OC2CC2)cc1-c1cc(N2CCCC[C@@H]2C(F)(F)F)[nH]c(=O)c1] 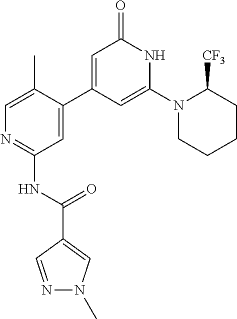 [SMILES: Cc1cnc(NC(=O)c2cnn(C)c2)cc1-c1cc(N2CCCC[C@@H]2C(F)(F)F)[nH]c(=O)c1] 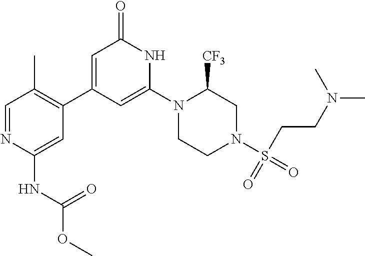 [SMILES: COC(=O)Nc1cc(-c2cc(N3CCN(S(=O)(=O)CCN(C)C)C[C@@H]3C(F)(F)F)[nH]c(=O)c2)c(C)cn1] 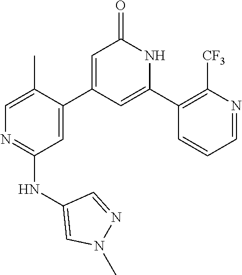 [SMILES: Cc1cnc(Nc2cnn(C)c2)cc1-c1cc(-c2cccnc2C(F)(F)F)[nH]c(=O)c1] 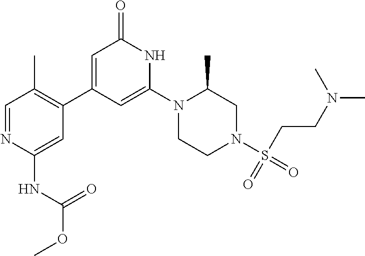 [SMILES: COC(=O)Nc1cc(-c2cc(N3CCN(S(=O)(=O)CCN(C)C)C[C@@H]3C)[nH]c(=O)c2)c(C)cn1] 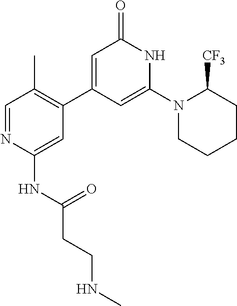 [SMILES: CNCCC(=O)Nc1cc(-c2cc(N3CCCC[C@@H]3C(F)(F)F)[nH]c(=O)c2)c(C)cn1] 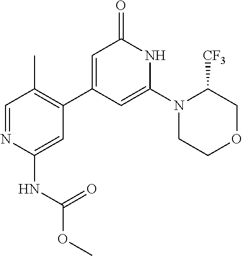 [SMILES: COC(=O)Nc1cc(-c2cc(N3CCOC[C@H]3C(F)(F)F)[nH]c(=O)c2)c(C)cn1] 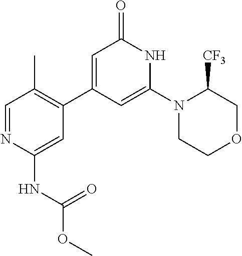 [SMILES: COC(=O)Nc1cc(-c2cc(N3CCOC[C@@H]3C(F)(F)F)[nH]c(=O)c2)c(C)cn1] 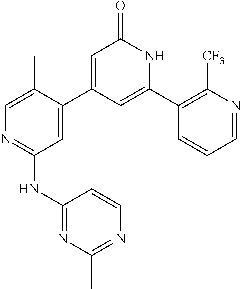 [SMILES: Cc1nccc(Nc2cc(-c3cc(-c4cccnc4C(F)(F)F)[nH]c(=O)c3)c(C)cn2)n1] 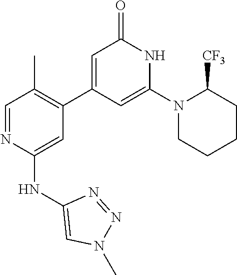 [SMILES: Cc1cnc(Nc2cn(C)nn2)cc1-c1cc(N2CCCC[C@@H]2C(F)(F)F)[nH]c(=O)c1] 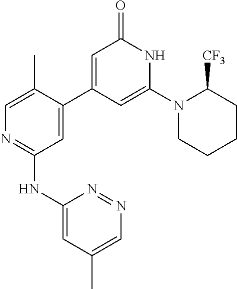 [SMILES: Cc1cnnc(Nc2cc(-c3cc(N4CCCC[C@@H]4C(F)(F)F)[nH]c(=O)c3)c(C)cn2)c1] 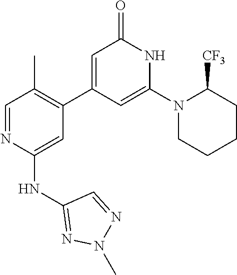 [SMILES: Cc1cnc(Nc2cnn(C)n2)cc1-c1cc(N2CCCC[C@@H]2C(F)(F)F)[nH]c(=O)c1] 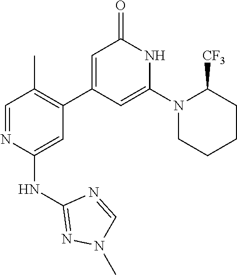 [SMILES: Cc1cnc(Nc2ncn(C)n2)cc1-c1cc(N2CCCC[C@@H]2C(F)(F)F)[nH]c(=O)c1] 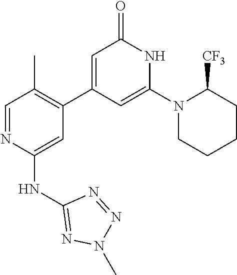 [SMILES: Cc1cnc(Nc2nnn(C)n2)cc1-c1cc(N2CCCC[C@@H]2C(F)(F)F)[nH]c(=O)c1] 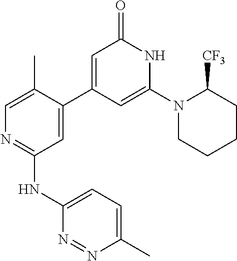 [SMILES: Cc1ccc(Nc2cc(-c3cc(N4CCCC[C@@H]4C(F)(F)F)[nH]c(=O)c3)c(C)cn2)nn1] 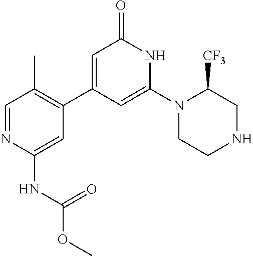 [SMILES: COC(=O)Nc1cc(-c2cc(N3CCNC[C@@H]3C(F)(F)F)[nH]c(=O)c2)c(C)cn1] 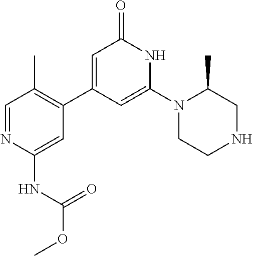 [SMILES: COC(=O)Nc1cc(-c2cc(N3CCNC[C@@H]3C)[nH]c(=O)c2)c(C)cn1] 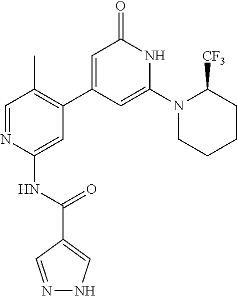 [SMILES: Cc1cnc(NC(=O)c2cn[nH]c2)cc1-c1cc(N2CCCC[C@@H]2C(F)(F)F)[nH]c(=O)c1] 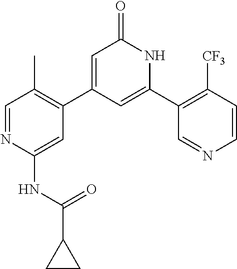 [SMILES: Cc1cnc(NC(=O)C2CC2)cc1-c1cc(-c2cnccc2C(F)(F)F)[nH]c(=O)c1] 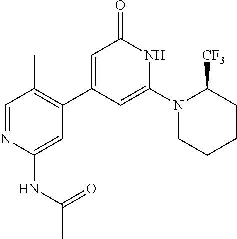 [SMILES: CC(=O)Nc1cc(-c2cc(N3CCCC[C@@H]3C(F)(F)F)[nH]c(=O)c2)c(C)cn1] 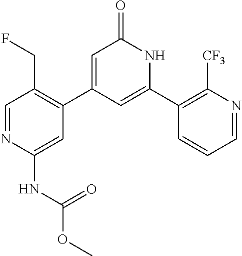 [SMILES: COC(=O)Nc1cc(-c2cc(-c3cccnc3C(F)(F)F)[nH]c(=O)c2)c(CF)cn1] 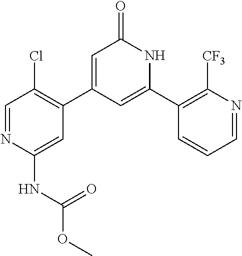 [SMILES: COC(=O)Nc1cc(-c2cc(-c3cccnc3C(F)(F)F)[nH]c(=O)c2)c(Cl)cn1] 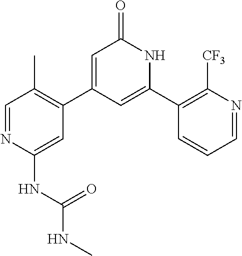 [SMILES: CNC(=O)Nc1cc(-c2cc(-c3cccnc3C(F)(F)F)[nH]c(=O)c2)c(C)cn1] 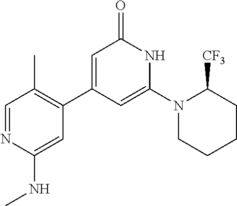 [SMILES: CNc1cc(-c2cc(N3CCCC[C@@H]3C(F)(F)F)[nH]c(=O)c2)c(C)cn1] 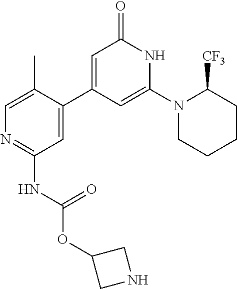 [SMILES: Cc1cnc(NC(=O)OC2CNC2)cc1-c1cc(N2CCCC[C@@H]2C(F)(F)F)[nH]c(=O)c1] 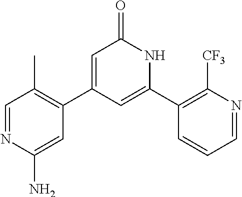 [SMILES: Cc1cnc(N)cc1-c1cc(-c2cccnc2C(F)(F)F)[nH]c(=O)c1] 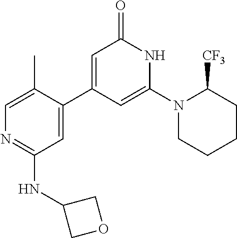 [SMILES: Cc1cnc(NC2COC2)cc1-c1cc(N2CCCC[C@@H]2C(F)(F)F)[nH]c(=O)c1] 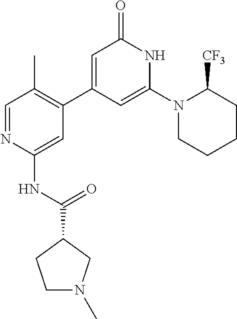 [SMILES: Cc1cnc(NC(=O)[C@H]2CCN(C)C2)cc1-c1cc(N2CCCC[C@@H]2C(F)(F)F)[nH]c(=O)c1] 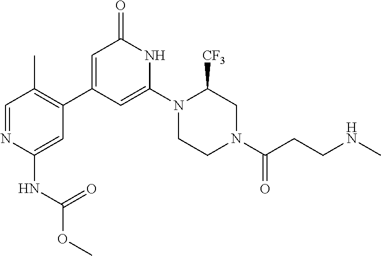 [SMILES: CNCCC(=O)N1CCN(c2cc(-c3cc(NC(=O)OC)ncc3C)cc(=O)[nH]2)[C@@H](C(F)(F)F)C1] 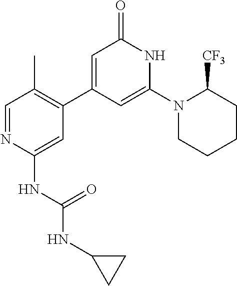 [SMILES: Cc1cnc(NC(=O)NC2CC2)cc1-c1cc(N2CCCC[C@@H]2C(F)(F)F)[nH]c(=O)c1] 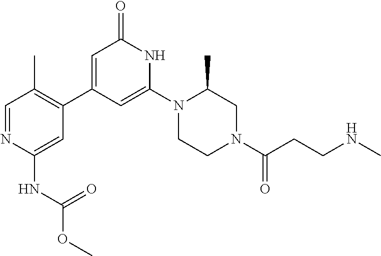 [SMILES: CNCCC(=O)N1CCN(c2cc(-c3cc(NC(=O)OC)ncc3C)cc(=O)[nH]2)[C@@H](C)C1] 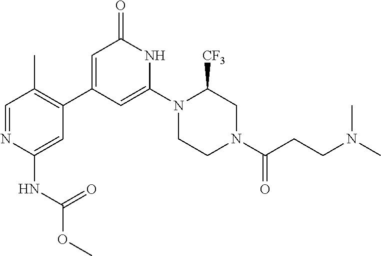 [SMILES: COC(=O)Nc1cc(-c2cc(N3CCN(C(=O)CCN(C)C)C[C@@H]3C(F)(F)F)[nH]c(=O)c2)c(C)cn1] 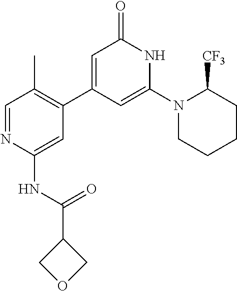 [SMILES: Cc1cnc(NC(=O)C2COC2)cc1-c1cc(N2CCCC[C@@H]2C(F)(F)F)[nH]c(=O)c1] 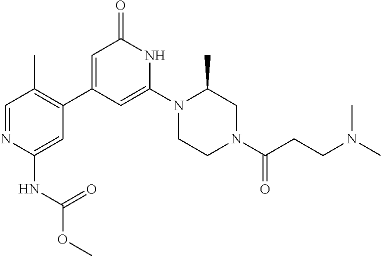 [SMILES: COC(=O)Nc1cc(-c2cc(N3CCN(C(=O)CCN(C)C)C[C@@H]3C)[nH]c(=O)c2)c(C)cn1] 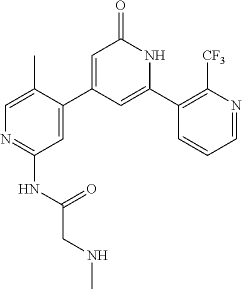 [SMILES: CNCC(=O)Nc1cc(-c2cc(-c3cccnc3C(F)(F)F)[nH]c(=O)c2)c(C)cn1] 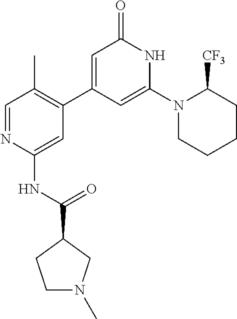 [SMILES: Cc1cnc(NC(=O)[C@@H]2CCN(C)C2)cc1-c1cc(N2CCCC[C@@H]2C(F)(F)F)[nH]c(=O)c1] 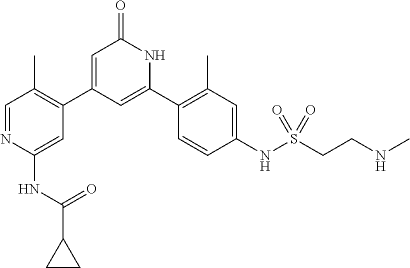 [SMILES: CNCCS(=O)(=O)Nc1ccc(-c2cc(-c3cc(NC(=O)C4CC4)ncc3C)cc(=O)[nH]2)c(C)c1] 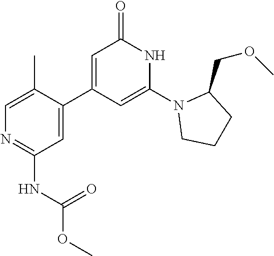 [SMILES: COC[C@H]1CCCN1c1cc(-c2cc(NC(=O)OC)ncc2C)cc(=O)[nH]1] 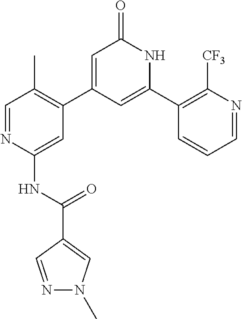 [SMILES: Cc1cnc(NC(=O)c2cnn(C)c2)cc1-c1cc(-c2cccnc2C(F)(F)F)[nH]c(=O)c1] 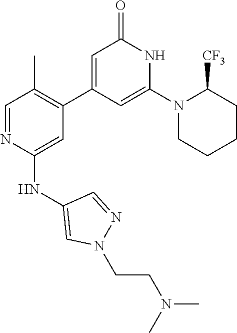 [SMILES: Cc1cnc(Nc2cnn(CCN(C)C)c2)cc1-c1cc(N2CCCC[C@@H]2C(F)(F)F)[nH]c(=O)c1] 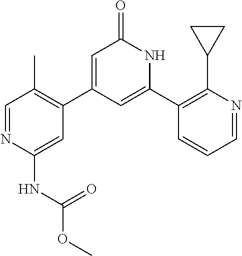 [SMILES: COC(=O)Nc1cc(-c2cc(-c3cccnc3C3CC3)[nH]c(=O)c2)c(C)cn1] 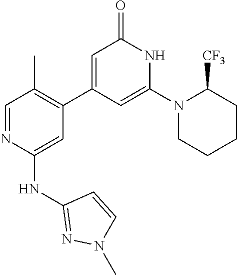 [SMILES: Cc1cnc(Nc2ccn(C)n2)cc1-c1cc(N2CCCC[C@@H]2C(F)(F)F)[nH]c(=O)c1] 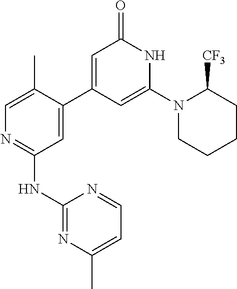 [SMILES: Cc1ccnc(Nc2cc(-c3cc(N4CCCC[C@@H]4C(F)(F)F)[nH]c(=O)c3)c(C)cn2)n1] 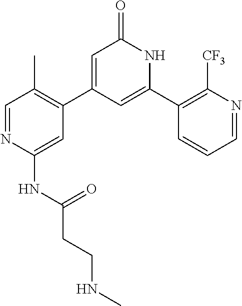 [SMILES: CNCCC(=O)Nc1cc(-c2cc(-c3cccnc3C(F)(F)F)[nH]c(=O)c2)c(C)cn1] 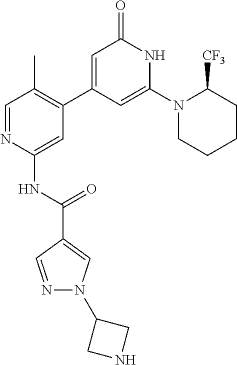 [SMILES: Cc1cnc(NC(=O)c2cnn(C3CNC3)c2)cc1-c1cc(N2CCCC[C@@H]2C(F)(F)F)[nH]c(=O)c1] 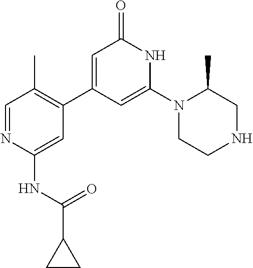 [SMILES: Cc1cnc(NC(=O)C2CC2)cc1-c1cc(N2CCNC[C@@H]2C)[nH]c(=O)c1] 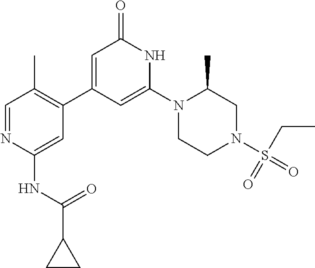 [SMILES: CCS(=O)(=O)N1CCN(c2cc(-c3cc(NC(=O)C4CC4)ncc3C)cc(=O)[nH]2)[C@@H](C)C1] 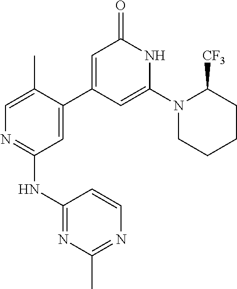 [SMILES: Cc1nccc(Nc2cc(-c3cc(N4CCCC[C@@H]4C(F)(F)F)[nH]c(=O)c3)c(C)cn2)n1] 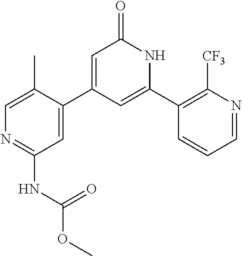 [SMILES: COC(=O)Nc1cc(-c2cc(-c3cccnc3C(F)(F)F)[nH]c(=O)c2)c(C)cn1] 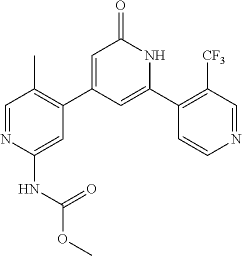 [SMILES: COC(=O)Nc1cc(-c2cc(-c3ccncc3C(F)(F)F)[nH]c(=O)c2)c(C)cn1] 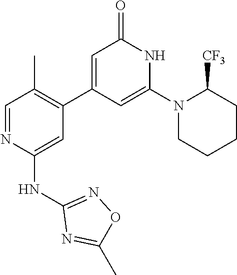 [SMILES: Cc1nc(Nc2cc(-c3cc(N4CCCC[C@@H]4C(F)(F)F)[nH]c(=O)c3)c(C)cn2)no1] 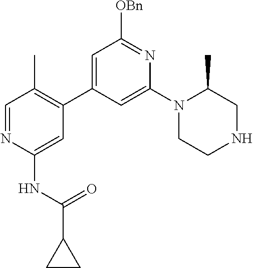 [SMILES: Cc1cnc(NC(=O)C2CC2)cc1-c1cc(OCc2ccccc2)nc(N2CCNC[C@@H]2C)c1] 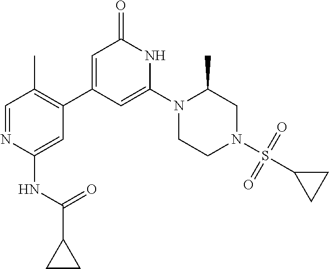 [SMILES: Cc1cnc(NC(=O)C2CC2)cc1-c1cc(N2CCN(S(=O)(=O)C3CC3)C[C@@H]2C)[nH]c(=O)c1] 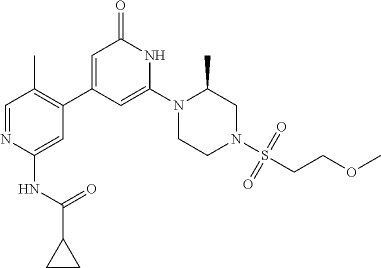 [SMILES: COCCS(=O)(=O)N1CCN(c2cc(-c3cc(NC(=O)C4CC4)ncc3C)cc(=O)[nH]2)[C@@H](C)C1] 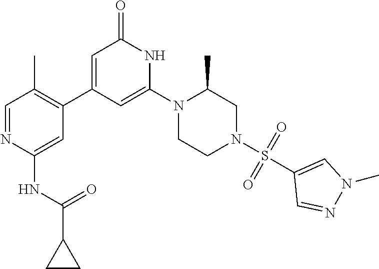 [SMILES: Cc1cnc(NC(=O)C2CC2)cc1-c1cc(N2CCN(S(=O)(=O)c3cnn(C)c3)C[C@@H]2C)[nH]c(=O)c1] 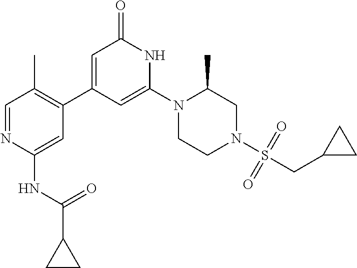 [SMILES: Cc1cnc(NC(=O)C2CC2)cc1-c1cc(N2CCN(S(=O)(=O)CC3CC3)C[C@@H]2C)[nH]c(=O)c1] 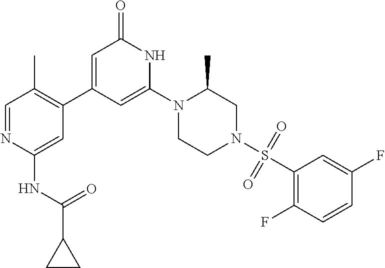 [SMILES: Cc1cnc(NC(=O)C2CC2)cc1-c1cc(N2CCN(S(=O)(=O)c3cc(F)ccc3F)C[C@@H]2C)[nH]c(=O)c1] 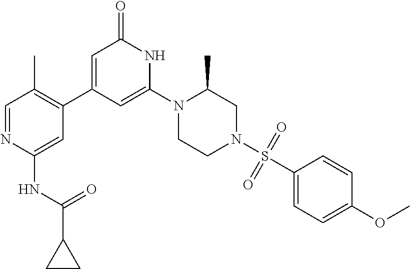 [SMILES: COc1ccc(S(=O)(=O)N2CCN(c3cc(-c4cc(NC(=O)C5CC5)ncc4C)cc(=O)[nH]3)[C@@H](C)C2)cc1] 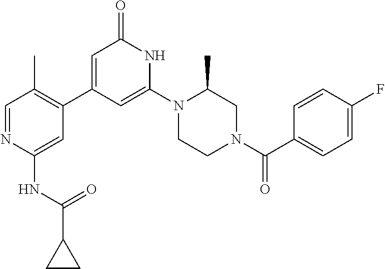 [SMILES: Cc1cnc(NC(=O)C2CC2)cc1-c1cc(N2CCN(C(=O)c3ccc(F)cc3)C[C@@H]2C)[nH]c(=O)c1] 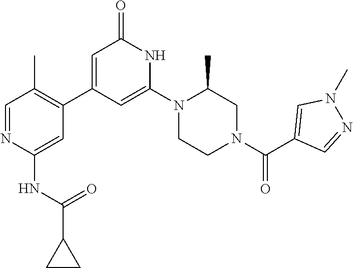 [SMILES: Cc1cnc(NC(=O)C2CC2)cc1-c1cc(N2CCN(C(=O)c3cnn(C)c3)C[C@@H]2C)[nH]c(=O)c1] 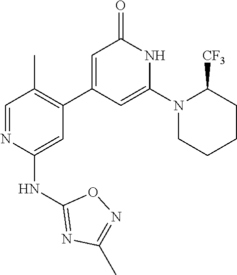 [SMILES: Cc1noc(Nc2cc(-c3cc(N4CCCC[C@@H]4C(F)(F)F)[nH]c(=O)c3)c(C)cn2)n1] 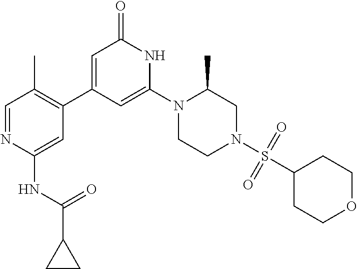 [SMILES: Cc1cnc(NC(=O)C2CC2)cc1-c1cc(N2CCN(S(=O)(=O)C3CCOCC3)C[C@@H]2C)[nH]c(=O)c1] 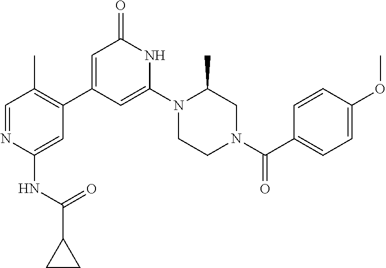 [SMILES: COc1ccc(C(=O)N2CCN(c3cc(-c4cc(NC(=O)C5CC5)ncc4C)cc(=O)[nH]3)[C@@H](C)C2)cc1] 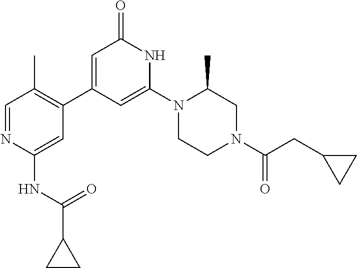 [SMILES: Cc1cnc(NC(=O)C2CC2)cc1-c1cc(N2CCN(C(=O)CC3CC3)C[C@@H]2C)[nH]c(=O)c1] 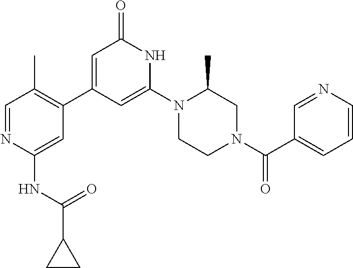 [SMILES: Cc1cnc(NC(=O)C2CC2)cc1-c1cc(N2CCN(C(=O)c3cccnc3)C[C@@H]2C)[nH]c(=O)c1] 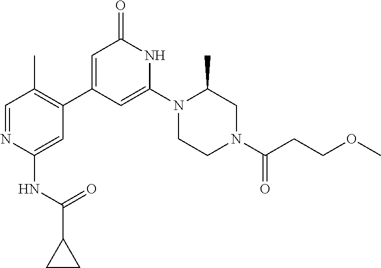 [SMILES: COCCC(=O)N1CCN(c2cc(-c3cc(NC(=O)C4CC4)ncc3C)cc(=O)[nH]2)[C@@H](C)C1] 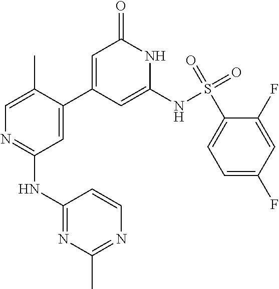 [SMILES: Cc1nccc(Nc2cc(-c3cc(NS(=O)(=O)c4ccc(F)cc4F)[nH]c(=O)c3)c(C)cn2)n1] 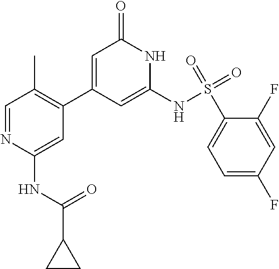 [SMILES: Cc1cnc(NC(=O)C2CC2)cc1-c1cc(NS(=O)(=O)c2ccc(F)cc2F)[nH]c(=O)c1]
and pharmaceutically acceptable salts, enantiomers, stereoisomers, and tautomers thereof.

48. A pharmaceutical composition comprising the compound of claim 1, or a pharmaceutically acceptable salt, enantiomer, stereoisomer, or tautomer thereof, and a pharmaceutically acceptable carrier or excipient.

49. A method of treating a cancer in a patient in need thereof, comprising administering to the patient a therapeutically effective amount of the compound of claim 1, or a pharmaceutically acceptable salt, enantiomer, stereoisomer, or tautomer thereof.

50. The method of claim 49, wherein the cancer is selected from the group consisting of breast cancer, such as triple negative breast cancer, bladder cancer, liver cancer, cervical cancer, pancreatic cancer, leukemia, lymphoma, renal cancer, colon cancer, glioma, prostate cancer, ovarian cancer, melanoma and lung cancer, gastrointestinal stromal tumors, esophageal cancer, gastric cancer, glioma, glioblastoma, ovarian cancer, head cancer, neck cancer, urothelial cancer, uterine cancer, prostate cancer, hepatic cancer, an osteosarcoma, a sarcoma, multiple myeloma, a cancer that is metastatic to bone, and a papillary thyroid carcinoma, as well as hypoxic tumors.

51-59. (canceled)


SEQUENCE LISTING (1 sequence)

SEQ ID NO: 1 - protein, 1123 aa, synthetic construct
MAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRHNQTSLYKKAGTMGEAEKFHYIYSCDLDINVQLKIGSLEGKREQKSYKAVLEDPMLKFSGLYQETCSDLYVTCQVFAEGKPLALPVRTSYKAFSTRWNWNEWLKLPVKYPDLPRNAQVALTIWDVYGPGKAVPVGGTTVSLFGKYGMFRQGMHDLKVWPNVEADGSEPTKTPGRTSSTLSEDQMSRLAKLTKAHRQGHMVKVDWLDRLTFREIEMINESEKRSSNFMYLMVEFRCVKCDDKEYGIVYYEKDGDESSPILTSFELVKVPDPQMSMENLVESKHHKLARSLRSGPSDHDLKPNAATRDQLNIIVSYPPTKQLTYEEQDLVWKFRYYLTNQEKALTKFLKCVNWDLPQEAKQALELLGKWKPMDVEDSLELLSSHYTNPTVRRYAVARLRQADDEDLLMYLLQLVQALKYENFDDIKNGLEPTKKDSQSSVSENVSNSGINSAEIDSSQIITSPLPSVSSPPPASKTKEVPDGENLEQDLCTFLISRACKNSTLANYLYWYVIVECEDQDTQQRDPKTHEMYLNVMRRFSQALLKGDKSVRVMRSLLAAQQTFVDRLVHLMKAVQRESGNRKKKNERLQALLGDNEKMNLSDVELIPLPLEPQVKIRGIIPETATLFKSALMPAQLFFKTEDGGKYPVIFKHGDDLRQDQLILQIISLMDKLLRKENLDLKLTPYKVLATSTKHGFMQFIQSVPVAEVLDTEGSIQNFFRKYAPSENGPNGISAEVMDTYVKSCAGYCVITYILGVGDRHLDNLLLTKTGKLFHIDFGYILGRDPKPLPPPMKLNKEMVEGMGGTQSEQYQEFRKQCYTAFLHLRRYSNLILNLFSLMVDANIPDIALEPDKTVKKVQDKFRLDLSDEEAVHYMQSLIDESVHALFAAVVEQIHKFAQYWRK